SEQUENCE LISTING (3 sequences)

SEQ ID NO: 1 - protein, 4 aa, synthetic construct
  REGION 1..4: note = Synthetic Construct
  SITE 2: note = D-Lysine
GKGG

SEQ ID NO: 2 - protein, 4 aa, synthetic construct
  REGION 1..4: note = Synthetic Construct
  MOD_RES 1: note = N-terminal amidation
GKGG

SEQ ID NO: 3 - protein, 5 aa, synthetic construct
  REGION 1..5: note = Synthetic Construct
  MOD_RES 1: note = N-terminal amidation
  SITE 3: note = D-Lysine
GGKGG

(19) United States
(12) Patent Application Publication
(10) Pub. No.: US 2025/0262312 A1
(43) Pub. Date: Aug. 21, 2025

(54) SELECTIVE DRUG RELEASE FROM INTERNALIZED CONJUGATES OF BIOLOGICALLY ACTIVE COMPOUNDS

(71) Applicant: Seagen Inc., Bothell, WA (US)

(72) Inventors: Noah BINDMAN, Seattle, WA (US); Nicole OKELEY, Redmond, WA (US); Peter SENTER, Seattle, WA (US); Divya AWASTHI, Seattle, WA (US)

(21) Appl. No.: 18/940,208

(22) Filed: Nov. 7, 2024

Related U.S. Application Data
(63) Continuation of application No. 17/026,048, filed on Sep. 18, 2020, now abandoned.
(60) Provisional application No. 62/902,888, filed on Sep. 19, 2019.

(51) Int. Cl. A61K 47/65; A61K 9/08; A61K 9/19; A61K 47/54; A61K 47/68; C07K 5/083

(52) U.S. Cl. CPC A61K 47/65; A61K 9/08; A61K 9/19; A61K 47/545; A61K 47/6817; A61K 47/6851; C07K 5/0808

(57) ABSTRACT

The invention relates to conjugates of biologically active compounds, wherein such a conjugate is comprised of a sequence of amino acids containing a tripeptide that confers selective cleavage by tumor tissue homogenate for release of free drug and/or improves biodistribution into the tumor tissue in comparison to normal tissue homogenate from the same species, wherein the normal tissue is the site of an adverse event associated with administration to a human subject in need thereof of a therapeutically effective amount of a comparator conjugate whose amino acid sequence is a dipeptide known to be selectively cleavable by Cathepsin B.

### CROSS-REFERENCE TO RELATED APPLICATION

This application claims priority to U.S. Provisional Application No. 62/902,888 filed on Sep. 19, 2019, the contents of which are incorporated herein by reference in their entirety.

### BACKGROUND OF THE INVENTION

The invention relates to Ligand Drug Conjugate (LDC) compounds and compositions thereof, including Antibody Drug Conjugates (ADCs), that have improved selectivity for targeted cells in comparison to non-targeted cells.

Traditional Ligand Drug Conjugates exhibit biological activity towards targeted cells, which display the targeted moiety that is recognized by the Ligand Unit of the Conjugate, by binding to the targeted moiety and then entering into the cell by internalization of the bound Conjugate. Selectivity for the targeted cells over non-targeted cells is primarily achieved by a traditional Ligand Drug Conjugate as a result of the targeted moiety being present in greater abundance on the targeted cells in comparison to non-targeted normal cells, which are cells not intended to be acted upon by the Conjugate. When conditional release of a conjugated compound, which is cytotoxic in free form, is to be affected by an intracellular protease, internalization of bound Conjugate is followed by enzymatic processing of a peptide-based Linker Unit of the Conjugate.

Reduction in premature release of the cytotoxic compound, which otherwise would cause undesired side effects, from traditional dipeptide-based Ligand Drug Conjugates is accomplished by optimizing for selectivity for a specific lysosomal protease that is believed to be upregulated in cancer cells. As the protease responsible for intracellular processing of the traditional Ligand Drug Conjugates is common to all cells, selectivity for the targeted cells is primarily due to the greater abundance of the targeted moiety on the cells intended to be acted upon by the Conjugate, notwithstanding the differing intracellular activity levels of the processing protease within targeted cancer cells and non-targeted normal cells. However, that approach does not take into consideration possible exposure differences of the released cytotoxic compound between tumor and normal tissue, which are presently exploited by the Ligand Drug Conjugates of the present invention.

Thus, the dipeptide sequences of traditional Ligand Drug Conjugates, which were designed to be selectively acted upon by an intracellular protease upregulated in cancer cells of the tumor tissue, are still capable of being acted upon by proteases confined within normal tissue. Such action can occur either within the microenvironment of the normal tissue or within cells of the normal tissue after immunologically specific or non-specific uptake into these cells, resulting in on-target or off-target toxicity, respectively. Those toxicities are a more acute problem to be solved for targeted delivery of highly cytotoxic compounds. It is therefore believed a Ligand Drug Conjugate with an improved peptide sequence that provides lower exposure to normal tissue in comparison to a traditional dipeptide-based Ligand Drug Conjugates, and hence reduces exposure to a cytotoxic compound released therefrom, while maintaining the efficacy provided by these traditional conjugates, would improve tolerability to therapy.

It is further believed that a Ligand Drug Conjugate having an improved peptide sequence that is more prone to proteolysis by tumor tissue over proteolysis by normal tissue in comparison to proteolysis of a traditional dipeptide-based Ligand Drug Conjugate by these tissues would also decrease exposure to the released cytotoxic compound, which would contribute to improving tolerability to therapy. Determining those proteolytic differences using tissue homogenates should capture those differences driven by the microenvironment of these tissues and/or subsequent to cellular internalization.

To provide the solution to that problem in the art, disclosed herein are Ligand Drug Conjugates having peptide-based Linker Units whose sequences result in more selective exposure of targeted cells of the tumor tissue to the cytotoxic compound released from the Conjugate in comparison to exposure of cells of normal tissue to the free cytotoxin such that tolerability to the Conjugate is improved while retaining the efficacy of the traditional dipeptide-based Conjugates in treating cancer in a mammalian subject. That difference in exposure may result from greater selectivity for proteolysis of Ligand Drug Conjugates having the selectivity conferring peptide sequences within tumor tissue over proteolysis within normal tissue in comparison to proteolysis of the traditional dipeptide-based Conjugate. Because altering the peptide sequence may also affect the physiochemical properties of the Conjugate compound, greater exposure from improved biodistribution into tumor tissue and not normal tissue and/or improved disposition once distributed into these tissues, which preferentially retains the Conjugate compound in tumor tissue and/or preferentially eliminates the Conjugate compound from normal tissue, respectively, can occur. Those biodistribution effects may even become the dominant factors over preferential proteolysis, which could be difficult to observe in vivo.

Thus, Conjugate compounds having peptide sequences providing enhanced exposure of released free cytotoxic compound to tumor tissue in comparison to normal tissue should exhibit reduced undesired toxicities due to the peptide sequences being overall less susceptible to proteolysis within normal tissue or cells thereof in comparison to those of the tumor and/or from improved pharmacokinetic properties for Conjugate compounds incorporating those peptide sequences that favor tumor tissue over normal tissue.

The Ligand Drug Conjugates of the present invention therefore have two levels of selectivity for targeted cells over non-targeted normal cells: (1) selective entry into targeted cells and (2) decreased exposure of normal tissue in comparison to tumor tissue to the Conjugate compound. From that second level of selectivity, reduction in normal tissue toxicities is expected to provide reduced adverse events associated with conventional targeted therapies.

### SUMMARY OF THE INVENTION

One principle embodiment of the invention provides a Ligand Drug Conjugate composition represented by Formula 1:

L-[LU-D′]ₚ (1)
or a salt thereof, in particular a pharmaceutically acceptable salt, wherein
L is a Ligand Unit;
LU is a Linker Unit; and
D′ represents from 1 to Drug Units (D) in each drug linker moiety of formula -LU-D′; and
subscript p is a number from 1 to 12, from 1 to 10 or from 1 to 8 or is about 4 or about 8,
wherein the Ligand Unit is of an antibody, or an antigen-binding fragment of an antibody, that is capable of selective binding to an antigen of tumor tissue for subsequent release of the Drug Unit as free cytotoxic compound,
wherein the drug linker moiety of formula -LU-D′ in each of the Ligand Drug Conjugate compounds of the composition has the structure of Formula 1A:

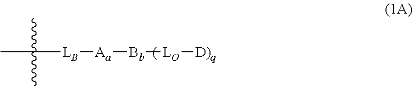

or a salt thereof, in particular a pharmaceutically acceptable salt,
wherein the wavy line indicates covalent attachment to L;
D is the Drug Unit of the cytotoxic compound;
L_{B} is a ligand covalent binding moiety;
A is a first optional Stretcher Unit;
subscript a is 0 or 1 indicating the absence of presence of A, respectively;
B is an optional Branching Unit;
subscript b is 0 or 1, indicating the absence of presence of B, respectively;
L_{O} is a secondary linker moiety, wherein the secondary linker has the formula of;

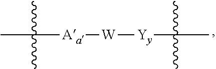

wherein the wavy line adjacent to Y indicates the site of covalent attachment of L_{O} to the Drug Unit and the wavy line adjacent to A′ indicates the site of covalent attachment to the remainder of the drug linker moiety;
A′ is a second optional Stretcher Unit, which in the absence of B becomes a subunit of A,
subscript a′ is 0 or 1, indicating the absence or presence of A′, respectively,
W is a Peptide Cleavable Unit, wherein the Peptide Cleavable Unit is a contiguous sequence of up to 12 (e.g., 3-12 or 3-10) amino acids, wherein the sequence is comprised of a selectivity conferring tripeptide that provides improved selectivity for exposure of tumor tissue over normal tissue to free cytotoxic compound released from the Ligand Drug Conjugate compounds of the composition in comparison to the cytotoxic compound released from Ligand Drug Conjugate compounds of a comparator Ligand-Drug Conjugate composition in which the peptide sequence of its Peptide Cleavable Unit is the dipeptide-valine-citrulline- or -valine-alanine-;
wherein the tumor and normal tissues are of rodent species and wherein the Formula 1 composition provides said improved exposure selectivity demonstrated by:
retaining efficacy in a tumor xenograft model of the comparator Ligand-Drug Conjugate conjugate composition when administered at the same effective amount and dose schedule previously determined for the comparator Ligand-Drug Conjugate conjugate composition, and
showing a reduction in plasma concentration of the free cytotoxic compound released from the Ligand Drug Conjugate compounds of the composition, and/or preservation of normal cells in tissue when administered at the same effective amount and dose schedule as in the tumor xenograft model to a non-tumor bearing rodent in comparison to the equivalent (e.g., same) administration of the comparator Ligand-Drug Conjugate composition in which the Ligand Units of both conjugate compositions are replaced by a non-binding antibody,
wherein cytotoxicity to cells in human tissue of the same type as the normal cells in the tissue of the non-tumor bearing rodent is responsible at least in part to an adverse event in a human subject to whom is administered a therapeutically effective amount of the comparator conjugate composition;
Y is a self-immolative Spacer Unit; and
subscript y is 0, 1 or 2 indicating the absence or presence of 1 or 2 of Y, respectively;
subscript q is an integer ranging from 1 to 4,
provided that subscript q is 1 when subscript b is 0 and subscript q is 2, 3 or 4 when subscript b is 1; and
wherein the Ligand Drug Conjugate compounds of the composition have the structure of Formula 1 in which subscript p is replaced by subscript p′, wherein subscript p′ is an integer from 1 to 12, 1 to 10 or 1 to 8 or is 4 or 8.

A related principle embodiment provides for a Drug Linker compound of Formula I:

LU′-(D′) (I)
or a salt thereof, in particular a pharmaceutically acceptable salt thereof, wherein LU′ is capable of providing a covalent bond between L and LU of Formula 1, and therefore is sometimes referred to as a Linker Unit precursor; and D′ represents from 1 to 4 Drug Units, wherein the Drug Linker compound is further defined by the structure of Formula IA:

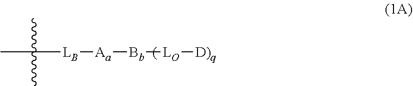

wherein L_{B}′ is capable of transformation to L_{B} of Formula 1A thereby forming a covalent bond to L of Formula 1, and therefore is sometimes referred to a ligand covalent binding precursor moiety, and the remaining variable groups of Formula IA are as defined for Formula 1A.

In some embodiments, provided herein is a Ligand Drug Conjugate composition represented by Formula 1:

L-[LU-D′]ₚ (1)
or a pharmaceutically acceptable salt thereof, wherein
L is a Ligand Unit;
LU is a Linker Unit;
D′ represents from 1 to 4 Drug Units (D) in each drug linker moiety of formula -LU-D′; and
subscript p is a number from 1 to 12, from 1 to 10 or from 1 to 8 or is about 4 or about 8,
wherein the Ligand Unit is from an antibody or an antigen-binding fragment of an antibody that is capable of selective binding to an antigen of tumor tissue for subsequent release of the Drug Unit(s) as free drug,
wherein the drug linker moiety of formula -LU-D′ in each of the Ligand Drug Conjugate compounds of the composition has the structure of Formula 1A:

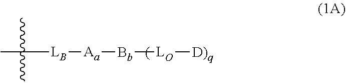

or a salt thereof, in particular a pharmaceutically acceptable salt,
wherein the wavy line indicates covalent attachment to L;
D is the Drug Unit;
L_{B} is a ligand covalent binding moiety;
A is a first optional Stretcher Unit;
subscript a is 0 or 1, indicating the absence or presence of A, respectively;
B is an optional Branching Unit;
subscript b is 0 or 1, indicating the absence or presence of B, respectively;
L_{O} is a secondary linker moiety, wherein the secondary linker has the formula of;

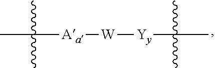

wherein the wavy line adjacent to Y indicates the site of covalent attachment of L_{O} to the Drug Unit and the wavy line adjacent to A′ indicates the site of covalent attachment to the remainder of the drug linker moiety;
A′ is a second optional Stretcher Unit, which in the absence of B becomes a subunit of A,
subscript a′ is 0 or 1, indicating the absence or presence of A′, respectively,
W is a Peptide Cleavable Unit, wherein the Peptide Cleavable Unit comprises a tripeptide having the sequence —P3-P2-P1-, wherein P1, P2, and P3 are each an amino acid, wherein:
   a first one of the amino acids P1, P2, or P3 is negatively charged;
   a second one of the amino acids P1, P2, or P3 has an aliphatic side chain with hydrophobicity no greater than that of leucine; and
   a third one of the amino acids P1, P2, or P3 has hydrophobicity lower than that of leucine,
   wherein the first one of the amino acids P1, P2, or P3 corresponds to any one of P1, P2, or P3, the second one of the amino acids P1, P2, or P3 corresponds to one of the two remaining amino acids P1, P2, or P3, and the third one of the amino acids P1, P2, or P3 corresponds to the last remaining amino acids P1, P2, or P3,
   provided that —P3-P2-P1- is not -Glu-Val-Cit- or -Asp-Val-Cit-;
Y is a self-immolative Spacer Unit;
subscript y is 0, 1 or 2 indicating the absence or presence of 1 or 2 of Y, respectively; and
subscript q is an integer ranging from 1 to 4,
provided that subscript q is 1 when subscript b is 0 and subscript q is 2, 3 or 4 when subscript b is 1; and
wherein the Ligand Drug Conjugate compounds of the composition have the structure of Formula 1 in which subscript p is replaced by subscript p′, wherein subscript p′ is independently an integer from 1 to 12, 1 to 10 or 1 to 8 or is 4 or 8.

In some embodiments, provided herein is the Ligand Drug Conjugate composition of Formula 1, wherein the Ligand Drug Conjugate compounds in the Ligand Drug Conjugate composition predominately have drug linker moieties of Formula 1H:

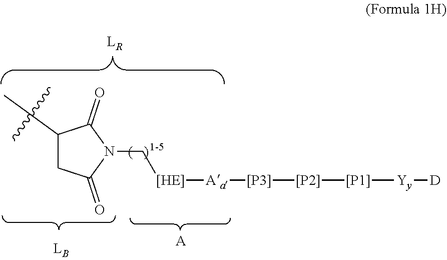

or pharmaceutically acceptable salts thereof, and optionally having a minority of Ligand Drug Conjugate compounds in which one or more of the drug linker moieties in each of such compounds has its succinimide ring in hydrolyzed form and wherein
HE is a Hydrolysis Enhancing Unit;
A′ is a subunit, when present, of the indicated first Stretcher Unit (A); subscript a′ is 0 or 1, indicating the absence or presence of A′; and
the wavy line indicates the site of covalent binding to a sulfur atom of the Ligand Unit.

In some embodiments, which may be combined with any of the preceding embodiments, provided herein is the Ligand Drug Conjugate composition wherein HE is —(C═O).

In some embodiments, which may be combined with any of the preceding embodiments, provided herein is the Ligand Drug Conjugate composition wherein —Y_{y}-D has the structure of:

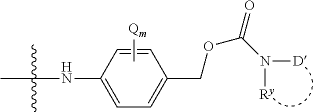

wherein —N(R^{y})D′ represents D, wherein D′ is the remainder of D;
the wavy line indicates the site of covalent attachment to P1;
the dotted line indicates optional cyclization of R^{y} to D′;
R^{y} is optionally substituted C₁-C₆ alkyl in absence of cyclization to D′ or optionally substituted C₁-C₆ alkylene when cyclized to D′;
each Q is independently selected from the group consisting of —C₁-C₈ alkyl, —O—(C₁-C₈ alkyl), halogen, nitro and cyano; and
subscript m is 0, 1 or 2.

In some embodiments, which may be combined with any of the preceding embodiments, provided herein is the Ligand Drug Conjugate composition wherein D is a cytotoxic drug wherein the cytotoxic drug is a secondary amine-containing auristatin compound wherein the nitrogen atom of the secondary amine is the site of covalent attachment to the drug linker moiety and the secondary amine-containing auristatin compound has the structure of Formula D_{F/E-3}:

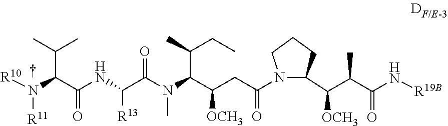

wherein the dagger indicates the site of covalent attachment of the nitrogen atom that provides the carbamate functional group;
one of R¹⁰ and R¹¹ is hydrogen and the other is methyl;
R¹³ is isopropyl or —CH₂—CH(CH₃)₂; and
R^{19B} is —CH(CH₃)—CH(OH)-Ph, —CH(CO₂H)—CH(OH)—CH₃, —CH(CO₂H)—CH₂Ph, —CH(CH₂Ph)-2-thiazolyl, —CH(CH₂Ph)-2-pyridyl, —CH(CH₂-p-Cl-Ph), —CH(CO₂Me)-CH₂Ph, —CH(CO₂Me)-CH₂CH₂SCH₃, —CH(CH₂CH₂SCH₃)C(═O)NH-quinol-3-yl, —CH(CH₂Ph)C(═O)NH-p-Cl-Ph, or
R^{19B} has the structure of

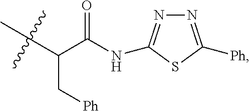 [SMILES: *C(Cc1ccccc1)C(=O)Nc1nnc(-c2ccccc2)s1]

wherein the wavy line indicates covalent attachment to the remainder of the auristatin compound.

In some embodiments, which may be combined with any of the preceding embodiments, provided herein is the Ligand Drug Conjugate composition wherein the secondary amine-containing auristatin compound is monomethylauristatin E (MMAE) or monomethylauristatin F (MMAF).

In some embodiments, which may be combined with any of the preceding embodiments, provided herein is the Ligand Drug Conjugate composition wherein subscript q is 1 and the Ligand Drug Conjugate compounds in the Ligand Drug Conjugate composition predominately have drug linker moieties of Formula 1H-MMAE:

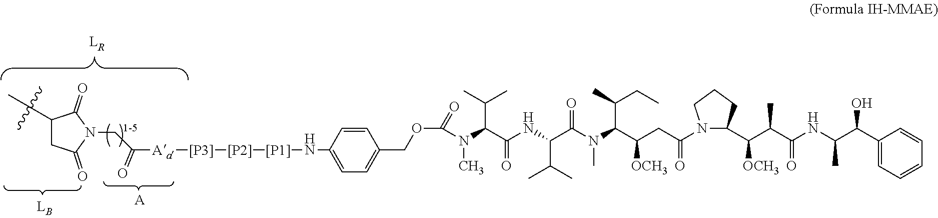

or a pharmaceutical acceptable salt thereof, and optionally having a minority of Ligand Drug Conjugate compounds in which one or more of the drug linker moieties in each of such compounds has its the succinimide ring in hydrolyzed form and wherein:
subscript a′ is 0, and A′ is absent; and
the wavy line indicates the site of covalent binding to a sulfur atom of the Ligand Unit.

In some embodiments, which may be combined with any of the preceding embodiments, provided herein is the Ligand Drug Conjugate composition wherein the Peptide Cleavable Unit is a tripeptide having the sequence —P3-P2-P1-, wherein P1, P2, and P3 are each an amino acid, wherein: the P3 amino acid of the tripeptide is in the D-amino acid configuration; one of the P2 and P1 amino acids has an aliphatic side chain with hydrophobicity lower than that of leucine; and the other of the P2 and P1 amino acids is negatively charged. In some embodiments, the P3 amino acid is D-Leu or D-Ala. Ins ome embodiments, one of the P2 or P1 amino acid has an aliphatic side chain with hydrophobicity no greater than that of valine, and the other of the P2 or P1 amino acid is negatively charged at plasma physiological pH. In some embodiments, P2 amino acid has an aliphatic side chain with hydrophobicity no greater than that of valine, and the P1 amino acid is negatively charged at plasma physiological pH. In some embodiments, —P2-P1- is -Ala-Glu- or -Ala-Asp-. In some embodiments, —P3-P2-P1- is -D-Leu-Ala-Asp-, -D-Leu-Ala-Glu-, -D-Ala-Ala-Asp-, or -D-Ala-Ala-Glu-. In some embodiments, the P3 amino acid is D-Leu or D-Ala, the P2 amino acid is Ala, Glu, or Asp, and the P1 amino acid is Ala, Glu, or Asp.

In some embodiments, provided herein is the Ligand Drug Conjugate composition wherein the compound has the structure of:

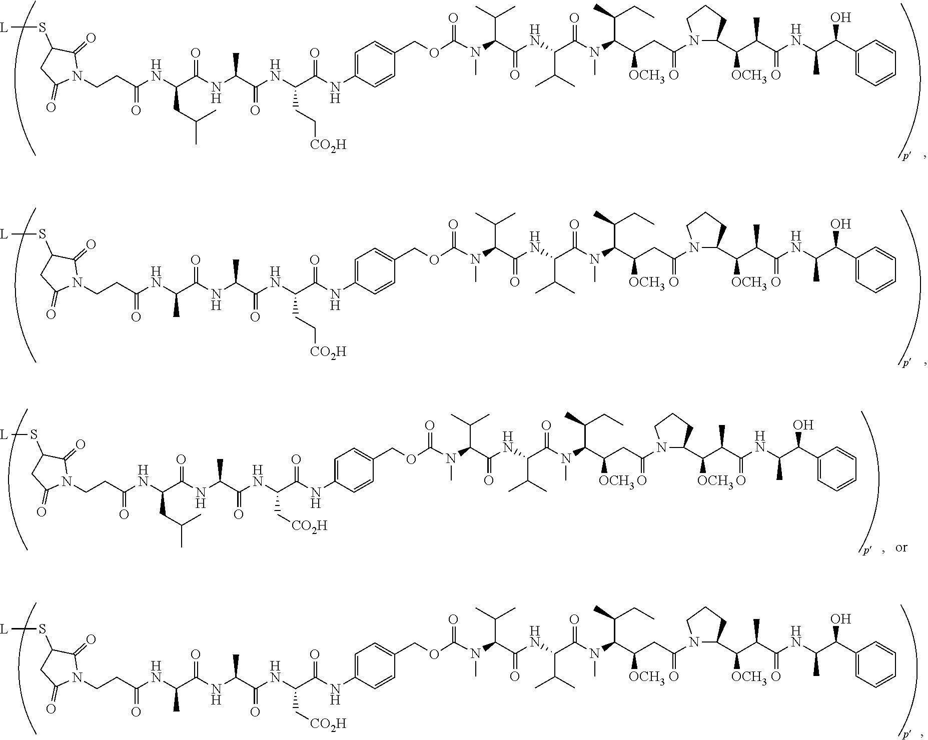

or a pharmaceutically acceptable salt thereof,
wherein L is a Ligand Unit, and subscript p′ is an integer from 1 to 24.

In some embodiments, which may be combined with any of the preceding embodiments, provided herein is the Ligand Drug Conjugate composition wherein L is an antibody Ligand Unit of an intact antibody or an antigen-binding fragment thereof. In some embodiments, the intact antibody or fragment thereof is capable of selectively binding to a cancer cell antigen. In some embodiments, the intact antibody is a chimeric, humanized or human antibody, wherein the antibody is capable of selectively binding to a cancer cell antigen or the antibody is a non-binding control antibody thereby defining a non-binding control Conjugate composition.

In some embodiments, which may be combined with any of the preceding embodiments, provided herein is the Ligand Drug Conjugate composition wherein subscript p ranges from about 2 to about 12, or from about 2 to about 10, or from about 2 to about 8, in particular subscript p is about 2, about 4 or about 8.

In some embodiments, which may be combined with any of the preceding embodiments, provided herein are pharmaceutically acceptable formulations, wherein the formulation comprises an effective amount of a Ligand Drug Conjugate composition or an equivalent amount of a non-binding control Conjugate described herein and at least one pharmaceutically acceptable excipient. In some embodiments, the least one pharmaceutically acceptable excipient is a liquid carrier that provides a liquid formulation, wherein the liquid formulation is suitable for lyophilization or administration to a subject in need thereof. In some embodiments, the formulation is a solid from lyophilization or a liquid formulation described herein, wherein the at least one excipient of the solid formulation is a lyoprotectant

In some embodiments, provided herein is a Drug Linker compound of Formula IA:

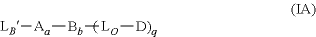

or a salt thereof, wherein
D is a Drug Unit;
L_{B}′ is a ligand covalent binding precursor moiety;
A is a first optional Stretcher Unit;
subscript a is 0 or 1, indicating the absence or presence of A, respectively;
B is an optional Branching Unit;
subscript b is 0 or 1, indicating the absence or presence of B, respectively;
L_{O} is a secondary linker moiety, wherein the secondary linker has the formula of;

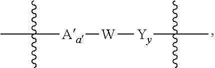

wherein the wavy line adjacent to Y indicates the site of covalent attachment of L_{O} to the Drug Unit and the wavy line adjacent to A′ indicates the site of covalent attachment to the remainder of the Drug Linker compound;
A′ is a second optional Stretcher Unit, which in the absence of B becomes a subunit of A;
subscript a′ is 0 or 1, indicating the absence or presence of A′, respectively,
W is a Peptide Cleavable Unit, wherein the Peptide Cleavable Unit comprises a tripeptide having the sequence —P3-P2-P1-, wherein P1, P2, and P3 are each an amino acid, wherein:
   a first one of the amino acids P1, P2, or P3 is negatively charged;
   a second one of the amino acids P1, P2, or P3 has an aliphatic side chain with hydrophobicity no greater than that of leucine; and
   a third one of the amino acids P1, P2, or P3 has hydrophobicity lower than that of leucine,
   wherein the first one of the amino acids P1, P2, or P3 corresponds to any one of P1, P2, or P3, the second one of the amino acids P1, P2, or P3 corresponds to one of the two remaining amino acids P1, P2, or P3, and the third one of the amino acids P1, P2, or P3 corresponds to the last remaining amino acids P1, P2, or P3,
   provided that —P3-P2-P1- is not -Glu-Val-Cit- or -Asp-Val-Cit-;
Y is a self-immolative Spacer Unit;
subscript y is 0, 1 or 2 indicating the absence or presence of 1 or 2 of Y, respectively; and
subscript q is an integer ranging from 1 to 4, provided that subscript q is 1 when subscript b is 0 and subscript q is 2, 3 or 4 when subscript b is 1.

In some embodiments, provided herein is the Drug Linker compound of Formula IA, wherein the Drug Linker compound has the structure of Formula IH:

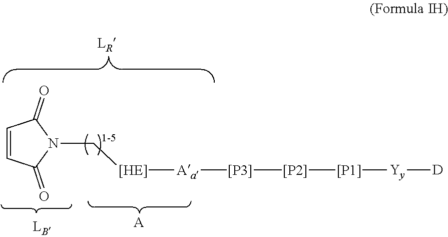

or salt thereof, wherein:
HE is a Hydrolysis Enhancing Unit; and
A′ is a subunit, when present, of the indicated first Stretcher Unit (A); subscript a′ is 0 or 1, indicating the absence or presence of A′.

In some embodiments, which may be combined with any of the preceding embodiments, provided herein is the Drug Linker compound wherein HE is —(C═O).

In some embodiments, which may be combined with any of the preceding embodiments, provided herein is the Drug Linker compound wherein —Y_{y}-D has the structure of:

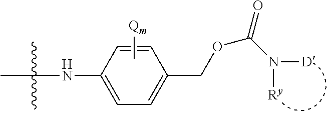

wherein —N(R^{y})D′ represents D, wherein D′ is the remainder of D;
the wavy line indicates the site of covalent attachment to P1;
the dotted line indicates optional cyclization of R^{y} to D′;
R^{y} is optionally substituted C₁-C₆ alkyl in absence of cyclization to D′ or optionally substituted C₁-C₆ alkylene when cyclized to D′;
each Q is independently selected from the group consisting of —C₁-C₈ alkyl, —O—(C₁-C₈ alkyl), halogen, nitro and cyano; and
subscript m is 0, 1 or 2.

In some embodiments, which may be combined with any of the preceding embodiments, provided herein is the Drug Linker compound wherein D is a cytotoxic drug wherein the cytotoxic drug is a secondary amine-containing auristatin compound wherein the nitrogen atom of the secondary amine is the site of covalent attachment to the drug linker moiety and the secondary amine-containing auristatin compound has the structure of Formula D_{F/E-3}:

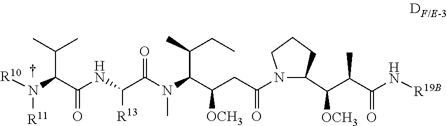

wherein the dagger indicates the site of covalent attachment of the nitrogen atom that provides the carbamate functional group;
one of R¹⁰ and R¹¹ is hydrogen and the other is methyl;
R¹³ is isopropyl or —CH₂—CH(CH₃)₂; and
R^{19B} is —CH(CH₃)—CH(OH)-Ph, —CH(CO₂H)—CH(OH)—CH₃, —CH(CO₂H)—CH₂Ph, —CH(CH₂Ph)-2-thiazolyl, —CH(CH₂Ph)-2-pyridyl, —CH(CH₂-p-Cl-Ph), —CH(CO₂Me)-CH₂Ph, —CH(CO₂Me)-CH₂CH₂SCH₃, —CH(CH₂CH₂SCH₃)C(═O)NH-quinol-3-yl, —CH(CH₂Ph)C(═O)NH-p-Cl-Ph, or
R^{19B} has the structure of

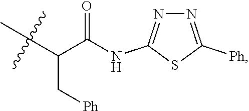 [SMILES: *C(Cc1ccccc1)C(=O)Nc1nnc(-c2ccccc2)s1]

wherein the wavy line indicates covalent attachment to the remainder of the auristatin compound.

In some embodiments, which may be combined with any of the preceding embodiments, provided herein is the Drug Linker compound wherein the secondary amine-containing auristatin compound is monomethylauristatin E (MMAE) or monomethylauristatin F (MMAF).

In some embodiments, which may be combined with any of the preceding embodiments, provided herein is the Drug Linker compound wherein the Drug Linker compound has the structure of Formula IH-MMAE:

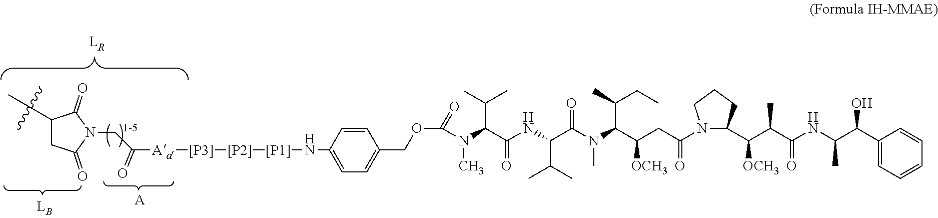

or a salt thereof, wherein
subscript a′ is 0, and A′ is absent.

In some embodiments, which may be combined with any of the preceding embodiments, provided herein is the Drug Linker compound wherein the Peptide Cleavable Unit is a tripeptide having the sequence —P3-P2-P1-, wherein P1, P2, and P3 are each an amino acid, wherein: the P3 amino acid of the tripeptide is in the D-amino acid configuration; one of the P2 and P1 amino acids has an aliphatic side chain with hydrophobicity lower than that of leucine; and the other of the P2 and P1 amino acids is negatively charged. In some embodiments, the P3 amino acid is D-Leu or D-Ala. Ins ome embodiments, one of the P2 or P1 amino acid has an aliphatic side chain with hydrophobicity no greater than that of valine, and the other of the P2 or P1 amino acid is negatively charged at plasma physiological pH. In some embodiments, P2 amino acid has an aliphatic side chain with hydrophobicity no greater than that of valine, and the P1 amino acid is negatively charged at plasma physiological pH. In some embodiments, —P2-P1- is -Ala-Glu- or -Ala-Asp-. In some embodiments, —P3-P2-P1- is -D-Leu-Ala-Asp-, -D-Leu-Ala-Glu-, -D-Ala-Ala-Asp-, or -D-Ala-Ala-Glu-. In some embodiments, the P3 amino acid is D-Leu or D-Ala, the P2 amino acid is Ala, Glu, or Asp, and the P1 amino acid is Ala, Glu, or Asp.

In some embodiments, provided herein is the Drug Linker compound wherein the Drug Linker compound has the structure of:

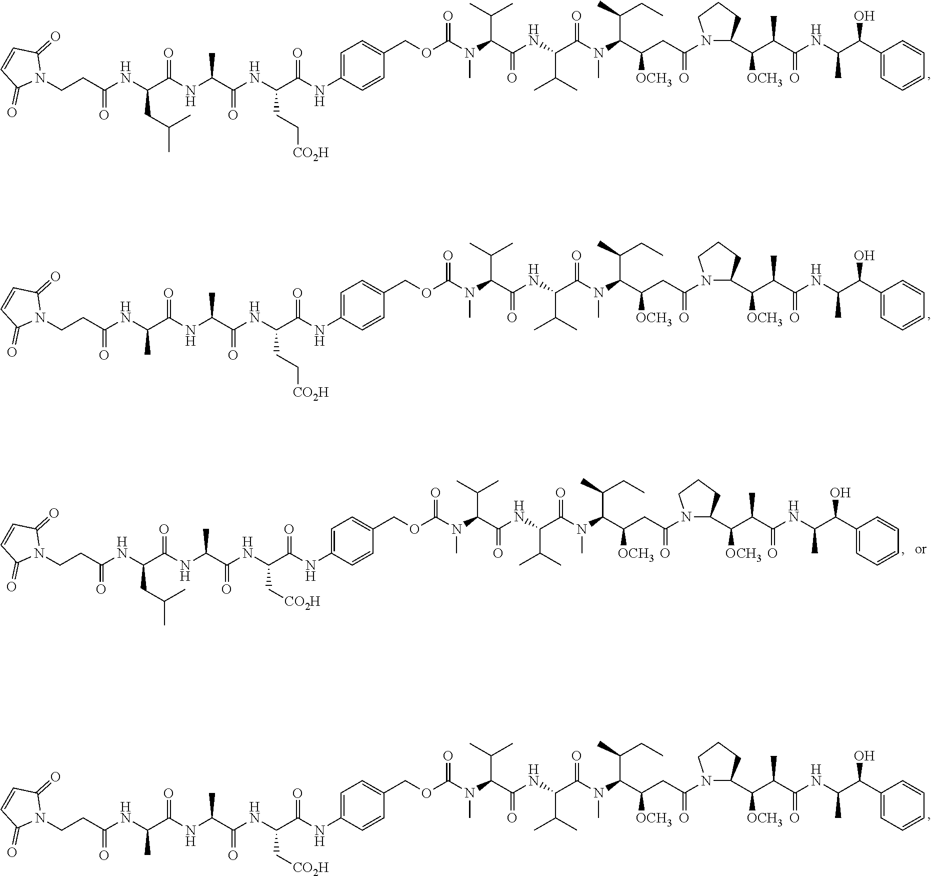

or a salt thereof.

In some embodiments, provided herein is a Linker compound of Formula IA-L:

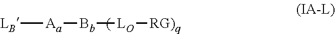

or a salt thereof, wherein
RG is a reactive group;
L_{B}′ is a ligand covalent binding precursor moiety;
A is a first optional Stretcher Unit;
subscript a is 0 or 1, indicating the absence or presence of A, respectively;
B is an optional Branching Unit;
subscript b is 0 or 1, indicating the absence or presence of B, respectively;
L_{O} is a secondary linker moiety, wherein the secondary linker has the formula of;

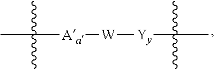

wherein the wavy line adjacent to Y indicates the site of covalent attachment of L_{O} to the Drug Unit and the wavy line adjacent to A′ indicates the site of covalent attachment to the remainder of the Drug Linker compound;
A′ is a second optional Stretcher Unit, which in the absence of B becomes a subunit of A;
subscript a′ is 0 or 1, indicating the absence or presence of A′, respectively,
W is a Peptide Cleavable Unit, wherein the Peptide Cleavable Unit comprises a tripeptide having the sequence —P3-P2-P1-, wherein P1, P2, and P3 are each an amino acid, wherein:
   a first one of the amino acids P1, P2, or P3 is negatively charged;
   a second one of the amino acids P1, P2, or P3 has an aliphatic side chain with hydrophobicity no greater than that of leucine; and
   a third one of the amino acids P1, P2, or P3 has hydrophobicity lower than that of leucine,
   wherein the first one of the amino acids P1, P2, or P3 corresponds to any one of P1, P2, or P3, the second one of the amino acids P1, P2, or P3 corresponds to one of the two remaining amino acids P1, P2, or P3, and the third one of the amino acids P1, P2, or P3 corresponds to the last remaining amino acids P1, P2, or P3,
   provided that —P3-P2-P1- is not -Glu-Val-Cit- or -Asp-Val-Cit-;
Y is a self-immolative Spacer Unit;
subscript y is 0, 1 or 2 indicating the absence or presence of 1 or 2 of Y, respectively; and
subscript q is an integer ranging from 1 to 4, provided that subscript q is 1 when subscript b is 0 and subscript q is 2, 3 or 4 when subscript b is 1.

In some embodiments, provided herein is the Linker compound, wherein the Peptide Cleavable Unit is a tripeptide having the sequence —P3-P2-P1-, wherein P1, P2, and P3 are each an amino acid, wherein: the P3 amino acid of the tripeptide is in the D-amino acid configuration; one of the P2 and P1 amino acids has an aliphatic side chain with hydrophobicity lower than that of leucine; and the other of the P2 and P1 amino acids is negatively charged.

In some embodiments, provide herein is the Linker compound wherein the Linker compound has the structure of Formula IA-L-3:

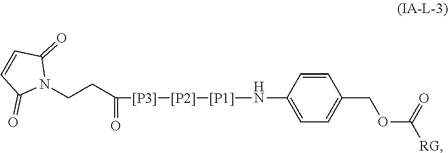

or a salt thereof.

In some embodiments, provided herein is a Linker compound wherein the Linker compound has the structure of:

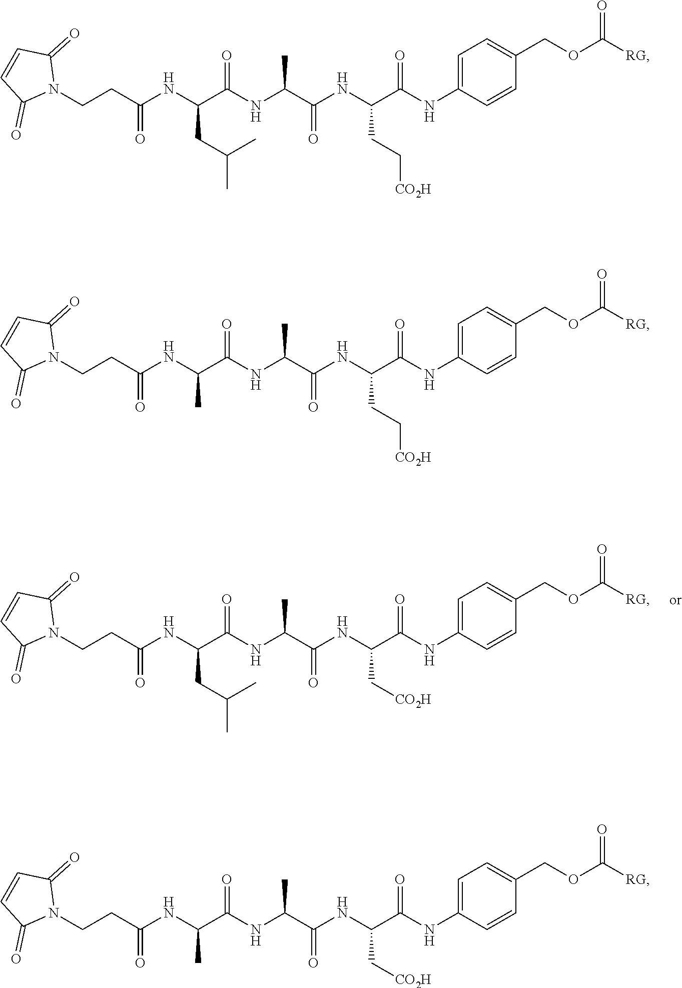

or a salt thereof.

Those and other embodiments of the invention are described in more detail in the following “Detailed Description of the Invention” and “Claims”.

### BRIEF DESCRIPTION OF THE DRAWINGS

FIGS. 1A, 1B, 1C, and 1D. Tumor volume vs days post implant in a xenograft model treated with a series of 4-loaded ADCs having varying tripeptide sequences as the Peptide Cleavable Unit with drug-linker moieties represented by the formula of mp-P₃-P₂—P₁—PABC-MMAE at sub-curative doses compared to a subcurative dose of a 4-loaded ADC targeting the same cancer cell antigen and having drug-linker moieties represented by the formula of mc-val-cit-PABC-MMAE. Compounds in FIG. 1A were tested at 4 mg/kg. Compounds in FIG. 1B and FIG. 1D were tested at 3 mg/kg. Compounds in FIG. 1C were tested at 6 mg/kg.

FIG. 2. Neutrophil counts after day 4 of 10 mg/Kg administration of a series of 4-loaded non-binding control conjugates having varying tripeptide sequences as the Peptide Cleavable Unit with drug-linker moieties represented by the formula of mp-P₃-P₂—P₁—PABC-MMAE in comparison to 4-loaded non-binding conjugates having drug-linker moieties represented by the formula of mc-val-cit-PABC-MMAE or mp-val-cit-PABC-MMAE.

FIG. 3. Reticulocyte counts in rat plasma after day 4 from 10 mg/Kg administration to non-tumor bearing animals of a series of 4-loaded non-binding conjugates having varying tripeptide sequences as the Peptide Cleavable Unit with drug-linker moieties represented by the formula of mp-P₃-P₂—P₁—PABC-MMAE in comparison to 4-loaded non-binding conjugates having drug-linker moieties represented by the formula of mc-val-cit-PABC-MMAE or mp-val-cit-PABC-MMAE.

FIG. 4. Histopathology of bone marrow of rat after administration to non-tumor bearing animals at day 4 of vehicle or 10 mg/Kg of 4-loaded non-binding conjugates having varying tripeptide sequences as the Peptide Cleavable Unit with drug-linker moieties represented by the formula of mp-P₃-P₂—P₁—PABC-MMAE in comparison to a 4-loaded non-binding conjugate having drug-linker moieties represented by the formula of mc-val-cit-PABC-MMAE.

FIGS. 5A and 5B. Free MMAE in rat plasma at various time points subsequent to administration to non-tumor bearing animals of vehicle and 10 mg/Kg of 4-loaded non-binding conjugates having varying tripeptide sequences as the Peptide Cleavable Unit with drug-linker moieties represented by the formula of mp-P₃-P₂—P₁—PABC-MMAE in comparison to a 4-loaded non-binding conjugate having drug-linker moieties represented by the formula of mc-val-cit-PABC-MMAE.

FIGS. 6A and 6B. Percentage of drug cleaved from the heavy chain of 4-loaded non-targeted conjugates having varying tripeptide sequences as the Peptide Cleavable Unit with drug-linker moieties represented by the formula of mp-P₃-P₂—P₁—PABC-MMAE in comparison to a 4-loaded non-targeted conjugate having drug-linker moieties represented by the formula of mp-val-cit-PABC-MMAE in vitro by neutrophil elastase (FIG. 6A) or by Cathepsin B (FIG. 6B).

FIGS. 7, 8, and 9. Aggregation of a series of 4-loaded non-targeted conjugates having varying tripeptide sequences as the Peptide Cleavable Unit with drug-linker moieties represented by the formula of mp-P₃-P₂—P₁—PABC-MMAE after a 96 h incubation in rat plasma (FIG. 7), cyno plasma (FIG. 8), or human plasma (FIG. 9).

FIG. 10. Aggregation of non-targeted MMAF ADCs in rat plasma at various time points.

FIG. 11. Correlation of reticulocyte depletion by non-targeted ADCs in rats and ADC aggregation in rat plasma after a 96 h incubation.

FIG. 12. Correlation of reticulocyte depletion by non-targeted ADCs in rats and ADC aggregation in cyno plasma after a 96 h incubation.

FIG. 13. Correlation of reticulocyte depletion by non-targeted ADCs in rats and ADC aggregation in human plasma after a 96 h incubation.

FIG. 14. Concentration of antibody in extracellular bone marrow compartment of rats administered non-targeted ADCs.

FIG. 15. Amount of free MMAE in bone marrow cells of rats administered non-targeted ADCs.

FIG. 16. Reticulocyte depletion on days 5 and 8 after dose by non-targeted tripeptide ADCs after administration in rats at 20 mg/kg.

FIG. 17. Neutrophil depletion on days 5 and 8 after dose by non-targeted tripeptide ADCs after administration in rats at 20 mg/kg.

FIG. 18. Histology of bone on days 5 and 8 after dose by non-targeted tripeptide ADCs after administration in rats at 20 mg/kg.

FIG. 19. Correlation between cLogP of the linkers and the aggregation of the corresponding h00 conjugate in rat plasma after a 96 h incubation (expressed as % HMW=% high molecular weight species).

FIG. 20. Correlation between reticulocyte depletion caused by non-targeted ADCs in rats and ADC aggregation in rat plasma after a 96 h incubation (expressed as % HMW=% high molecular weight species).

FIG. 21. Correlation between reticulocyte depletion caused by non-targeted ADCs in rats and ADC aggregation in human plasma after a 96 h incubation (expressed as % HMW=% high molecular weight species).

FIG. 22. Correlation between reticulocyte depletion caused by non-targeted ADCs in rats and ADC aggregation in cyno plasma after a 96 h incubation (expressed as % HMW=% high molecular weight species).

### DETAILED DESCRIPTION OF THE INVENTION

### General

The present invention is based, in part, on the unexpected finding that protease activities in tumor tissue are sufficiently different from that of non-targeted normal tissue for providing additional selectivity towards cancer cells that are targeted by a Ligand Drug Conjugate having a protease activateable peptide sequence for conditional release of its conjugated cytotoxic compound. That difference is exploited by the protease cleavable peptide sequences disclosed herein, when those sequences are incorporated into a peptide cleavable Linker Unit of a Ligand Drug Conjugate compound. It is believed that sequences having that property in some instances provide Conjugate compounds whose biodistribution and/or sensitivity to proteolysis to release free cytotoxic compound favor tumor tissue in comparison to normal tissue.

### 1. DEFINITIONS

Unless otherwise stated or implied by context, terms that are used herein have the meanings defined below. Unless otherwise contraindicated or implied, e.g., by including mutually exclusive elements or options, in those definitions and throughout this specification, the terms “a” and “an” mean one or more and the term “or” means and/or where permitted by context. Thus, as presented in the specification and the appended claims, the singular forms “a,” “an,” and “the” include plural referents unless the context clearly dictates otherwise.

At various locations in the present disclosure, e.g., in any disclosed embodiments or in the claims, reference is made to compounds, compositions, or methods that “comprise” one or more specified components, elements or steps. Invention embodiments also specifically include those compounds, compositions, compositions or methods that are, or that consist of, or that consist essentially of those specified components, elements or steps. The term “comprised of” is used interchangeably with the term “comprising” and are stated as equivalent terms. For example, disclosed compositions, devices, articles of manufacture or methods that “comprise” a component or step are open-ended, and they include or read on those compositions or methods plus an additional component(s) or step(s). However, those terms do not encompass unrecited elements that would destroy the functionality of the disclosed compositions, devices, articles of manufacture or methods for its intended purpose. Similarly, disclosed compositions, devices, articles of manufacture or methods that “consist of” a component or step are closed, and they would not include or read on those compositions or methods having appreciable amounts of an additional component(s) or an additional step(s). Furthermore, the term “consisting essentially of” admits for the inclusion of unrecited elements that have no material effect on the functionality of the disclosed compositions, devices, articles of manufacture or methods for its intended purpose as further defined herein. The section headings used herein are for organizational purposes only and are not to be construed as limiting the subject matter described. Unless otherwise indicated, conventional methods of mass spectroscopy, NMR, HPLC, protein chemistry, biochemistry, recombinant DNA techniques, and pharmacology are employed.

“About”, as the term is used herein, unless otherwise stated or implied by context, in connection with a numeric value or range of values to describe a particular property of a compound or composition, indicate that the value or range of values may deviate to an extent deemed reasonable to one of ordinary skill in the art while still describing the particular property. Reasonable deviations include those that are within the accuracy or precision of the instrument(s) used in measuring, determining or deriving the particular property. Specifically, the term “about” when used in this context, indicate that the numeric value or range of values can vary by 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, or 0.01% of the recited value or range of values, typically by 10% to 0.5%, more typically by 5% to 1%, while still describing the particular property.

With respect to subscript p, which denotes the average number of drug linker moieties in a Ligand Drug Conjugate composition as further defined herein, the term “about” reflects the accepted uncertainty in the art for determining that value from a distribution of Ligand Drug Conjugate compounds within that composition as determined by standard methods of size exclusion, HIC chromatography or HPLC-MS.

“Essentially retains”, “essentially retaining” and like terms, as used herein, unless otherwise stated or implied by context, refers to a property, characteristic, function or activity of a compound or composition or moiety thereof that has not detectably changed or is within experimental error of determination of that same activity, characteristic or property of a compound or composition or moiety of related structure.

“Substantially retains”, “substantially retaining” and like terms, as used herein, unless otherwise stated or implied by context, refers to a measured value of a physical property or characteristic of a compound or composition or moiety thereof that may be statistically different from the determination of that same physical property of another compound or composition or moiety of related structure, but which such difference does not translate to a statistically significant or meaningful difference in biological activity or pharmacological property in a suitable biological test system for evaluating that activity or property (i.e., biological activity or property is retained or is essentially retained). Thus, the phrase “substantially retains” is made in reference to the effect that a physical property or characteristic of a compound or composition has on a physiochemical or pharmacological property or biological activity that is explicitly associated with that physical property or characteristic.

“Negligibly”, “negligible” and like terms, as used herein, unless otherwise stated or implied by context, is an amount of an impurity below the level of quantification by HPLC analysis. Depending on context, those terms may alternatively mean that no statistically significant difference is observed between measured values or outcomes or are within experimental error of the instrumentation used to obtain those values. Negligible differences in values of a parameter determined experimentally do not imply that an impurity characterized by that parameter is present in negligible amount.

“Predominately containing”, “predominately having” and like terms, as used herein, unless otherwise stated or implied by context, refers to the major component of a mixture. When the mixture is of two components, then the major component represents more than 50% by weight of the mixture. With a mixture of three or more components the predominant component is the one present in greatest amount in the mixture and may or may not represent a majority of the mass of the mixture.

“Electron-withdrawing group”, as the term is used herein, unless otherwise stated or implied by context, refers to a functional group or electronegative atom that draws electron density away from an atom to which it is bonded either inductively and/or through resonance, whichever is more dominant (i.e., a functional group or atom may be electron-donating through resonance but may overall be electron withdrawing inductively), and tends to stabilize anions or electron-rich moieties. The electron-withdrawing effect is typically transmitted inductively, albeit in attenuated form, to other atoms attached to the bonded atom that has been made electron-deficient by the electron-withdrawing group (EWG), thus reducing the electron density of a more remote reactive center.

An electron-withdrawing group (EWG) is typically selected from the group consisting of —C(═O)R′, —CN, —NO₂, —CX₃, —X, —C(═O)OR′, —C(═O)NH₂, —C(═O)N(R′)R^{op}, —C(═O)R′, —C(═O)X, —S(═O)₂R^{op}, —S(═O)₂OR′, —SO₃H₂, —S(═O)₂NH₂, —S(═O)₂N(R′)R^{op}, —PO₃H₂, —P(═O)(OR′)(OR^{op})₂, —NO, —NH₂, —N(R′)(R^{op}), —N(R^{op})₃⁺, and salts thereof, wherein X is —F, —Br, —Cl, or —I, R^{op} is, at each occurrence, independently selected from a grouping previously described for optional substituents and R′ is —H or R^{op}, wherein R^{op} is a previously defined. In some aspects, each R^{op} is independently C₁-C₁₂ alkyl, C₁-C₈alkyl, C₁-C₆ alkyl or C₁-C₄ alkyl, or is independently selected from the group consisting of C₁-C₆ alkyl and optionally substituted phenyl, and R′ is hydrogen. An EWG can also be an aryl (e.g., phenyl) or heteroaryl depending on its substitution and certain electron deficient heteroaryl groups (e.g., pyridyl). Thus, in some aspects, an “electron-withdrawing group” further encompasses electron-deficient C₅-C₂₄ heteroaryls and C₆-C₂₄ aryls that are substituted with electron-withdrawing substituents. More typically, an electron-withdrawing group is independently selected from the group consisting of —C(═O)R′, —CN, —NO₂, —CX₃, and —X, wherein X is halogen, typically from the group consisting of —F and —Cl and R′ is H, C₁-C₆ alkyl or C₁-C₄ alkyl. Depending on its substituents, an optionally substituted alkyl moiety may also be an electron withdrawing group and thus in such cases these aspects would be encompassed by the term for an electron-withdrawing group.

“Electron-donating group”, as the term is used herein, unless otherwise stated or implied by context, refers to a functional group or electropositive atom that increases electron density of an atom to which it is bonded either inductively and/or through resonance, whichever is more dominant (i.e., a functional group or atom may be electron-withdrawing inductively but may overall be electron-donating through resonance), and tends to stabilize cations or electron poor systems. The electron-donating effect is typically transmitted through resonance to other atoms attached to the bonded atom that has been made electron rich by the electron-donating group (EDG) thus increasing the electron density of a more remote reactive center. Typically, an electron donating group is selected from the group consisting of —OH, —OR′, —NH₂, —NHR′, and N(R′)₂, wherein each R′ is an independently selected from C₁-C₁₂ alkyl, typically C₁-C₆ alkyl. Depending on its substituents, a C₆-C₂₄ aryl, C₅-C₂₄ heteroaryl, or unsaturated C₁-C₁₂ alkyl moiety may also be an electron-donating group, and in some aspects, such moieties are encompassed by the term for an electron-donating group.

“Compound” as the term is used herein, unless otherwise stated or implied by context, refers to and encompasses the chemical compound itself, either named or represented by structure, and salt form(s) thereof, whether explicitly stated or not, unless context makes clear that such salt forms are to be excluded. Compound salts include zwitterionic salt forms and acid addition and base addition salt forms having organic counterions or inorganic counterions and salt forms involving two or more counterions, which may be the same or different. In some aspects, the salt form is a pharmaceutically acceptable salt form of the compound. The term “compound” further encompasses solvate forms of the compound, in which solvent is noncovalently associated with the compound or is reversibly associated covalently with the compound, as when a carbonyl group of the compound is hydrated to form a gem-diol. Solvate forms include those of the compound itself and its salt form(s) and are inclusive of hemisolvates, monosolvates, disolvates, including hydrates; and when a compound can be associated with two or more solvent molecules, the two or more solvent molecules may be the same or different. In some instances, a compound of the invention will include an explicit reference to one or more of the above forms, e.g., salts and solvates, which does not imply any solid state form of the compound; however, this reference is for emphasis only, and is not to be construed as excluding any other of the forms as identified above. Furthermore, when explicit reference to a salt and/or solvate form of a compound or a Ligand Drug Conjugate composition is not made, that omission is not to be construed as excluding the salt and/or solvate form(s) of the compound or Conjugate unless context make clear that such salt and/or solvate forms are to be excluded.

“Optical isomer”, as the term is used herein, unless otherwise stated or implied by context, refers to a related compound in comparison to a reference compound both having identical atom connectivities but differing structurally by one or more chiral centers in opposite stereochemical configuration(s).

“Moiety”, as the term is used herein, unless otherwise stated or implied by context, means a specified segment, fragment, or functional group of a molecule or compound. Chemical moieties are sometimes indicated as chemical entities that are embedded in or appended to (i.e., a substituent or variable group) a molecule, compound or chemical formula.

Unless indicated otherwise or implied by context, for any substituent group or moiety described herein by a given range of carbon atoms, the designated range means that any individual number of carbon atoms is described. Thus, reference to, e.g., “optionally substituted C₁-C₄ alkyl” or “optionally substituted C₂-C₆ alkenyl” specifically means that a 1, 2, 3, or 4 carbon alkyl moiety, optionally substituted, as defined herein, is present, or a 2, 3, 4, 5, or 6 carbon alkenyl moiety, optionally substituted, as defined herein, is present, respectively. All such numerical designations are expressly intended to disclose all of the individual carbon atom groups; and thus “optionally substituted C₁-C₄ alkyl” includes, methyl, ethyl, 3-carbon alkyls, and 4-carbon alkyls, including all of their positional isomers, whether substituted or unsubstituted. Thus, when an alkyl moiety is substituted, the numerical designations refer to an unsubstituted base moiety and are not intended to include carbon atoms not directly attached to the base moiety that may be present in the substituents of that base moiety. For esters, carbonates, carbamates, and ureas as defined herein that are identified by a given range of carbon atoms, the designated range includes the carbonyl carbon of the respective functional group. Thus, a C₁ ester refers to a formate ester and a C₂ ester refers to an acetate ester.

The organic substituents, moieties, and groups described herein, and for other any other moieties described herein, usually will exclude unstable moieties except where such unstable moieties are transient species that one can use to make a compound with sufficient chemical stability for the one or more of the uses described herein. Substituents, moieties or groups by operation of the definitions provided herein that results in those having a pentavalent carbon are specifically excluded.

“Alkyl” as the term is used herein, by itself or as part of another term, unless otherwise stated or implied by context, refers to methyl or a collection of contiguous carbon atoms, one of which is monovalent, wherein one or more of the carbon atoms are saturated (i.e., is comprised of one or more sp³ carbons) and are covalently linked together in normal, secondary, tertiary or cyclic arrangements, i.e., in a linear, branched, cyclic arrangement or some combination thereof. When the contiguous saturated carbon atoms are in a cyclic arrangement such alkyl moieties are, in some aspects, referred to as carbocyclyls as further defined herein.

When referring to an alkyl moiety or group as an alkyl substituent, that alkyl substituent to a Markush structure or another organic moiety with which it is associated is methyl or that chain of contiguous carbon atoms covalently attached to the structure or moiety through a sp³ carbon of the alkyl substituent. An alkyl substituent, as used herein, therefore contains at least one saturated moiety and may also be substituted with cycloalkyl or aromatic or heteroaromatic moieties or groups or by an alkenyl or alkynyl moiety resulting in an unsaturated alkyl. Thus, an optionally substituted alkyl substituent may additionally contain one, two, three or more independently selected double bonds and/or triple bonds or may be substituted by alkenyl or alkynyl moieties or some combination thereof to define an unsaturated alkyl substituent and may be substituted by other moieties that include appropriate optional substituents as described herein. The number of carbon atoms in a saturated alkyl can vary and typically is 1-50, 1-30 or 1-20, and more typically is 1-8 or 1-6, and in an unsaturated alkyl moiety or group typically varies between 3-50, 3-30 or 3-20, and more typically varies between 3-8.

A saturated alkyl moiety contains saturated, acyclic carbon atoms (i.e., acyclic sp³ carbons) and no sp² or sp carbon atoms, but may be substituted with an optional substituent as described herein, provided that such substitution is not through an sp³, sp² or sp carbon atom of the optional substituent as that would affect the identity of the base alkyl moiety so substituted in carbon atom number except when the optional substituent is a Basic Unit as defined herein. Unless otherwise indicated or implied by context, the term “alkyl” will indicate a saturated, non-cyclic hydrocarbon radical, wherein the hydrocarbon radical has the indicated number of covalently linked saturated carbon atoms so that terms such as “C₁-C₆ alkyl” or “C₁-C₆ alkyl” means an alkyl moiety or group containing 1 saturated carbon atom (i.e., is methyl) or 2, 3, 4, 5 or 6 contiguous, non-cyclic saturated carbon atoms and “C₁-C₈ alkyl” refers to an alkyl moiety or group having 1 saturated carbon atom or 2, 3, 4, 5, 6, 7 or 8 contiguous saturated, non-cyclic carbon atoms. Typically a saturated alkyl is a C₁-C₆ or C₁-C₄ alkyl moiety containing no sp² or sp carbon atoms in its contiguous carbon chain, with the latter sometimes referred to as lower alkyl and in some aspects will refer to a saturated C₁-C₈ alkyl moiety having from 1 to 8 contiguous acyclic sp³ carbon atoms containing no sp² or sp carbon atoms in its contiguous carbon chain when the number of carbon atoms is not indicated. In other aspects when a range of contiguous carbon atoms defines the term “alkyl” but without specifying it as saturated or unsaturated, then that term encompasses saturated alkyl with the specified range and unsaturated alkyl in which the lower limit of the range is increased by two carbon atoms. For example, the term “C₁-C₈ alkyl without limitation to a saturated alkyl includes saturated C₁-C₈ alkyl and C₃-C₈ unsaturated alkyl.

When a saturated alkyl substituent, moiety or group is specified, species include those derived from removing a hydrogen atom from a parent alkane (i.e., an alkyl moiety is monovalent) and may include methyl, ethyl, 1-propyl (n-propyl), 2-propyl (iso-propyl, —CH(CH₃)₂), 1-butyl (n-butyl), 2-methyl-1-propyl (iso-butyl, —CH₂CH(CH₃)₂), 2-butyl (sec-butyl, —CH(CH₃)CH₂CH₃), 2-methyl-2-propyl (t-butyl, —C(CH₃)₃), amyl, isoamyl, sec-amyl and other linear and branch chain alkyl moieties.

“Alkylene,” as the term is used herein, by itself of as part of another term, unless otherwise stated or implied by context, refers to a saturated, branched or straight chain hydrocarbon diradical, substituted or unsubstituted, wherein one or more of the carbon atoms is saturated (i.e., is comprised of one or more sp³ carbons), of the stated number of carbon atoms ranging from 1 to 50 or 1 to 30, typically 1 to 20 or 1 to 12 carbon atoms, more typically 1 to 8, 1 or 6, or 1 to 4 carbon atoms and having two radical centers (i.e., is divalent) derived by the removal of two hydrogen atoms from the same or two different saturated (i.e., sp³) carbon atoms of a parent alkane. An alkylene moiety, in some aspects, is an alkyl radical as described herein in which a hydrogen atom has been removed from another of its saturated carbons or from the radical carbon of an alkyl radical to form a diradical. In other aspects, an alkylene moiety is or is further encompassed by a divalent moiety derived from removing a hydrogen atom from a saturated carbon atom of a parent alkyl moiety and are exemplified without limitation by methylene (—CH₂—), 1,2-ethylene (—CH₂CH₂—), 1,3-propylene (—CH₂CH₂CH₂—), 1,4-butylene (—CH₂CH₂CH₂CH₂—), and like diradicals. Typically, an alkylene is a branched or straight chain hydrocarbon containing only sp³ carbons (i.e., is fully saturated notwithstanding the radical carbon atoms) and, in some aspects, is unsubstituted. In other aspects, an alkylene contains an internal site of unsaturation(s) in the form of one or more double and/or triple bond functional groups, typically 1 or 2 such functional groups, more typically 1, so that the terminal carbons of the unsaturated alkylene moiety are monovalent sp³ carbon atoms. In still other aspects, the alkylene is substituted with 1 to 4, typically 1 to 3, or 1 or 2 substituents, as defined herein for optional substituents, at saturated carbon atom(s) of a saturated alkylene moiety or saturated and/or unsaturated carbon atom(s) of an unsaturated alkylene moiety, excluding alkyl, arylalkyl, alkenyl, alkynyl and any other moiety when the resulting substituted alkylene would differ by the number of contiguous non-aromatic carbon atoms relative to the unsubstituted alkylene, except when the optional substituent is a Basic Unit as defined herein.

“Carbocyclyl” as the term is used herein, by itself of as part of another term, unless otherwise stated or implied by context, refers to a radical of a monocyclic, bicyclic or tricyclic ring system, wherein each of the atoms forming the ring system (i.e., skeletal atoms) is a carbon atom and wherein one or more of these carbon atoms in each ring of the cyclic ring system is saturated (i.e., is comprised of one or more sp³ carbons). Thus, a carbocyclyl is a cyclic arrangement of saturated carbons but may also contain unsaturated carbon atom(s) and therefore its carbocyclic ring may be saturated or partially unsaturated or may be fused with an aromatic moiety, wherein the points of fusion to the cycloalkyl and aromatic rings are to adjacent unsaturated carbons of the carbocyclyl moiety and adjacent aromatic carbon atoms of the aromatic moiety.

Unless otherwise specified, a carbocyclyl can be substituted (i.e. optionally substituted) with moieties described for alkyl, alkenyl, alkynyl, aryl, arylalkyl, alkylaryl and the like or can be substituted with another cycloalkyl moiety. Cycloalkyl moieties, groups or substituents include cyclopropyl, cyclopentyl, cyclohexyl, adamantly or other cyclic moieties that have only carbon atoms in their cyclic ring systems.

When carbocyclyl is used as a Markush group (i.e., a substituent) the carbocyclyl is attached to a Markush formula or another organic moiety with which it is associated through a carbon atom that is involved in the carbocyclic ring system of the carbocyclyl moiety provided that carbon is not an aromatic carbon. When an unsaturated carbon atom of an alkene moiety comprising the carbocyclyl substituent is attached to a Markush formula with which it is associated that carbocyclyl is sometimes referred to as a cycloalkenyl substituent. The number of carbon atoms in a carbocyclyl substituent is defined by the total number of skeletal atoms of its carbocyclic ring system. That number can vary and typically ranges from 3 to 50, 1-30 or 1-20, and more typically 3-8 or 3-6 unless otherwise specified, e.g., C₃-C₈ carbocyclyl means an carbocyclyl substituent, moiety or group containing 3, 4, 5, 6, 7 or 8 carbocyclic carbon atoms and C₃-C₆ carbocyclyl means an carbocyclyl substituent, moiety or group containing 3, 4, 5 or 6 carbocyclic carbon atoms. A carbocyclyl may be derived by the removal of one hydrogen atom from a ring atom of a parent cycloalkane or cycloalkene. Representative C₃-C₈ carbocyclyls include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl.

Therefore, carbocyclyl substituents, moieties or groups typically have 3, 4, 5, 6, 7, 8 carbon atoms in its carbocyclic ring system and may contain exo or endo-cyclic double bonds or endo-cyclic triple bonds or a combination of both wherein the endo-cyclic double or triple bonds, or the combination of both, do not form a cyclic conjugated system of 4n+2 electrons. A bicyclic ring system may share two carbon atoms and a tricyclic ring system may share a total of 3 or 4 carbon atoms. In some aspects, a carbocyclyl is a C₃-C₈ or C₃-C₆ carbocyclyl that may be substituted (i.e. optionally substituted) with one or more, 1 to 4, typically 1 to 3, or 1 or 2 moieties described herein for alkyl, alkenyl, alkynyl, aryl, arylalkyl, and alkylaryl and/or with other moieties, including substituent(s) as defined herein for optional substituents, and in some aspects is unsubstituted. In other aspects, a cycloalkyl moiety, group or substituent is a C₃-C₆ cycloalkyl selected from the group consisting of cyclopropyl, cyclopentyl and cyclohexyl, or is a C₃-C₈ cycloalkyl that encompasses that group and is further encompasses other cyclic moieties that have no more than 8 carbon atoms in their cyclic ring systems. When the number of carbon atoms is not indicated, a carbocyclyl moiety, group or substituent has from 3 to 8 carbon atoms in its carbocylic ring system.

“Carbocyclo”, as the term is used herein by itself or as part of another term, unless otherwise stated or implied by context, refers to an optionally substituted carbocyclyl as defined above wherein another hydrogen atom of its cycloalkyl ring system has been removed (i.e., it is divalent) and is a C₃-C₅₀ or C₃-C₃₀ carbocyclo, typically a C₃-C₂₀ or C₃-C₁₂ carbocyclo, more typically a C₃-C₈ or C₃-C₆ carbocyclo and in some aspects is unsubstituted or an optionally substituted C₃, C₅ or C₆ carbocyclo. When the number of carbon atoms is not indicated, a carbocyclo moiety, group or substituent has from 3 to 8 carbon atoms in its carbocylic ring system.

In some aspects, that other hydrogen atom is removed from the monovalent carbon atom of the cycloalkyl to provide a divalent carbon atom, which in some instances is a spiro carbon atom that interrupts an alkyl moiety with that carbocyclic carbon atom. In such instances, the spiro carbon atom is attributed to the carbon atom count of the interrupted alkyl moiety and the carbocyclo ring system with the carbocyclo indicated as being incorporated into the alkyl moiety. In those aspects, a carbocyclo moiety, group or substituent is a C₃-C₆ carbocyclo in the form of a spiro ring system and is selected from the group consisting of cycloprop-1,1-diyl, cyclobutyl-1,1-diyl, cyclopent-1,1-diyl and cyclohex-1,1-diyl, or is a C₃-C₈ carbocyclo, which encompasses that group and is further encompassed by other divalent cyclic moieties that have no more than 8 carbon atoms in their cyclic ring systems. A carbocyclo may be a saturated or an unsaturated carbocyclo, and/or may be unsubstituted or unsubstituted in the same manner as described for a carbocyclyl moiety. If unsaturated, one or both monovalent carbon atoms of the carbocyclo moiety may be sp² carbon atoms from the same or a different double bond functional group or both monovalent carbon atoms may be adjacent or non-adjacent sp³ carbon atoms.

“Alkenyl” as the term is used herein, by itself or as part of another term, unless otherwise stated or implied by context, refers to an organic moiety, substituent or group that comprises one or more double bond functional groups (e.g., a —CH═CH— moiety) or 1, 2, 3, 4, 5 or 6 or more, typically 1, 2 or 3 of such functional groups, more typically one such functional group, and in some aspects may be substituted (i.e., is optionally substituted) with an aryl moiety or group such as phenyl, or may contain non-aromatic linked normal, secondary, tertiary or cyclic carbon atoms, i.e., linear, branched, cyclic or any combination thereof as part of the base moiety unless the alkenyl substituent, moiety or group is a vinyl moiety (e.g., a —CH═CH₂ moiety). An alkenyl moiety, group or substituent having multiple double bonds may have the double bonds arranged contiguously (i.e., a 1,3-butadienyl moiety) or non-contiguously with one or more intervening saturated carbon atoms or a combination thereof, provided that a cyclic, contiguous arrangement of double bonds do not form a cyclic conjugated system of 4n+2 electrons (i.e., is not aromatic).

An alkenyl moiety, group or substituent contains at least one sp² carbon atom in which that carbon atom is divalent and is doubly bonded to another organic moiety or Markush structure to which it is associated, or contains at least two sp² carbon atoms in conjugation to each other in which one of the sp² carbon atoms is monovalent and is singly bonded to another organic moiety or Markush structure to which it is associated. Typically, when alkenyl is used as a Markush group (i.e., is a substituent) the alkenyl is singly bonded to a Markush formula or another organic moiety with which it is associated through a sp² carbon of an alkene functional group of the alkenyl moiety. In some aspects, when an alkenyl moiety is specified, species encompasses those corresponding to any of the optionally substituted alkyl or carbocyclyl, groups moieties or substituents described herein that has one or more endo double bonds in which a sp² carbon atom thereof is monovalent and monovalent moieties derived from removal of a hydrogen atom from a sp² carbon of a parent alkene compound. Such monovalent moieties are exemplified without limitation by vinyl (—CH═CH₂), allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, and cyclohexenyl. In some aspects, the term alkenyl encompasses those and/or other linear, cyclic and branched chained, all carbon-containing moieties containing at least one double bond functional group in which one of the sp² carbon atoms is monovalent.

The number of carbon atoms in an alkenyl moiety is defined by the number of sp² carbon atoms of the alkene functional group(s) that defines it as an alkenyl substituent and the total number of contiguous non-aromatic carbon atoms appended to each of these sp² carbons not including any carbon atom of the other moiety or Markush structure for which the alkenyl moiety is a variable group and carbon atoms from any optional substituent to the alkenyl moiety. That number ranges from 1 to 50 or 1 to 30, typically 1 to 20 or 1 to 12, more typically, 1 to 8, 1 to 6 or 1 to 4 carbon atoms when the double bond functional group is doubly bonded to a Markush structure (e.g. ═CH₂), or ranges from 2 to 50, typically 2 to 30, 2 to 20 or 2 to 12, more typically 2 to 8, 2 to 6 or 2 to 4 carbon atoms, when the double bond functional group is singly bonded to the Markush structure (e.g., —CH═CH₂). For example, C₂-C₈ alkenyl or C2-C8 alkenyl means an alkenyl moiety containing 2, 3, 4, 5, 6, 7 or 8 carbon atoms in which at least two are sp² carbon atoms in conjugation with each other with one of these carbon atoms being monovalent, and C₂-C₆ alkenyl or C2-C6 alkenyl means an alkenyl moiety containing 2, 3, 4, 5 or 6 carbon atoms in which at least two are sp² carbons that are in conjugation with each other with one of these carbon atoms being monovalent. In some aspects, an alkenyl substituent or group is a C₂-C₆ or C₂-C₄ alkenyl moiety having only two sp² carbons that are in conjugation with each other with one of these carbon atoms being monovalent, and in other aspects that alkenyl moiety is unsubstituted or is substituted with 1 to 4 or more, typically 1 to 3, more typically 1 or 2, independently selected moieties as disclosed herein, including substituents as defined herein for optional substituents, excluding alkyl, arylalkyl, heteroarylalkyl, alkenyl, alkynyl and any other moiety when the substituted alkenyl would differ by the number of contiguous non-aromatic carbon atoms relative to the unsubstituted alkenyl, wherein the substitution(s) may be at any of the alkenyl moiety's contiguous sp² carbon and sp³ carbon atoms, if any. Typically, an alkenyl substituent is a C₂-C₆ or C₂-C₄ alkenyl moiety having only two sp² carbons that are in conjugation with each other. When the number of carbon atoms is not indicated, an alkenyl moiety has from 2 to 8 carbon atoms.

“Alkenylene” as the term is used herein, by itself of as part of another term, unless otherwise stated or implied by context, refers to an organic moiety, substituent or group that comprises one or more double bond moieties, as previously described for alkenyl, of the stated number of carbon atoms and has two radical centers derived by the removal of two hydrogen atoms from the same or two different sp² carbon atoms of an alkene functional group or removal of two hydrogen atoms from two separate alkene functional groups in a parent alkene. In some aspects, an alkenylene moiety is that of an alkenyl radical as described herein in which a hydrogen atom has been removed from the same or different sp² carbon atom of a double bond functional group of the alkenyl radical, or from a sp² carbon from a different double bonded moiety to provide a diradical. Typically, alkenylene moieties encompass diradicals containing the structure of —C═C— or —C═C—X¹—C═C— wherein X¹ is absent or is an optionally substituted saturated alkylene as defined herein, which is typically a C₁-C₆ alkylene, which is more typically unsubstituted. The number of carbon atoms in an alkenylene moiety is defined by the number of sp² carbon atoms of its alkene functional group(s) that defines it as an alkenylene moiety and the total number of contiguous non-aromatic carbon atoms appended to each of its sp² carbons not including any carbon atoms of the other moiety or Markush structure in which the alkenyl moiety is a present as a variable group. That number, unless otherwise specified, ranges from 2 to 50 or 2 to 30, typically from 2 to 20 or 2 to 12, more typically from 2 to 8, 2 to 6 or 2 to 4 carbon atoms. For example, C₂-C₈ alkenylene or C2-C8 alkenylene means an alkenylene moiety containing 2, 3, 4, 5, 6, 7 or 8 carbon atoms, in which at least two are sp² carbons in which one is divalent or both are monovalent, that are in conjugation with each other and C₂-C₆ alkenylene or C2-C6 alkenylene means an alkenyl moiety containing 2, 3, 4, 5 or 6 carbon atoms in which at least two are sp² carbons, in which at least two are sp² carbons in which one is divalent or both are monovalent, that are in conjugation with each other. In some aspects, an alkenylene moiety is a C₂-C₆ or C₂-C₄ alkenylene having two sp² carbons that are in conjugation with each other in which both sp² carbon atoms are monovalent, and in some aspects is unsubstituted. When the number of carbon atoms is not indicated, an alkenylene moiety has from 2 to 8 carbon atoms and is unsubstituted or substituted in the same manner described for an alkenyl moiety.

“Alkynyl” as the term is used herein, by itself or as part of another term, unless otherwise stated or implied by context, refers to an organic moiety, substituent or group that comprises one or more triple bond functional groups (e.g., a —C≡C— moiety) or 1, 2, 3, 4, 5, or 6 or more, typically 1, 2, or 3 of such functional groups, more typically one such functional group, and in some aspects may be substituted (i.e., is optionally substituted) with an aryl moiety such as phenyl, or by an alkenyl moiety or linked normal, secondary, tertiary or cyclic carbon atoms, i.e., linear, branched, cyclic or any combination thereof unless the alkynyl substituent, moiety or group is —C≡CH). An alkynyl moiety, group or substituent having multiple triple bonds may have the triple bonds arranged contiguously or non-contiguously with one or more intervening saturated or unsaturated carbon atoms or a combination thereof, provided that a cyclic, contiguous arrangement of triple bonds do not form a cyclic conjugated system of 4n+2 electrons (i.e., is not aromatic).

An alkynyl moiety, group or substituent contains at least two sp carbon atom in which the carbon atoms are conjugation to each other and in which one of the sp carbon atoms is singly bonded, to another organic moiety or Markush structure to which it is associated. When alkynyl is used as a Markush group (i.e., is a substituent) the alkynyl is singly bonded to a Markush formula or another organic moiety with which it is associated through a triple-bonded carbon (i.e., a sp carbon) of a terminal alkyne functional group. In some aspects when an alkynyl moiety, group or substituent is specified, species encompasses are any of the optionally substituted alkyl or carbocyclyl, groups moieties or substituents described herein that has one or more endo triple bonds and monovalent moieties derived from removal of a hydrogen atom from a sp carbon of a parent alkyne compound. Such monovalent moieties are exemplified without limitation by —C—CH, and —C≡C—CH₃, and —C≡C-Ph.

The number of carbon atoms in an alkynyl substituent is defined by the number of sp carbon atoms of the alkene functional group that defines it as an alkynyl substituent and the total number of contiguous non-aromatic carbon atoms appended to each of these sp carbons not including any carbon atom of the other moiety or Markush structure for which the alkenyl moiety is a variable group. That number can vary ranging from 2 to 50, typically 2 to 30, 2 to 20, or 2 to 12, more typically 2 to 8, 2 to 6, or 2 to 4 carbon atoms, when the triple bond functional group is singly bonded to the Markush structure (e.g., —CH≡CH). For example, C₂-C₈ alkynyl or C2-C8 alkynyl means an alkynyl moiety containing 2, 3, 4, 5, 6, 7, or 8 carbon atoms in which at least two are sp carbon atoms in conjugation with each other with one of these carbon atoms being monovalent, and C₂-C₆ alkynyl or C2-C6 alkynyl means an alkynyl moiety containing 2, 3, 4, 5, or 6 carbon atoms in which at least two are sp carbons that are in conjugation with each other with one of these carbon atoms being monovalent. In some aspects, an alkynyl substituent or group is a C₂-C₆ or C₂-C₄ alkynyl moiety having two sp carbons that are in conjugation with each other with one of these carbon atoms being monovalent, and in other aspects that alkynyl moiety is unsubstituted. When the number of carbon atoms is not indicated, an alkynyl moiety, group or substituent has from 2 to 8 carbon atoms. An alkynyl moiety may be substituted or unsubstituted in the same manner as described for an alkenyl moiety, except that substitution at the monovalent sp carbon is not permitted.

“Aryl” as the term is used herein, by itself or as part of another term, unless otherwise stated or implied by context, refers to an organic moiety, substituent or group having an aromatic or fused aromatic ring system with no ring heteroatoms comprising or consisting of 1, 2, 3 or 4 to 6 aromatic rings each of which are independently optionally substituted, typically consisting of 1 to 3 aromatic rings, more typically 1 or 2 aromatic rings each of which are independently optionally substituted, wherein the rings are composed of only carbon atoms that participate in a cyclically conjugated system of 4n+2 electrons (Hückel rule), typically 6, 10 or 14 electrons, some of which may additionally participate in exocyclic conjugation with a heteroatom (cross-conjugated, e.g., quinone). Aryl substituents, moieties or groups are typically formed by six, eight, ten or more contiguous aromatic carbon atoms up to 24 to include C₆-C₂₄ aryl and in some aspects is a C₆-C₂₀ or C₆-C₁₂ aryl. Aryl substituents, moieties or groups are optionally substituted and in some aspects are unsubstituted or substituted with 1, 2, 3 or more, typically 1 or 2, independently selected substituents as defined herein for alkyl, alkenyl, alkynyl or other moiety described herein including another aryl or a heteroaryl to form a biaryl and other optional substituents as defined herein. In other aspects, aryls are C₆-C₁₀ aryls such as phenyl and naphthalenyl and phenanthryl. As aromaticity in a neutral aryl moiety requires an even number or electrons, it will be understood that a given range for that moiety will not encompass species with an odd number of aromatic carbons. When aryl is used as a Markush group (i.e., a substituent) the aryl is attached to a Markush formula or another organic moiety with which it is associated through an aromatic carbon of the aryl group.

“Heterocyclyl” as the term is used herein, by itself or as part of another term, unless otherwise stated or implied by context, refers to a carbocyclyl in which one or more, but not all of the skeletal carbon atoms with their attached hydrogen atoms within the carbocyclic ring system are replaced by independently selected heteroatoms or heteroatom moieties, optionally substituted where permitted, including without limitation N/NH, O, S, Se, B, Si and P, wherein two or more heteroatoms or heteroatom moieties, typically 2, may be adjacent to each other or separated by one or more carbon atoms within the same ring system, typically by 1 to 3 carbon atoms. Those heteroatoms or heteroatom moieties typically are N/NH, O and S. A heterocyclyl typically contains a monovalent skeletal carbon atom or a monovalent heteroatom or heteroatom moiety and has a total of one to ten heteroatoms and/or heteroatom moieties, typically a total of 1 to 5, or more typically a total of 1 to 3, or 1 or 2, provided that not all of the skeletal atoms in any one of the heterocyclic ring(s) in the heterocyclyl are heteroatoms and/or heteroatom moieties (i.e. at least one carbon atom is not replaced in each ring with at least one having been replaced in one of the rings), wherein each heteroatom or heteroatom moiety in the ring(s), optionally substituted where permitted, is independently selected from the group consisting of N/NH, O and S, with the proviso that any one ring does not contain two adjacent O or S atoms. Exemplary heterocyclyls and heteroaryls are collectively referred to as heterocycles, are provided by Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. 1960, 82:5545-5473 particularly 5566-5573).

When heterocyclyl is used as a Markush group (i.e., a substituent) a saturated or partially unsaturated heterocyclic ring of the heterocyclyl is attached to a Markush structure or other moiety with which it is associated through a carbon atom or a heteroatom of that heterocyclic ring, where such attachment does not result in an unstable or disallowed formal oxidation state of that carbon atom or heteroatom. A heterocyclyl in that context is a monovalent moiety in which a heterocyclic ring of the heterocyclic ring system defining it as a heterocyclyl is non-aromatic, but may be fused with a carbocyclic, aryl or heteroaryl ring and includes phenyl- (i.e., benzo) fused heterocyclic moieties.

A heterocyclyl is a C₃-C₅₀ or C₃-C₃₀ carbocyclyl, typically a C₃-C₂₀ or C₃-C₁₂ carbocyclyl, more typically a C₃-C₈ or C₃-C₆ carbocyclyl wherein 1, 2 or 3 or more, but not all of its carbons of its cycloalkyl ring system are replaced along with its attached hydrogens, typically 1, 2, 3 or 4, more typically 1 or 2, are replaced with a heteroatom or heteroatom moiety independently selected from the group consisting of N/NH, O and S, optionally substituted where permitted, and thus is a C₃-C₅₀ or C₃-C₃₀ heterocyclyl, typically a C₃-C₂₀ or C₃-C₁₂ heterocyclyl, more typically a C₃-C₆, or C₅-C₆ heterocyclyl, in which the subscript indicates the total number of skeletal atoms (inclusive of its carbon atoms and heteroatoms) of the heterocyclic ring system(s) of the heterocyclyl. In some aspects, a heterocyclyl contains 0 to 2 N, 0 to 2 O or 0 to 1 S skeletal heteroatoms, optionally substituted or some combination thereof provided at least one of said heteroatoms is present in a heterocyclic ring system of the heterocyclyl. A heterocyclyl may be saturated or partially unsaturated and/or unsubstituted or substituted at a skeletal carbon atom with an oxo (═O) moiety, as in pyrrolidin-2-one, and/or at a skeletal heteroatom with one or two oxo moieties so as to contain an oxidized heteroatom as exemplified, but not limited to, —N(═O), —S(═O)— or —S(═O)₂—. A fully saturated or partially unsaturated heterocyclyl may be substituted or further substituted with an alkyl, (hetero)aryl, (hetero)arylalkyl, alkenyl, alkynyl or other moiety as described herein, including optional substituents as defined herein or a combination of 2, 3 or more, typically 1 or 2, such substituents. In certain aspects, heterocyclyl is selected from the group consisting of pyrrolidinyl, piperidinyl, morpholinyl and piperazinyl.

“Heterocyclo”, as the term is used herein, by itself or as part of another term, unless otherwise stated or implied by context, refers to a heterocyclyl moiety, group or substituent as defined above wherein a hydrogen atom from its monovalent carbon atom, a hydrogen atom from a different skeletal atom (carbon or nitrogen atom if the latter is present), or an electron from a skeletal nitrogen atom, where permitted, is removed or an electron from a nitrogen ring atom that is not already monovalent is removed and is replaced with a bond (i.e., it is divalent). In some aspects, the replaced second hydrogen is that of the monovalent carbon atom of the parent heterocyclyl thus forming a spiro carbon atom, which in some instances may interrupt an alkyl moiety with that carbocyclic carbon atom. In such instances, the spiro carbon atom is attributed to the carbon atom count of the interrupted alkyl moiety with the heterocyclo indicated as being incorporated into the alkyl moiety.

“Heteroaryl” as the term is used herein, by itself or as part of another term, unless otherwise stated or implied by context, refers to an aryl moiety, group or substituent as defined herein in which one or more but not all of the aromatic carbons of an aromatic ring system of an aryl is replaced by a heteroatom. A heteroaryl typically contains a total one to four skeletal heteroatoms in the ring(s) of the heteroaryl ring system, provided that not all of the skeletal atoms of any one ring system in the heteroaryl are heteroatoms, which are optionally substituted where permitted, and have 0 to 3 N, 1 to 3 N or 0 to 3 N skeletal heteroatoms, typically 0 to 1 O, and/or 0 to 1 S skeletal heteroatoms, provided that at least one skeletal heteroatom is present. A heteroaryl may be monocyclic, bicyclic or polycyclic. A polycyclic heteroaryl is typically a C₅-C₅₀ or C₅-C₃₀ heteroaryl, more typically a C₅-C₂₀ or C₅-C₁₂ heteroaryl, a bicyclic heteroaryl is typically a C₅-C₁₀ heteroaryl, and a monocyclic heteroaryl is a typically is C₅-C₆ heteroaryl, in which the subscript indicates the total number of skeletal atoms (inclusive of its carbon atoms and heteroatoms) of the aromatic ring system(s) of the heteroaryl. In some aspects, a heteroaryl is a bicyclic aryl moiety wherein one 1, 2, 3, 4 or more, typically 1, 2 or 3, of the carbon atoms of the aromatic ring(s) and their attached hydrogen atoms of a parent bicyclic aryl moiety are replaced by an independently selected heteroatom or heteroatom moiety, or is a monocyclic aryl moiety wherein one 1, 2, 3 or more, typically 1 or 2, of the carbon atoms of the aromatic ring(s) and their attached hydrogen atoms of a parent monocyclic aryl moiety are replaced by an independently selected heteroatom or heteroatom moiety, wherein the heteroatom or heteroatom moiety is optionally substituted where permitted, including N/NH, O and S, provided that not all of the skeletal atoms of any one aromatic ring system in the parent aryl moiety are replaced by heteroatoms and more typically are replaced by oxygen (—O—), sulfur (—S—) nitrogen (═N—) or —NR—, so that the nitrogen heteroatom is optionally substituted, wherein R is —H, a nitrogen protecting group or optionally substituted C₁-C₂₀ alkyl or is an optionally substituted C₆-C₂₄ aryl or C₅-C₂₄ heteroaryl to form a heterobiaryl. In other aspects, 1, 2 or 3 of the carbon atoms of the aromatic ring(s) and their attached hydrogen atoms of a parent aryl moiety are replaced by nitrogen substituted with another organic moiety in a manner which retains the cyclic conjugated system. In still other aspects, the aromatic carbon radical of a parent aryl moiety is replaced with an aromatic nitrogen radical. In either of those aspects, the nitrogen, sulfur or oxygen heteroatom participates in the conjugated system either through pi-bonding with an adjacent atom in the ring system or through a lone pair of electrons on the heteroatom. In still other aspects, a heteroaryl has the structure of a heterocyclyl as defined herein in which its ring system has been aromatized.

Typically, a heteroaryl is monocyclic, which, in some aspects, has a 5-membered or 6-membered heteroaromatic ring system. A 5-membered heteroaryl is a monocyclic C₅-heteroaryl containing 1 to 4 aromatic carbon atoms and the requisite number of aromatic heteroatoms within its heteroaromatic ring system. A 6-membered heteroaryl is a monocyclic C₆ heteroaryl containing 1 to 5 aromatic carbon atoms and the requisite number of aromatic heteroatoms within its heteroaromatic ring system. Heteroaryls that are 5-membered have four, three, two or one aromatic heteroatom(s), and heteroaryls that are 6-membered include heteroaryls having five, four, three, two or one aromatic heteroatom(s).

C₅-heteroaryls, also referred to as 5-membered heteroaryl, are monovalent moieties derived from removing a hydrogen atom from a skeletal aromatic carbon or an electron from a skeletal aromatic heteroatom, where permitted, from a parent aromatic heterocycle compound, which is some aspects is selected from the group consisting of pyrrole, furan, thiophene, oxazole, isoxazole, thiazole, isothiazole, imidazole, pyrazole, triazole and tetrazole. In other aspects, the parent heterocycle is selected from the group consisting of thiazole, imidazole, oxazole, and triazole and is typically thiazole or oxazole, more typically thiazole.

C₆ heteroaryls, which are 6-membered, are monovalent moieties derived from removing a hydrogen atom from an aromatic carbon or an electron from an aromatic heteroatom, where permitted, from a parent aromatic heterocycle compound, which is certain aspects is selected from the group consisting of pyridine, pyridazine, pyrimidine, and triazine. A heteroaryl may be substituted or further substituted with an alkyl, (hetero)arylalkyl, alkenyl or alkynyl, or with an aryl or another heteroaryl to form a biaryl, or with other moieties as described herein, including optional substituents as defined herein, or a combination of 2, 3 or more, typically 1 or 2, such substituents.

“Arylalkyl” or “heteroarylalkyl” as the terms are used herein, by itself or as part of another term, refers to an aryl or heteroaryl moiety bonded to an alkyl moiety, i.e., (aryl)-alkyl-, where alkyl and aryl groups are as described above. Typically, an arylalkyl is a (C₆-C₂₄ aryl)-C₁-C₁₂ alkyl-moiety, group or substituent, and heteroarylalkyl is a (C₅-C₂₄ heteroaryl)-C₁-C₁₂ alkyl-moiety, group or substituent. When (hetero)arylalkyl is used as a Markush group (i.e., a substituent) the alkyl moiety of the (hetero)arylalkyl is attached to a Markush formula with which it is associated through a sp³ carbon of its alkyl moiety. In some aspects, an arylalkyl is a (C₆-C₂₄ aryl)-C₁-C₁₂ alkyl- or a (C₆-C₂₀ aryl)-C₁-C₂₀ alkyl-, typically a (C₆-C₁₂ aryl)-C₁-C₁₂ alkyl- or (C₆-C₁₀ aryl)-C₁-C₁₂ alkyl-, more typically a (C₆-C₁₀ aryl)-C₁-C₆ alkyl-exemplified without limitation, by C₆H₅—CH₂—, C₆H₅—CH(CH₃)CH₂— and C₆H₅—CH₂—CH(CH₂CH₂CH₃)—. An (hetero)arylalkyl may be unsubstituted or substituted in the same manner as described for (hetero)aryl and/or alkyl moieties.

“Arylene,” or “heteroarylene” as the terms are used herein, by itself or as part of another term, unless otherwise stated or implied by context, is an aromatic or heteroaromatic diradical moiety that forms two covalent bonds (i.e., it is divalent) within another organic moiety, for which the bonds are in the ortho, meta, or para configuration. Arylene and some heteroarylenes include divalent species by removal of a hydrogen atom from a parent aryl or heteroaryl moiety, group or substituent as defined herein. Other heteroarylenes are divalent species in which hydrogen atoms have been removed from two different aromatic carbon atoms of a parent aromatic heterocycle to form a diradical species, or from removal of a hydrogen atom from an aromatic carbon atom or heteroatom and of another hydrogen atom or electron from a different aromatic heteroatom from a parent aromatic heterocycle to form a diradical species in which one aromatic carbon atom and one aromatic heteroatom is monovalent or two different aromatic heteroatoms are each monovalent. Heteroarylene further include those in which heteroatom(s) and/or heteroatom moiety(ies) replace one or more but not all of the aromatic carbon atoms of a parent arylene.

Non-limiting exemplary arylenes, which are optionally substituted at the remaining positions, are phenyl-1,2-ene, phenyl-1,3-ene, and phenyl-1,4-ene, as shown in the following structures:

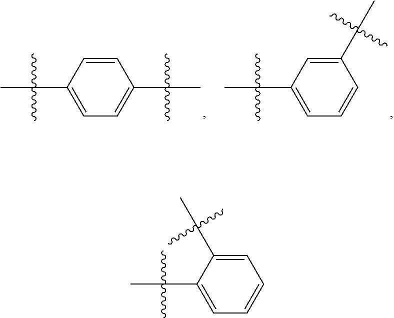 [SMILES: *c1ccc(*)cc1 ; *c1cccc(*)c1 ; *c1ccccc1*]

“Heteroalkyl,” as the term is used herein by itself or in combination with another term, unless otherwise stated or implied by context, refers to an optionally substituted straight or branched chain hydrocarbon, fully saturated or containing from 1 to 3 degrees of unsaturation and having 1 to 12 carbon atom and 1 to 6 heteroatoms, typically 1 to 5 heteroatoms, more typically one or two heteroatoms or heteroatom moieties, selected from the group consisting of O, N/NH, Si and S, optionally substituted where permitted, and includes each nitrogen and sulfur atom independently optionally oxidized to an N-oxide, a sulfoxide or sulfone, or wherein one or more of the nitrogen atoms is optionally substituted or quaternized. The heteroatom(s) or heteroatom moiety(ies) 0, N/NH, S, and/or Si may be placed at any interior position of the heteroalkyl group or at a terminal position of the optionally substituted alkyl group of the heteroalkyl. In some aspects, the heteroalkyl is fully saturated or contains 1 degree of unsaturation and contain 1 to 6 carbon atoms and 1 to 2 heteroatoms, and in other aspects that heteroalkyl is unsubstituted. Non-limiting examples are —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —NH—CH₂—CH₂—NH—C(O)—CH₂—CH₃, —CH₂—CH₂—S(O)₂—CH₃, —CH═CHO—CH₃, —Si(CH₃)₃, —CH₂—CH═NO—CH₃, and —CH═CH—N(CH₃)—CH₃. Up to two heteroatoms may be consecutive, as exemplified by —CH₂—NH—OCH₃ and —CH₂—O—Si(CH₃)₃.

A heteroalkyl is typically denoted by the number of its contiguous heteroatom(s) and non-aromatic carbon atoms, which includes those contiguous carbon atom(s) attached to the heteroatom(s), unless indicated otherwise (e.g., as described for aminoalkyl) or by context. Thus, —CH₂—CH₂—O—CH₃ and —CH₂—CH₂—S(O)—CH₃ are both C₄-heteroalkyls and —CH₂—CH═NO—CH₃, and —CH═CH—N(CH₃)₂ are both C₅ heteroalkyls. A heteroalkyl may be unsubstituted or substituted (i.e., optionally substituted) at its heteroatom or heteroatom component with any one of the moieties described herein, including an optional substituent as defined herein, and/or at its alkyl component with 1 to 4 or more, typically 1 to 3 or 1 or 2 independently selected moieties as described herein, including optional substituent(s) as defined herein, excluding alkyl, (hetero)arylalkyl, alkenyl, alkynyl, another heteroalkyl or any other moiety when the substituted alkenyl would differ by the number of contiguous non-aromatic carbon atoms relative to the unsubstituted aminoalkyl.

An aminoalkyl as defined herein is an exemplary heteroalkyl in which a terminal carbon atom of an alkyl moiety other than its monovalent carbon atom is replaced by an amino group. When indicated as a substituent to a Markush structure or other organic moiety to which it is associated, the monovalent carbon atom of the alkyl moiety is attached to another organic moiety with which it is to be associated, which typically is a different carbon atom to that attached to the amino group. An aminoalkyl differs from other heteroalkyls by denotation in numbering by only indicating the number of contiguous carbon atoms of its alkylene moiety.

“Heteroalkylene” as the term is used herein by itself or in combination with another term, unless otherwise stated or implied by context, means a divalent group derived from a heteroalkyl (as discussed above), by removal of a hydrogen atom or a heteroatom electron form a parent heteroalkyl to provide a divalent moiety exemplified by, but not limited to, —CH₂—CH₂—S—CH₂—CH₂— and —CH₂—S—CH₂—CH₂—NH—CH₂—. For a heteroalkylene, heteroatom(s) thereof may be interior to or may occupy either or both termini of its optionally substituted alkylene chain so that one or both of these heteroatoms are monovalent. When a heteroalkylene is a component of a Linker Unit both orientations of that component within the Linker Unit is permitted unless indicated or implied by context. A heteroalkylene is typically denoted by the number of its contiguous heteroatom(s) and non-aromatic carbon atoms, which includes those contiguous carbon atom(s) attached to the heteroatom(s), unless indicated otherwise or by context. A alkylene diamine is a heteroalkylene in which the two monovalent carbon atoms of an alkylene are replaced by amino groups so that each of their nitrogen atoms is monovalent and differs from other heteroalkylenes by denotation in numbering by only indicating the number of contiguous carbon atoms of its alkylene moiety.

“Aminoalkyl” as the term is used herein by itself or in combination with another term, unless otherwise stated or implied by context, refers to a moiety, group or substituent having a basic nitrogen bonded to one radical terminus of an alkylene moiety as defined above to provide a primary amine in which the basic nitrogen is not further substituted, or to provide a secondary or tertiary amine in which the basic amine is further substituted by one or two independent selected optional substituted C₁-C₁₂ alkyl moieties, respectively, as described above. In some aspects, the optionally substituted alkyl is a C₁-C₈ alkyl or C₁-C₆ alkyl and in other aspects that alkyl is unsubstituted. In still other aspects, the basic nitrogen together with its substituents defines an optionally substituted C₃-C₈ heterocyclyl containing the basic nitrogen as a skeletal atom, typically in the form of a nitrogen-containing C₃-C₆ or C₅-C₆ heterocyclyl, optionally substituted. When aminoalkyl is used as a variable group to a Markush structure, the alkylene moiety of the aminoalkyl is attached to a Markush formula with which it is associated through a sp³ carbon of that moiety, which, in some aspects, is the other radical terminus of the aforementioned alkylene. An aminoalkyl is typically denoted by the number of contiguous carbon atoms of its alkylene moiety. Thus, a C₁ aminoalkyl is exemplified without limitation by —CH₂NH₂, —CH₂NHCH₃ and —CH₂N(CH₃)₂ and a C₂ amino alkyl is exemplified without limitation by —CH₂CH₂NH₂, —CH₂CH₂NHCH₃ and —CH₂CH₂N(CH₃)₂.

“Optionally substituted alkyl”, “optionally substituted alkenyl”, “optionally substituted alkynyl”, “optionally substituted arylalkyl”, “optionally substituted heterocycle”, “optionally substituted aryl”, “optionally substituted heteroaryl”, “optionally substituted heteroarylalkyl” and like terms as used herein, unless otherwise stated or implied by context, refer to an alkyl, alkenyl, alkynyl, arylalkyl, heterocycle, aryl, heteroaryl, heteroarylalkyl, or other substituent, moiety or group as defined or disclosed herein wherein hydrogen atom(s) of that substituent, moiety or group has been optionally replaced with different moiety(ies) or group(s), or wherein an alicyclic carbon chain that comprise one of those substituents, moiety or group is interrupted by replacing carbon atom(s) of that chain with different moiety(ies) or group(s). In some aspects, an alkene functional group replaces two contiguous sp³ carbon atoms of an alkyl substituent, provided that the radical carbon of the alkyl moiety is not replaced, so that the optionally substituted alkyl becomes an unsaturated alkyl substituent.

Optional substituents replacing hydrogen(s) in any one of the foregoing substituents, moieties, or groups is independently selected from the group consisting of C₆-C₂₄ aryl, C₅-C₂₄ heteroaryl, hydroxyl, C₁-C₂₀ alkoxy, C₆-C₂₄ aryloxy, cyano, halogen, nitro, C₁-C₂₀ fluoroalkoxy, and amino, which encompasses —NH₂ and mono-, di-, and tri-substituted amino groups, and the protected derivatives thereof, or is selected from the group consisting of —X, —OR′, —SR′, —NH₂, —N(R′)(R^{op}), —N(R^{op})₃, ═NR′, —CX₃, —CN, —NO₂, —NR′C(═O)H, —NR′C(═O)R^{op}, —NR′C(═O)R^{op}, —C(═O)R′, —C(═O)NH₂, —C(═O)N(R′)R^{op}, —S(═O)₂R^{op}, —S(═O)₂NH₂, —S(═O)₂N(R′)R^{op}, —S(═O)₂NH₂, —S(═O)₂N(R′)R^{op}, —S(═O)₂OR′, —S(═O)R^{op}, —OP(═O)(OR′)(OR^{op}), —OP(OH)₃, —P(═O)(OR′)(OR^{op}), —PO₃H₂, —C(═O)R′, —C(═S)R^{op}, —CO₂R′, —C(═S)OR^{op}, —C(═O)SR′, —C(═S)SR′, —C(═S)NH₂, —C(═S)N(R′)(R^{op})₂, —C(═NR′)NH₂, —C(═NR′)N(R′)R^{op}, and salts thereof, wherein each X is independently selected from the group consisting of halogens: —F, —Cl, —Br, and —I; and wherein each R^{op} is independently selected from the group consisting of C₁-C₂₀ alkyl, C₂-C₂₀ alkenyl, C₂-C₂₀ alkynyl, C₆-C₂₄ aryl, C₃-C₂₄ heterocyclyl, C₅-C₂₄ heteroaryl, a protecting group, and a prodrug moiety or two of R^{op} together with the heteroatom to which they are attached defines a C₃-C₂₄ heterocyclyl; and R′ is hydrogen or R^{op}, wherein R^{op} is selected from the group consisting of C₁-C₂₀ alkyl, C₆-C₂₄ aryl, C₃-C₂₄ heterocyclyl, C₅-C₂₄ heteroaryl, and a protecting group.

Typically, optional substituents that are present are selected from the group consisting of —X, —OH, —OR^{op}, —SH, —SR^{op}, —NH₂, —NH(R^{op}), —NR′(R^{op})₂, —N(R^{op})₃, ═NH, ═NR^{op}, —CX₃, —CN, —NO₂, —NR′C(═O)H, NR′C(═O)R^{op}, —CO₂H, —C(═O)H, —C(═O)R^{op}, —C(═O)NH₂, —C(═O)NR′R^{op}, —S(═O)₂R^{op}, —S(═O)₂NH₂, —S(═O)₂N(R′)R^{op}, —S(═O)₂NH₂, —S(═O)₂N(R′)(R^{op}), —S(═O)₂OR′, —S(═O)R^{op}, —C(═S)R^{op}, —C(═S)NH₂, —C(═S)N(R′)R^{op}, —C(═NR′)N(R^{op})₂, and salts thereof, wherein each X is independently selected from the group consisting of —F and —Cl, wherein R^{op} is typically selected from the group consisting of C₁-C₆ alkyl, C₆-C₁₀ aryl, C₃-C₁₀ heterocyclyl, C₅-C₁₀ heteroaryl, and a protecting group; and R′ is independently selected from the group typically consisting of hydrogen, C₁-C₆ alkyl, C₆-C₁₀ aryl, C₃-C₁₀ heterocyclyl, C₅-C₁₀ heteroaryl, and a protecting group, independently selected from R^{op}.

More typically, optional substituents that are present are selected from the group consisting of —X, —R^{op}, —OH, —OR^{op}, —NH₂, —NH(R^{op}), —N(R^{op})₂, —N(R^{op})₃, —CX₃, —NO₂, —NHC(═O)H, —NHC(═O)R^{op}, —C(═O)NH₂, —C(═O)NHR^{op}, —C(═O)N(R^{op})₂, —CO₂H, —CO₂R^{op}, —C(═O)H, —C(═O)R^{op}, —C(═O)NH₂, —C(═O)NH(R^{op}), —C(═O)N(R^{op})₂, —C(═NR′)NH₂, —C(═NR′)NH(R^{op}), —C(═NR′)N(R^{op})₂, a protecting group and salts thereof, wherein each X is —F, wherein R^{op} is independently selected from the group consisting of C₁-C₆ alkyl, C₆-C₁₀ aryl, C₅-C₁₀ heteroaryl and a protecting group; and R′ is selected from the group consisting of hydrogen, C₁-C₆ alkyl and a protecting group, independently selected from R^{op}.

In some aspects, an optional alkyl substituent that is present is selected from the group consisting of —NH₂, —NH(R^{op}), —N(R^{op})₂, —N(R^{op})₃, —C(═NR′)NH₂, —C(═NR′)NH(R^{op}), and —C(═NR′)N(R^{op})₂, wherein R′ and R^{op} is as defined for any one of the R′ or R^{op} groups above. In some of those aspects, the R′ and/or R^{op} substituents together with the nitrogen atom to which they are attached provide for the basic functional group of a Basic Unit (BU), as when R^{op} is independently selected from the group consisting of hydrogen and C₁-C₆ alkyl. Alkylene, carbocyclyl, carbocyclo, aryl, arylene, heteroalkyl, heteroalkylene, heterocyclyl, heterocyclo, heteroaryl, and heteroarylene groups as described above are similarly substituted or are unsubstituted, with exceptions, if any, described in the definitions of these moieties.

Other optional substituents replace a carbon atom in the acyclic carbon chain of an alkyl or alkylene moiety, group or substituent to provide for a C₃-C₁₂ heteroalkyl or C₃-C₁₂ heteroalkylene and for that purpose is typically selected from the group consisting of —O—, —C(═O)—, —C(═O)O—, —S—, —S(═O)—, —S(═O)₂—, —NH—, —NHC(═O)—, —C(═O)NH—, S(═O)₂NH—, —NHS(═O)₂—, —OC(═O)NH—, and —NHC(═O)O, optionally substituted in which —NH— is an optionally substituted heteroatom moiety by replacement of its hydrogen atom by an independently selected substituent from a group previously described for an —NH— optional substituent.

“Optionally substituted heteroatom”, as the term is used herein by itself or in combination with another term, unless otherwise stated or implied by context, refers to a heteroatom or heteroatom moiety within a functional group or other organic moiety in which the heteroatom is not further substituted or is substituted by any one of the aforementioned moieties having a monovalent carbon atom including, but not limited to alkyl, cycloalkyl, alkenyl, aryl, heterocyclyl, heteroaryl, heteroalkyl and (hetero)arylalkyl-or is oxidized by substitution with one or two ═O substituents. In some aspects, “optionally substituted heteroatom” refers an aromatic or non-aromatic —NH— moiety that is unsubstituted or in which the hydrogen atom is replaced by any one of the aforementioned substituents. In other aspects, “optionally substituted heteroatom” refers to an aromatic skeletal nitrogen atom of a heteroaryl in which an electron of that heteroatom is replaced by any one of the aforementioned substituents. For encompassing both of those aspects, the nitrogen heteroatom is sometime referred to as an optionally substituted N/NH.

Therefore, in some aspects, an optional substituent of a nitrogen atom that is present is selected from the group consisting of C₁-C₂₀ alkyl, C₂-C₂₀ alkenyl, C₂-C₂₀ alkynyl, C₆-C₂₄ aryl, C₅-C₂₄ heteroaryl, (C₆-C₂₄ aryl)-C₁-C₂₀ alkyl-, and (C₅-C₂₄ heteroaryl)-C₁-C₂₀ alkyl-, optionally substituted, as those terms are defined herein. In other aspects, optional substituents of a nitrogen atom that are present are independently selected from the group consisting of C₁-C₁₂ alkyl, C₂-C₁₂ alkenyl, C₂-C₁₂ alkynyl, C₆-C₂₄ aryl, C₅-C₂₄ heteroaryl, (C₆-C₂₄ aryl)-C₁-C₁₂ alkyl-, and (C₅-C₂₄ heteroaryl)-C₁-C₁₂ alkyl-, optionally substituted, from the group consisting of C₁-C₈ alkyl, C₂-C₈ alkenyl, C₂-C₈ alkynyl, C₆-C₁₀ aryl, C₅-C₁₀ heteroaryl, (C₆-C₁₀ aryl)-C₁-C₈ alkyl-, and (C₅-C₁₀ heteroaryl)-C₁-C₈alkyl, or from the group consisting of C₁-C₆ alkyl, C₂-C₆ alkenyl, C₂-C₆ alkynyl, C₆-C₁₀ aryl, C₅-C₁₀ heteroaryl, (C₆-C₁₀ aryl)-C₁-C₆ alkyl-, and (C₅-C₁₀ heteroaryl)-C₁-C₆ alkyl-.

When the optionally substituted nitrogen atom is the point of covalent attachment of a Peptide Cleavable Unit to a PAB or PAB-type moiety of a self-immolative Spacer Unit, sometimes designated as J, an optional substituent of that nitrogen atom when present is limited to one having a monovalent sp³ carbon atom attached thereto that does not adversely impact the electron donating ability of the nitrogen atom, as compared to the unsubstituted nitrogen atom, once its electron donating ability is restored on cleavage of the Cleavable Unit, so as to allow for self-immolation to occur for release of the Drug Unit as free drug.

“O-linked moiety”, as the term is used herein by itself or in combination with another term, unless otherwise stated or implied by context, refers to a moiety, group or substituent that is attached to a Markush structure or another organic moiety with which it is associated directly through an oxygen atom of the O-linked moiety. A monovalent O— linked moiety has that attachment through a monovalent oxygen and is typically —OH, —OC(═O)R^{b} (acyloxy), wherein R^{b} is —H, optionally substituted saturated C₁-C₂₀ alkyl, optionally substituted unsaturated C₁-C₂₀ alkyl, optionally substituted C₃-C₂₀ cycloalkyl, wherein the cycloalkyl moiety is saturated or partially unsaturated, optionally substituted C₃-C₂₀ alkenyl, optionally substituted C₂-C₂₀ alkynyl, optionally substituted C₆-C₂₄ aryl, optionally substituted C₅-C₂₄ heteroaryl or optionally substituted C₃-C₂₄ heterocyclyl, or R^{b} is optionally substituted C₁-C₁₂ alkyl, optionally substituted C₃-C₁₂ cycloalkyl, optionally substituted C₃-C₁₂ alkenyl or optionally substituted C₂-C₁₂ alkynyl, and wherein an monovalent O-linked moiety further encompasses ether groups which are C₁-C₁₂ alkyloxy (i.e., C₁-C₁₂ aliphatic ether) moieties, optionally substituted, wherein the alkyl moiety is saturated or unsaturated.

In other aspects, a monovalent O-linked moiety is a monovalent moiety selected from the group consisting of optionally substituted phenoxy, optionally substituted C₁-C₈ alkyloxy (i.e., C₁-C₈ aliphatic ether) and —OC(═O)R^{b}, wherein R^{b} is optionally substituted C₁-C₈ alkyl, which is typically saturated or is an unsaturated C₃-C₈ alkyl, optionally substituted.

In still other aspects, an O-linked moiety is a monovalent moiety selected from the group consisting of —OH, and saturated C₁-C₆ alkyl ether, unsaturated C₃-C₆ alkyl ether, optionally substituted, and —OC(═O)R^{b}, wherein R^{b} is typically C₁-C₆ saturated alkyl, C₃-C₆ unsaturated alkyl, C₃-C₆ cycloalkyl, C₂-C₆ alkenyl, or phenyl, optionally substituted, or is selected from that group excluding —OH and/or phenyl, or R^{b} is a monovalent moiety selected from the group consisting of C₁-C₆ saturated alkyl, C₃-C₆ unsaturated alkyl and C₂-C₆ alkenyl, optionally substituted, or a monovalent O-linked moiety is an unsubstituted O-linked substituent selected from the group consisting of saturated C₁-C₆ alkyl ether, unsaturated C₃-C₆ alkyl ether, and —OC(═O)R^{b}, wherein R^{b} is an unsubstituted, saturated C₁-C₆ alkyl or unsubstituted, unsaturated C₃-C₆ alkyl.

Other exemplary O-linked substituents are provided by definitions for carbamate, ether and carbonate as disclosed herein in which the monovalent oxygen atom of the carbamate, ether or carbonate functional group is bonded to the Markush structure or other organic moiety with which it is associated.

In other aspects, an O-linked moiety to carbon is divalent and encompasses ═O and —X—(CH₂)ₙ—Y—, wherein X and Y independently are S and O and subscript n is 2 or 3, to form a spiro ring system with the carbon to which X and Y are both attached.

“Halogen” as the term is used herein by itself or in combination with another term, unless otherwise stated or implied by context, refers to fluorine, chlorine, bromine or iodine and is typically —F or —Cl.

“Protecting group” as the term is used herein by itself or in combination with another term, unless otherwise stated or implied by context, refers to a moiety that prevents or substantially reduces the ability of the atom or functional group to which it is linked from participating in unwanted reactions. Typical protecting groups for atoms or functional groups are given in Greene (1999), “Protective groups in organic synthesis, 3^{rd}ed.”, Wiley Interscience. Protecting groups for heteroatoms such as oxygen, sulfur and nitrogen are sometime used to minimize or avoid their unwanted reactions with electrophilic compounds. Other times the protecting group is used to reduce or eliminate the nucleophilicity and/or basicity of the unprotected heteroatom. Non-limiting examples of protected oxygen are given by —OR^{PR}, wherein R^{PR} is a protecting group for hydroxyl, wherein hydroxyl is typically protected as an ester (e.g., acetate, propionate or benzoate). Other protecting groups for hydroxyl avoid its interference with the nucleophilicity of organometallic reagents or other highly basic reagents, for which purpose hydroxyl is typically protected as an ether, including without limitation alkyl or heterocyclyl ethers, (e.g., methyl or tetrahydropyranyl ethers), alkoxymethyl ethers (e.g., methoxymethyl or ethoxymethyl ethers), optionally substituted aryl ethers, and silyl ethers (e.g., trimethylsilyl (TMS), triethylsilyl (TES), tert-butyldiphenylsilyl (TBDPS), tert-butyldimethylsilyl (TBS/TBDMS), triisopropylsilyl (TIPS) and [2-(trimethylsilyl)ethoxy]-methylsilyl (SEM)). Nitrogen protecting groups include those for primary or secondary amines as in —NHR^{PR} or —N(R^{PR})₂, wherein least one of R^{PR} is a nitrogen atom protecting group or both R^{PR} together define a nitrogen atom protecting group.

A protecting group is a suitable for protecting when it is capable of preventing or substantially avoiding unwanted side-reactions and/or premature loss of the protecting group under reaction conditions required to effect desired chemical transformation(s) elsewhere in the molecule and during purification of the newly formed molecule when desired, and can be removed under conditions that do not adversely affect the structure or stereochemical integrity of that newly formed molecule. In some aspects, suitable protecting groups are those previously described for protecting functional groups. In other aspects, a suitable protecting group is a protecting group used in peptide coupling reactions. For example, a suitable protecting group for the basic nitrogen atom of an acyclic or cyclic Basic Unit is an acid-labile carbamate protecting group such as t-butyloxycarbonyl (BOC).

“Ester” as the term is used herein by itself or in combination with another term, unless otherwise stated or implied by context, refers to a substituent, moiety or group having the structure of —C(═O)—O— to define an ester functional group in which the carbonyl carbon atom of that structure is not directly connected to another heteroatom but is directly connected to hydrogen or another carbon atom of an organic moiety with which it is associated, and wherein the monovalent oxygen atom is either attached to the same organic moiety at a different carbon atom to provide a lactone or to a Markush structure or to some other organic moiety. Typically, esters in addition to the ester functional group comprise or consist of an organic moiety containing 1 to 50 carbon atoms, typically 1 to 20 carbon atoms or more typically 1 to 8, 1 to 6 or 1 to 4 carbon atoms and 0 to 10 independently selected heteroatoms (e.g., O, S, N, P, Si, but usually O, S and N), typically 0 to 2 heteroatoms, wherein the organic moiety is bonded to the —C(═O)—O— structure (i.e., through the ester functional group) so as to provide structure having the formula of organic moiety-C(═O)—O— or —C(═O)—O— organic moiety.

When an ester is a substituent or variable group of a Markush structure or other organic moiety with which it is associated, that substituent is bonded to the structure or other organic moiety through the monovalent oxygen atom of the ester functional group so that it is an monovalent O-linked substituent, which sometimes referred to as an acyloxy. In such instances, the organic moiety attached to the carbonyl carbon of the ester functional group typically is a C₁-C₂₀ alkyl, C₂-C₂₀ alkenyl, C₂-C₂₀ alkynyl, C₆-C₂₄ aryl, C₅-C₂₄ heteroaryl, C₃-C₂₄ heterocyclyl or is a substituted derivative of any one of these, e.g., having 1, 2, 3 or 4 substituents, more typically is C₁-C₁₂ alkyl, C₂-C₁₂ alkenyl, C₂-C₁₂ alkynyl, C₆-C₁₀ aryl, C₅-C₁₀ heteroaryl, C₃-C₁₀ heterocyclyl or a substituted derivative of one any of these, e.g., having 1, 2, or 3 substituents or is C₁-C₈ alkyl, C₂-C₈ alkenyl, C₂-C₈ alkynyl, or phenyl or a substituted derivative of any one of these, e.g., having 1 or 2 substituents, wherein each independently selected substituent is as defined herein for optional alkyl substituents, or is unsubstituted C₁-C₆ alkyl or unsubstituted C₂-C₆ alkenyl.

Exemplary esters by way of example and not limitation, are acetate, propionate, isopropionate, isobutyrate, butyrate, valerate, isovalerate, caproate, isocaproate, hexanoate, heptanoate, octanoate, phenylacetate esters and benzoate esters or have the structure of —OC(═O)R^{b} in which R^{b} is as defined for acyloxy O-linked substituents and is typically selected from the group consisting of methyl, ethyl, propyl, iso-propyl, 2-methyl-prop-1-yl, 2,2-dimethyl-prop-1-yl, prop-2-ene-1-yl, and vinyl.

“Ether” as the term is used herein by itself or in combination with another term, unless otherwise stated or implied by context, refers to an organic moiety, group or substituent that comprises 1, 2, 3, 4 or more —O— (i.e., oxy) moieties that are not bonded to carbonyl moiety(ies), typically 1 or 2, wherein no two —O— moieties are immediately adjacent (i.e., directly attached) to each other. Typically, an ether contains the formula of —O-organic moiety wherein organic moiety is as described for an organic moiety bonded to an ester functional group or is as described herein for an optionally substituted alkyl group. When ether is recited as a substituent or variable group of a Markush structure or other organic moiety with which it is associated, the oxygen of the ether functional group is attached to a Markush formula with which it is associated and is sometimes designated as an “alkoxy” group, which is an exemplary O-linked substituent. In some aspects, an ether O-linked substituent is a C₁-C₂₀ alkoxy or a C₁-C₁₂ alkoxy, optionally substituted with 1, 2, 3 or 4 substituents, typically 1, 2 or 3, and in other aspects is a C₁-C₈ alkoxy or C₁-C₆ alkoxy, optionally substituted with 1 or 2 substituents, wherein each independently selected substituent is as defined herein for optional alkyl substituents, and in still other aspects an ether O-linked substituent is an unsubstituted, saturated or unsaturated C₁-C₄ alkoxy such as, by way of example and not limitation, methoxy, ethoxy, propoxy, iso-propoxy, butoxy and allyloxy (i.e., —OCH₂CH═CH₂).

“Amide” as the term is used herein by itself or in combination with another term, unless otherwise stated or implied by context, refers to a moiety having an optionally substituted functional group having the structure of R—C(═O)N(R^{c})— or —C(═O)N(R^{c})₂ to which no other heteroatom is directly attached to the carbonyl carbon and wherein each R^{c} is independently hydrogen, a protecting group or an independently selected organic moiety, and R is hydrogen or an organic moiety, wherein organic moiety, independently selected from R^{c}, is as described herein for an organic moiety bonded to an ester functional group or is as described herein for an optionally substituted alkyl group. When an amide is recited as a substituent or variable group of a Markush structure or other organic moiety with which it is associated, the amide nitrogen atom or carbonyl carbon atom of the amide functional group is bonded to that structure or other organic moiety. Amides are typically prepared by condensing an acid halide, such an acid chloride, with a molecule containing a primary or secondary amine. Alternatively, amide coupling reactions well-known in the art of peptide synthesis, which in some aspects proceeds through an activated ester of a carboxylic acid-containing molecule, are used. Exemplary preparations of amide bonds through peptide coupling methods are provided in Benoiton (2006) “Chemistry of peptide synthesis”, CRC Press; Bodansky (1988) “Peptide synthesis: A practical textbook” Springer-Verlag; Frinkin, M. et al. “Peptide Synthesis” Ann. Rev. Biochem. (1974) 43: 419-443. Reagents used in the preparation of activated carboxylic acids is provided in Han, et al. “Recent development of peptide coupling agents in organic synthesis” Tet. (2004) 60: 2447-2476.

Thus, in some aspects, amides are be prepared by reacting a carboxylic acid with an amine in the presence of a coupling agent. As used herein, “in the presence of a coupling agent” includes contacting the carboxylic acid with the coupling agent thereby converting the acid to its activated derivative, such as an activated ester or a mixed anhydride, with or without isolation of the resulting activated derivative of the acid, followed by or simultaneously contacting the resulting activated derivative with the amine. In some instances, the activated derivative is prepared in situ. In other instances, the activated derivative may be isolated to remove any undesired impurities.

“Carbonate” as the term is used herein by itself or in combination with another term, unless otherwise stated or implied by context, means a substituent, moiety or group that contains a functional group having the structure —O—C(═O)—O— which defines a carbonate functional group. Typically, carbonate groups as used herein are comprised of an organic moiety bonded to the —O—C(═O)—O— structure, wherein the organic moiety is as described herein for an organic moiety bonded to an ester functional group, e.g., organic moiety-O—C(═O)—O—. When carbonate is recited as a substituent or variable group of a Markush structure or other organic moiety with which it is associated, one of the monovalent oxygen atoms of the carbonate functional group is attached to that structure or organic moiety and the other is bonded to a carbon atom of another organic moiety as previously described for an organic moiety bonded to an ester functional group or is as described herein for an optionally substituted alkyl group. In such instances, carbonate is an exemplary O-linked substituent.

“Carbamate” as the term is used herein by itself or in combination with another term, unless otherwise stated or implied by context, means a substituent, moiety or group that contains an optionally substituted carbamate functional group structure represented by —O—C(═O)N(R^{c})— or —O—C(═O)N(R^{c})₂, or —O—C(═O)NH(optionally substituted alkyl)- or —O—C(═O)N(optionally substituted alkyl)₂ in which the independently selected optionally substituted alkyl(s) are exemplary carbamate functional group substituents, and typically are C₁-C₁₂ alkyl or C₁-C₈ alkyl, optionally substituted, more typically C₁-C₆ alkyl or C₁-C4 alkyl, optionally substituted, wherein each R^{c} is independently selected, wherein independently selected R^{c} is hydrogen, a protecting group or an organic moiety, wherein the organic moiety is as described herein for an organic moiety bonded to an ester functional group or is as described herein for an optionally substituted alkyl group. Typically, carbamate groups are additionally comprised of an organic moiety, independently selected from R^{c}, wherein the organic moiety is as described herein for an organic moiety bonded to an ester functional group, bonded through the —O—C(═O)—N(R^{c})— structure, wherein the resulting structure has the formula of organic moiety-O—C(═O)—N(R^{c})— or —O—C(═O)—N(R^{c})-organic moiety. When carbamate is recited as a substituent or variable group of a Markush structure or other organic moiety with which it is associated, the monovalent oxygen (O-linked) or nitrogen (N-linked) of the carbamate functional group is attached to a Markush formula with which it is associated. The linkage of the carbamate substituent is either explicitly stated (N- or O-linked) or implicit in the context to which this substituent is referred. O-linked carbamates described herein are exemplary monovalent O-linked substituents.

“Ligand Drug Conjugate”, as the term is used herein, unless otherwise stated or implied by context, refers to a construct comprised of a Ligand Unit (L) incorporating or corresponding in structure to a targeting agent and a Drug Unit (D) incorporating or corresponding in structure to free drug, wherein L and D are bonded to each other through a Linker Unit (LU), wherein the Ligand Drug Conjugate is capable of selective binding to a targeted moiety of a targeted cell. The term Ligand Drug Conjugate (LDC) in one aspect refers to a plurality (i.e., composition) of individual Conjugate compounds having the same or differing to some extent by the number of auristatin Drug Units conjugated to each Ligand Unit and/or the location on the Ligand Unit to which the Drug Units are conjugated. In some aspects the term refers to a collection (i.e., population or plurality) of Conjugate compounds having essentially the same Ligand Unit, and the same Drug Unit and Linker Unit, which in some aspects have variable loading and/or distribution of auristatin drug linker moieties attached to each antibody residue (as, for example, when the number of Drug Units of any two Ligand Drug Conjugate compounds in a plurality of such compounds is the same but the locations of their sites of attachment to the Ligand Unit are different). In those instances, a Ligand Drug Conjugate is described by the averaged drug loading of the Conjugate compounds.

The average number Drug Units per Ligand Unit in a Ligand Drug Conjugate composition is an averaged number for a population of Ligand Drug Conjugate compounds, sometimes designated by subscript p, which in some aspects reflects a distribution of these compounds differing primarily by the number of conjugated Drug Units to the Ligand Unit and/or by their location on the Ligand Unit to which they are conjugated.

A Ligand Drug Conjugate compound, by itself or within a Ligand Drug Conjugate composition, of the present invention is typically represented by the structure of Formula 1:

L-[LU-(D′)]ₚ′ (1)
or a salt thereof, which in some aspects is a pharmaceutically acceptable salt, wherein L is a Ligand Unit; LU is a Linker Unit; subscript p′ is a integer ranging from 1 to 24; and D′ represents from 1 to 4 Drug Units. In some aspects a Ligand Unit incorporates or corresponds in structure to an antibody or an antigen-binding fragment thereof thereby defining an antibody Ligand Unit. In those aspects, an antibody Ligand Unit is capable of selective binding to an antigen of a targeted cell for subsequent release of free drug, wherein the targeted antigen in one aspect is a cancer cell antigen selectively recognized by an antibody Ligand Unit and is capable of internalization into said cancer cell along with the bound ADC compound upon said binding for initiating intracellular release of free drug subsequent to said internalization. In any of those aspects each drug linker moiety in a Ligand Drug Conjugate compound has the structure of Formula 1A:

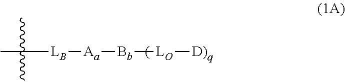

or a salt thereof, which is some aspects is a pharmaceutically acceptable salt, wherein D in each drug linker moiety is the Drug Unit; the wavy line indicates covalent binding to L; L_{B} is an ligand covalent binding moiety; A is a first optional Stretcher Unit; subscript a is 0 or 1 indicating the absence of presence of A, respectively; B is an optional Branching Unit; subscript b is 0 or 1, indicating the absence of presence of B, respectively; L_{O} is an secondary linker moiety; D is the Drug Unit, wherein the Drug Unit corresponds in structure to free drug; and subscript q is an integer ranging from 1 to 4,
wherein a Ligand Drug Conjugate composition comprised of a distribution or collection of Ligand Drug Conjugate compounds is represented by structure of Formula 1 in which subscript p′ is replaced by subscript p, wherein subscript p is a number ranging from about 2 to about 24.

“Ligand Unit” as the term is used herein, unless otherwise stated or implied by context, refers to a targeting moiety of a Ligand Drug Conjugate composition or compound that is capable of binding selectively to its cognate targeted moiety and incorporates or corresponds to the structure of a targeting agent. A Ligand Unit (L) includes without limitation those from receptor ligands, antibodies to cell-surface antigens, and transporter substrates. In some aspects, the receptor, antigen or transporter to be bound by a Conjugate compound of a Ligand Drug Conjugate composition is present in greater abundance on abnormal cells in contrast to normal cells so as to effect a desired improvement in tolerability or reduce the potential occurrence or severity of one or more adverse events that are associated with administration of a drug in unconjugated form. In other aspects, the receptor, antigen or transporter to be bound to the Ligand Unit of a Ligand Drug Conjugate compound is present in greater abundance on normal cells in the vicinity of abnormal cells in contrast to normal cells that are distant from the site of the abnormal cells, so as to selectively expose the nearby abnormal cells to free drug. Various aspects of Ligand Units, including antibody Ligand Units, are further described by embodiments of the invention.

“Targeting agent” as used herein, unless otherwise stated or implied by context, refers to an agent that is capable of selective binding to a targeted moiety and which substantially retains that capability when it is incorporated into a Ligand Drug Conjugate as a Ligand Unit. The Ligand Unit of a Ligand Drug Conjugate therefore corresponds in structure to the targeting agent so that the Ligand Unit is the targeting moiety of the Conjugate. In some aspects, the targeting agent is an antibody or fragment thereof that selectively binds to an accessible antigen that is characteristic of an abnormal cell or is present in higher copy number in comparison to normal cells or is an accessible antigen that is particular to the surrounding environment in which these cells are found to an extent that achieves an improved tolerability in comparison to administration of free drug. In other aspects, the targeting agent is a receptor ligand that selectively binds to an accessible receptor characteristic of, or in greater abundance on, abnormal cells, or to an accessible receptor on nominally normal cells that are peculiar to environment surrounding the abnormal cells. Typically, a targeting agent is an antibody as defined herein that binds selectively to a targeted moiety of an abnormal mammalian cell, more typically a targeted moiety of an abnormal human cell.

“Targeted moiety” as defined herein is a moiety to be selectively recognized by a targeting agent or the targeting moiety of a Ligand Drug Conjugate, which is its Ligand Unit that incorporates or corresponds in structure to the targeting agent. In some aspects, a targeted moiety is present on, within, or in the vicinity of abnormal cells and is typically present in greater abundance or copy number on these cells in comparison to normal cells or to the environment of normal cells distant from the site of the abnormal cells so as to provide for improved tolerability relative to administration of free drug or reduces the potential for one or more adverse events from that administration. In some aspects, the targeted moiety is an antigen accessible to selective binding by an antibody, which is an exemplary targeting agent that that been incorporated into or corresponds in structure to an antibody Ligand Unit in an Antibody Drug Conjugate composition or compound thereof. In other aspects, the targeting moiety is that of a ligand for an extracellularly accessible cell membrane receptor, which in some aspects is internalized upon binding of the cognate targeting moiety by the Ligand Unit of a Ligand Drug Conjugate compound, wherein the Ligand Unit incorporates or corresponds in structure to the receptor ligand, and in other aspects the receptor is capable of passive or facilitative transport of the Ligand Drug Conjugate compound subsequent to its binding to the cell-surface receptor. In some aspects, the targeted moiety is present on abnormal mammalian cells or on mammalian cells characteristic of the environment of such abnormal cells. In some of those aspects, the targeted moiety is an antigen of an abnormal mammalian cell, more typically a targeted moiety of an abnormal human cell.

“Targeted cells”, as the term is used herein, unless otherwise stated or implied by context, are the intended cells to which Ligand Drug Conjugate is designed to interact in order to inhibit the proliferation or other unwanted activity of abnormal cells. In some aspects, the targeted cells are hyper-proliferating cells or hyper-activated immune cells, which are exemplary abnormal cells. Typically, those abnormal cells are mammalian cells and more typically are human cells. In other aspects, the targeted cells are within the vicinity of the abnormal cells so that action of the Ligand Drug Conjugate on the nearby cells has an intended effect on the abnormal cells. For example, the nearby cells may be epithelial cells that are characteristic of the abnormal vasculature of a tumor. Targeting of those vascular cells by a Ligand Drug Conjugate compound will either have a cytotoxic or a cytostatic effect on these cells, which is believed to result in inhibition of nutrient delivery to the nearby abnormal cells of the tumor. Such inhibition indirectly has a cytotoxic or cytostatic effect on the abnormal cells and may also have a direct cytotoxic or cytostatic effect on the nearby abnormal cells by releasing its drug payload in the vicinity of these cells.

“Antibody Drug Conjugate”, as the term is used herein, unless otherwise stated or implied by context, is a subset of Ligand Drug Conjugates of Formula 1 and therefore refers to a construct comprised of an antibody Ligand Unit (L) incorporating or corresponding to an antibody or antigen-binding fragment thereof, and a Drug Unit (D) incorporating or corresponding in structure to a biologically active compound, often referred to as free drug, wherein L and D are bonded to each other through a Linker Unit (LU), wherein the Antibody Drug Conjugate is capable of selective binding to a targeted antigen of a targeted cell, which in some aspects is an antigen of an abnormal cell such as a cancer cell, through its targeting antibody Ligand Unit.

The term Antibody Drug Conjugate (ADC) in one aspect refers to a plurality (i.e., composition) of individual Conjugate compounds having the same or differing to some extent by the number of Drug Units conjugated to each antibody Ligand Unit and/or the locations on the antibody Ligand Unit to which the Drug Units are conjugated. In some aspects the term refers to a distribution or collection (i.e., population or plurality) of Conjugate compounds having the same drug-linker moieties and antibody Ligand Units, allowing for mutational amino acid variations and varying glycosylation patterns as described herein occurring during production of antibodies from cell culture, which in some aspects have variable loading and/or distribution of the drug linker moieties attached to each antibody residue (as, for example, when the number of Drug Units of any two Antibody Drug Conjugate compounds in a plurality of such compounds is the same but the locations of their sites of attachment of the drug linker moieties to the targeting antibody Ligand Unit differ). In those instances, an Antibody Drug Conjugate is described by the averaged drug loading of the Conjugate compounds.

The average number Drug Units per antibody Ligand Unit, or antigen-binding fragment thereof, in an Antibody Drug Conjugate composition having intact drug linker moieties in which the Linker Units are unbranched is an averaged number for a population of Antibody Drug Conjugate compounds and in some aspects reflects a distribution of these compounds differing primarily by the number of conjugated Drug Units to the antibody Ligand Unit and/or by their location. When the Linker Units are branched then the average number reflects the distribution of drug linker moieties for a population of Antibody Drug Conjugate compounds. In either context p is a number ranging from about 2 to about 24 or about 2 to about 20 and is typically about 2, about 4, or about 10 or about 8. In other contexts, p represents the number of Drug Units that are covalently bonded to a single antibody Ligand Unit of an Antibody Drug Conjugate within a population of Antibody Drug Conjugate compounds in which the compounds of that population in some aspects primarily differ by the number and/or locations of the Drug Units or drug linker moieties. In that context p is designated as p′ and is an integer ranging from 1 to 24 or from 1 to 20, typically from 1 to 12 or 1 to 10, and more typically from 1 to 8. In other aspects, essentially all of the available reactive functional groups of an antibody targeting agent form covalent bonds to drug linker moieties to provide an antibody Ligand Unit attached to the maximum number of drug linker moieties, so that the p value of the Antibody Drug Conjugate composition is the same or nearly the same as each of the p′ values for each of the Antibody Drug Conjugate compounds of the composition, so that only minor amounts of Antibody Drug Conjugate compounds with lower p′ values are present, if at all, as detected using an appropriate chromatographic method, such as electrophoresis, HIC, reverse phase HPLC or size-exclusion chromatography.

The average number of Drug Units or drug linker moieties per antibody Ligand Unit in a preparation from a conjugation reaction in some aspects is characterized by conventional chromatographic means as described above in conjunction with mass spectroscopy detection. In other aspects, the quantitative distribution of conjugate compounds in terms of p′ values are determined. In those instances, separation, purification, and characterization of homogeneous Antibody Drug Conjugate compounds in which p′ is a certain value from an Antibody Drug Conjugate composition from those with other Drug Unit or drug linker moiety loadings is achievable by means such as an aforementioned chromatographic method.

“Drug Linker compound” as the term is used herein, unless otherwise stated or implied by context, refers to a compound having an Drug Unit covalently attached to a Linker Unit precursor (LU′), wherein LU′ is comprised of L_{B}′ sometimes referred to as a ligand covalent binding precursor (L_{B}′) moiety because that moiety contains a reactive or activatable functional group, wherein that reactive functional group or activateable functional group subsequent to activation is capable of reacting with a targeting agent to form a covalent bond between a ligand covalent binding moiety (L_{B}) and a Ligand Unit, thus providing a drug linker moiety of Formula 1A for an Ligand Drug Conjugate compound of Formula 1, in particular a covalent bond to an antibody Ligand Unit, which incorporates or corresponds in structure to an antibody,

A Drug Linker compound of the present invention typically has the general formula of Formula I:

LU′-(D′) (I)
or a salt thereof, which in some aspects is a pharmaceutically acceptable salt, wherein LU′ is a LU precursor; and D′ represents from 1 to 4 Drug Units, wherein the Drug Linker compound is further defined by the structure of Formula IA:

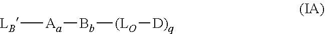

wherein L_{B}′ is comprised of the reactive or activateable functional group and the remaining variable groups are as defined for Formula 1A.

“Cytotoxic agent” as the term is used herein, unless otherwise stated or implied by context, is a compound capable of inducing cell death or inhibiting the proliferation or continued survival of cells, which typically are abnormal mammalian cells, in vitro or in vivo. Cytostatic agents, which primarily exert a therapeutic effect by inhibiting proliferation of abnormal cells and not by direct cell killing, are encompassed by the definition of cytotoxic agent. In some aspects, a cytotoxic agent is the free drug resulting from release of a Drug Unit from an Antibody Drug Conjugate.

“Drug Unit” as the phrase is used herein, unless otherwise stated or implied by context, refers to a residue of a drug covalently attached to a Linker Unit (LU) in a drug linker moiety of a Ligand Drug Conjugate (LDC) or is covalently attached to the Linker Unit precursor (LU′) of a Drug Linker compound and is releasable from the drug linker moiety or Drug linker compound as free drug. The free drug may be directly incorporated into a Drug Unit, or a component of the free drug may be covalently attached to LU or LU′ or an intermediate thereof followed by further elaboration to complete the structure of the Drug Unit. The term “Drug,” as used herein alone or in connection with another term (such as “Drug Unit”), is not intended to imply that a compound is approved, approvable, or intended to be approved by a government agency for a medical or veterinary treatment.

In some aspects the free drug incorporated into a Drug Unit is a cytotoxic compound, typically one that has a secondary aliphatic amine as the conjugation handle, and includes auristatin compounds as defined herein.

“Auristatin drug”, “auristatin compound” and like terms as used herein, unless otherwise stated or implied by context, refer to a peptide-based tubulin disrupting agent having cytotoxic, cytostatic or anti-inflammatory activity that is comprised of a dolaproline and a dolaisoleucine residue or amino acid residues related thereto.

Some exemplary auristatins have the structure of D_{E} or D_{F}:

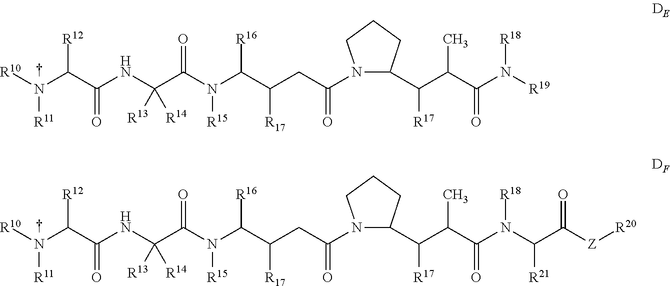

wherein Z is —O—, —S—, or —N(R¹⁹)—, and wherein R¹⁰-R²¹ are as defined in embodiments for auristatin Drug Units and the indicated nitrogen atom (†) is that of a secondary amine (e.g., one of R¹, R¹¹ is hydrogen and the other is —CH₃). In those aspects the auristatin is incorporated into a Drug Unit through a carbamate functional group comprised of that nitrogen atom. That carbamate functional group is an exemplary second Spacer Unit (Y′) and is capable of undergoing self-immolation, which is turn is attached to a PAB or PAB-type Spacer Unit (Y) so that subscript y in any one of the drug linker moieties described herein is 2.

Other exemplary auristatins include, but are not limited to AE, AFP, AEB, AEVB, MMAF, and MMAE and those further described in the embodiments of the invention. The synthesis and structure of auristatins are described in U.S. Patent Application Publication Nos. 2003-0083263, 2005-0238649 2005-0009751, 2009-0111756, and 2011-0020343; International Patent Publication No. WO 04/010957, International Patent Publication No. WO 02/088172, and U.S. Pat. Nos. 7,659,241 and 8,343,928. Their structures and methods of their syntheses disclosed therein are specifically incorporated by reference herein.

“Salt thereof” as the phrase is used herein, unless otherwise stated or implied by context, refers to a salt form of a compound (e.g., a Drug, a Drug Linker compound or a LDC compound). A salt form of a compound is of one or more internal salt forms and/or involves the inclusion of another molecule such as an acetate ion, a succinate ion or other counterion. The counterion in a salt form of a compound is typically an organic or inorganic moiety that stabilizes the charge on the parent compound. A salt form of a compound has one or more than one charged atom in its structure. In instances where multiple charged atoms are part of the salt form, multiple counter ions and/or multiple charged counter ions are present. Hence, a salt form of a compound typically has one or more charged atoms corresponding to those of the non-salt form of the compound and one or more counterions. In some aspects, the non-salt form of a compound contains at least one amino group or other basic moiety, and accordingly in the presence of an acid, an acid addition salt with the basic moiety is obtained. In other aspects, the non-salt form of a compound contains at least one carboxylic acid group or other acidic moiety, and accordingly in the presence of a base, a carboxylate or other anionic moiety is obtained.

Exemplary counteranion and countercations in compound salt forms include, but are not limited to, sulfate, trifluoroacetate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p toluenesulfonate, and pamoate (i.e., 1,1′ methylene bis-(2-hydroxy-3-naphthoate)) salts.

Selection of a salt form of a compound is dependent on properties the drug product must exhibit, including adequate aqueous solubility at various pH values, depending upon the intended route(s) of administration, crystallinity with flow characteristics and low hygroscopicity (i.e., water absorption versus relative humidity) suitable for handling and required shelf life by determining chemical and solid-state stability under accelerated conditions (i.e., for determining degradation or solid-state changes when stored at 40° C. and 75% relative humidity).

A “pharmaceutically acceptable salt” is a salt form of a compound that is suitable for administration to a subject as described herein and in some aspects includes countercations or counteranions as described by P. H. Stahl and C. G. Wermuth, editors, Handbook of Pharmaceutical Salts: Properties, Selection and Use, Weinheim/Ziirich:Wiley-VCH/VHCA, 2002.

“Antibody” as the term is used herein is used in the broadest sense, unless otherwise stated or implied by context, and specifically encompasses intact monoclonal antibodies, polyclonal antibodies, monospecific antibodies, multispecific antibodies (e.g., bispecific antibodies), and antibody fragments that exhibit the desired biological activity which requires the antibody fragment to have the requisite number of sites for attachment to the desired number of drug-linker moieties and be capable of specific and selective binding to the targeted cancer cell antigen. The native form of an antibody is a tetramer and typically consists of two identical pairs of immunoglobulin chains, each pair having one light chain and one heavy chain. In each pair, the light and heavy chain variable regions (VL and VH) are together primarily responsible for binding to an antigen. The light chain and heavy chain variable domains consist of a framework region interrupted by three hypervariable regions, also called “complementarity determining regions” or “CDRs”. In some aspects, the constant regions are recognized by and interact with the immune system (see, e.g., Janeway et al., 2001, Immunol. Biology, 5th Ed., Garland Publishing, New York) so as to exert an effector function. An antibody includes any isotype (e.g., IgG, IgE, IgM, IgD, and IgA) or subclass thereof (e.g., IgG1, IgG2, IgG3, IgG4, IgA1 and IgA2). The antibody is derivable from any suitable species. In some aspects, the antibody is of human or murine origin. Such antibodies include human, humanized or chimeric antibodies.

In some aspects, the antibody is in reduced form in which the antibody has undergone reduction of its hinge disulfide bonds. The antibody is then incorporated into an Antibody Drug Conjugate as an antibody Ligand Unit by reaction of one or more of the cysteine thiols obtained by that reduction with an appropriate electrophile of a Drug Linker compound resulting in covalent binding of a drug linker moiety to the antibody Ligand Unit or of a Linker intermediate that is further elaborated to its final form as the drug linker moiety.

“Monoclonal antibody” as used herein refers to an antibody obtained from a population of substantially homogeneous antibodies, i.e., the individual antibodies comprising the population are identical except for possible naturally occurring mutations that may be present in minor amounts and/or differences in glycosylation patterns. Monoclonal antibodies are highly specific, being directed against a single antigenic site. The modifier “monoclonal” indicates the character of the antibody as being obtained from a substantially homogeneous population of antibodies and is not to be construed as requiring production of the antibody by any particular method.

“Selective binding” and “selectively binds” as the terms are used herein, unless otherwise stated or implied by context, refers to an antibody, a fragment thereof, or an antibody Ligand Unit of an Antibody Drug Conjugate that is capable of binding in an immunologically selective and specific manner with its cognate cancer cell antigen and not with a multitude of other antigens. Typically, the antibody or antigen-binding fragment thereof binds its targeted cancer cell antigen with an affinity of at least about 1×10⁻⁷ M, and preferably about 1×10⁻⁸ M to 1×10⁻⁹ M, 1×10⁻¹⁰ M, or 1×10⁻¹¹ M and binds to that predetermined antigen with an affinity that is at least two-fold greater than its affinity for binding to a non-specific antigen (e.g., BSA, casein) other than for a closely-related antigen, wherein said affinities are substantially retained when the antibody or antigen-binding fragment thereof corresponds to or is incorporated into an Antibody Drug Conjugate as an antibody Ligand Unit.

“Antigen” as the term is used herein, unless otherwise stated or implied by context, is a moiety that is capable of specific binding by an unconjugated antibody or an antigen-binding fragment thereof or to an Antibody Drug Conjugate compound, which is comprised of an antibody Ligand Unit that incorporates or corresponds in structure to the unconjugated antibody. In some aspects, the antigen is an extracellularly accessible cell-surface protein, glycoprotein, or carbohydrate preferentially displayed by abnormal cells in comparison to normal cells distant from the site of the abnormal cells, in particular, a protein or glycoprotein. In those aspects, the cell-surface antigen is capable of internalization upon selective binding by a Conjugate compound of an Antibody Drug Conjugate composition. Subsequent to internalization, intracellular processing of a Linker Unit of an Antibody Drug Conjugate compound of the composition releases its Drug Unit as free drug. Antigens associated with hyper-proliferating cells that are cell-surface accessible to an Antibody Drug Conjugate compound include by way of example and not limitation to a cancer specific antigen as described herein.

Typically, the antigen is associated with a cancer. In some of those aspects the antigen is preferentially displayed by cancer cells in comparison to normal cells that are not localized to the abnormal cells, in particular, the cancer cells displaying the antigen are mammalian cancer cells. In other aspects, the cancer cell antigen is an extracellularly accessible antigen preferentially displayed by nearby normal cells that are peculiar to the environment of the cancer cells in comparison to normal cells distant from the site of the cancer cells. For example, the nearby cells may be epithelial cells that are characteristic of the abnormal vasculature of a tumor. Targeting of those vascular cells by an Antibody Drug Conjugate will have a cytotoxic or a cytostatic effect on these cells, which is believed to result in inhibition of nutrient delivery to the nearby cancer cells of the tumor. Such inhibition will indirectly have a cytotoxic or cytostatic effect on the cancer cells and may also have a direct cytotoxic or cytostatic effect on nearby cancer cells subsequent to release of its Drug Unit as free drug subsequent to immunological selective binding by an Antibody Drug Conjugate (ADC) compound. In either of those aspects, the cell-surface antigen is capable of internalization to allow for intracellular delivery of free drug on its release from the Conjugate into the targeted cell.

Preferred internalizable antigens are those expressed on the surface of cancer cells with a copy number of 10,000 per cell or more, 20,000 per cell or more or 40,000 per cell or more. Antigens associated with cancer cells that are cell-surface accessible to an ADC and are internalizable include an antigen expressed on Hodgkin's Lymphoma cells, particularly those of Reed-Sternberg cells, as exemplified by Karpas 299 cells and certain cancer cells of high grade lymphomas sometimes referred to a Ki-1 lymphomas. Other antigens include cancer cells of renal cell adenocarcinoma, as exemplified by 789-0 cells, cancer cells of B-cell lymphomas or leukemias, including non-Hodgkin's lymphoma, chronic lymphocytic leukemia (CLL) and acute lympholytic leukemia (ALL), as exemplified by CHO cells, cancer cells of acute myeloid leukemia (AML), as exemplified by HL-60, and certain transporter receptors that are ubiquitously expressed on these and other cancer cells.

“Linker Unit” as the term is used herein, unless otherwise stated or implied by context, refers to an organic moiety in a Ligand Drug Conjugate intervening between and covalently attached to a Drug Unit and a Ligand Unit (L), as these terms are defined herein, or is an organic moiety in a Drug Linker compound that is covalently attached to a Drug Unit and has a reactive functional group or moiety for interaction with a targeting agent to form a covalent bond between L, which incorporates or corresponds in structure to the targeting agent, and the Linker Unit (LU). As the Linker Unit in a Drug Linker is capable of forming such a bond, it is considered a precursor to a Linker Unit in a Ligand Drug Conjugate and is sometimes so indicated as LU′. A Linker Unit is comprised of a primary linker (L_{R}) and a secondary linker (L_{O}) that intervenes between L_{R} and D within a drug linker moiety of a Ligand Drug Conjugate compound or between L_{R} and D of a Drug Linker compound, which in the latter instance may be represented as L_{R}′ to explicitly indicate that is a precursor to L_{R} in a Ligand Drug Conjugate.

“Primary linker” as the term is used herein, unless otherwise stated or implied by context, refers to a required component of a Linker Unit (LU) in Ligand Drug Conjugate that is covalently attached to the Ligand Unit and the remainder of LU. One component of the primary linker (L_{R}) is a ligand covalent binding (L_{B}) moiety, which in some aspects of Ligand Drug Conjugates (LDCs) and Drug Linker compounds described herein provides for a self-stabilizing (L_{SS}) linker, thereby defining a L_{SS} primary linker, and in other aspects of LDCs provides for a self-stabilized (L_{S}) linker derivable from L_{SS}, thereby defining a L_{S} primary linker, as these terms are further described herein. The primary linker optionally contains a Branching Unit (B) and a first optional Stretcher Unit (A), dependent on the values of subscripts a and b in Formula 1A, provided that A is present when L_{R} is a L_{SS} or a L_{S} primary linker.

A L_{SS} primary linker in a LDC or Drug Linker compound is characterized by a succinimide (M²) or maleimide (M¹) moiety, respectively, in proximity to a Basic Unit, while a L_{S} primary linker in a LDC composition or compound thereof is characterized by a succinic acid amide (M³) moiety in proximity to a Basic Unit. An L_{SS} or L_{S} primary linker of the present invention is also characterized by a first optional Stretcher Unit (A) that is present and comprised of an optionally substituted C₁-C₁₂ alkylene moiety bonded to the imide nitrogen of the maleimide or succinimide ring system of M¹ or M² or the amide nitrogen of M³, wherein the alkylene moiety in some aspects is substituted by an acyclic Basic Unit and may be further substituted by optional substituents, or in other aspects is optionally substituted and incorporates a cyclic Basic Unit that is optionally substituted.

A maleimide (M¹) moiety of a ligand covalent binding precursor of a L_{SS} primary linker in a Drug Linker Compound, sometimes shown as L_{SS}′ to explicitly indicate that it is a precursor to L_{SS} in a Ligand Drug Conjugate, is capable of reacting with a sulfur atom of a reactive thiol functional group of a targeting agent resulting in a thio-substituted succinimide moiety (M²) in a ligand covalent binding moiety of a L_{SS} primary linker of an Ligand Drug Conjugate, wherein the thio-substituent is a Ligand Unit incorporating or corresponding in structure to the targeting agent. In aspects in which the targeting agent is an antibody or antigen-binding fragment thereof, the antibody becomes bonded to M² through a sulfur atom of a cysteine residue derived from disulfide bond reduction or introduced through genetic engineering. As a result, the antibody or antigen-binding fragment thereof is covalently bonded to the L_{SS} primary linker as an antibody Ligand Unit. Subsequent hydrolysis of M² in a L_{SS} primary linker results in a L_{S} primary linker in which M² is converted to a succinic acid amide moiety (M³). That linker moiety may exist as a mixture of two regioisomers (M^{3A} and M^{3B}), depending on the relative reactivity of the two carbonyl groups of the succinimide ring system to hydrolysis.

“Ligand covalent binding moiety” as the term is used herein, unless otherwise stated or implied by context, refers to a moiety of a Linker Unit (LU) in Ligand Drug Conjugate that interconnects its Ligand Unit (L) and the remainder of the Linker Unit and is derived from reaction between the corresponding ligand covalent binding precursor (L_{B}′) moiety of a Linker Unit precursor (LU′) in a Drug Linker compound and a targeting agent, such as an antibody or antigen-binding fragment thereof. For example, when L_{B}′ is comprised of a maleimide moiety (M¹), reaction of that moiety with a reactive thiol functional group of a targeting agent converts L_{B}′ to a ligand covalent binding (L_{B}) moiety so that a thio-substituted succinimide moiety is obtained. When the targeting agent is an antibody or antigen-binding fragment thereof, the thio-substituent is comprised of a sulfur atom of an antibody Ligand Unit, which in some aspects is provided by a cysteine residue obtained by interchain disulfide bond reduction or genetic engineering.

In another example, when L_{B}′ is comprised of an activated carboxylic acid functional group, reaction of that functional group with a reactive amino group of a targeting agent, such as an epsilon amino group of a lysine residue in an antibody or antigen-binding fragment thereof, converts the functional group to an amide, wherein that amide functional group resulting from that reaction is shared between L_{B} and the attached Ligand Unit, which in the case of an antibody or antigen-binding fragment is an antibody Ligand Unit. Other L_{B} moieties and their conversion from L_{B}′-containing moieties are described in the embodiments of the invention. In yet another example, a targeting agent having a reactive amino group is derivitized with a bi-functional molecule to provide an intermediate, which in some instances results in a reactive thiol functional group, that is condensed with a L_{B}′ moiety. As a result of that condensation the L_{B} moiety so formed has atoms attributable to the bi-functional molecule and L_{B}′.

“Ligand covalent binding precursor moiety” is a moiety of a Linker Unit of a Drug Linker compound or Intermediate thereof that comprised of a reactive or activatable functional group, wherein the reactive functional group or activateable functional group subsequent to activation is capable of covalent binding to a targeting agent, such as an antibody or antigen-binding fragment thereof, during the preparation of a Ligand Drug Conjugate (LDC), including an Antibody Drug Conjugate (ADC), whereupon the ligand binding moiety precursor (L_{B}′) moiety is converted to a ligand covalent binding (L_{B}) moiety. In some aspects, a L_{B}′ moiety has a functional group capable of reacting with a nucleophile or electrophile native to an antibody or antigen-binding fragment thereof, or is introduced into the antibody or antigen binding fragment by chemical transformation or genetic engineering (vide supra) for its conversion to an antibody Ligand Unit. In some of those aspects, the nucleophile is an N-terminal amino group of a light or heavy chain of an antibody or antigen-binding fragment thereof, or the epsilon amino group of a lysine residue of that light or heavy chain.

In other aspects, the nucleophile is the sulfhydryl group of a cysteine residue introduced by genetic engineering into a light or heavy chain of an antibody or antigen-binding fragment thereof or from chemical reduction of an interchain disulfide of the antibody or antigen-binding fragment. In still some aspects, the electrophile is an aldehyde introduced by selective oxidation of a carbohydrate moiety in a glycan component of an antibody or antigen-binding fragment thereof, or is a ketone from an unnatural amino acid introduced into a light or heavy chain of an antibody or antigen-binding fragment thereof using a genetically engineered tRNA/tRNA synthetase pair. Those and other methods for introducing a reactive functional group to provide for a conjugation site in an antibody are reviewed by Behrens and Liu “Methods for site-specific drug conjugation to antibodies” mAB (2014) 6(1): 46-53.

“Secondary linker”, “secondary linker moiety” and like terms as used herein, unless otherwise stated or implied by context, refer to an organic moiety in a Linker Unit (LU), wherein the secondary linker (L_{O}) is a component of LU that interconnects a Drug Unit to a primary linker (L_{R}) and contains a ligand covalent binding (L_{B}) moiety, a first optional Stretcher Unit and/or an optional Branching Unit (B) and in some aspects provides for a self-stabilizing (L_{SS}) primary linker of a Ligand Drug Conjugate (LDC), such as an Antibody Drug Conjugate (ADC), or of a Drug Linker compound useful for the preparation of the Conjugate, or provides for a self-stabilized (L_{S}) primary linker of a LDC/ADC compound upon hydrolysis of L_{SS}. In instances when L_{R} is L_{SS} or L_{S}, the first optional Stretcher Unit is present. In those aspects, L_{R} is attached to L_{O} through a heteroatom or functional group from the first optional Stretcher Unit (A) that is present.

A secondary linker of a Ligand Drug Conjugate compound or a Drug Linker compound typically has the structure of:

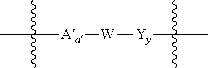

when subscript b is 0 wherein the wavy line adjacent to A′ indicates the site of covalent attachment of L_{O} to the primary linker; the wavy line adjacent to Y indicates the site of covalent attachment of L_{O} to the Drug Unit; A′ is a second optional Spacer Unit, or in some aspects is a subunit of a first optional Stretcher Unit that is present, subscript a′ is 0 or 1, indicating the absence or presence of A′, respectively; Y is a Spacer Unit, and subscript y is 0, 1 or 2, indicating the absence or presence of one or two Spacer Units, respectively; and W is a Peptide Cleavable Unit, wherein the Peptide Cleavable Unit provides for a recognition site that has overall greater selectivity for proteases of tumor tissue homogenate in comparison to proteases in normal tissue homogenate, wherein the tumor tissue is comprised of targeted cancer cells and the normal tissue is comprised of non-targeted normal cells for which off-target cytotoxicity by the Ligand Drug Conjugate is responsible at least in part for an adverse event often associated with administration of a therapeutically effective amount to a mammalian subject in need thereof. When subscript b is 0, A′, when present, becomes a subunit of A in which case the secondary linker has the structure of —W—Y_{y}—. In either of those aspects W, Y and D are arranged in a linear configuration with respect to the remainder of LU/LU′, as represented by —W—Y_{y}-D, in which W is the Peptide Cleavable Unit and subscript y is 0, 1 or 2. When subscript y is 1 or 2, protease cleavage is followed by self-immolation of a self-immolative Spacer Unit attached to W so as to release D or Y′-D, if a second Spacer Unit (Y′) is present, which decomposes to complete release of D as free drug.

A secondary linker (L_{O}) bonded to D in a Linker Unit as exemplified when only one Drug Unit is attached to LU in which W is a Peptide Cleavable Unit is typically represented by the structure of

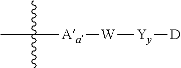

when subscript b is 1 or

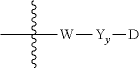

due to A′_{a′} being treated as a subunit of a first optional Stretcher unit when subscript b is 0 and subscript a′ is 1;
wherein D is a Drug Unit and the remaining variable groups are as defined herein for L_{O};
and a drug linker moiety or a Drug Linker compound comprised of that secondary linker typically has the structure of Formula 1B and Formula IB, respectively:

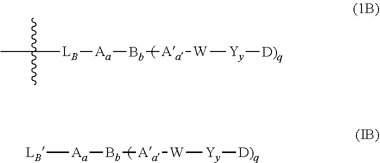

wherein L_{B} is a ligand covalent binding moiety as defined herein, which is a component of a primary linker (L_{R}) of a Linker Unit (LU) of a drug linker moiety of a Ligand Drug Conjugate compound; and L_{B}′ is a ligand covalent binding moiety as defined herein, which is a component of a primary linker (L_{R}′) of a Linker Unit (LU′) in a Drug Linker compound, and are sometimes referred to as a ligand covalent binding moiety precursor, a primary linker precursor and a Linker Unit precursor for L_{R}, L_{B} and LU, respectively, of a Ligand Drug Conjugate when the Drug Linker Compound is used in the preparation of the Ligand Drug Conjugate; A is a first optional Stretcher Unit; subscript a is 0 or 1, indicating the absence or presence of A, respectively; B is an optional Branching Unit, subscript b is 0 or 1, indicating the absence or presence of B, respectively, wherein A′ is a subunit of A when subscript b is 0, subscript a is 1 and subscript a′ is 1; subscript q ranges from 1 to 4, wherein L_{B}/L_{B}′ and A and B, when present, are components of L_{R}/L_{R}′ and provided that subscript q ranges from 2 to 4 when subscript b is 1, and subscript q is 1 when subscript b is 0; and the remaining variable groups are as defined herein for L_{O}.

“Maleimide moiety” as used herein, unless otherwise stated or implied by context, refers to a component of a primary linker of a Drug Linker compound, which in some aspects is a component of a self-stabilizing linker, wherein that primary linker is sometimes represented as L_{R}′ or L_{SS}′ to explicitly indicated that it is a precursor to L_{R}/L_{SS} in a Ligand Drug Conjugate. A maleimide moiety (M¹) is capable of participating in Michael addition (i.e., 1,4-conjugate addition) by a sulfur atom of a reactive thiol functional group of targeting agent, such as an antibody or antigen-binding fragment thereof, to provide a thio-substituted succinimide (M²) moiety, wherein the thio substituent is a Ligand Unit that incorporates or corresponds to the structure of the targeting agent as exemplified herein for an antibody Ligand Unit of an Antibody Drug Conjugate composition or compound thereof. That M¹ moiety of a Drug Linker compound is attached to the remainder of the primary linker, typically to a first optional Stretcher Unit (A) that is present as the M¹ moiety is a component of L_{SS}′ or to a secondary linker (L_{O}) if both A and B are absent, through its imide nitrogen atom.

Other than the imide nitrogen atom, an M¹ moiety is typically unsubstituted, but may be asymmetrically substituted at the cyclic double bond of its maleimide ring system. Such substitution can result in regiochemically preferred conjugate addition of a sulfur atom of a reactive thiol functional group of a targeting agent to the less hindered or more electronically deficient double bonded carbon atom (dependent on the more dominant contribution) of the maleimide ring system. That conjugate addition results in a succinimide (M²) moiety, which is thio-substituted by the Ligand Unit though a sulfur atom from a thiol functional group provided by the targeting agent.

“Succinimide moiety” as used herein, unless otherwise stated or implied by context, refers one type of ligand covalent binding (L_{B}) moiety in of primary linker, which in turn is a component of a Linker Unit of a Ligand Drug Conjugate, such as an Antibody Drug Conjugate, and results from Michael addition of a sulfur tom of a reactive thiol functional group of an antibody or antigen-binding fragment thereof to the maleimide ring system of a maleimide moiety (M¹), which is one type of ligand covalent binding precursor (L_{B}′) moiety in a Drug Linker compound or a M¹-containing intermediate thereof. A succinimide (M²) moiety is therefore comprised of a thio-substituted succinimide ring system that has its imide nitrogen atom substituted with the remainder of the primary linker, which typically would be a first optional Stretcher Unit (A) that is present. In some aspects, that nitrogen atom is attached to the first optional Stretcher Unit (A) that is present through an optionally substituted C₁-C₁₂ alkylene moiety comprising that Unit. When the primary linker is a self-stabilizing linker, that alkylene moiety incorporates a cyclic Basic Unit into a first optional Stretcher Unit that is present or is substituted by an acyclic Basic Unit as described elsewhere, and is otherwise optionally substituted, and has its M² moiety optionally substituted with substituent(s) at its succinimide ring system, which may have been present on the M¹ precursor.

Thus, the optionally substituted C₁-C₁₂ alkylene moiety of A, in optional combination with [HE], which is an optional hydrolysis-enhancing unit, is either covalently attached directly to the optional secondary linker (L_{O}) that is present, when subscript b is 0 or indirectly to L_{O} through —[HE]-B— when subscript b is 1 in a drug linker moiety of Formula 1B or the Drug Linker compound of Formula IB. In those instances in which subscript b is 0, subscript a is 1 and subscript a′ is 1, A is represented by the formula -A₁[HE]-A₂-, wherein A₁ is a first subunit of A and is comprised of the optionally substituted C₁-C₁₂ alkylene moiety in optional combination with HE, and A′, previously indicated as a component of L_{O}, becomes A₂, which is now the second subunit of A. In those instances when subscript b is 1 and subscript a is 1 and subscript a′ is 1, A′ is a component of the secondary linker and A is a single unit in optional combination with [HE] or is optionally comprised of two subunits, which is represented by -A[HE]-A_{O}-, wherein A_{O} is an optional subunit of A. When A_{O} is present, A is also represented by the formula -A₁[HE]-A₂-.

When present in a self-stabilizing linker (L_{SS}) in a Ligand Drug Conjugate compound, hydrolysis of the succinimide ring system of the thio-substituted succinimide (M²) moiety, which is pH controllable due to the nearby presence of the basic functional group of the acyclic or cyclic Basic Unit, provides in some instances regiochemical isomers of succinic acid-amide (M³) moieties in a self-stabilized linker (L_{S}) due to its asymmetric substitution by the thio substituent. The relative amounts of those isomers will be due at least in part to differences in reactivity of the two carbonyl carbons of M², which can be attributed at least in part to any substituent(s) that were present in the M¹ precursor. Hydrolysis is also expected to occur to some extent when L_{R} having a M² moiety that does not contain a Basic Unit but is highly variable in comparison to the controlled hydrolysis provided by the Basic Unit.

In some aspects, those optional substituents on the succinimide ring system of M² are not present and the first optional Stretcher Unit is present and is comprised of an optionally substituted C₁-C₁₂ alkylene moiety optionally attached to [HE], which is an optional hydrolysis-enhancing unit, at a position distal to its attachment site to the imide nitrogen atom. In that aspect, A is a single unit or is further comprised of A′, which is an optional subunit of A that is present when subscript b is 0 and subscript a′ is 1, and is attached to [HE] that is also present so that A has the formula of -A[HE]-A′- or when subscript b is 1 and subscript a′ is 1, A′ is a component that is present of the secondary linker so that A is represented by the formula of -A[HE]-A_{O}-.

“Succinic acid-amide moiety” as used herein, unless otherwise stated or implied by context, refers to component of a self-stabilized linker (L_{S}) of a Linker Unit within a Ligand Drug Conjugate, such as an Antibody Drug Conjugate, and has the structure of a succinic amide hemi-acid residue with substitution of its amide nitrogen by another component of L_{S}, wherein that component is typically a first optional Stretcher Unit (A) or subunit thereof that is present and is comprised of an C₁-C₁₂ alkylene moiety optionally attached to [HE]. The possible structures for A when subscript b is 0 and subscript a is 0 or 1 are indicated by the formulae of -A[HE]-A′_{a′}-, in which A′ previously associated with the secondary linker is either absent so that subscript a′ is 0 or when subscript a′ is 1 A′ is present as a subunit of A. When that subunit is present, A is represented by the formula of A₁[HE]-A₂-, wherein A₁ is the first subunit of A, which is comprised of the optionally substituted C₁-C₁₂ alkylene moiety optionally attached to [HE], and A₂ is the second subunit of A, previously indicated as A′. The possible structures for A when subscript b is 1 and subscript a is 1 are indicated by the formula of -A[HE]-A_{O}-, in which A_{O} is an optional subunit of A when present. When that subunit is absent A is a single discrete unit and when A_{O} is present A is represented by the formula of A₁[HE]-A₂-, wherein A₁ is the first subunit of A, which is comprised of the optionally substituted C₁-C₁₂ alkylene moiety optionally attached to [HE], and A₂, previously indicated as A_{O}, is the second subunit of A.

In some aspects, the alkylene moiety incorporates a cyclic Basic Unit and in other aspects is substituted by an acyclic Basic Unit and in either aspect is otherwise optionally substituted, wherein the succinic acid amide (M³) moiety has further substitution by L-S—, wherein L is a Ligand Unit such as an antibody Ligand Unit incorporating or corresponding in structure to a targeting agent such as an antibody or antigen-binding fragment thereof and S is a sulfur atom from that targeting agent, antibody or antigen-binding fragment. A M³ moiety results from the thio-substituted succinimide ring system of a succinimide (M²) moiety in self-stabilizing primary linker having undergone breakage of one of its carbonyl-nitrogen bonds by hydrolysis, which is assisted by the Basic Unit.

Thus, a M³ moiety has a free carboxylic acid functional group and an amide functional group whose nitrogen heteroatom is attached to the remainder of the primary linker and is substituted by L-S— at the carbon that is alpha to that carboxylic acid or amide functional group, depending on the site of hydrolysis of its M² precursor. Without being bound by theory, it is believed the aforementioned hydrolysis resulting in a M³ moiety provides a Linker Unit (LU) in an Ligand Drug Conjugate that is less likely to suffer premature loss from the Conjugate of its targeting Ligand Unit (L) through elimination of the thio substituent.

“Self-stabilizing linker” as used herein, unless otherwise stated or implied by context, refers to a primary linker of a Linker Unit (LU) in a Ligand Drug Conjugate, such as an Antibody Drug Conjugate, having a M²-containing component or a primary linker of a Linker Unit precursor (LU′) in a Drug Linker compound having a M¹-containing component, wherein that component may be designated as L_{SS}′ to indicate that it is a precursor to the M²-containing component of L_{SS} in an LDC. The self-stabilizing linker subsequently undergoes conversion under controlled hydrolysis conditions to the corresponding self-stabilized linker (L_{S}). That hydrolysis is facilitated by the Basic Unit component of L_{SS}, such that an LDC/ADC comprised of L_{SS} becomes more resistant to premature loss of its Ligand Unit by virtue of its Linker Unit (LU) now being comprised of L_{S}. The L_{SS} primary linker, in addition to its M¹ or M² moiety, is further comprised of a first optional Stretcher Unit (A) that is required to be present, wherein A is comprised of an C₁-C₁₂ alkylene moiety optionally in combination with [HE], wherein that combination is sometimes designated as A₁ when A is further comprised of an optional subunit (A_{O}) that is present when subscript b is 1 or A is further comprised of A′ when subscript b is 0 and subscript a′ is 1, wherein with either value of subscript b that additionally present subunit is designated a A₂. When A may exist as a single discrete unit or in the form of two discrete units, both possibilities are represented by the formula of -A[HE]-A_{O}-, when subscript b is 1 or A[HE]-A′_{a′} when subscript b is 0, which for either value of subscript b becomes -A[HE]- or -A₁[HE]-A₂-, depending on the absence or presence, respectively, of a second subunit. In either variation of A within L_{SS}, its alkylene moiety incorporates a cyclic Basic Unit or is substituted by an acyclic Basic Unit and is otherwise optionally substituted.

Thus, when the primary linker of a Drug Linker compound is L_{SS}, sometimes shown as L_{SS}′ to indicate that it is a precursor of L_{SS} in a Ligand Drug Conjugate, that primary linker contains a first optional Stretcher Unit (A) that is required to be present and a maleimide (M¹) moiety through which a targeting agent is to be attached, which in the case of an antibody or antigen-binding fragment thereof provides an antibody Ligand Unit. In those aspects, the C₁-C₁₂ alkylene moiety of A of L_{SS} is attached to the imide nitrogen of the maleimide ring system of M¹ and to the remainder of the Linker Unit, the latter of which optionally occurs through [HE]-A_{O}-B— when subscript b is 1 or [HE]-A′_{a′}- when subscript b is 0, depending on the absence or presence of A_{O}/A′ and [HE]. In some of those aspects, [HE], which is a hydrolysis-enhancing moiety, consists or is comprised of an optionally substituted electron withdrawing heteroatom or functional group, which in some aspects in addition to BU may enhance the hydrolysis rate of the M² moiety in the corresponding L_{SS} moiety of a LDC/ADC compound. After incorporation of the Drug Linker compound into an LDC/ADC compound, L_{SS} now contains a succinimide (M²) moiety that is thio-substituted by the Ligand Unit (i.e., attachment of the Ligand Unit to its drug linker moiety has occurred through Michael addition of a sulfur atom of a reactive thiol functional group of a targeting agent to the maleimide ring system of M¹).

In some aspects, a cyclized Basic unit (cBU) corresponds in structure to an acyclic Basic Unit through formal cyclisation to the basic nitrogen of that Unit so that the cyclic Basic Unit structure is incorporated into the first optional Stretcher Unit that is present as an optionally substituted spiro C₄-C₁₂ heterocyclo. In such constructs, the spiro carbon is attached to the maleimide imide nitrogen of M¹, and hence to that nitrogen in M², and is further attached to the remainder of the L_{SS} primary linker, which is comprised of the afore-described first optional Stretcher Unit (A) that is present optionally through -[HE]-A_{O}- or [HE]-A_{a′}-, in a drug linker moiety of Formula 1B or a Drug Linker compound of Formula IB. In those aspects, a cyclic BU assists in the hydrolysis of the succinimide moiety of M² to its corresponding ring-opened form(s) represented by M³ in qualitatively similar manner to that of an acyclic Basic Unit, which may also be enhanced by [HE].

In some aspects, L_{B}′-A-B_{b}— of a L_{SS} primary linker, which is sometimes shown as L_{SS}′ to explicitly indicate that it is a precursor to a self-stabilizing (L_{SS}) primary linker in a Drug Linker compound of Formula IB, is represented by the general formula of M¹-A(BU)—[HE]-A_{O}-B— when subscript b is 1 or M¹-A(BU)—[HE]-A′_{a′}- when subscript b is 0, wherein M¹ is a maleimide moiety and A is a C₁-C₁₂ alkylene that incorporates or is substituted by BU and is otherwise optionally substituted and is in optional combination with [HE], which is an optional hydrolysis-enhancing moiety, wherein that formula for becomes M¹-A(BU)—[HE]-B— or M¹-A(BU)[HE]- when A is a single discreet unit or M¹-A₁(BU)—[HE]-A₂-B— or M¹-A₁(BU)—[HE]-A₂- when A is of two subunits, wherein A₁ and A₂ are the subunits of A.

In other aspects, a L_{SS} primary linker in a drug linker moiety of Formula 1B of an ADC of Formula 1A, is represented by the general formula of -M²-A(BU)—[HE]-A_{O}-B—, when subscript b is 1 or -M²-A(BU)—[HE]-A_{a′}- when subscript b is 0, wherein M² is a succinimide moiety, A is a first optional Stretcher Unit that is present and is comprised of an C₁-C₁₂ alkylene that incorporates or is substituted by BU and is otherwise optionally substituted and is in optional combination with [HE], which is an optional hydrolysis-enhancing moiety, and A_{O}/A′ is an optional subunit of A. When A is a single discreet unit, L_{SS} is represented by the formula of -M²-A(BU)—[HE]-B— or -M²-A(BU)—[HE]- and when A is of two subunits, L_{SS} is represented by the formula of -M²-A₁(BU)—[HE]-A₂- or -M²-A₁(BU)—[HE]-A₂-B— when subscript b is 0 or 1, respectively.

In still other aspects, a L_{S} primary linker in a drug linker moiety of Formula 1B of a LDC/ADC of Formula 1A is represented by the general formula of -M³-A(BU)—[HE]-A_{O}-B—, when subscript b is 1 or -M³-A(BU)—[HE]-A_{a′}- when subscript b is 0, wherein M³ is a succinimide acid amide moiety and A is a C₁-C₁₂ alkylene that incorporates or is substituted by BU, and is otherwise optionally substituted, and is in optional combination with [HE], which is an optional hydrolysis-enhancing moiety, and A_{O}/A′ is an optional subunit of A, wherein -A(BU)—[HE]-A_{O}- or -A(BU)—[HE]-A_{a′}- becomes -A(BU)—[HE]-when A is a single discreet unit or -A₁(BU)—[HE]-A₂- when A is or is comprised of two subunits.

Exemplary, but non-limiting -L_{B}-A- structures comprising a L_{SS} primary linker within a drug linker moiety of Formula 1B for some Ligand Drug Conjugates of Formula 1 are represented by:

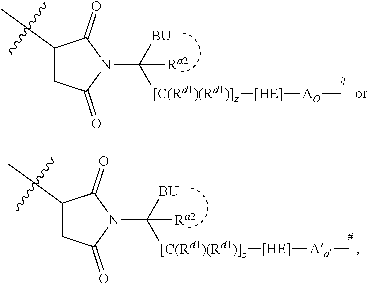

wherein the wavy line indicates the site of covalent attachment to a Ligand Unit, the pound sign (#) in the upper structure for which subscript b is 1 indicates the site of covalent attachment in Formula 1B to a Branching Unit (B) or in the lower structure in which subscript b is 0 to W of an optional secondary linker (L_{O}) that is present and wherein the dotted curved line indicates optional cyclization which is present when BU is a cyclic Basic Unit or is absent when BU is an acyclic Basic Unit, wherein [HE] is an optional hydrolysis-enhancing moiety, A_{O}/A′ is an optional subunit of A, subscript z is 0 or an integer ranging from 1 to 6; each R^{d1} is independently selected from the group consisting of hydrogen and optionally substituted C₁-C₆ alkyl, or two of R^{d1}, the carbon atom(s) to which they are attached and any intervening carbon atoms define an optionally substituted C₃-C₈ carbocyclo, and the remaining R^{d1}, if any, are independently hydrogen or optionally substituted C₁-C₆; and R^{a2} is —H or an optionally substituted C₁-C₈ alkyl when BU is an acyclic Basic Unit, and when BU a cyclic Basic Unit, R^{a2} is required to be other than —H and along with the carbon atom to which BU and R^{a2} are attached define an optionally substituted spiro C₄-C₁₂ heterocyclo having a skeletal secondary or tertiary basic nitrogen atom, such that the acyclic or cyclic BU is capable of increasing the rate of hydrolysis of the shown succinimide (M²) moiety to provide a succinic acid amide (M³) moiety at a suitable pH in comparison to the corresponding Conjugate in which R^{a2} is hydrogen and BU is replaced by hydrogen, and for a cyclic Basic Unit substantially retains the increase in the rate of hydrolysis of the drug linker moiety corresponding to that of the LDC/ADC in which in R^{a2} is hydrogen and BU is an acyclic BU over the aforementioned Conjugate in which R^{a2} is hydrogen and BU is replaced by hydrogen.

Exemplary, but non-limiting, L_{B}′-A- structures comprising L_{SS}′, which are sometimes present in Drug Linker compounds of Formula I used as intermediates in the preparation of Ligand Drug Conjugate compositions, are represented by:

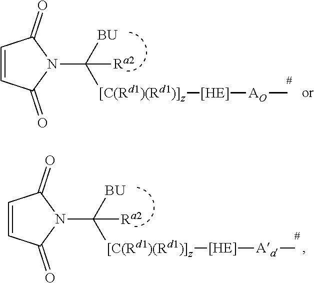

wherein BU and the other variable groups are as defined above for L_{B}-A-structures of LDCs/ADCs having L_{SS} primary linkers. When a Drug Linker compound having a self-stabilizing linker precursor (L_{SS}′), which is comprised of a maleimide moiety, is used in the preparation of an LDC/ADC, that L_{SS}′ moiety is converted into an L_{SS} primary linker comprised of a succinimide moiety. Prior to condensation with a reactive thiol functional group from a targeting agent such as an antibody or antigen-binding fragment thereof, the basic nitrogen atom of BU is typically protonated or protected by an acid-labile protecting group.

“Self-stabilized linker” is an organic moiety derived from a M²-containing moiety of a self-stabilizing linker (L_{SS}) in a Ligand Drug Conjugate, such as an Antibody Drug Conjugate, that has undergone hydrolysis under controlled conditions so as to provide a corresponding M³-moiety of a self-stabilized linker (L_{S}), wherein that LU component is less likely to reverse the condensation reaction of a targeting moiety with a M¹-containing moiety that provided the original M²-containing L_{SS} moiety. In addition to the M³ moiety, a self-stabilized linker (L_{S}) is comprised of a first optional Stretcher Unit (A) that is present and incorporates a cyclic Basic Unit or is substituted by an acyclic Basic Unit, wherein A is covalently attached to M³ and the remainder of the L_{S} primary linker (i.e., B) or to a secondary linker (L_{O}) when B is absent. The M³ moiety is obtained from conversion of a succinimide moiety (M²) of L_{SS} in an Ligand Drug Conjugate, wherein the M² moiety has a thio-substituted succinimide ring system resulting from Michael addition of a sulfur atom of a reactive thiol functional group of a targeting agent to the maleimide ring system of M¹ of a L_{SS}′ moiety in a Drug Linker compound, wherein that M²-derived moiety has reduced reactivity for elimination of its thio-substituent in comparison to the corresponding substituent in M². In those aspects, the M²-derived moiety has the structure of a succinic acid-amide (M³) moiety corresponding to M² wherein M² has undergone hydrolysis of one of its carbonyl-nitrogen bonds of its succinimide ring system, which is assisted by the basic functional group of BU due to its appropriate proximity as a result of that attachment. The product of that hydrolysis therefore has a carboxylic acid functional group and an amide functional group substituted at its amide nitrogen atom, which corresponds to the imide nitrogen atom in the M²-containing L_{SS} precursor to L_{S}, with the remainder of the primary linker, which is will include at minimum the optional Stretcher Unit that is present. In some aspects, the basic functional group is a primary, secondary or tertiary amine of an acyclic Basic Unit or secondary or tertiary amine of a cyclic Basic Unit. In other aspects, the basic nitrogen of BU is a heteroatom of an optionally substituted basic functional group as in a guanidino moiety. In either aspect, the reactivity of the basic functional group of BU for base-catalyzed hydrolysis is controlled by pH by reducing the protonation state of the basic nitrogen atom.

Thus, a self-stabilized linker (L_{S}) typically has the structure of an M³ moiety covalently bond to a first optional Stretcher Unit that is present and incorporating a cyclic Basic Unit or substituted by an acyclic Basic Unit. In some aspects, A is a discrete single unit and in other aspects is of two or more subunits, typically represented by A₁-A₂ if two subunits are present with A/A₁ optionally in combination with [HE]. Stretcher Unit A in turn is covalently bonded to B of the L_{S} primary linker or to W of L_{O} with its M³, A, A′_{a′}/B and BU components arranged in the manner represented by the general formula of -M³-A(BU)—[HE]-A′_{a′}- or M³-A(BU)—[HE]-A_{O}-B—, in which subscript b is 0 or 1, respectively. When A is a single discreet unit, L_{S} is represented by -M³-A(BU)—[HE]-B— when subscript b is 1 or -M³-A(BU)—[HE]- and when A is of two subunits represent L_{S} is represented by -M³-A₁(BU)-A₂- or -M³-A₁(BU)-A₂-B— in which subscript b is 0 or 1, respectively, wherein BU represents either type of Basic Unit (cyclic or acyclic).

Exemplary non-limiting structures of -L_{B}-A- in L_{SS} and L_{S} primary linkers for LDCs/ADCs in which L_{B} is M² or M³; and A(BU)/A₁(BU), and [HE] within these structures are arranged in the manner indicated above in which BU is an acyclic Basic Unit is shown by way of example but not limitation by the structures of:

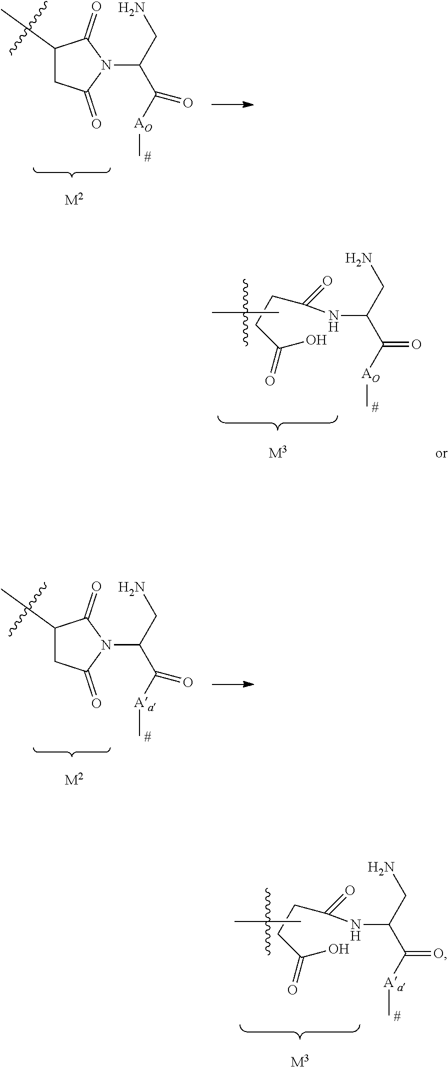

wherein the —CH(CH₂NH₂)C(═O)— moiety is A, when A is a single discreet unit so that A_{O} or A′ is absent or A is A₁-A₂- when A_{O}/A′ is present as A₂, and wherein A/A₁ is substituted by BU, wherein BU is an acyclic Basic Unit, which is —CH₂NH₂, having the basic nitrogen atom optionally protonated, and —C(═O)— within that moiety is the optional hydrolysis enhancing moiety [HE] that is present and wherein the hash mark in the upper structure indicates covalent attachment to B and the hash mark in the lower structure indicates covalent attachment to W of L_{O}. Those exemplary structures contain a succinimide (M²) moiety or a succinic acid-amide (M³) moiety, respectively, the latter of which results from succinimide ring hydrolysis of M² assisted by —CH₂NH₂ in the conversion of L_{SS} to L_{S}.

Exemplary non-limiting structures of -L_{B}-A- in L_{SS} and L_{S} primary linkers for LDCs/ADCs in which L_{B} is M² or M³; and A(BU)/A₁(BU), A_{O}/A′ and [HE] within these structures are arranged in the manner indicated above in which BU is a cyclic Basic Unit is shown by way of example but not limitation by the structures of:

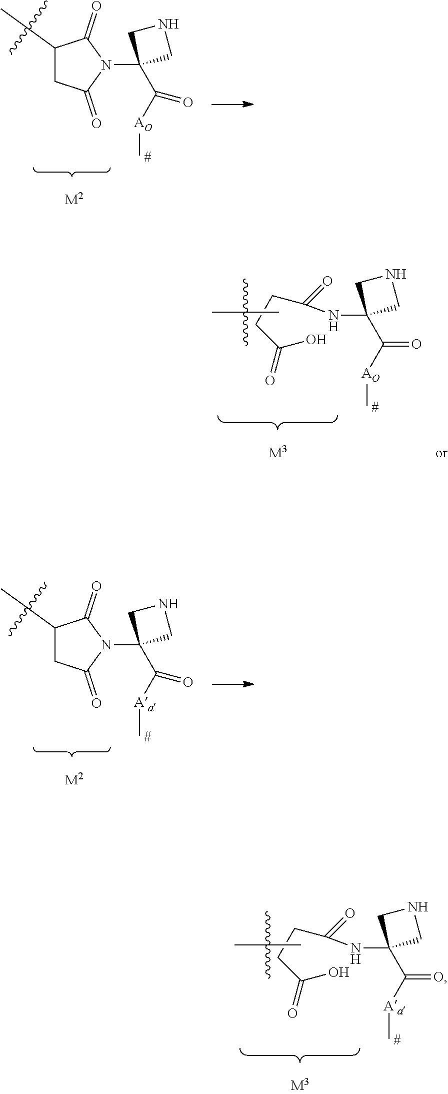

wherein these -M²-A(BU)—[HE]-A_{O}/A′_{a′}- and -M³-A(BU)—[HE]-A_{O}/A′_{a′}-structures become -M²-A(BU)—[HE]- and -M³-A(BU)—[HE]-, when A_{O} is absent or subscript a′ is 0 so that A is present as a single discreet unit or become -M²-A₁(BU)—[HE]-A₂- and -M³-A¹(BU)—[HE]-A₂- when A_{O}/A′ is present as a subunit of A indicated as A₂ and wherein in either structure BU is a cyclic Basic Unit in the form of an optionally protonated azetidin-3,3-diyl, the structure of which is an exemplary heterocyclo Basic Unit incorporated into A/A₁. That heterocyclo corresponds to the aminoalkyl of an acyclic Basic Unit in an -A₁(BU)— or -A(BU)— moiety in which the basic nitrogen of the acyclic Basic Unit has been formally cyclized at least in part back through R^{a2} to the carbon atom that is alpha to the succinimide nitrogen of M² to which the acyclic Basic Unit is attached.

The wavy line in each of the above -L_{B}-A- structures indicates the site of covalent attachment of a sulfur atom of a Ligand Unit derived from a reactive thiol functional group of a targeting agent upon Michael addition of that sulfur atom to the maleimide ring system of an M¹ moiety in a structurally corresponding Drug Linker compound or M¹-containing intermediate thereof. The hash mark (#) in the upper structure indicates the site of covalent attachment to B, which is the remainder of the L_{SS} or L_{S} primary linker and in the lower structure indicates the site of covalent attachment to W of L_{O}. Since the succinimide ring system of M² is asymmetrically substituted due to its thio substituent, regiochemical isomers of succinic acid-amide (M³) moieties as defined herein differing in position relative to the liberated carboxylic acid group may result on M² hydrolysis. In the above structures, the carbonyl functional group shown adjacent to A_{O} exemplifies a hydrolysis enhancer [HE] as defined herein.

The above -M³-A(BU)—[HE]-A_{O}/A′_{a′}-, -M³-A(BU)— and -M³-A₁(BU)—[HE]-A₂- moieties wherein BU is acyclic or cyclic Basic Unit represent exemplary -L_{B}-A-structures that comprise self-stabilized linker (L_{S}) primary linkers, so named because these structures are less likely to eliminate the thio substituent of the Ligand Unit, and thus cause loss of that targeting moiety, in comparison to the corresponding L_{SS} moieties comprised of formula -M²-A(BU)—[HE]-A_{O}/A′_{a′}-, -M²-A(BU)— or -M²-A₁(BU)—[HE]-A₂- from which they are derived. Without being bound by theory, it is believed the increased stability results from the greater conformational flexibility in M³ in comparison to M², which no longer constrains the thio substituent in a conformation favorable for E2 elimination.

“Basic Unit” as used herein, unless otherwise stated or implied by context, refers to an organic moiety within a self-stabilizing linker (L_{SS}) primary linker, as described herein, which is carried forward into a corresponding L_{S} moiety by BU participating in base catalyzed hydrolysis of the succinimide ring system within a M² moiety comprising L_{SS} (i.e., catalyzes addition of a water molecule to one of the succinimide carbonyl-nitrogen bonds). In some aspects, the base-catalyzed hydrolysis is initiated under controlled conditions tolerable by the targeting Ligand Unit attached to L_{SS}. In other aspects, the base-catalyzed hydrolysis is initiated on contact of the Drug Linker compound comprised of L_{SS}′ with a targeting agent in which Michael addition of a sulfur atom of a reactive thiol functional group of the targeting agent competes with hydrolysis of the M¹ moiety of L_{SS}′ of the Drug Linker compound. Without being bound by theory, the following aspects describe various considerations for design of a suitable Basic Unit. In one such aspect, the basic functional group of an acyclic Basic Unit and its relative position in L_{SS} with respect to its M² component are selected for the ability of BU to hydrogen bond to a carbonyl group of M², which effectively increases its electrophilicity and hence its susceptibility to water attack. In another such aspect, those selections are made so that a water molecule, whose nucleophilicity is increased by hydrogen bonding to the basic functional group of BU, is directed to an M² carbonyl group. In a third such aspect, those selections are made so the basic nitrogen on protonation does not increase the electrophilicity of the succinimide carbonyls by inductive electron withdrawal to an extent that would promote premature hydrolysis requiring compensation from an undesired excess of Drug Linker compound. In a further such aspect, some combination of those mechanistic effects contributes to catalysis for controlled hydrolysis of L_{SS} to L_{S}.

Typically, an acyclic Basic Unit, which may act through any of the above mechanistic aspects, is comprised of 1 carbon atom or 2 to 6 contiguous carbon atoms, more typically of 1 carbon atom or 2 or 3 contiguous carbon atoms, wherein the carbon atom(s) connect the basic amino functional group of the acyclic Basic Unit to the remainder of the L_{SS} primary linker to which it is attached. In order for that basic amine nitrogen atom to be in the required proximity to assist in the hydrolysis of a succinimide (M²) moiety to its corresponding ring-opened succinic acid amide (M³) moiety, the amine-bearing carbon chain of an acyclic Basic Unit is typically attached to A of the -L_{B}-A-moiety of L_{SS} at the alpha carbon of the C₁-C₁₂ alkylene of that moiety relative to the site of attachment of A to the succinimide nitrogen of M² (and hence to the maleimide nitrogen of its corresponding M¹-A- structure). Typically, that alpha carbon in an acyclic Basic Unit has the (S) stereochemical configuration or the configuration corresponding to that of the alpha carbon of L-amino acids.

As previously described, BU in acyclic form or BU in cyclized form is typically connected to M¹ or M² of L_{SS} or M³ of L_{S} through an otherwise optionally substituted C₁-C₁₂ alkylene moiety in which that moiety incorporates the cyclized Basic Unit or is substituted by the acyclic Basic Unit and is bonded to the maleimide or succinimide nitrogen of M¹ or M², respectively, or the amide nitrogen atom of M³. In some aspects, the otherwise optionally substituted C₁-C₁₂ alkylene moiety incorporating the cyclic Basic Unit is covalently bonded to [HE] and typically occurs through intermediacy of an ether, ester, carbonate, urea, disulfide, amide carbamate or other functional group, more typically through an ether, amide or carbamate functional group. Likewise, BU in acyclic form is typically connected to M¹ or M² of L_{SS} or M³ of L_{S} through the otherwise optionally substituted C₁-C₁₂ alkylene moiety of A in L_{B}′-A-, in which L_{B}′ is M¹, or -L_{B}-A-, in which L_{B} is M² or M³, that is substitution by the acyclic Basic unit at the same carbon of the C₁-C₁₂ alkylene moiety that is attached to the imino nitrogen atom of the maleimide or succinimide ring system of M¹ or M² or the amide nitrogen of M³, which results from hydrolysis of the succinimide ring system of M².

In some aspects, a cyclic Basic Unit incorporates the structure of an acyclic BU by formally cyclizing an acyclic Basic Unit to an otherwise optionally substituted C₁-C₁₂ alkyl (R^{a2}), independently selected from that of A/A₁, that is bonded to the same alpha carbon as the acyclic Basic Unit, thus forming a spirocyclic ring system so that a cyclic Basic Unit is incorporated into the structure of A/A₁ rather than being a substituent of A/A₁ as when BU is acyclic. In those aspects, the formal cyclization is to the basic amine nitrogen of an acyclic Basic Unit thus providing a cyclic Basic Unit as an optionally substituted symmetrical or asymmetrical spiro C₄-C₁₂ heterocyclo, depending on the relative carbon chain lengths in the two alpha carbon substituents, in which the basic nitrogen is now a basic skeletal heteroatom. In order for that cyclization to substantially retain the basic properties of the acyclic Basic Unit in a cyclic Basic Unit, the basic nitrogen atom of the acyclic Basic Unit nitrogen should be that of a primary or secondary amine and not a tertiary amine since that would result in a quaternized skeletal nitrogen in the heterocyclo of the cyclic Basic Unit. In that aspect of formal cyclization of an acyclic Basic Unit to a cyclic Basic Unit, in order to substantially retain the ability of the basic nitrogen to assist in hydrolysis of M² to M³ in conversion of L_{SS} to L_{S}, the resulting structure of the cyclic Basic Unit in these primary linkers will typically have its basic nitrogen located so that no more than three, and typically one or two, intervening carbon atoms between the basic nitrogen atom and the spiro carbon of the spiro C₄-C₁₂ heterocyclo component. Cyclic Basic Units incorporated into A/A₁ and the L_{SS} and L_{S} primary linkers having these as components are further described by the embodiments of the invention.

“Hydrolysis-enhancing moiety” as used herein, unless otherwise stated or implied by context, refers to an electron withdrawing group or moiety that is optionally present within a first optional Stretcher Unit (A) in L_{B}′-A- or -L_{B}-A- of an L_{SS} primary linker and its hydrolysis product L_{S}. A hydrolysis-enhancing [HE] moiety, when present as component of A/A₁ of L_{SS} in a drug linker moiety of an LDC/ADC in which A/A₁ is bonded to the imide nitrogen of an M² moiety in some aspects increases or has minimal effects on the electrophilicity of the succinimide carbonyl groups in that moiety, depending on its proximity to that M² moiety due to the electron withdrawing effect of [HE], to facilitate its conversion to a M³ moiety of a L_{S} primary linker With A/A₁ incorporating or substituted by a cyclic Basic Unit or an acyclic Basic Unit, respectively, the potential effect of [HE] on the carbonyl groups of M² for increasing the hydrolysis rate to M³ by induction and the aforementioned effect(s) of either type of BU, are adjusted so that premature hydrolysis of M¹ does not occur to an appreciable extent during preparation of a Ligand Drug Conjugate from a Drug Linker compound comprised of the L_{B}′-A- structure of formula M¹-A(BU)—[HE]-A_{O}/A′_{a′}-, with the two variations represented by the formulae of M¹-A(BU)— and M¹-A₁(BU)—[HE]-A₂-, in which A/A₁ is in combination with [HE]. Instead, the combined effects of BU and [HE] to promote hydrolysis, which covert the -L_{B}-A- structure of general formula -M²-A(BU)—[HE]-A_{O}/A′_{a′}-, or more specifically of formula -M²-A(BU)— or -M²-A₁(BU)-A₂-, of a Ligand Drug Conjugate compound to its corresponding -M³-A(BU)—[HE]-A_{O}/A′_{a′}-, -M³-A(BU)— or M³-A₁(BU)—[HE]-A₂- formula, under controlled conditions (as when pH is purposely increased so as to decrease the protonation state of the Basic Unit) are such that an undue molar excess of Drug Linker compound to compensate for hydrolysis of its M¹ moiety is not required. Therefore, Michael addition of the sulfur atom of a reactive thiol functional group of the targeting agent to the maleimide ring system of M¹, which provides a targeting Ligand Unit attached to a succinimide ring system of M², typically occurs at a rate that effectively competes with M¹ hydrolysis. Without being bound by theory, it is believed that at low pH, as for example when the basic amine of BU is in the form of a TFA salt, premature hydrolysis of M¹ in a Drug Linker product is much slower than when the pH is raised to that suitable for base catalysis using an appropriate buffering agent and that an acceptable molar excess of Drug Linker compound can adequately compensate for any loss due to premature M¹ hydrolysis that does occur during the time course for completion or near completion of the Michael addition of a sulfur atom of a targeting agent's reactive thiol functional group to a Drug Linker compound's M¹ moiety.

As previously discussed, enhancement of carbonyl hydrolysis by either type of Basic Unit is dependent on the basicity of its functional group and the distance of that basic functional group in relation to the M¹/M² carbonyl groups. Typically, [HE] is a carbonyl moiety or other carbonyl-containing functional group located distal to the end of the C₁-C₁₂ alkylene of A/A₁ that is bonded to M², or M³ derived therefrom and also provides for covalent attachment to A₂ or to the optional secondary linker this is present, when B is absent and A is a single discreet unit. Carbonyl-containing functional groups other than ketone include esters, carbamates, carbonates and ureas. When [HE] is a carbonyl-containing functional group other than ketone in a drug linker moiety of an ADC having a L_{SS} primary linker, the carbonyl moiety of that functional group, which is shared with A/A₁, is typically bonded to the otherwise optionally substituted C₁-C₁₂ alkylene of A/A₁ distal to its attachment site to the imide nitrogen atom of M² as when [HE] is —C(═O)—X—, wherein X is —O— or optionally substituted —NH—. In some aspects, the [HE] moiety may be sufficiently distant from the imide nitrogen to which of A/A₁ is covalently bonded so that no discernable or minor effect on hydrolytic sensitivity of the succinimide carbonyl-nitrogen bonds of an M²-containing moiety is observable, but instead is driven primarily by BU.

“Stretcher Unit” as used herein, unless otherwise stated or implied by context, refers to an optional organic moiety in a primary or secondary linker of a Linker Unit in a Drug Linker compound or drug linker moiety of Ligand Drug Conjugate, such as an Antibody Drug Conjugate, that physically separates the targeting Ligand Unit (L) from an optional secondary linker when that linker is present is present. When the Linker Unit is comprised of an L_{SS} or L_{S} primary linker a first optional Stretcher is present since it provides the Basic Unit for these types of primary linkers. The presence of a first optional Stretcher Unit (A) in L_{R} may also be required in any type of primary linker when there is insufficient steric relief from the Ligand Unit absent that optional Stretcher Unit to allow for efficient processing of the secondary linker for release of the Drug Unit as a free drug. Alternatively, or in addition to steric relief, those optional components may be included for synthetic ease in preparing a Drug Linker compound. In some aspects when subscript b is 1 a first or second optional Stretcher Unit (A or A′, respectively) is a single unit or can contain multiple subunits (as for example when A has two subunits represented by -A₁-[HE]-A₂-). In other aspects when subscript b is 0 typically, A is one distinct unit or has two distinct subunits when subscript b is 0 and subscript a′ is 1. In still other aspects B/A′ has 2 to 4 independently selected distinct subunits.

In some aspects, when L_{R} is L_{SS}/L_{S}, in addition to covalent attachment to M¹ of a Drug Linker compound or M²/M³ of a drug linker moiety in a LDC/ADC compound, A is bonded to a Branching Unit (B), or W of an optional secondary linker (L_{O}) that is present optionally through A_{O}/A′_{a′} as in A[HE](A_{O}/A′ is absent) or A₁-[HE]-A₂ (A_{O}/A′ present), represented in general as A-[HE]-A_{O}/A_{a′}-, in which A/A₁ and A_{O}/A_{a′} when present as A₂ is also a component of L_{SS}/L_{S}.

In some aspects, A or A′ or a subunit of either of these Stretcher Units has the formula of -L^{P}(PEG)- in which L^{P} is a Parallel Connecter Unit and PEG is a PEG Unit as defined elsewhere. Thus, in some of those aspects a Linker Unit in drug linker moiety of a Ligand Drug Conjugate or Drug Linker compound in which subscript b is 0 and subscript a′ is 1 contains the formula of -A₁-[HE]-L^{P}(PEG)- in which A′ is -L^{P}(PEG)- and is present as A₂. In other of those aspects in which subscript b is 1 and A_{O} is present as A₂, a Linker Unit in drug linker moiety of a Ligand Drug Conjugate or Drug Linker compound contains the formula of -A₁-[HE]-L^{P}(PEG)-B—. In still other aspects subscript b is 1 and subscript a′ is 1, a Ligand Drug Conjugate or Drug Linker compound contains the formula of -A-[HE]-A_{O}-B-L^{P}(PEG) in which A′ is L^{P}(PEG)

In some aspects when subscript a is 1 so that a first optional Stretcher Unit (A) is present, that Unit typically has at least one carbon atom, wherein that atom connects L_{B}/L_{B}′ to [HE]. In some of those aspects in which L_{B}′ is that of a L_{SS}′ primary linker of a Drug Linker compound, that Stretcher Unit is comprised of C₁-C₁₂ alkylene moiety substituted by or incorporating a Basic Unit and is otherwise optionally substituted and has one of its radical carbon atoms attached to the maleimide nitrogen atom and the other to [HE], wherein [HE] is an optional hydrolysis enhancing moiety that is present. In other aspects, when L_{R}′ is other than L_{SS}′, but nonetheless is comprised of a maleimide moiety or some other L_{B}′ moiety, L_{B}′ is attached to an optional first Stretcher Unit (A), which in some aspects is an optionally substituted C₁-C₁₂ alkylene, which is optionally in combination with [HE]. Thus, in some aspects in which L_{R}′ is L_{SS}′ the first optional Stretcher Unit is present and is comprised of a C₁-C₁₂ alkylene moiety, [HE] and an optional subunit (A_{O} when subscript b is 1 or A′_{a′} when subscript b is 0), all of which are components of L_{R}‘ when L_{R}’ is L_{SS}, wherein A is attached to B, which is a component of L_{R}′ or W, which is a component of L_{O}, distal to the attachment site of the C₁-C₁₂ alkylene moiety to the imide nitrogen atom. In other aspects, when subscript a is 1 and A is present as a single discreet unit or of two subunits, A has the general formula of -A-[HE]-A_{O}/A_{a′}- wherein A_{O}/A′_{a′} is an optional subunit of A, or more specifically has the formula of -A₁-[HE]-A₂- when A_{O} is present as a second subunit of A and subscript b is 1 or when subscript a′ is 1 and subscript b is 0 so that A′ is present as a second subunit of A. In such aspects, A_{O}/A₂ or A′/A₂ is an α-amino acid, a β-amino acid or other amine-containing acid residue.

“Branching Unit” as used herein, unless otherwise stated or implied by context, refers to a tri-functional or multi-functional organic moiety that is an optional component of a Linker Unit (LU). A Branching Unit (B) is present in a primary linker of drug linker moiety of Formula 1A of LDC/ADC of Formula 1A, when multiple -L_{O}-D moieties are present is a single drug linker moiety. In an LDC/ADC having the afore-described generalized formula, the absence or presence of a Branching Unit is indicated by subscript b of B_{b} in which subscript b is 0 or 1, respectively. A Branching Unit is at least trifunctional in order to be incorporated into a primary linker. Drug Linker or LDC/ADC compounds having a Branching Unit, which is due to multiple -L_{O}-D moieties per drug linker moiety of formula -LU-D, typically have each secondary linker (L_{O}) containing the formula -A′_{a′}-W—Y_{y}—, wherein A′ is a second optional Stretcher Unit; subscripts a′ is 0 or 1, indicating the absence or presence of A′, respectively; W is a Peptide Cleavable Unit; Y is a Spacer Unit; and subscript y is 0, 1 or 2, indicating the absence or presence of one or two Spacer Units, respectively.

In some aspects, a natural or un-natural amino acid residue or residue of another amine-containing acid compound having a functionalized side chain serves as a trifunctional Branching Unit for attachment of two -L_{O}-D moieties. In some of those aspects B is a lysine, glutamic acid or aspartic acid residue in the L- or D-configuration in which the epsilon-amino, gamma-carboxylic acid or beta-carboxylic acid functional group, respectively, along with their amino and carboxylic acid termini, interconnects B within the remainder of LU. A Branching Unit of greater functionality for attachment of 3 or 4-L_{O}-D moieties is typically comprised of the requisite number of tri-functional subunits.

“Natural amino acid” as used herein, unless otherwise stated or implied by context, refers to a naturally occurring amino acid, namely, arginine, glutamine, phenylalanine, tyrosine, tryptophan, lysine, glycine, alanine, histidine, serine, proline, glutamic acid, aspartic acid, threonine, cysteine, methionine, leucine, asparagine, isoleucine, and valine or a residue thereof, in the L or D-configuration, unless otherwise specified or implied by context.

“Un-natural amino acid” as used herein, unless otherwise stated or implied by context, refers to an alpha-amino-containing acid or residue thereof, which has the backbone structure of a natural amino acid, but has a side chain group attached to the alpha carbon that is not present in natural amino acids.

“Non-classical amino acid” as used herein, unless otherwise stated or implied by context, refers to an amine-containing acid compound that does not have its amine substituent bonded to the carbon alpha to the carboxylic acid and therefore is not an alpha-amino acid. Non-classical amino acids include β-amino acids in which a methylene is inserted between the carboxylic acid and amino functional groups in a natural amino acid or an un-natural amino acid.

“Peptide” as used herein, unless otherwise stated or implied by context, refers to a polymer of two or more amino acids wherein carboxylic acid group of one amino acid forms an amide bond with the alpha-amino group of the next amino acid in the peptide sequence. Methods for preparing amide bonds in polypeptides are additionally provided in the definition of amide. Peptides may be comprised of naturally occurring amino acids in the L- or D-configuration and/or unnatural and/or non-classical amino acids.

“Protease” as defined herein refers to a protein capable of enzymatic cleavage of a carbonyl-nitrogen bond such as an amide bond typically found in a peptide. Proteases are classified into major six classes: serine proteases, threonine proteases, cysteine proteases, glutamic acid proteases, aspartic acid proteases and metalloproteases so named for the catalytic residue in the active site that is primarily responsible for cleaving the carbonyl-nitrogen bond of its substrate. Proteases are characterized by various specificities, which are dependent of identities of the residues at the N-terminal and/or C-terminal side of the carbonyl-nitrogen bond and their various distributions (intracellular and extracellular).

Regulatory proteases are typically intracellular proteases that are required for the regulation of cellular activities that sometimes becomes aberrant or dysregulated in abnormal or other unwanted cells. In some instances, when a Peptide Cleavable Unit is directed to a protease having preferential distribution intracellularly, that protease is a regulatory protease, which is involved in cellular maintenance or proliferation. Those proteases include cathepsins. Cathepsins include the serine proteases, Cathepsin A, Cathepsin G, aspartic acid proteases Cathepsin D, Cathepsin E and the cysteine proteases, Cathepsin B, Cathepsin C, Cathepsin F, Cathepsin H, Cathepsin K, Cathepsin L1, Cathepsin L2, Cathepsin O, Cathepsin S, Cathepsin W and Cathepsin Z.

“Peptide Cleavable Unit” as used herein, unless otherwise stated or implied by context, refers to an organic moiety within a secondary linker of a Ligand Drug Conjugate compound's drug linker moiety or a Drug Linker compound that provides for a recognition site for a protease and is capable of enzymatically releasing its conjugated Drug Unit (D) as free drug upon enzymatic action of that protease.

A recognition site for cleavage by a protease is sometimes limited to those recognized by proteases found in abnormal cells, such as cancer cells, or within nominally normal cells targeted by the Ligand Drug Conjugate that are particular to the environment of the nearby abnormal cells, but which may also be found within normal cells. For that purpose, the peptide is typically resistant to circulating proteases in order to minimize premature release of free drug or precursor thereof that otherwise could cause off-target adverse events from systemic exposure to that drug. In some aspects, the peptide will have one or more D-amino acids or an unnatural or non-classical amino acids in order to have that resistance. In some of those aspects the sequence will comprise a dipeptide or tripeptide in which the P2′ site contains a D-amino acid and the P1′ site contains one of the 20 naturally occurring L-amino acids other than L-proline.

In those aspects, the reactive site is more likely operated upon enzymatically subsequent to immunologically selective binding to the targeted antigen. In some of those aspects, the targeted antigen is on abnormal cells so that the recognition site is more likely operated upon enzymatically subsequent to cellular internalization of a Ligand Drug Conjugate compound into targeted abnormal cells. Consequently, those abnormal cells should display the targeted antigen in higher copy number in comparison to normal cells to mitigate on-target adverse events. In other of those aspects, the targeted antigen is on normal cells that are within and are peculiar to the environment of abnormal cells so that the recognition site is more likely operated upon enzymatically subsequent to cellular internalization of a Ligand Drug Conjugate compound into these targeted normal cells. Consequently, those normal cells should display the targeted antigen in higher copy number in comparison to normal cells distant from the site of the cancer cells to mitigate on-target adverse events.

In any one of the above aspects, protease reactivity towards the recognition site is greater within tumor tissue homogenate in comparison to normal tissue homogenate. That greater reactivity in some aspects is due to a greater amount of intracellular protease activity within the targeted cells of the tumor tissue as compared to intracellular protease activity in normal cells of the normal tissue and/or reduced protease activity in the interstitial space of normal tissue in comparison to that activity of Peptide Cleavable Units of traditional Ligand Drug Conjugates. In those aspects, the intracellular protease is a regulatory protease and the peptide bond of the Peptide Cleavable Unit is capable of being selectively cleaved by an intracellular regulatory protease in comparison to serum proteases in addition to being selectively cleaved by proteases of tumor tissue homogenate in comparison to proteases in normal tissue homogenate.

A secondary linker containing a Peptide Cleavable Unit typically has the formula of -A′_{a′}-W—Y_{y}—, wherein A′ is a second optional Spacer Unit when subscript b is 1; subscript a′ is 0 or 1, W is a Peptide Cleavable Unit; Y is an optional Spacer Unit; and subscript y is 0, 1 or 2. When subscript b is 0 and subscript a′ is 1, A′ becomes a subunit of A so that the secondary linker has the formula of —W—Y_{y}—. For either formula of the secondary linker which protease action on the peptide sequence comprising the Peptide Cleavable Unit results in direct release of D when subscript y is 0 or when subscript y is 1 results in a drug-linker fragment of formula Y-D as the precursor to free drug, in which Y typically undergoes self-immolation to provide free drug, or when subscript y is 2 results in a first drug-linker fragment of formula Y—Y′-D, in which Y is a first Spacer Unit that undergoes self-immolation to provide a second drug linker fragment of formula Y′-D, in which Y′ is a second Spacer Unit that decomposes to complete release of D as free drug.

In some aspects, Drug Linker compounds in which the secondary linker contains a Peptide Cleavable Unit are represented by the structures of Formula IC:

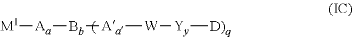

and corresponding drug linker moieties of Ligand Drug Conjugates are represented by the structures of Formula 1D or Formula 1E:

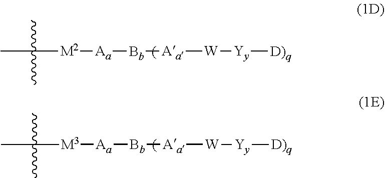

wherein W is the Peptide Cleavable Unit and M¹-Aₐ-B_{b}— of Formula IC, -M²-Aₐ-B_{b}— of Formula 1D and -M³-Aₐ-B_{b}— of Formula 1E are primary linkers, wherein M¹ is a maleimide moiety; M² is a succinimide moiety; M³ is a succinic acid amide moiety; Y is an optional Spacer Unit so that subscript y is 0 or 1 or Y_{y} is —Y—Y′ so that subscript y is 2 and Y and Y′ are a first and second Spacer Unit, respectively, and the remaining variable groups are as defined for Drug Linker compounds of Formula IA and for drug linker moieties of Formula 1A. L_{SS}′ primary linkers of Drug Linker compounds, which contain an M¹ moiety, and L_{SS} primary linkers of drug linker moieties in some LDCs/ADCs, which contain M² moieties, of the present invention are those formulae in which A or a subunit thereof is substituted by or incorporates a Basic Unit. Other primary linkers are L_{S} primary linkers that are derived from the above M²-containing L_{SS} primary linker of Formula 1C by hydrolysis of their succinimide moieties to provide M³-containing moieties of Formula 1D.

In any one of the above aspects, the amide bond that is specifically cleaved by a protease produced by or within a targeted cell is to the amino group of the Spacer Unit (Y) or Drug Unit, if Y is absent Thus, protease action on the peptide sequence in W results in release of D as free drug or its precursor Y_{y}-D, which spontaneously fragments to provide free drug.

“Spacer Unit” as used herein, unless otherwise stated or implied by context, refers to a moiety in a secondary linker (L_{O}) of formula -A′_{a′}-W—Y_{y}— in which subscript y is 1 or 2, indicating the presence of 1 or 2 Spacer Units, within a Drug Linker compound or the Linker Unit of a drug linker moiety of a Ligand Drug Conjugate, wherein A′ is a second optional Spacer Unit, which is some aspects as described herein becomes part of a primary linker to which the secondary linker is covalently attached as a subunit of a first optional Spacer Unit that is present, subscript a′ is 0 or 1 indicating the absence or presence of A′; Y is a Spacer Unit and W is a Peptide Cleavable Unit of formula —Pₙ . . . [P3]-[P2]-[P1]- or —Pₙ . . . [P3]-[P2]-[P1]-[P-1]-, wherein subscript n ranges from 0 to 12 (e.g., 0-10, 3-12 or 3-10) and P1, P2 and P3 are amino acid residues that confer selectivity for protease cleavage by tumor tissue homogenate over normal tissue homogenate as described herein. When subscript y is 1, a Spacer Unit is covalently bonded to W and to a Drug Unit (D), or when subscript y is 2 to another such moiety (Y′) covalently bonded to D. Protease action upon W initiates release D as free drug as further described by the embodiments of the invention.

“Self-immolating moiety” as used herein refers to a bifunctional moiety within a self-immolative Spacer Unit (Y) wherein the self-immolative moiety is covalently attached to a heteroatom of D, or to a shared functional group between Y and D, optionally substituted where permitted, and is also covalently attached to a Peptide Cleavable Unit through another optionally substituted heteroatom (J), wherein J is —NH— or an appropriately substituted nitrogen atom within an amide functional group, so that the self-immolative moiety incorporates these drug linker components into a normally stable tripartite molecule unless activated.

On cleavage of the peptide bond between P1/P-1 and Y, D or a first drug linker fragment, which is Y′-D, spontaneously separates from the tripartite molecule by self-destruction of the self-immolative moiety of its self-immolative Spacer Unit. In some aspects, a component of a self-immolative moiety Spacer Unit intervening between Y′-D or D and the optionally substituted heteroatom J of Y bonded to W has the formula of —C₆-C₂₄ arylene-C(R⁸)(R⁹)—, —C₅-C₂₄ heteroarylene-C(R⁸)(R⁹)—, —C₆-C₂₄ arylene-C(R⁸)═C(R⁹)— or —C₅-C₂₄ heteroarylene-C(R⁸)═C(R⁹)—, optionally substituted, wherein R⁸ and R⁹ are as described by the embodiments of the invention, and typically is C₆-C₁₀ arylene-CH₂— or C₅-C₁₀ heteroarylene-CH₂—, in which the (hetero)arylene is optionally substituted, wherein the component of the self-immolative moiety Spacer Unit is capable of undergoing fragmentation to form a imino-quinone methide or related structure by 1,4 or 1,6-elimination with concomitant release of D or Y′-D on cleavage of the protease cleavable bond between J and W. In some aspects, a self-immolative Spacer Unit having the aforementioned component bonded to J is exemplified by an optionally substituted p-aminobenzyl alcohol (PAB) moiety, ortho or para-aminobenzylacetals, or other aromatic compounds that are electronically similar to the PAB group (i.e., PAB-type) such as 2-aminoimidazol-5-methanol derivatives (see, e.g., Hay et al., 1999, Bioorg. Med. Chem. Lett. 9:2237) or those in which the phenyl group of the p-aminobenzyl alcohol (PAB) moiety is replaced by a heteroarylene.

Without being bound by theory an aromatic carbon of an arylene or heteroarylene group of a PAB or PAB-type moiety of a self-immolative Spacer Unit that is incorporated into a Linker Unit is substituted by J wherein the electron-donating heteroatom of J is attached to the cleavage site of W so that the electron-donating capacity of that heteroatom is attenuated (i.e., its EDG ability is masked by incorporation of a self-immolative moiety of a Self-immolative Spacer Unit into a Linker Unit). The other substituent of the hetero(arylene) is a benzylic carbon that is attached to an optionally substituted heteroatom of D an optionally substituted functional group shared between Y and D or a second Spacer Unit (Y′) bonded to the Drug Unit (D), wherein the benzylic carbon is attached to another aromatic carbon atom of the central arylene or heteroarylene, wherein the aromatic carbon bearing the attenuated electron-donating heteroatom is adjacent to (i.e., 1,2-relationship), or two additional positions removed (i.e., 1,4-relationship) from that benzylic carbon atom. The functionalized EDG heteroatom is chosen so that upon processing of the cleavage site of W the electron-donating capacity of the masked heteroatom is restored thus triggering a 1,4- or 1,6-elimination to expel -D as free drug from the benzylic substituent, or when Y′-D is released subsequent self-immolation of Y′ provides free drug, to elicit a therapeutic effect. Exemplary self-immolative moieties and self-immolative Spacer Unit having those self-immolative moieties are exemplified by the embodiments of the invention.

Other examples of self-immolative groups include, but are not limited to, aromatic compounds that are electronically similar to the PAB group such as 2-aminoimidazol-5-methanol derivatives (see, e.g., Hay et al., 1999, Bioorg. Med. Chem. Lett. 9:2237) and ortho or para-aminobenzylacetals. Spacers can be used that undergo cyclization upon amide bond hydrolysis, such as substituted and unsubstituted 4-aminobutyric acid amides (see, e.g., Rodrigues et al., 1995, Chemistry Biology 2:223), appropriately substituted bicyclo[2.2.1] and bicyclo[2.2.2] ring systems (see, e.g., Storm et al., 1972, J. Amer. Chem. Soc. 94:5815) and 2-aminophenylpropionic acid amides (see, e.g., Amsberry et al., 1990, J. Org. Chem. 55:5867). Elimination of amine-containing drugs that are substituted at the a-position of glycine (see, e.g., Kingsbury et al., 1984, J. Med. Chem. 27:1447) are also examples of self-immolative groups. In one embodiment, the Spacer unit is a branched bis(hydroxymethyl)styrene (BHMS) unit, as described in WO 2007/011968, which can be used to incorporate and release multiple drugs. Additional self-immolative spacers are described in WO 2005/082023.

“Methylene Carbamate Unit” as used herein, unless otherwise stated or implied by context, refers to an organic moiety capable of self-immolation and intervenes between a first self-immolative Spacer Unit and a Drug Unit within a Linker Unit of a Ligand Drug Conjugate or Drug linker compound and as such is an exemplary second Spacer Unit.

A Methylene Carbamate (MAC) Unit bonded to a Drug Unit is represented by formula III:

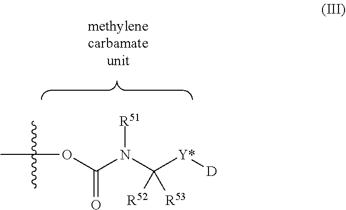

or a pharmaceutically acceptable salt thereof, wherein the wavy line indicates covalent attachment of the methylene carbamate unit to a first self-immolative Spacer Unit (Y); D is a Drug Unit having a functional group (e.g., hydroxyl, thiol, amide or amine functional group) that is incorporated into the methylene carbamate unit; T* is a heteroatom from said functional group, which includes oxygen, sulfur, or nitrogen as optionally substituted —NH—. Upon cleavage of a Linker Unit comprised of a MAC Unit, a first self-immolative Spacer Unit (Y) bonded to that MAC Unit as the second self-immolative Spacer Unit (Y′) undergoes fragmentation to release —Y′-D of formula III. The MAC Unit then spontaneous decomposes to complete release D as free drug, the presumed mechanism for which is indicated by the embodiments of the invention.

“PEG Unit” as used herein refers to a group comprising a polyethylene glycol moiety (PEG) having a repetition of ethylene glycol subunits having the formula of

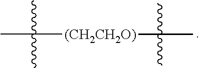

PEGs include polydisperse PEGs, monodisperse PEGs and discrete PEGs. Polydisperse PEGs are a heterogeneous mixture of sizes and molecular weights whereas monodisperse PEGs are typically purified from heterogeneous mixtures and are therefore provide a single chain length and molecular weight. Discrete PEGs are compounds that are synthesized in step-wise fashion and not via a polymerization process. Discrete PEGs provide a single molecule with defined and specified chain length.

A PEG Unit comprises at least 2 subunits, at least 3 subunits, at least 4 subunits, at least 5 subunits, least 6 subunits, at least 7 subunits, at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits. Some PEG Units comprise up to 72 subunits.

“PEG Capping Unit” as used herein is a nominally unreactive organic moiety or functional group that terminates the free and untethered end of a PEG Unit and in some aspects is other than hydrogen. In those aspects a PEG Capping Unit is methoxy, ethoxy, or other C₁-C₆ ether, or is —CH₂—CO₂H, or other suitable moiety. The ether, —CH₂—CO₂H, —CH₂CH₂CO₂H, or other suitable organic moiety thus acts as a “cap” for the terminal PEG subunit of the PEG Unit.

“Parallel Connector Unit” as used herein, unless otherwise stated or implied by context, refers to an organic moiety of a Drug Linker compound or a Ligand Drug Conjugate compound's drug linker moiety, which is typically present in its Linker Unit as a subunit of a first or second Stretcher Unit, wherein the Parallel Connector Unit (L^{P}) is capable of orienting the PEG Unit attached thereto in parallel orientation to a Drug Unit that is hydrophobic, referred herein as a hydrophobic Drug Unit, so as to reduce at least in part the hydrophobicity of that Drug Unit. Structures of L^{P} and associated PEG Units and PEG Capping Units are described by WO 2015/5057699, which are specifically incorporated by reference herein, and in some aspects, L^{P} is a tri-functional α-amino acid, β-amino acid or other tri-functional amine-containing acid residue.

“Intracellularly cleaved”, “intracellular cleavage” and like terms used herein refer to a metabolic process or reaction within a targeted cell occurring upon a Ligand Drug Conjugate or the like, whereby covalent attachment through its Linker Unit between the Drug Unit and the Ligand Unit of the Conjugate is broken, resulting in release of D as free drug within the targeted cell. As described herein, in some embodiments D is initially released as an adduct of the Drug Unit with one or more self-immolative spacers, which self-immolative spacers subsequently spontaneously separate from the Drug Unit to release D as the free drug.

“Hematological malignancy” as used herein, unless otherwise stated or implied by context, refers to a blood cell tumor that originates from cells of lymphoid or myeloid origin and is synonymous with the term “liquid tumor”. Hematological malignancies may be categorized as indolent, moderately aggressive or highly aggressive.

“Lymphoma” as used herein, unless otherwise stated or implied by context, refers to is hematological malignancy that usually develops from hyper-proliferating cells of lymphoid origin. Lymphomas are sometimes classified into two major types: Hodgkin lymphoma (HL) and non-Hodgkin lymphoma (NHL). Lymphomas may also be classified according to the normal cell type that most resemble the cancer cells in accordance with phenotypic, molecular or cytogenic markers. Lymphoma subtypes under that classification include without limitation mature B-cell neoplasms, mature T cell and natural killer (NK) cell neoplasms, Hodgkin lymphoma and immunodeficiency-associated lympho-proliferative disorders. Lymphoma subtypes include precursor T-cell lymphoblastic lymphoma (sometimes referred to as a lymphoblastic leukemia since the T-cell lymphoblasts are produced in the bone marrow), follicular lymphoma, diffuse large B cell lymphoma, mantle cell lymphoma, B-cell chronic lymphocytic lymphoma (sometimes referred to as a leukemia due to peripheral blood involvement), MALT lymphoma, Burkitt's lymphoma, mycosis fungoides and its more aggressive variant Sézary's disease, peripheral T-cell lymphomas not otherwise specified, nodular sclerosis of Hodgkin lymphoma, and mixed-cellularity subtype of Hodgkin lymphoma.

“Leukemia” as used herein, unless otherwise stated or implied by context, refers to a hematological malignancy that usually develops from hyper-proliferating cells of myeloid origin, and include without limitation, acute lymphoblastic leukemia (ALL), acute myelogenous leukemia (AML), chronic lymphocytic leukemia (CLL), chronic myelogenous leukemia (CML) and acute monocyctic leukemia (AMoL). Other leukemias include hairy cell leukemia (HCL), T-cell lymphatic leukemia (T-PLL), large granular lymphocytic leukemia and adult T-cell leukemia.

“Hyper-proliferating cells” as used herein, unless otherwise stated or implied by context, refer to abnormal cells that are characterized by unwanted cellular proliferation or an abnormally high rate or persistent state of cell division or other cellular activity that is unrelated or uncoordinated with that of the surrounding normal tissues. In some aspects, hyper-proliferating cells are hyper-proliferating mammalian cells. In other aspects, hyper-proliferating cells are hyper-stimulated immune cells as defined herein whose persistent state of cell division or activation occurs after the cessation of the stimulus that may have initially evoked the change in their cell division. In other aspects, the hyper-proliferating cells are transformed normal cells or cancer cells and their uncontrolled and progressive state of cell proliferation may result in a tumor that is benign, potentially malignant (premalignant) or frankly malignant. Hyperproliferation conditions resulting from transformed normal cells or cancer cells include, but are not limited to, those characterized as a precancer, hyperplasia, dysplasia, adenoma, sarcoma, blastoma, carcinoma, lymphoma, leukemia or papilloma. Precancers are usually defined as lesions that exhibit histological changes and are associated with an increased risk of cancer development and sometimes have some, but not all, of the molecular and phenotypic properties that characterize the cancer. Hormone associated or hormone sensitive precancers include without limitation, prostatic intraepithelial neoplasia (PIN), particularly high-grade PIN (HGPIN), atypical small acinar proliferation (ASAP), cervical dysplasia and ductal carcinoma in situ. Hyperplasias generally refers to the proliferation of cells within an organ or tissue beyond that which is ordinarily seen that may result in the gross enlargement of an organ or in the formation of a benign tumor or growth. Hyperplasias include, but are not limited to, endometrial hyperplasia (endometriosis), benign prostatic hyperplasia and ductal hyperplasia.

“Normal cells” as used herein, unless otherwise stated or implied by context, refer to cells undergoing coordinated cell division related to maintenance of cellular integrity of normal tissue or replenishment of circulating lymphatic or blood cells that is required by regulated cellular turnover, or tissue repair necessitated by injury, or to a regulated immune or inflammatory response resulting from pathogen exposure or other cellular insult, where the provoked cell division or immune response terminates on completion of the necessary maintenance, replenishment or pathogen clearance. Normal cells include normally proliferating cells, normal quiescent cells and normally activated immune cells. Normal cells include normal quiescent cells, which are noncancerous cells in their resting Gₒ state and have not been stimulated by stress or a mitogen or are immune cells that are normally inactive or have not been activated by pro-inflammatory cytokine exposure.

“Abnormal cells” as the term is used herein, unless otherwise stated or implied by context, refers to normal cells that have become dysfunctional either in disproportionate response to external stimuli or from failure to appropriately regulate their spontaneous intracellular activity, which in some instances has a mutational origin. Abnormal cells include hyper-proliferating cells and hyper-stimulated immune cells, as these term are defined elsewhere. Those cells when present in an organism typically interfere with the functioning of otherwise normal cells causing harm to the organism and over time will increase in destructive capacity. Abnormal cells include cancer cells, hyperactivate immune cells and other unwanted cells of the organism. Abnormal cells may also refer to nominally normal cells that are in the environment of outwardly abnormal cells, but which nonetheless support the proliferation and/or survival of these other abnormal cells, such as tumor cells, so that targeting the nominally normal cells indirectly inhibits the proliferation and/or survival of the tumor cells.

“Hyper-stimulated immune cells” as used herein, unless otherwise stated or implied by context, refer to cells involved in innate or adaptive immunity characterized by an abnormally persistent proliferation or inappropriate state of stimulation that occurs after the cessation of the stimulus that may have initially evoked the change in proliferation or stimulation or that occurs in the absence of any external insult. Oftentimes, the persistent proliferation or inappropriate state of stimulation results in a chronic state of inflammation characteristic of a disease state or condition. In some instances, the stimulus that may have initially evoked the change in proliferation or stimulation is not attributable to an external insult but is internally derived, as in an autoimmune disease. In some aspects, a hyper-stimulated immune cell is a pro-inflammatory immune cell that has been hyper-activated through chronic pro-inflammatory cytokine exposure.

In some aspects of the invention, a Ligand Drug Conjugate compound of a Ligand Drug Conjugate composition binds to an antigen preferentially displayed by pro-inflammatory immune cells that are abnormally proliferating or are inappropriately or persistently activated. Those immune cells include classically activated macrophages or Type 1 T helper (Th1) cells, which produce interferon-gamma (INF-γ), interleukin-2 (IL-2), interleukin-10 (IL-10), and tumor necrosis factor-beta (TNF-β), which are cytokines that are involved in macrophage and CD8⁺ T cell activation.

“Bioavailability” unless otherwise stated or implied by context, refers to the systemic availability (i.e., blood/plasma levels) of a given amount of a drug administered to a patient. Bioavailability is an absolute term that indicates measurement of both the time (rate) and total amount (extent) of drug that reaches the general circulation from an administered dosage form.

“Subject” unless otherwise stated or implied by context, refers to a human, non-human primate or mammal having a hyper-proliferation, inflammatory or immune disorder or other disorder attributable to abnormal cells or is prone to such a disorder who would benefit from administering an effective amount of a Ligand Drug Conjugate. Non-limiting examples of a subject include human, rat, mouse, guinea pig, monkey, pig, goat, cow, horse, dog, cat, bird and fowl. Typically, the subject is a human, non-human primate, rat, mouse or dog.

“Carrier” unless otherwise stated or implied by context refers to a diluent, adjuvant or excipient, with which a compound is administered. Such pharmaceutical carriers can be liquids, such as water and oils, including those of petroleum, animal, vegetable or synthetic origin, such as peanut oil, soybean oil, mineral oil, sesame oil. The carriers can be saline, gum acacia, gelatin, starch paste, talc, keratin, colloidal silica, urea. In addition, auxiliary, stabilizing, thickening, lubricating and coloring agents can be used. In one embodiment, when administered to a subject, the compound or compositions and pharmaceutically acceptable carriers are sterile. Water is an exemplary carrier when the compounds are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. The present compositions, if desired, can also contain minor amounts of wetting or emulsifying agents, or pH buffering agents.

“Salt form” as used herein, unless otherwise indicated by context, refers to a charged compound in ionic association with a countercation(s) and/or counteranions so as to form an overall neutral species. In some aspects, a salt form of a compound occurs through interaction of the parent compound's basic or acid functional group with an external acid or base, respectively. In other aspects the charged atom of the compound that is associated with a counteranion is permanent in the sense that spontaneous disassociation to a neural species cannot occur without altering the structural integrity of the parent compound as when a nitrogen atom is quaternized. Accordingly, a salt form of a compound may involve a quaternized nitrogen atom within that compound and/or a protonated form of a basic functional group and/or ionized carboxylic acid of that compound each of which is in ionic association with a counteranion.

In some aspects a salt form may result from interaction of a basic functional group and an ionized acid functional group within the same compound or involve inclusion of a negatively charged molecule such as an acetate ion, a succinate ion or other counteranion. Thus, a compound in salt form may have more than one charged atom in its structure. In instances where multiple charged atoms of the parent compound are part of the salt form, that salt from can have multiple counter ions so that a salt form of a compound may have one or more charged atoms and/or one or more counterions. The counterion may be any charged organic or inorganic moiety that stabilizes an opposite charge on the parent compound.

A protonated salt form of a compound is typically obtained when a basic functional group of a compound, such as a primary, secondary or tertiary amine or other basic amine functional group interacts with an organic or inorganic acid of suitable pKa for protonation of the basic functional group, or when an acid functional group of a compound with a suitable pKₐ, such as a carboxylic acid, interacts with a hydroxide salt, such as NaOH or KOH, or an organic base of suitable strength, such as triethylamine, for deprotonation of the acid functional group. In some aspects, a compound in salt form contains at least one basic amine functional group, and accordingly acid addition salts can be formed with this amine group, which includes the basic amine functional group of a cyclic or acyclic Basic Unit. A suitable salt form in the context of a Drug Linker compound is one that does not unduly interfere with the condensation reaction between a targeting agent and the Drug Linker compound that provides a Ligand drug Conjugate.

“Pharmaceutically acceptable salt” as used herein, unless otherwise indicated by context, refers to a salt form of a compound in which its counterion is acceptable for administration of the salt form to an intended subject and include inorganic and organic countercations and counteranions. Exemplary pharmaceutically acceptable counteranions for basic amine functional groups, such as those in cyclic or acyclic Basic Units, include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, mesylate, besylate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

Typically, a pharmaceutically acceptable salt is selected from those described in P. H. Stahl and C. G. Wermuth, editors, Handbook of Pharmaceutical Salts: Properties, Selection and Use, Weinheim/Ziirich:Wiley-VCH/VHCA, 2002. Salt selection is dependent on properties the drug product must exhibit, including adequate aqueous solubility at various pH values, depending upon the intended route(s) of administration, crystallinity with flow characteristics and low hygroscopicity (i.e., water absorption versus relative humidity) suitable for handling and required shelf life by determining chemical and solid-state stability as when in a lyophilized formulation under accelerated conditions (i.e., for determining degradation or solid-state changes when stored at 40° C. and 75% relative humidity).

“Inhibit”, “inhibition of” and like terms, unless otherwise stated or implied by context, means to reduce by a measurable amount, or to prevent entirely an undesired activity or outcome. In some aspects, the undesired outcome or activity is related to abnormal cells and includes hyper-proliferation, or hyper-stimulation or other dysregulated cellular activity underlying a disease state. Inhibition of such a dysregulated cellular activity by a Ligand Drug Conjugate is typically determined relative to untreated cells (sham treated with vehicle) in a suitable test system as in cell culture (in vitro) or in a xenograft model (in vivo). Typically, a Ligand Drug Conjugate that targets an antigen that is not present or has low copy number on the abnormal cells of interest or is genetically engineered to not recognize any known antigen is used as a negative control.

“Treat”, “treatment,” and like terms, unless otherwise indicated by context, refer to a therapeutic treatment, including prophylactic measures to prevent relapse, wherein the object is to inhibit or slow down (lessen) an undesired physiological change or disorder, such as the development or spread of cancer or tissue damage from chronic inflammation. Typically, beneficial or desired clinical benefits of such therapeutic treatments include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival or quality of life as compared to expected survival or quality of life if not receiving treatment. Those in need of treatment include those already having the condition or disorder as well as those prone to have the condition or disorder.

In the context of cancer, the term “treating” includes any or all of inhibiting growth of tumor cells, cancer cells, or of a tumor; inhibiting replication of tumor cells or cancer cells, inhibiting dissemination of tumor cells or cancer cell, lessening of overall tumor burden or decreasing the number of cancerous cells, or ameliorating one or more symptoms associated with cancer.

“Therapeutically effective amount” as the term is used herein, unless otherwise stated or implied by context, refers to an amount of free drug or Ligand Drug Conjugate having a Drug Unit, which is released as a free drug, effective to treat a disease or disorder in a mammal. In the case of cancer, the therapeutically effective amount of the free drug or Ligand Drug Conjugate may reduce the number of cancer cells; reduce the tumor size, inhibit (i.e., slow to some extent and preferably stop) cancer cell infiltration into peripheral organs, inhibit (i.e., slow to some extent and preferably stop) tumor metastasis, inhibit, to some extent, tumor growth, and/or relieve to some extent one or more of the symptoms associated with the cancer. To the extent the free drug or Ligand Drug Conjugate may inhibit growth and/or kill existing cancer cells, it may be cytostatic or cytotoxic. For cancer therapy, efficacy can, for example, be measured by assessing the time to disease progression (TTP) determining the response rate (RR) and/or overall survival (OS).

In the case of immune disorders resulting from hyper-stimulated immune cells, a therapeutically effective amount of the drug may reduce the number of hyper-stimulated immune cells, the extent of their stimulation and/or infiltration into otherwise normal tissue and/or relieve to some extent one or more of the symptoms associated with a dysregulated immune system due to hyper-stimulated immune cells. For immune disorders due to hyper-stimulated immune cells, efficacy can, for example, be measured by assessing one or more inflammatory surrogates, including one or more cytokines levels such as those for IL-1β, TNFα, INFγ and MCP-1, or numbers of classically activated macrophages.

In some aspects of the invention, a Ligand Drug Conjugate compound associates with an antigen on the surface of a targeted cell (i.e., an abnormal cell such as a hyper-proliferating cell or a hyper-stimulated immune cell), and the Conjugate compound is then taken up inside the targeted cell through receptor-mediated endocytosis. Once inside the cell, one or more Cleavage Units within a Linker Unit of the Conjugate are cleaved, resulting in release of Drug Unit (D) as free drug. The free drug so released is then able to migrate within the cytosol and induce cytotoxic or cytostatic activities, or in the case of hyper-stimulated immune cells may alternatively inhibit pro-inflammatory signal transduction. In another aspect of the invention, the Drug Unit (D) is released from a Ligand Drug Conjugate compound outside the targeted cell but within the vicinity of the targeted cell so that the resulting free drug from that release is localized to the desired site of action and is able to subsequently penetrate the cell rather than being prematurely released at distal sites.

### 2. EMBODIMENTS

Various embodiments of the invention are described below followed by a more detailed discussion of the components, e.g., groups, reagents, and steps that are useful in the processes of the present invention. Any of the selected embodiments for the components of the processes can apply to each and every aspect of the invention as described herein or they may relate to a single aspect. In some aspects, the selected embodiments may be combined in any combination appropriate for describing an auristatin Ligand Drug Conjugate, Drug Linker compound or Intermediate thereof having a hydrophobic auristatin F Drug Unit.

### 2.1 Ligand Drug Conjugates

A Ligand Drug Conjugate (LDC) compound of the present invention is compound having a Drug Unit connected to a Ligand Unit through an intervening Linker Unit (LU) in which LU is comprised of a Peptide Cleavable Unit that is more susceptible to proteolytic cleavage by tumor tissue homogenate compared to normal tissue homogenate to effect release D as free drug, and typically has the structure of Formula 1:

L-[LU-(D′)]ₚ′ (1)

or a salt thereof, in particular a pharmaceutically acceptable salt thereof, wherein L is the Ligand Unit; LU is the Linker Unit; D′ represents from 1 to 4 Drug Units, incorporating or corresponding in structure to the same free drug for each drug linker moiety of formula -LU-(D)′; and subscript p′ is an integer ranging from 1 to 24, wherein the Ligand Unit is capable of selective binding to an antigen of targeted abnormal cells, wherein the targeted antigen is capable of internalization along with bound Conjugate compound for subsequent intracellular release of free drug, wherein each drug linker moiety in the Ligand Drug Conjugate compound has the structure of Formula 1A:

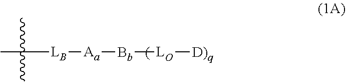

or a salt thereof, in particular, a pharmaceutically acceptable salt, wherein the -L_{B}-Aₐ-B_{b}— moiety of a drug linker moiety of Formula 1A in general represents the primary linker (L_{R}) of the Linker Unit (LU) of Formula 1
wherein the wavy line indicates covalent attachment to L; L_{B} is a Ligand covalent binding moiety; A is a first optional Stretcher Unit; subscript a is 0 or 1 indicating the absence of presence of A, respectively; B is an optional Branching Unit; subscript b is 0 or 1, indicating the absence of presence of B, respectively; D is the Drug Unit; and subscript q is an integer ranging from 1 to 4; and L_{O} is a secondary linker moiety having the structure of:

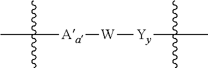

wherein the wavy line adjacent to A′ indicates the site of covalent attachment of L_{O} to the primary linker; the wavy line adjacent to Y indicates the site of covalent attachment of L_{O} to the Drug Unit; A′ is a second optional Spacer Unit, subscript a′ is 0 or 1, indicating the absence or presence of A′, respectively, W is a Peptide Cleavable Unit, Y is a Spacer Unit, and y is 0, 1 or 2, indicating the absence or presence of 1 or 2 Spacer Units, respectively.

A Ligand Drug Conjugate composition is comprised of a distribution or collection of Ligand Drug Conjugate compounds and is represented by the structure of Formula 1 in which subscript p′ is replaced by subscript p, wherein subscript p is an number ranging from about 2 to about 24.

A traditional Ligand Drug Conjugate is also represented by Formula 1, but having a Peptide Cleavable Unit (W) comprised of a dipeptide covalently attached either directly to D or indirectly through Y, in which the dipeptide is designed to be selective for a specific intracellular protease whose activity is upregulated in abnormal cells relative to that of normal cells. In contrast, Conjugates of the present invention are based upon the unexpected finding that the overall protease activity within tissue comprised of the abnormal cells may be differentiated from that activity within normal tissue comprised of the normal cells by an appropriately designed Cleavable Unit while remaining resistant to cleavage by freely circulating proteases. For the Conjugates of the present invention that differentiation is achieved by a Peptide Cleavable Unit incorporating certain tripeptides, wherein these peptides have been identified by a screening method described herein in which protease activity from a tissue homogenate comprised of abnormal cells is compared to that of a normal tissue homogenate, wherein the normal tissue is known to be the source of on-target and/or off-target adverse event(s) experienced by a mammalian subject when administered a therapeutically effective amount of a traditional Ligand Drug Conjugate.

Thus, in a principle embodiment of the invention, W is a Peptide Cleavable Unit comprised of a tripeptide that provides for a recognition site that is selectively acted upon by one or more intracellular proteases of targeted abnormal cells in comparison to freely circulating proteases and is also selectively acted upon by proteases within a tumor tissue homogenate in comparison to proteases within a normal tissue homogenate. For the treatment of a cancer a tripeptide sequence for the Peptide Cleavable Unit is selected so that proteases of normal tissue known to be the source of on-target and/or off-target adverse events from administration of a therapeutically effective amount of a traditional Ligand Drug Conjugate are less likely to act upon the Conjugate having that tripeptide-based Cleavable Unit than proteases of tumor tissue so as to provide greater selectivity for targeting cancer cells. That selection is based upon the lower overall protease activity in the homogenate of the normal tissue compared to homogenate of the tumor tissue of the cancer. In contrast to the improved Conjugates of the present invention, traditional Ligand Drug Conjugate containing a dipeptide Cleavable Unit have been designed to be selectively acted upon by cathepsin B, which is an intracellular protease whose activity is upregulated in cancer cells, and primarily rely upon immunological specificity for selectivity targeting cancer cells over normal cells. Improved Conjugates of the present invention have an additional level of selectivity by being less prone to protease action within normal tissue as compared to the tumor tissue in which the targeted cancer cells reside.

In some embodiments, a drug linker moiety of Formula 1A will have the structure represented by Formula 1B:

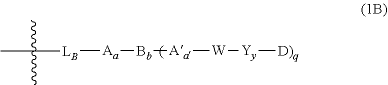

wherein L_{B} is a ligand covalent binding moiety as defined herein for a primary linker (L_{R}) in the Linker Unit (LU) of a drug linker moiety or Drug Linker compound; A and B are a first optional Stretcher Unit and an optional Branching Unit, respectively, of L_{R}; subscript q ranges from 1 to 4; and the remaining variable groups are as defined herein for L_{O}.

In some of those embodiments W contains a tripeptide that is directly attached to the Drug Unit so that subscript y is 0. When subscript y is 1, the tripeptide is attached to a self-immolative Spacer Unit so that cleavage by the protease provides a drug linker fragment of formula Y-D in which Y undergoes self-immolation so as to complete release of the free drug. When subscript y is 2, the tripeptide is attached to a first self-immolative Spacer Unit (Y) so that cleavage by the protease provides a first drug linker fragment of formula Y—Y′-D in which Y′ and is a second Spacer Unit and is followed by self-immolation of the first Spacer Unit so as to provide a second drug linker fragment of formula Y′-D that decomposes to complete the release of the free drug.

Exemplary Ligand Drug Conjugate compounds having drug linker moieties of Formula 1B in which the tripeptide of the Peptide Cleavable Unit (W) is directly attached to the Drug Unit or to an intervening Spacer Unit have the structure of Scheme 1a, wherein P1, P2, and P3 are amino acid residues of the tripeptide sequence and D is attached to a p-amino benzyl alcohol residue through a carbamate or carbonate functional group that together represent Y_{y} in which subscript y is 2. In those exemplary Ligand Drug Conjugate compounds the carbonyl functional group of the amide bond adjacent to P1 is from the C-terminus of the tripeptide sequence wherein that amide bond is the site of protease cleavage (indicated by the arrow) and the amino group of the amide bond adjacent to P3 is from the N-terminus of the tripeptide sequence. Cleavage of the amide functional group to P1 results in a first drug linker fragment having the structure shown in Scheme 1 a, which undergoes self-immolation to provide a second drug linker fragment that spontaneously decomposes with release of CO₂ to complete release of D as free drug of formula H-T*-D* having a hydroxy or amine group, the oxygen atom or nitrogen moiety —NH— of which is presented by T*, wherein D* represents the remainder of the free drug.

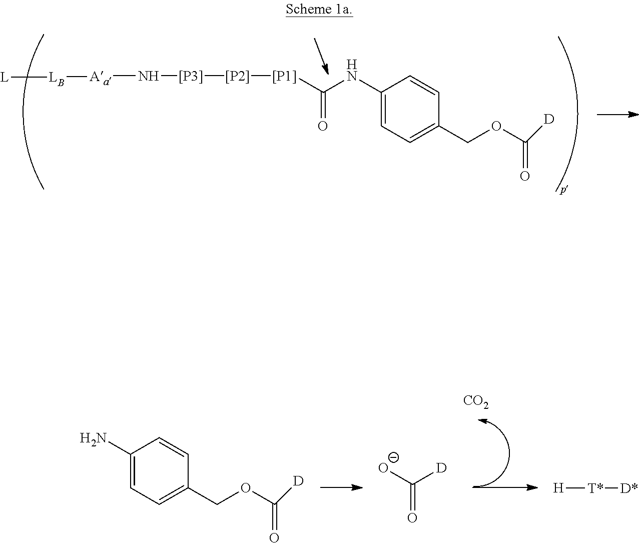

In those embodiments, one or more amino acids designated as P4, P5, etc. may be present between the primary linker of formula -L_{B}-A′_{a′}- and P3 as part of the peptide sequence comprising the tripeptide that confers selectivity for intracellular proteolysis over proteolysis by freely circulating proteases and proteolysis by tumor tissue homogenate over proteolysis by normal tissue homogenate. The mechanism of free drug release from Ligand Drug Conjugates having such extended peptide sequences is analogous to that of Scheme 1a.

In other embodiments an amino acid residue designated as P-1 intervenes between the specificity-conferring tripeptide of W and D or —Y_{y}-D so that D or the drug linker fragment initially released from protease action at the specificity-conferring tripeptide comprises that amino acid, and thus requires further processing by an intracellular endopeptidase to allow for self-immolation of the Spacer Unit(s) to occur. For those embodiments, exemplary Ligand Drug Conjugate compounds having drug linker moieties of Formula 1B in which the specificity-conferring tripeptide of the Peptide Cleavable Unit is not directly attached to the Drug Unit or to an intervening Spacer Unit have the structure shown in Scheme 1b. Protease cleavage of the susceptible amide bond between P1 and P-1 (indicated by the arrow) provides a drug linker fragment in which a first self-immolative Spacer Unit (Y) is present as an amino acid residue that provides for a substrate of an endopeptidase with attachment to the self-immolative moiety of Y, which is the para-amino benzyl alcohol residue having attachment to D through a carbamate or carbonate function group. Together the amino acid-para-amino benzyl alcohol residue and the carbamate or carbonate functional group represent Y_{y} in which subscript y is 2. After endopeptidase removal of P-1, self-immolation occurs as in Scheme 1a for release of D as free drug of formula H-T*-D*.

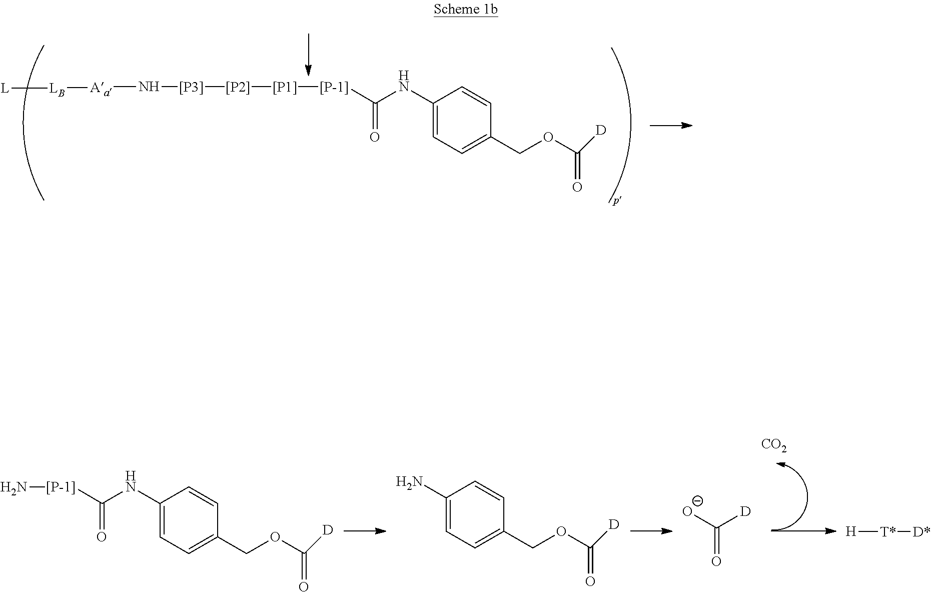

As before one or more amino acids designated as P4, P5, etc. may be present between the primary linker of formula -L_{B}-A′_{a′}- and P3 as part of the peptide sequence comprising the tripeptide that confers selectivity for intracellular proteolysis over proteolysis by freely circulating proteases and proteolysis by tumor tissue homogenate over proteolysis by normal tissue homogenate. Although P-1 in Scheme 1b is formally part of a first self-immolative Spacer Unit (Y), for convenience it will be associated with the tripeptide sequence so that W is a tetrapeptide in SEQ IDs describing such Peptide Cleavable Units. Those Units and other components of Ligand Drug Conjugates of the present invention, are further discussed as follows.

### 2.2.1 Ligand Unit

A Ligand Unit (L) of a Ligand Drug Conjugate is the targeting moiety of the Conjugate that selectively binds to a targeted moiety. In some embodiments the Ligand Unit selectively binds to a cell component (a Cell Binding Agent), which serves as the targeted moiety, or to other target molecules of interest. The Ligand Unit acts to target and present the Drug Unit of the Ligand Drug Conjugate to the particular target cell population with which the Ligand Unit interacts in order to selectively release D as a free drug. Targeting agents that provide for Ligand Units include, but are not limited to, proteins, polypeptides and peptides. Exemplary Ligand Units include, but are not limited to, those provided by proteins, polypeptides and peptides such as antibodies, e.g., full-length antibodies and antigen binding fragments thereof, interferons, lymphokines, hormones, growth factors and colony-stimulating factors. Other suitable Ligand Units are those from vitamins, nutrient-transport molecules, or any other cell binding molecule or substance. In some embodiments a Ligand Unit is from non-antibody protein targeting agent. In other embodiments, a Ligand Unit is from a protein targeting agent such as an antibody. Preferred targeting agents are larger molecular weight proteins, e.g., Cell Binding Agents having a molecular weight of at least about 80 Kd.

A targeting agent reacts with a ligand covalent binding precursor (L_{B}′) moiety of a primary linker precursor (L_{R}′) of a Drug Linker compound to form a Ligand Unit covalently attached to a ligand covalent binding (L_{B}) moiety of a primary linker (L_{R}) of a drug-linker moiety of Formula 1A. The targeting agent has or is modified to have the appropriate number of attachment sites to accommodate the requisite number of drug-linker moieties, defined by subscript p, whether they be naturally occurring or non-naturally occurring (e.g., engineered). For example, for the value of subscript p to be from 6 to 14, a targeting agent must be capable of forming a bond to 6 to 14 drug-linker moieties. The attachment sites can be naturally occurring or engineered into the targeting agent. A targeting agent can form a bond to the L_{SS} moiety of the Linker Unit of a Drug Linker compound via a reactive or activateable heteroatom or a heteroatom-containing functional group of the targeting agent. Reactive or activateable heteroatoms or a heteroatom-containing functional groups that may be present on a targeting agent include sulfur (in one embodiment, from a thiol functional group of an targeting agent), C═O (in one embodiment, from a carbonyl, carboxyl or hydroxyl group of a targeting agent) and nitrogen (in one embodiment, from a primary or secondary amino group of a targeting agent). Those heteroatoms can be present on the targeting agent in the targeting agent's natural state, for example a naturally occurring antibody, or can be introduced into the targeting agent via chemical modification or genetic engineering.

In one embodiment, a targeting agent has a thiol functional group (e.g., of a cysteine residue) and the Ligand Unit therefrom is attached to a drug linker moiety of a Ligand Drug Conjugate compound via the thiol functional group's sulfur atom.

In another embodiment, the targeting agent has lysine residues that can react with an activated ester, including but are not limited to, N-hydroxysuccinimide, pentafluorophenyl, and p-nitrophenyl esters, of L_{R} of the Linker Unit of a Drug Linker compound and thus results in an amide bond between the nitrogen atom from the Ligand Unit and the C═O functional group from the Linker Unit of the Drug Linker compound.

In yet another embodiment, the targeting agent has one or more lysine residues that can be chemically modified to introduce one or more thiol functional groups. The Ligand Unit from that targeting agent is attached to the Linker Unit via the introduced thiol functional group's sulfur atom. The reagents that can be used to modify lysines include, but are not limited to, N-succinimidyl S-acetylthioacetate (SATA) and 2-Iminothiolane hydrochloride (Traut's Reagent).

In another embodiment, the targeting agent can have one or more carbohydrate groups that can be chemically modified to have one or more thiol functional groups. The Ligand Unit from that targeting agent is attached to the Linker Unit via the introduced thiol functional group's sulfur atom, or the targeting agent can have one or more carbohydrate groups that can be oxidized to provide an aldehyde (—CHO) group (see, e.g., Laguzza, et al., 1989, J. Med. Chem. 32(3):548-55). The corresponding aldehyde can then react with an L_{SS} moiety of a Drug Linker compound having nucleophilic nitrogen. Other reactive sites on L_{R} that can react with a carbonyl group on a targeting agent include, but are not limited to, hydrazine and hydroxylamine. Other protocols for the modification of proteins for the attachment of drug linker moieties are described in Coligan et al., Current Protocols in Protein Science, vol. 2, John Wiley & Sons (2002) (incorporated herein by reference).

In preferred embodiments, the reactive group of L_{R} of a Drug Linker compound is a maleimide (M¹) moiety and covalent attachment of L to L_{R} is accomplished through a thiol functional group of a targeting agent so that a thio-substituted succinimide (M²) moiety is formed through Michael addition. The thiol functional group can be present on the targeting agent in the targeting agent's natural state, for example a naturally occurring residue, or can be introduced into the targeting agent via chemical modification and/or genetic engineering.

It has been observed for bioconjugates that the site of drug conjugation can affect numerous parameters including ease of conjugation, drug-linker stability, effects on biophysical properties of the resulting bioconjugates, and in-vitro cytotoxicity. With respect to drug-linker stability, the site of conjugation of a drug-linker to a ligand can affect the ability of the conjugated drug-linker moiety to undergo an elimination reaction and for the drug linker moiety to be transferred from the Ligand Unit of a bioconjugate to an alternative reactive thiol present in the milieu of the bioconjugate, such as, for example, a reactive thiol in albumin, free cysteine, or glutathione when in plasma. Such sites include, for example, the interchain disulfides as well as select cysteine engineered sites. The Ligand-Drug Conjugates described herein can be conjugated to thiol residues at sites that are less susceptible to the elimination reaction (e.g., positions 239 according to the EU index as set forth in Kabat) in addition to other sites.

In preferred embodiments, the Ligand Unit (L) is of an antibody or antigen-binding fragment thereof, thereby defining an antibody Ligand Unit of an Antibody Drug Conjugate (ADC), wherein the antibody Ligand Unit is capable of selective binding to a targeted antigen of a cancer cell for subsequent release of D as free drug, wherein the targeted antigen is capable of internalization into said cancer cell upon said binding in order to initiate intracellular release of free drug.

Useful antibodies include polyclonal antibodies, which are heterogeneous populations of antibody molecules derived from the sera of immunized animals. Other useful antibodies are monoclonal antibodies, which are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art which provides for production of antibody molecules by continuous cell lines in culture.

Useful monoclonal antibodies include, but are not limited to, human monoclonal antibodies, humanized monoclonal antibodies, or chimeric human-mouse (or other species) monoclonal antibodies. The antibodies include full-length antibodies and antigen binding fragments thereof. Human monoclonal antibodies may be made by any of numerous techniques known in the art (e.g., Teng et al., 1983, Proc. Natl. Acad. Sci. USA. 80:7308-7312; Kozbor et al., 1983, Immunology Today 4:72-79; and Olsson et al., 1982, Meth. Enzymol. 92:3-16).

The antibody can be a functionally active fragment, derivative or analog of an antibody that immunospecifically binds to targeted cells (e.g., cancer cell antigens, viral antigens, or microbial antigens) or other antibodies bound to tumor cells or matrix. In this regard, “functionally active” means that the fragment, derivative or analog is able to immunospecifically binds to target cells. To determine which CDR sequences bind the antigen, synthetic peptides containing the CDR sequences can be used in binding assays with the antigen by any binding assay method known in the art (e.g., the BIA core assay) (See, e.g., Kabat et al., 1991, Sequences of Proteins of Immunological Interest, Fifth Edition, National Institute of Health, Bethesda, Md; Kabat E et al., 1980, J. Immunology 125(3):961-969).

Other useful antibodies include fragments of antibodies such as, but not limited to, F(ab′)₂ fragments, Fab fragments, Fvs, single chain antibodies, diabodies, triabodies, tetrabodies, scFv, scFv-FV, or any other molecule with the same specificity as the antibody.

Additionally, recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as for example, those having a variable region derived from a murine monoclonal and human immunoglobulin constant regions. (See, e.g., U.S. Pat. Nos. 4,816,567; and 4,816,397, which are incorporated herein by reference in their entirety). Humanized antibodies are antibody molecules from non-human species having one or more complementarity determining regions (CDRs) from the non-human species and a framework region from a human immunoglobulin molecule. (See, e.g., U.S. Pat. No. 5,585,089, which is incorporated herein by reference in its entirety). Such chimeric and humanized monoclonal antibodies can be produced by recombinant DNA techniques known in the art, for example using methods, each of which is specifically incorporated herein by reference, as described in International Publication No. WO 87/02671; European Patent Publication No. 0 184 187; European Patent Publication No. 0 171 496; European Patent Publication No. 0 173 494; International Publication No. WO 86/01533; U.S. Pat. No. 4,816,567; European Patent Publication No. 012 023; Berter et al., Science (1988) 240:1041-1043; Liu et al., Proc. Natl. Acad. Sci. (USA) (1987) 84: 3439-3443; Liu et al., J. Immunol. (1987) 139: 3521-3526; Sun et al. Proc. Natl. Acad. Sci. (USA) (1987) 84: 214-218; Nishimura et al. Cancer. Res. (1987) 47: 999-1005; Wood et al., Nature (1985) 314:446-449; Shaw et al., J. Natl. Cancer Inst. (1988) 80: 1553-1559; Morrison, Science (1985) 229:1202-1207; Oi et al. BioTechniques (1986) 4: 214; U.S. Pat. No. 5,225,539; Jones et al., Nature (1986) 321: 552-525; Verhoeyan et al., Science (1988) 239: 1534; and Beidler et al., J. Immunol. (1988) 141: 4053-4060.

Completely human antibodies are particularly preferred and can be produced using transgenic mice that are incapable of expressing endogenous immunoglobulin heavy and light chains genes, but which can express human heavy and light chain genes.

Antibodies include analogs and derivatives that are either modified, i.e., by the covalent attachment of any type of molecule if such covalent attachment permits the antibody to retain its antigen binding immunospecificity. For example, but not by way of limitation, derivatives and analogs of the antibodies include those that have been further modified, e.g., by glycosylation, acetylation, PEGylation, phosphorylation, amidation, derivitization by known protecting/blocking groups, proteolytic cleavage, linkage to a cellular antibody unit or other protein, etc. Any of numerous chemical modifications can be carried out by known techniques including, but not limited to, specific chemical cleavage, acetylation, formylation, metabolic synthesis in the presence of tunicamycin, etc. Additionally, the analog or derivative can contain one or more unnatural amino acids.

Antibodies can have modifications (e.g., substitutions, deletions or additions) in amino acid residues that interact with Fc receptors. In particular, antibodies can have modifications in amino acid residues identified as involved in the interaction between the anti-Fc domain and the FcRn receptor (see, e.g., International Publication No. WO 97/34631, which is incorporated herein by reference in its entirety).

In specific embodiments, known antibodies for the treatment of cancer are used. In some embodiments, the antibody will selectively bind to a cancer antigen of a hematological malignancy.

### 2.2.2 Primary Linkers

In one group of embodiments, a Ligand Drug Conjugate is comprised of one or more drug linker moieties of formula -L_{R}-L_{O}-D, wherein L_{O} is -A′_{a′}-W—Y_{y}— as described herein, wherein L_{R} is a primary linker, A′ is a second optional Stretcher Unit, a′ is 0 or 1, indicating the absence or presence of A′, respectively, Y is a Spacer Unit, subscript y is 0, 1 or 2, indicating the absence or presence of 1 or 2 Spacer Units, respectively, D is a Drug Unit, and W is a Peptide Cleavable Unit, wherein the Peptide Cleavable Unit is a sequence of up to 12 (e.g., 3-12 or 3-10) contiguous amino acids, wherein the sequence comprises a tripeptide that is more susceptible to proteolytic cleavage by a homogenate of tumor tissue as compared to a homogenate of normal tissue for initiating release of D as free drug, wherein cytotoxicity towards cells of the normal tissue due to unintended release of the free drug within and/or in the vicinity of these cells is associated with an adverse event from administration of an effective amount of a comparator Ligand Drug Conjugate to a subject in need thereof in which the sequence of amino acids of its Peptide Cleavable Unit is the dipeptide -valine-citrulline- and/or wherein the tripeptide increases the bioavailability of the Ligand Drug Conjugate to the detriment of its bioavailability to the normal tissue in comparison to the comparator Conjugate. In some of those embodiments -L_{R}- is -L_{B}-Aₐ-B_{b}— in which L_{B} is a ligand covalent binding moiety, A is a first optional Stretcher Unit, subscript a is 0 or 1, indicating the absence or presence of A, respectively, B is an optional Branching Unit, and subscript b is 0 or 1, indicating the absence or presence of B, respectively.

In some embodiments, a drug linker moiety has the structure of

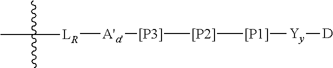

or a salt thereof, in particular a pharmaceutically acceptable salt, wherein L_{R}, A′, a′, Y, y and D retain their previous meanings and P1, P2 and P3 are amino acid residues that together provide selectivity for proteolysis by tumor tissue homogenate over proteolysis by normal tissue homogenate and/or provide increased bioavailability to tumor tissue to the detriment of normal tissue in comparison to a comparator Ligand Drug Conjugate in which the amino acid sequence of the Peptide Cleavable Unit is the dipeptide -valine-citrulline-, wherein proteolytic cleavage occurs at the covalent bond between P1 and Y if subscript y is 1 or 2 or at the covalent bond between P1 and D if subscript y is 0 and wherein the tumor and normal tissue are of the same species.

As described elsewhere, other embodiments contain an additional amino acid residue between P1 and Y or D, depending on the value of subscript y, which is designated as P-1, so that selective endopeptidase action by a proteolytic enzyme(s) of tumor tissue homogenate occurs at the amide bond between P1 and P-1 to release a drug linker fragment of formula —[P-1]-Y_{y}-D. Release of free drug from that fragment would occur from exopeptidase action of a proteolytic enzyme to remove the P-1 amino acid residue to directly provide free drug if subscript y is 0 (i.e., Y is absent).

In some embodiments in which an additional amino acid residue between P1 and Y or D, is present, a drug linker moiety has the structure of:

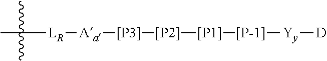

or a salt thereof, in particular a pharmaceutically acceptable salt, wherein L_{R}, A′, a′, Y, y and D retain their previous meanings and P1, P2 and P3 are amino acid residues, optionally with P-1, that together provide selectivity for proteolysis by tumor tissue homogenate over proteolysis by normal tissue homogenate, wherein proteolytic cleavage occurs at the covalent bond between P1 and P-1 to release a linker fragment having the structure of [P-1]-Y_{y}-D.

In some of those embodiments when subscript y is 0, the [P-1]-D residue resulting from endo-peptidase cleavage of the amide bond between the P1 and P-1 amino acids also exerts cytotoxic activity. In other embodiments, subscript y is 1 or 2 so that exopeptidase action to remove the P-1 amino acid residue provides another drug linker fragment of formula —Y_{y}-D, which spontaneously fragments to provide free drug.

In other embodiments one or more amino acid residues, designated P4, P5 . . . Pn, wherein subscript n ranges up to 12 (e.g., 3-12 or 3-10), are between P3 and L_{R} or A′, depending on the value of subscript a′, which is some embodiments is in addition to the Peptide Cleavable Unit containing a P-1 amino acid residue. In either instance, the additional P4, P5 . . . Pₙ amino acid residues are selected so as to not alter the cleavage site that provides the —Y_{y}-D or —[P-1]-Y_{y}-D fragment, but instead are selected to confer a desired physiochemical and/or pharmokinetic property to the Ligand Drug Conjugate, such as improved solubility for decreasing aggregation.

In some embodiments in which there is additional amino acid residue(s)N-terminus to P3 or additionally have a P-1 between P1 and Y or D, a drug linker moiety has the structure of:

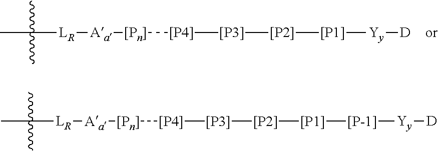

or a salt thereof, in particular a pharmaceutically acceptable salt, wherein L_{R}, A′, a′, Y, y and D retain their previous meanings and P-1 and P1, P2, P3 . . . Pₙ are amino acid residues, wherein subscript n ranges up to 12 (e.g., 3-12 or 3-10) and P1, P2 and P3, optionally with P-1, together provide selectivity for proteolysis by tumor tissue homogenate over proteolysis by normal tissue homogenate, wherein proteolytic cleavage occurs at the covalent bond between P1 and Y_{y}-D or between and P1 and P-1 to release a linker fragment having the structure of Y_{y}-D or [P-1]-Y_{y}-D, respectively, in which the later subsequently undergoes exopeptidase cleavage to release the linker fragment having the structure of Y_{y}-D. In both instances the Y_{y}-D linker fragment undergoes spontaneous decomposition to complete release of D as free drug.

In any one of those embodiments when subscript b is 0, L_{R} of a drug linker moiety has the formula of -L_{B}-Aₐ-, wherein L_{B} is a ligand covalent binding moiety and A is a first optional Stretcher Unit. In such embodiments if a is 1 and subscript a′ is 1, then A′ is present as subunit of A and therefore is considered a component of the primary linker.

In some preferred embodiments in which subscript b is 0 and subscript a is 1, L_{R} of formula -L_{B}-A- is a self-stabilizing linker (L_{SS}) moiety or a self-stabilized linker (L_{S}) moiety obtained from controlled hydrolysis of the succinimide (M²) moiety of L_{SS}. Exemplary L_{SS} and L_{S} primary linkers of a drug linker moiety of a Ligand Drug Conjugate composition, or Conjugate compound thereof, having either type of primary linker is represented by the structures of:

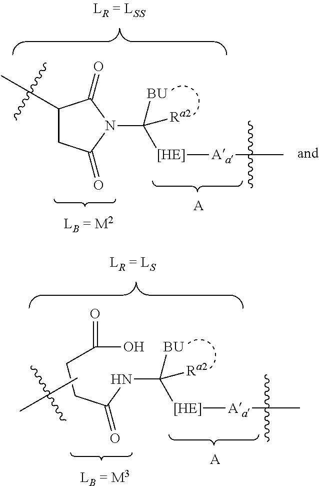

respectively, or a salt thereof, in particular a pharmaceutically acceptable salt, wherein the wavy line indicates the site of covalent attachments to A′ or W, depending on the value of subscript a′; A′ is an optional subunit of A; [HE] is an optional Hydrolysis Enhancing Unit, which is a component provided by A; BU is a Basic Unit; R^{a2} is an optionally substituted C₁-C₁₂ alkyl group; and the dotted curved line indicates optional cyclization so that in the absence of said cyclization, BU is an acyclic Basic Unit having a primary, secondary or tertiary amine functional group as the basic function group of the acyclic Basic Unit, or in the presence of said cyclization, BU is a cyclized Basic Unit in which R^{a2} and BU together with the carbon atom to which both are attached, define an optionally substituted spiro C₃-C₂₀ heterocyclo containing a skeletal basic nitrogen atom of a secondary or tertiary amine functional group as the basic function group of the cyclic Basic Unit,
wherein the basic nitrogen atom of the acyclic Basic Unit or cyclic Basic Unit is optionally suitably protected by a nitrogen protecting group, dependent on the degree of substitution of the basic nitrogen atom or is optionally protonated.

In other preferred embodiments in which subscript b is 0 and subscript a is 1 the primary linker of formula -L_{B}-A- does not contain a Basic Unit, which are exemplified by the structure of:

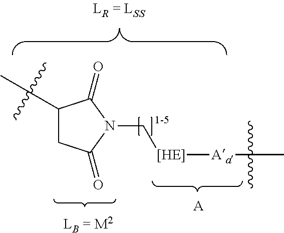

or a salt thereof, in particular, a pharmaceutically acceptable salt, wherein the variable groups are as previously described for L_{SS} or L_{S} primary linkers.

Representative L-L_{R}- structures, in which L_{R} is covalently attached to a Ligand Unit (L) of a LDC, are the following:

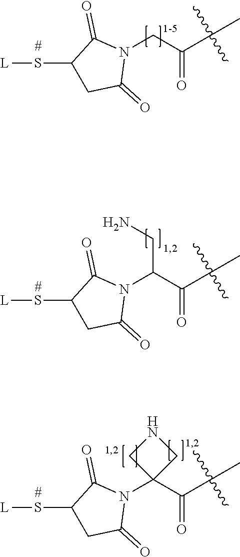

and salts thereof, in particular, pharmaceutically acceptable salts, and structures in which the succinimide ring system is hydrolyzed to a ring opened form, wherein the indicated (#) sulfur atom is from the Ligand Unit; and wherein the wavy line indicates the site of covalent attachment to the remainder of the Conjugate structure.

Other representative L-L_{R}- structures are the following:

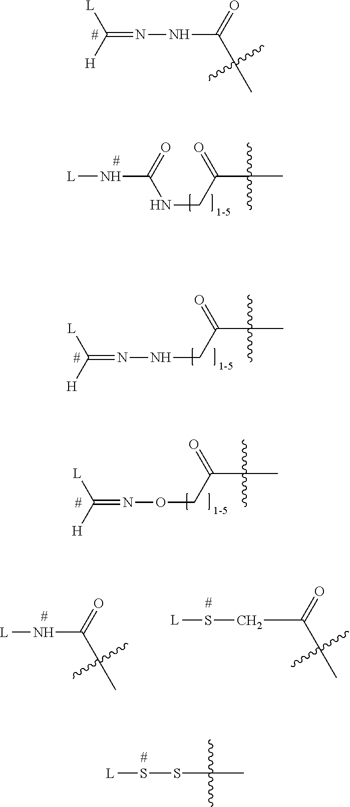

wherein the indicated (#) nitrogen, carbon or sulfur atom is from the Ligand Unit; and wherein the wavy line indicates the site of covalent attachment to the remainder of the Conjugate structure.

In another group of embodiments, a Drug Linker Compound, which is useful in preparing a Ligand Drug Conjugate as described in the previous group of embodiments, has the formula of L_{R}′-A′_{a′}-W—Y_{y}-D as described herein, wherein L_{R}′ is a primary linker of the Drug Linker Compound, which is converted to the primary linker L_{R} of a drug linker moiety of a Ligand Drug Conjugate when the Drug Linker compound is used in the preparation of that Conjugate, A′ is a second optional Stretcher Unit, a′ is 0 or 1, indicating the absence or presence of A′, respectively, wherein when L_{R}′ does not contain a Branching Unit and subscript a′ is 1, A′ is considered part of L_{R}′ as a subunit of A which is present as a component of L_{R}′, Y is a Spacer Unit, subscript y is 0, 1 or 2, indicating the absence or presence of 1 or 2 Spacer Units, respectively, D is a Drug Unit, and W is a Peptide Cleavable Unit comprising a tripeptide that is more susceptible to proteolytic cleavage by a homogenate of tumor tissue as compared to a homogenate of normal tissue, wherein cytotoxicity towards cells of the normal tissue due to unintended release of D as free drug within and/or in the vicinity of these cells is associated with an adverse event from administration of the Ligand Drug Conjugate intended for targeting the cancer cells of the tumor tissue. In some of those embodiments L_{R}′- is L_{B}′-Aₐ-B_{b}— wherein L_{B}′ is a ligand covalent binding moiety of the primary linker of the Drug Linker compound, sometimes referred to as ligand covalent binding precursor moiety since it is a precursor to a ligand covalent binding moiety (L_{B}) of a primary linker (L_{R}) of a drug linker moiety of a Ligand Drug Conjugate when the Drug Linker compound is used in the preparation of that Conjugate, A is a first optional Stretcher Unit, subscript a is 0 or 1, indicating the absence or presence of A, respectively, B is an optional Branching Unit, and subscript b is 0 or 1, indicating the absence or presence of B, respectively.

In some embodiments, a Drug Linker compound has the structure of

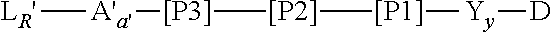

or a salt thereof, in particular a pharmaceutically acceptable salt, wherein L_{R}′, A′, a′, Y, y and D retain their previous meanings and P1, P2 and P3 are amino acid residues that together provide selectivity for proteolysis by tumor tissue homogenate over proteolysis by normal tissue homogenate, wherein proteolytic cleavage occurs at the covalent bond between P1 and Y if subscript y is 1 or 2 or at the covalent bond between P1 and D if subscript y is 0.

As described elsewhere, other embodiments contain an additional amino acid residue between P1 and Y or D, depending on the value of subscript y, which is designated as P-1, so that selective endopeptidase action by a proteolytic enzyme(s) of tumor tissue homogenate occurs at the amide bond between P1 and P-1 to release a drug linker fragment of formula —[P-1]-Y_{y}-D. Release of free drug from that fragment would occur from exopeptidase action of a proteolytic enzyme to remove the P-1 amino acid residue to directly provide free drug if subscript y is 0 (i.e., Y is absent).

In some embodiments in which an additional amino acid residue between P1 and Y or D, is present, a Drug Linker Compound has the structure of:

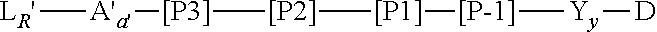

or a salt thereof, in particular a pharmaceutically acceptable salt, wherein L_{R}′, A′, a′, Y, y and D retain their previous meanings and P1, P2 and P3 are amino acid residues, optionally with P-1, that together provide selectivity for proteolysis by tumor tissue homogenate over proteolysis by normal tissue homogenate, wherein proteolytic cleavage occurs at the covalent bond between P1 and P-1 to release a linker fragment having the structure of [P-1]-Y_{y}-D.

In some of those embodiments when subscript y is 0, the [P-1]-D residue resulting from endo-peptidase cleavage of the amide bond between the P1 and P-1 amino acids also exerts cytotoxic activity. In other embodiments, subscript y is 1 or 2 so that exopeptidase action to remove the P-1 amino acid residue provides another drug linker fragment of formula —Y_{y}-D, which spontaneously fragments to provide free drug.

In other embodiments one or more amino acid residues, designated P4, P5 . . . Pn, wherein subscript n ranges up to 12 (e.g., 3-12 or 3-10), are between P3 and L_{R} or A′, depending on the value of subscript a′, which is some embodiments is in addition to the Peptide Cleavable Unit containing a P-1 amino acid residue. In either instance, the additional P4, P5 . . . Pₙ amino acid residues are selected so as to not alter the cleavage site that provides the —Y_{y}-D or —[P-1]-Y_{y}-D fragment, but instead are selected to confer a desired physiochemical and/or pharmokinetic property to the Ligand Drug Conjugate, such as improved solubility for decreasing aggregation.

In some embodiments in which there is additional amino acid residue(s)N-terminus to P3 or additionally have a P-1 between P1 and Y or D, a Drug Linker compound has the structure of:

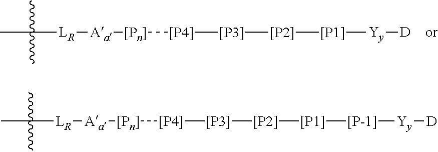

or a salt thereof, in particular a pharmaceutically acceptable salt, wherein L_{R}′, A′, a′, Y, y and D retain their previous meanings and P-1 and P1, P2, P3 . . . Pₙ are amino acid residues, wherein subscript n ranges up to 12 (e.g., 3-12 or 3-10) and P1, P2 and P3, optionally with P-1, together provide selectivity for proteolysis by tumor tissue homogenate over proteolysis by normal tissue homogenate, wherein proteolytic cleavage occurs at the covalent bond between P1 and Y_{y}-D or between and P1 and P-1 to release a linker fragment having the structure of Y_{y}-D or [P-1]-Y_{y}-D, respectively, in which the later subsequently undergoes exopeptidase cleavage to release the linker fragment having the structure of Y_{y}-D. In both instances the Y_{y}-D linker fragment undergoes spontaneous decomposition (also referred to as self-immolation) to complete release of D as free drug.

In any one of those embodiments when subscript b is 0, L_{R}′ of a Drug Linker compound has the formula of L_{B}′-Aₐ-, wherein L_{B}′ is a ligand covalent binding precursor moiety and A is a first optional Stretcher Unit. In such embodiments if subscript a is 1 and subscript a′ is 1, then A′ is present as subunit of A and therefore is considered a component of the primary linker.

In some preferred embodiments in which subscript b is 0 and subscript a is 1, L_{R}′ of formula L_{B}′-A- of a Drug Linker compound is a self-stabilizing linker precursor (L_{SS}′) moiety so named since it converts to self-stabilizing linker (L_{SS}) moiety of a Ligand Drug Conjugate when the Drug Linker compound is used in the preparation of the Conjugate. Exemplary L_{SS}′ primary linkers of a Drug Linker compound are represented by the structures of:

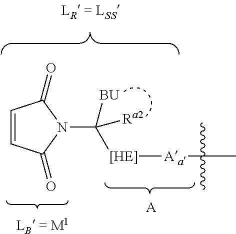

or a salt thereof, in particular a pharmaceutically acceptable salt, wherein the wavy line indicates the site of covalent attachments to A′ or W, depending on the value of subscript a′; A′ is an optional subunit of A; [HE] is an optional Hydrolysis Enhancing Unit, which is a component provided by A; BU is a Basic Unit; R^{a2} is an optionally substituted C₁-C₁₂ alkyl group; and the dotted curved line indicates optional cyclization so that in the absence of said cyclization, BU is an acyclic Basic Unit having a primary, secondary or tertiary amine functional group as the basic function group of the acyclic Basic Unit, or in the presence of said cyclization BU is a cyclized Basic Unit in which R^{a2} and BU together with the carbon atom to which both are attached, define an optionally substituted spiro C₃-C₂₀ heterocyclo containing a skeletal basic nitrogen atom of a secondary or tertiary amine functional group as the basic function group of the cyclic Basic Unit, wherein the basic nitrogen atom of the acyclic Basic Unit or cyclic Basic Unit is optionally suitably protected by a nitrogen protecting group, dependent on the degree of substitution of the basic nitrogen atom or is optionally protonated.

In other preferred embodiments in which subscript b is 0 and subscript a is 1 the primary linker of formula L_{B}-A- does not contain a Basic Unit, which are exemplified by the structure of:

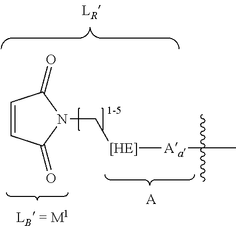

or a salt thereof, in particular, a pharmaceutically acceptable salt, wherein the variable groups are as previously described for L_{SS} or L_{S} primary linkers.

Representative L_{R}′- structures of a Drug Linker compound are the following:

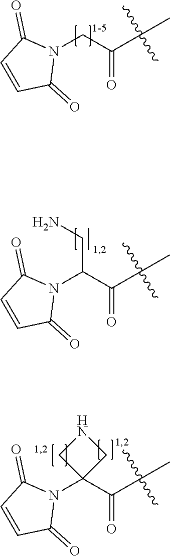

and salts thereof, in particular pharmaceutically acceptable salts, wherein the wavy line indicates the site of covalent attachment to the remainder of LU′ of the Drug Linker compound structure and wherein the basic nitrogen atom in the second or third structure is optionally protonated as an acid addition salt or is optionally protected. When protected, the protecting group is preferably an acid-labile protecting group such as BOC.

### 2.2.3 Peptide Cleavable Units

In some embodiments a Peptide Cleavable Unit (W) of a Ligand Drug Conjugate is a peptide sequence containing a tripeptide directly attached to D or indirectly through one or two self-immolative Spacer Units, wherein the tripeptide is recognized by at least one intracellular protease, preferably by more than one, wherein the at least one protease is upregulated in tumor cells in comparison to normal cells, and is more susceptible to proteolysis by a homogenate of tumor tissue comprised of the tumor cells to be targeted by the Ligand Drug Conjugate in comparison to a homogenate of normal tissue wherein cytotoxicity to the normal tissue is associated with an adverse event from administration of a comparator Ligand Drug Conjugate. In other embodiments, the tripeptide improves the biodistribution of the Conjugate to the tumor tissue to the detriment of biodistribution to the normal tissue, which in some of these embodiments is in addition to the selectivity for proteolysis by tumor tissue homogenate in comparison to proteolysis by normal tissue homogenate. In either one of those embodiments, the normal tissue is sometimes bone marrow and the adverse event to be ameliorated is neutropenia. In another embodiment, the normal tissue is bone marrow, liver, kidney, esophageal, breast, or corneal tissue and the adverse event to be ameliorated is neutropenia. In some embodiments, the tripeptide is directly attached to D or indirectly attached to D through one or two self-immolative Spacer Units. In other embodiments, the Peptide Cleavable Unit (W) comprising a tripeptide as described herein is directly attached to D or indirectly attached to D through one or two self-immolative Spacer Units via an amino acid that is not part of the tripeptide.

The Peptide Cleavable Unit (W) of the comparator Conjugate is typically a dipeptide that confers selectivity for a specific intracellular protease that is upregulated in cancer cells over freely circulating proteases, wherein the specific protease is capable of cleaving the amide bond between the C-terminal amino acid of the dipeptide and the amino group of a self-immolative Spacer Unit (Y) to initiate release of the Drug Unit as free drug.

In some embodiments, the Ligand Drug Conjugate comprising the tripeptide as disclosed herein shows improved tolerability in comparison to a comparator Ligand Drug Conjugate in which the Peptide Cleavable Unit is a dipeptide that confers selectivity for a specific intracellular protease that is upregulated in cancer cells over freely circulating proteases, wherein the specific protease is capable of cleaving the amide bond between the C-terminal amino acid of the dipeptide and the amino group of a self-immolative Spacer Unit (Y) to initiate release of the Drug Unit as free drug. In some embodiments, the dipeptide is known to be selectively cleavable by Cathepsin B. In some embodiments, the dipeptide in the comparator Ligand-Drug Conjugate is -valine-citrulline- or -valine-alanine-. In some embodiments, the dipeptide in the comparator Ligand-Drug Conjugate is -valine-citrulline-. In some embodiments, the dipeptide in the comparator Ligand-Drug Conjugate is -valine-alanine-. In some embodiments, tolerability refers to the degree to which adverse events associated with the Ligand-Drug Conjugate's administration affect the ability or desire of the patient to adhere to the dose or intensity of therapy. As such, improved tolerability may be achieved by reducing the occurrence or severity of the adverse events.

Without being bound by theory, aggregated Ligand Drug Conjugate compounds are more likely to be distributed in a normal tissue (e.g., bone marrow), wherein the normal tissue is known to be the source of on-target and/or off-target adverse event(s) experienced by a mammalian subject when administered a therapeutically effective amount of a Ligand Drug Conjugate. In some embodiments, the improved tolerability is demonstrated by the decreased aggregation rate of the Ligand Drug Conjugate comprising the tripeptide in comparison to the comparator Ligand Drug Conjugate. In some embodiments, the aggregation rates of the Ligand Drug Conjugate comprising the tripeptide and the comparator Ligand Drug Conjugate are determined by measuring the concentrations of high molecular weight aggregates after incubating the conjugates in rat plasma, cynomolgus monkey plasma, or human plasma at a same concentration for 12, 24, 36, 48, 60, 72, 84, or 96 hours.

In some embodiments, the improved tolerability of the Ligand Drug Conjugate comprising the tripeptide is demonstrated by an improved selectivity for exposure of a tumor tissue over a normal tissue to free cytotoxic compound released from the Ligand Drug Conjugate comprising the tripeptide in comparison to the cytotoxic compound released from the comparator Ligand Drug Conjugate. In some embodiments, the tumor tissue and the normal tissue are from a rodent species (e.g., rat or mouse) or a primate species (e.g., cynomolgus monkey or human). In some embodiments, when the tumor tissue and the normal tissue are from a species different from human, the normal tissue is of the same tissue type in human and wherein cytotoxicity to cells of that tissue is responsible at least in part to an adverse event in a human subject to whom is administered a therapeutically effective amount of the comparator Ligand Drug Conjugate. In some embodiments, the normal tissue is bone marrow, liver, kidney, esophageal, breast, or corneal tissue. In some embodiments, the normal tissue is bone marrow.

In some embodiments, the improved exposure selectivity is demonstrated by a reduction in plasma concentration of the free cytotoxic compound released from the Ligand Drug Conjugate comprising the tripeptide in comparison to the comparator Ligand Drug Conjugate when the conjugates are administered at a same dose. In some embodiments, the Ligand Drug Conjugate comprising the tripeptide retains efficacy (e.g., achieves substantially same reduction in tumor volume in comparison with the comparator Ligand Drug Conjugate) in a tumor xenograft model when administered at the same effective amount and dose schedule previously determined for the comparator Ligand-Drug Conjugate.

In some embodiments, the improved exposure selectivity is demonstrated by decreased non-target mediated cytoxicity or preservation of normal cells in the normal tissue in comparison to the comparator Ligand-Drug Conjugate when the conjugates are administered at a same dose. In some embodiments, the normal tissue is bone marrow, liver, kidney, esophageal, breast, or corneal tissue. In some embodiments, the normal tissue is bone marrow. In some embodiments, the decreased non-target mediated cytoxicity or preservation of normal cells in the normal tissue is demonstrated by bone marrow histology (e.g., reduced loss of nuclei staining of mononuclear cells). In some embodiments, the decreased non-target mediated cytoxicity or preservation of normal cells is demonstrated by reduction in neutrophil and/or reticulocyte loss and/or more rapid rebound from that loss. In some embodiments, the decreased non-target mediated cytoxicity or preservation of normal cells is demonstrated by a reduction in neutrophil loss. In some embodiments, the decreased non-target mediated cytoxicity or preservation of normal cells is demonstrated by a reduction in reticulocyte loss. In some embodiments, the Ligand Drug Conjugate comprising the tripeptide retains efficacy in a tumor xenograft model when administered at the same effective amount and dose schedule previously determined for the comparator Ligand-Drug Conjugate. In some embodiments, when comparing the exposure selectivity between the Ligand Drug Conjugate comprising the tripeptide and the comparator Ligand Drug Conjugate, the Ligand Units of both conjugates are replaced by a non-binding antibody.

In some embodiments, provided are Ligand-Drug Conjugates (e.g., ADCs) that are less active than the comparator Ligand Drug Conjugate (e.g., dipeptide ADC containing -val-cit-), either in vivo or in vitro, but are also significantly less toxic. Without being bound by theory, the Ligand-Drug Conjugate is not required to be as active because the therapeutic window will still be increased if it is less active and less toxic.

In preferred embodiments, the amide bond between the carboxylic acid of the C-terminal amino acid of the tripeptide and the amino group of a self-immolative Spacer Unit (Y) is cleavable by at least one, preferably by more than one, intracellular protease to initiate release of a Drug Unit as free drug. When the Drug Unit is that of MMAE, the drug linker moieties of the comparator Conjugate have the formula of mc-val-cit-PABC-MMAE or mp-val-cit-PABC-MMAE, which have the structures of:

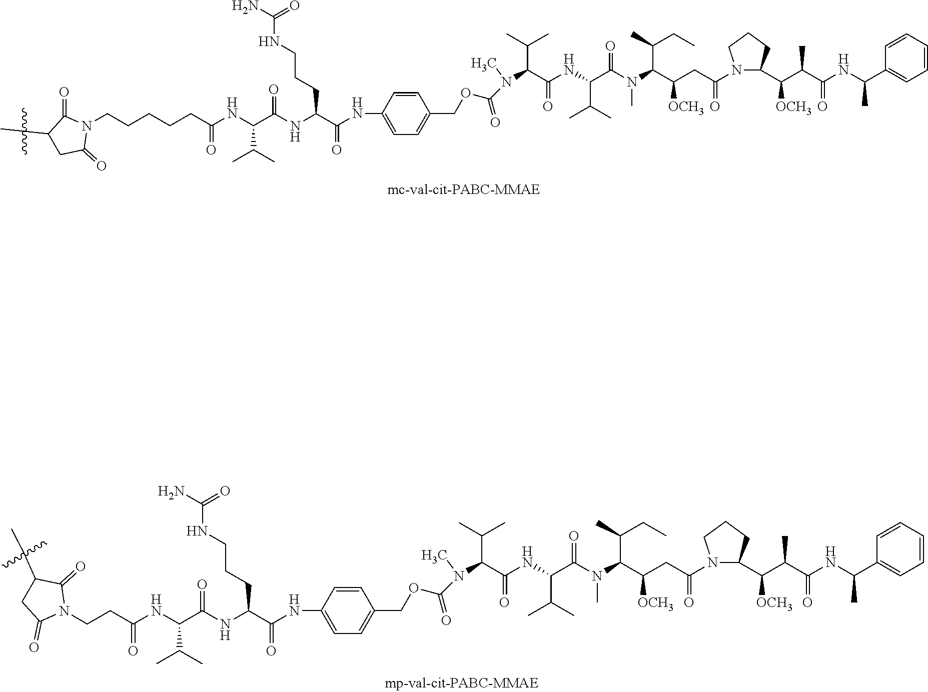 [SMILES: *C1CC(=O)N(CCCCCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)c3ccccc3)OC)C(C)C)C(C)C)cc2)C(C)C)C1=O ; *C1CC(=O)N(CCC(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)c3ccccc3)OC)C(C)C)C(C)C)cc2)C(C)C)C1=O]

In other embodiments a Peptide Cleavable Unit (W) of a Ligand Drug Conjugate is a peptide sequence comprised of a tetrapeptide residue directly attached to D or indirectly through at least one self-immolative Spacer Unit, wherein the tetrapeptide sequence —P3-P2-P1-[P-1]- is recognized by at least one intracellular protease, preferably by more than one, wherein the at least one intracellular protease is upregulated within tumor cells in comparison to normal cells, and is more selective for proteolysis by a homogenate of tumor tissue that are comprised of the tumor cells to be targeted by the Ligand Drug Conjugate in comparison to a homogenate of normal tissue wherein cytotoxicity to the normal tissue is associated with an adverse event from administration of a comparator Ligand Drug Conjugate. The Peptide Cleavable Unit of the comparator Conjugate is a dipeptide that confers selectivity for a specific intracellular protease over freely circulating proteases. In those tetrapeptide embodiments said selectivity is primarily attributed to the N-terminal tripeptide sequence of the tetrapeptide.

In preferred embodiments in which the peptide sequence is comprised of the tetrapeptide residue, the amide bond between the carboxylic acid of the C-terminal amino acid and the remaining amino acid residue of that tetrapeptide sequence is cleavable by the at least one intracellular protease to initiate release of free drug by first releasing an amino acid-containing linker fragment that subsequently undergoes exopeptidase removal of its amino acid component to provide a second linker fragment. Thus, the P1-[P-1] bond in the tetrapeptide —P3-P2-P1-[P-1]- is cleaved to release the drug linker fragment of —[P-1]—Y_{y}-D. The second linker fragment then undergoes self-immolation of its Spacer Unit(s) that had intervened between D and the tetrapeptide of W to complete release of D as free drug.

In any one of the above embodiments the at least one protease, which is preferably upregulated within targeted cancer cells, includes certain cathepsins such as Cathepsin B. In other embodiments the P1-D, P1-Y— or P1-[P-1] bond is cleavable by a non-excreted intracellular protease or collection of such intracellular proteases of targeted cancer cells and one or more extracellular proteases that are associated with or are upregulated within the tissue microenvironment of tumor cells and which are absent or are present at reduced levels in the tissue microenvironment of normal cells, wherein cytotoxicity towards these normal cells is typically associated with an adverse event from administration of an effective amount of a comparator Conjugate in which the Peptide Cleavable Unit is a dipeptide that confers selectivity for an intracellular protease over freely circulating proteases. In other embodiments the P1-D, P1-Y— or P1-[P-1] bond is cleavable by a non-excreted intracellular protease or collection of such intracellular proteases of targeted cancer cells and is less susceptible to proteolysis by extracellular protease(s) that are associated with normal tissue in comparison to a comparator Conjugate in which the Peptide Cleavable Unit is the aforementioned dipeptide. In some of those embodiments, the secreted protease within normal tissue is a neutrophil protease such as those selected from the group consisting of Neu Elastase, cathepsin G and proteinase 3.

In other preferred embodiments a tripeptide in a Ligand Drug Conjugate of the present invention confers global selectivity for proteolysis by a homogenate of tumor tissue that is comprised of the tumor cells to be targeted by the Ligand Drug Conjugate in comparison to a homogenate of normal tissue wherein cytotoxicity to the normal tissue is associated with an adverse event from administration of a comparator Ligand Drug Conjugate. The Peptide Cleavable Unit (W) in drug linker moieties of the comparator Conjugate is the aforementioned dipeptide that confers selectivity for a specific intracellular protease upregulated in cancer cells of the tumor tissue over freely circulating proteases. Other preferred tripeptides increase the biodistribution of the Conjugate into tumor tissue to the detriment of biodistribution into normal tissue wherein cytotoxicity to the normal tissue is associated with an adverse event from administration of a comparator Ligand Drug Conjugate in which W is a dipeptide that confers selectivity for a specific intracellular protease over freely circulating proteases. When the Drug Unit is that of MMAE the drug linker moieties of the comparator Conjugate have the formula of me-val-cit-PABC-MMAE or mp-val-cit-PABC-MMAE.

It was determined that Ligand Drug Conjugates having linkers containing certain 3-residue amino acid sequences have advantageous properties, such as reduced toxicity in one or more normal tissues (which may be due to differential proteolysis) and improved biophysical properties (e.g., reduced aggregation, longer residence time prior to clearance). These advantageous properties may be obtained in Ligand Drug Conjugates having linkers containing a 3-amino acid sequence in which the N-terminal amino acid of the 3-residue sequence is a D-amino acid, and the central and C-terminal residues of the 3-residue sequence are, in either order, an amino acid that is negatively charged (e.g., at plasma physiological pH) and an amino acid that is polar or that has an aliphatic side chain with hydrophobicity no greater than that of leucine. In some embodiments, the tripeptide contains an amino acid in the D-amino acid configuration. In some embodiments, the tripeptide contains D-Leu or D-Ala. In some embodiments, the tripeptide contains D-Leu. In some embodiments, the tripeptide contains D-Ala. In some embodiments, the tripeptide contains an amino acid having an aliphatic side chain with hydrophobicity no greater than that of leucine. In some embodiments, the tripeptide contains an amino acid having an aliphatic side chain with hydrophobicity no greater than that of valine. In some embodiments, the tripeptide contains alanine. In some embodiments, the tripeptide contains a polar amino acid. In some embodiments, the tripeptide contains serine. In some embodiments, the tripeptide contains an amino acid that is negatively charged (e.g., at plasma physiological pH). In some embodiments, the tripeptide contains an amino acid selected from the group consisting of aspartic acid and glutamic acid. In some embodiments, the P3 amino acid of the tripeptide is in the D-amino acid configuration. In some embodiments, the P3 amino acid is D-Leu or D-Ala. In some embodiments, the P3 amino acid is D-Leu. In some embodiments, the P3 amino acid is D-Ala. In some embodiments, the P2 amino acid of the tripeptide has an aliphatic side chain with hydrophobicity no greater than that of leucine. In some embodiments, the P2 amino acid has an aliphatic side chain with hydrophobicity no greater than that of valine. In some embodiments, P2 amino acid is alanine. In some embodiments, the P2 amino acid of the tripeptide is a polar amino acid. In some embodiments, P2 amino acid is serine. In some embodiments, the P2 amino acid of the tripeptide is negatively charged (e.g., at plasma physiological pH). In some embodiments, the P2 amino acid is selected from the group consisting of aspartic acid and glutamic acid. In some embodiments, the P1 amino acid of the tripeptide has an aliphatic side chain with hydrophobicity no greater than that of leucine. In some embodiments, the P1 amino acid has an aliphatic side chain with hydrophobicity no greater than that of valine. In some embodiments, P1 amino acid is alanine. In some embodiments, the P1 amino acid of the tripeptide is a polar amino acid. In some embodiments, P1 amino acid is serine. In some embodiments, the P1 amino acid of the tripeptide is negatively charged (e.g., at plasma physiological pH). In some embodiments, the P1 amino acid is selected from the group consisting of aspartic acid and glutamic acid. In some embodiments, one of the P2 or P1 amino acid of the tripeptide has an aliphatic side chain with hydrophobicity no greater than that of leucine (e.g., no greater than that of valine), and the other of the P2 or P1 amino acid is a polar amino acid or is negatively charged (e.g., at plasma physiological pH). In some embodiments, the P2 amino acid has an aliphatic side chain with hydrophobicity no greater than that of leucine (e.g., no greater than that of valine), and the P1 amino acid is a polar amino acid or is negatively charged (e.g., at plasma physiological pH). In some embodiments, the P1 amino acid has an aliphatic side chain with hydrophobicity no greater than that of leucine (e.g., no greater than that of valine), and the P2 amino acid is a polar amino acid or is negatively charged (e.g., at plasma physiological pH). In some embodiments, —P2-P1- is -Ala-Glu-. In some embodiments, —P2-P1- is -Ala-Asp-. In some embodiments, the P3 amino acid of the tripeptide is in the D-amino acid configuration, one of the P2 or P1 amino acid has an aliphatic side chain with hydrophobicity no greater than that of leucine (e.g., no greater than that of valine), and the other of the P2 or P1 amino acid is negatively charged (e.g., at plasma physiological pH). In some embodiments, the P3 amino acid is in the D-amino acid configuration, the P2 amino acid has an aliphatic side chain with hydrophobicity no greater than that of leucine (e.g., no greater than that of valine), and the P1 amino acid is negatively charged (e.g., at plasma physiological pH). In some embodiments, the P3 amino acid is in the D-amino acid configuration, the P1 amino acid has an aliphatic side chain with hydrophobicity no greater than that of leucine (e.g., no greater than that of valine), and the P2 amino acid is negatively charged (e.g., at plasma physiological pH). In some embodiments, —P3-P2-P1- is selected from the group consisting of -D-Leu-Ala-Asp-, -D-Leu-Ala-Glu-, -D-Ala-Ala-Asp-, and -D-Ala-Ala-Glu-.

In some embodiments, the tripeptide contains an amino acid selected from the group consisting of alanine, citrulline, proline, isoleucine, leucine and valine. In some embodiments, the tripeptide contains an amino acid in the D-amino acid configuration. In some embodiments, the tripeptide contains D-Leu. In some embodiments, the tripeptide contains D-Ala. In some embodiments, the tripeptide contains an amino acid in the D-amino acid configuration. In another embodiment, the tripeptide contains an amino acid selected from the group consisting of D-leucine and D-alanine. In another embodiment, tripeptide contains D-leucine. In another embodiment, tripeptide contains D-alanine. In some embodiments, the tripeptide contains an amino acid having a side chain with at least one charged (e.g., negatively charged at plasma physiological pH) substituent or at least one uncharged substituent with a permanent electric dipole moment and one or two additional amino acids having aliphatic side chains with hydrophobicity no greater than that of leucine. In some embodiments, the tripeptide contains an amino acid having an aliphatic side chain with hydrophobicity no greater than that of leucine, such as alanine or valine. In some embodiments, the tripeptide contains an amino acid having an aliphatic side chain with hydrophobicity no greater than that of valine, such as alanine. In some embodiments, the tripeptide is contains a polar amino acid, such as aspartic acid, glutamic acid, asparagine, glutamine, serine, threonine, tyrosine, citrulline, methionine sulfoxide, or γ-carboxy-glutamic acid. In some embodiments, the tripeptide contains an amino acid that is negatively charged (e.g., at plasma physiological pH), such as glutamic acid, aspartic acid, or γ-carboxy-glutamic acid. In some embodiments, the tripeptide contains an amino acid having a side chain with at least one charged substituent or at least one uncharged substituent with a permanent electric dipole moment, preferably greater than that of —C(O)NH₂. In some embodiments, the tripeptide contains an amino acid having a side chain with at least one charged substituent or at least one uncharged substituent with a permanent electric dipole moment, preferably greater than that of —NH—C(O)NH₂. In some embodiments, the tripeptide contains an amino acid selected from the group consisting of alanine, α-aminobutyric acid, α-aminoisobutyric acid, aspartic acid, citrulline, γ-carboxy-glutamic acid, glutamic acid, glutamine, glycine, leucine, norvaline proline, isoleucine, leucine, lysine, methionine sulfoxide, naphthylalanine, O-allyl tyrosine, phenylalanine, propargylglycine, 2-aminobut-3-ynoic acid, proline, selenomethionine, serine, threonine, and valine. In some embodiments, the tripeptide contains and amino acid selected the group consisting of alanine, aspartic acid, citrulline, γ-carboxyglutamic acid, glutamic acid, glutamine, glycine, leucine, proline, isoleucine, leucine, lysine, methionine sulfoxide, naphthylalanine, O-allyl tyrosine, phenylalanine, proline, selenomethionine, serine, threonine, and valine. It is understood that the amino acid in any of the embodiments herein can be a natural or un-natural amino acid. For example, alanine can be D-alanine or L-alanine and leucine can be D-leucine or L-leucine.

In more preferred tripeptides, the P3 amino acid is selected from the group consisting of alanine, citrulline, proline, isoleucine, leucine and valine preferably in the D-amino acid configuration with D-Leu particularly preferred. In another embodiment, the P3 amino acid is in the D-amino acid configuration. In another embodiment, the P3 amino acid in the tripeptide is selected from the group consisting of alanine, leucine, glutamic acid, lysine, O-allyl tyrosine, phenylalanine, proline, and threonine. In another embodiment, the P3 amino acid in the tripeptide is selected from the group consisting of D-alanine, D-leucine, glutamic acid, lysine, O-allyl tyrosine, phenylalanine, proline, and threonine. In another embodiment, the P3 amino acid in the tripeptide is D-leucine or D-alanine. In another embodiment, the P3 amino acid in the tripeptide is D-leucine. In another embodiment, the P3 amino acid in the tripeptide is D-alanine.

In other more preferred tripeptides, the P2 amino acid is a natural or un-natural amino acid having an aliphatic side chain with hydrophobicity no greater than that of leucine, with lower hydrophobicity more preferred with greater hydrophobicity of the P3 side chain. In another embodiment, the P2 amino acid is a natural or un-natural amino acid having an aliphatic side chain with hydrophobicity no greater than that of valine. In some embodiments, the P2 amino acid in the tripeptide is selected from the group consisting alanine, valine, leucine and methionine. In some embodiments, the P2 amino acid in a tripeptide is selected from the group consisting alanine, valine, and methionine. In some embodiments, the P2 amino acid in the tripeptide is alanine. In some of those preferred tripeptides P2 is selected from the group consisting of Abu, Aib, Ala, Gly, Leu, Nva, Pra, Egl and Val in which the un-natural amino acids have the structures of:

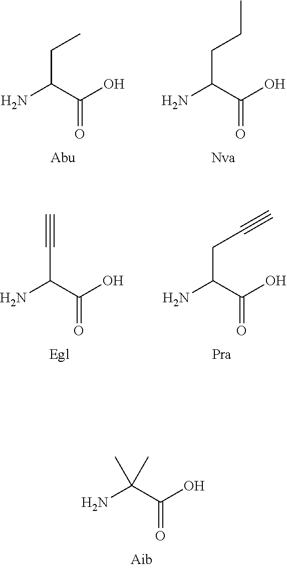

For Abu, Ala, Leu, Nva and Pra as the P2 amino acid residue the side chain is preferably in an L-configuration. In another embodiment, the P2 amino acid in the tripeptide is a polar amino acid. In some embodiments, the P2 amino acid in the tripeptide is selected from the group consisting of aspartic acid, glutamic acid, asparagine, glutamine, serine, threonine, tyrosine, citrulline, methionine sulfoxide, and γ-carboxy-glutamic acid. In another embodiment, the P2 amino acid in the tripeptide is negatively charged (e.g., at plasma physiological pH). In some embodiments, the P2 amino acid in the tripeptide is selected from the group consisting of aspartic acid, glutamic acid, and γ-carboxy-glutamic acid. In some embodiments, the P2 amino acid in the tripeptide is selected from the group consisting of aspartic acid and glutamic acid. In some embodiments, the P2 amino acid in the tripeptide is alanine. In some embodiments, the P2 amino acid in the tripeptide is serine. In some embodiments, the P2 amino acid in the tripeptide is selected from the group consisting alanine, valine, leucine, methionine, aspartic acid, glutamic acid, asparagine, glutamine, serine, threonine, tyrosine, citrulline, methionine sulfoxide, and γ-carboxy-glutamic acid.

In still other more preferred tripeptides, the P1 amino acid is a natural or un-natural amino acid having a side chain with at least one charged substituent or at least one uncharged substituent with a permanent electric dipole moment, preferably greater than that of —C(O)NH₂. In another embodiment, the P1 amino acid is a natural or un-natural amino acid having a side chain with at least one charged substituent or at least one uncharged substituent with a permanent electric dipole moment, preferably greater than that of —NH—C(O)NH₂. In some of those preferred tripeptides P1 is selected from the group consisting of Glu, Asp, γ-carboxy-glutamic acid, lysine, methionine sulfoxide, sometimes indicated as Met(O) and phospho-threonine in which the side chain is preferably in the L-stereochemical configuration, with Glu, Asp, γ-carboxy-glutamic acid and Met(O), more preferred and Glu particularly preferred. In some embodiments, the P1 amino acid in the tripeptide is selected from the group consisting of alanine, aspartic acid, citrulline, γ-carboxy-glutamic acid, glutamic acid, glutamine, leucine, lysine, methionine sulfoxide, and selenomethionine. In some embodiments, the P1 amino acid in the tripeptide is glutamic acid. In some embodiments, the P1 amino acid is a natural or un-natural amino acid having an aliphatic side chain with hydrophobicity no greater than that of leucine, with lower hydrophobicity more preferred with greater hydrophobicity of the P3 side chain. In another embodiment, the P1 amino acid is a natural or un-natural amino acid having an aliphatic side chain with hydrophobicity no greater than that of valine. In some embodiments, the P1 amino acid in the tripeptide is selected from the group consisting alanine, valine, leucine, and methionine. In some embodiments, the P1 amino acid in the tripeptide is selected from the group consisting alanine, valine, and methionine. In some embodiments, the P1 amino acid in a tripeptide is alanine. In another embodiment, the P1 amino acid in the tripeptide is a polar amino acid. In some embodiments, the P1 amino acid in the tripeptide is selected from the group consisting of aspartic acid, glutamic acid, asparagine, glutamine, serine, threonine, tyrosine, citrulline, methionine sulfoxide, and γ-carboxy-glutamic acid. In another embodiment, the P1 amino acid in the tripeptide is negatively charged (e.g., at plasma physiological pH). In some embodiments, the P1 amino acid in the tripeptide is selected from the group consisting of aspartic acid, glutamic acid, and γ-carboxy-glutamic acid. In some embodiments, the P1 amino acid in the tripeptide is selected from the group consisting of aspartic acid and glutamic acid. In some embodiments, the P1 amino acid in the tripeptide is alanine. In some embodiments, the P1 amino acid in the tripeptide is serine.

In another embodiment, the P3 amino acid in the tripeptide is selected from the group consisting of alanine, leucine, glutamic acid, lysine, O-allyl tyrosine, phenylalanine, proline, and threonine, the P2 amino acid in the tripeptide is selected from the group consisting alanine, valine, leucine, methionine, aspartic acid, glutamic acid, asparagine, glutamine, serine, threonine, tyrosine, citrulline, methionine sulfoxide, and γ-carboxy-glutamic acid, and the P1 amino acid in the tripeptide is selected from the group consisting of alanine, aspartic acid, citrulline, γ-carboxy-glutamic acid, glutamic acid, glutamine, leucine, lysine, methionine sulfoxide, and selenomethionine. In another embodiment, the P3 amino acid in the tripeptide is selected from the group consisting of alanine, leucine, glutamic acid, lysine, O-allyl tyrosine, phenylalanine, proline, and threonine, the P2 amino acid in the tripeptide is selected from the group consisting alanine, valine, leucine, methionine, aspartic acid, glutamic acid, asparagine, glutamine, serine, threonine, tyrosine, citrulline, methionine sulfoxide, and γ-carboxy-glutamic acid, and the P1 amino acid in the tripeptide is selected from the group consisting of aspartic acid and glutamic acid. In another embodiment, the P3 amino acid in the tripeptide is selected from the group consisting of alanine, leucine, glutamic acid, lysine, O-allyl tyrosine, phenylalanine, proline, and threonine, the P2 amino acid in the tripeptide is selected from the group consisting alanine, valine, leucine, methionine, aspartic acid, glutamic acid, asparagine, glutamine, serine, threonine, tyrosine, citrulline, methionine sulfoxide, and γ-carboxy-glutamic acid, and the P1 amino acid in the tripeptide is alanine.

In another embodiment, the P3 amino acid in the tripeptide is selected from the group consisting of alanine, leucine, glutamic acid, lysine, O-allyl tyrosine, phenylalanine, proline, and threonine, the P2 amino acid in the tripeptide is selected from the group consisting of aspartic acid and glutamic acid, and the P1 amino acid in the tripeptide is selected from the group consisting of alanine, aspartic acid, citrulline, γ-carboxy-glutamic acid, glutamic acid, glutamine, leucine, lysine, methionine sulfoxide, and selenomethionine. In another embodiment, the P3 amino acid in the tripeptide is selected from the group consisting of alanine, leucine, glutamic acid, lysine, O-allyl tyrosine, phenylalanine, proline, and threonine, the P2 amino acid in the tripeptide is selected from the group consisting of aspartic acid and glutamic acid, and the P1 amino acid in the tripeptide is selected from the group consisting of aspartic acid and glutamic acid. In another embodiment, the P3 amino acid in the tripeptide is selected from the group consisting of alanine, leucine, glutamic acid, lysine, O-allyl tyrosine, phenylalanine, proline, and threonine, the P2 amino acid in the tripeptide is selected from the group consisting of aspartic acid and glutamic acid, and the P1 amino acid in the tripeptide is alanine.

In another embodiment, the P3 amino acid in the tripeptide is selected from the group consisting of alanine, leucine, glutamic acid, lysine, O-allyl tyrosine, phenylalanine, proline, and threonine, the P2 amino acid in the tripeptide is alanine, and the P1 amino acid in the tripeptide is selected from the group consisting of alanine, aspartic acid, citrulline, γ-carboxy-glutamic acid, glutamic acid, glutamine, leucine, lysine, methionine sulfoxide, and selenomethionine. In another embodiment, the P3 amino acid in the tripeptide is selected from the group consisting of alanine, leucine, glutamic acid, lysine, O-allyl tyrosine, phenylalanine, proline, and threonine, the P2 amino acid in the tripeptide is alanine, and the P1 amino acid in the tripeptide is selected from the group consisting of aspartic acid and glutamic acid. In another embodiment, the P3 amino acid in the tripeptide is selected from the group consisting of alanine, leucine, glutamic acid, lysine, O-allyl tyrosine, phenylalanine, proline, and threonine, the P2 amino acid in the tripeptide is alanine, and the P1 amino acid in the tripeptide is alanine.

In another embodiment, the P3 amino acid in the tripeptide is D-leucine or D-alanine, the P2 amino acid in the tripeptide is selected from the group consisting alanine, valine, leucine, methionine, aspartic acid, glutamic acid, asparagine, glutamine, serine, threonine, tyrosine, citrulline, methionine sulfoxide, and γ-carboxy-glutamic acid, and the P1 amino acid in the tripeptide is selected from the group consisting of alanine, aspartic acid, citrulline, γ-carboxy-glutamic acid, glutamic acid, glutamine, leucine, lysine, methionine sulfoxide, and selenomethionine. In another embodiment, the P3 amino acid in the tripeptide is D-leucine or D-alanine, the P2 amino acid in the tripeptide is selected from the group consisting alanine, valine, leucine, methionine, aspartic acid, glutamic acid, asparagine, glutamine, serine, threonine, tyrosine, citrulline, methionine sulfoxide, and γ-carboxy-glutamic acid, and the P1 amino acid in the tripeptide is selected from the group consisting of aspartic acid and glutamic acid. In another embodiment, the P3 amino acid in the tripeptide is D-leucine or D-alanine, the P2 amino acid in the tripeptide is selected from the group consisting alanine, valine, leucine, methionine, aspartic acid, glutamic acid, asparagine, glutamine, serine, threonine, tyrosine, citrulline, methionine sulfoxide, and γ-carboxy-glutamic acid, and the P1 amino acid in the tripeptide is alanine.

In another embodiment, the P3 amino acid in the tripeptide is D-leucine or D-alanine, the P2 amino acid in the tripeptide is selected from the group consisting of aspartic acid and glutamic acid, and the P1 amino acid in the tripeptide is selected from the group consisting of alanine, aspartic acid, citrulline, γ-carboxy-glutamic acid, glutamic acid, glutamine, leucine, lysine, methionine sulfoxide, and selenomethionine. In another embodiment, the P3 amino acid in the tripeptide is D-leucine or D-alanine, the P2 amino acid in the tripeptide is selected from the group consisting of aspartic acid and glutamic acid, and the P1 amino acid in the tripeptide is selected from the group consisting of aspartic acid and glutamic acid. In another embodiment, the P3 amino acid in the tripeptide is D-leucine or D-alanine, the P2 amino acid in the tripeptide is selected from the group consisting of aspartic acid and glutamic acid, and the P1 amino acid in the tripeptide is alanine.

In another embodiment, the P3 amino acid in the tripeptide is D-leucine or D-alanine, the P2 amino acid in the tripeptide is alanine, and the P1 amino acid in the tripeptide is selected from the group consisting of alanine, aspartic acid, citrulline, γ-carboxy-glutamic acid, glutamic acid, glutamine, leucine, lysine, methionine sulfoxide, and selenomethionine. In another embodiment, the P3 amino acid in the tripeptide is D-leucine or D-alanine, the P2 amino acid in the tripeptide is alanine, and the P1 amino acid in the tripeptide is selected from the group consisting of aspartic acid and glutamic acid.

In some embodiments, the P3 amino acid in the tripeptide is selected from the group consisting of alanine, D-alanine, D-leucine, glutamic acid, L-leucine, O-allyl tyrosine, phenylalanine, proline, threonine, and valine.

In some embodiments, the P2 amino acid in the tripeptide is selected from the group consisting of α-aminoisobutyric acid, alanine, D-leucine, glutamic acid, glutamine, glycine, leucine, proline, serine, and valine.

In some embodiments, the P1 amino acid in the tripeptide is selected from the group consisting of alanine, aspartic acid, citrulline, gamma-carboxy-glutamic acid, glutamic acid, glutamine, leucine, and lysine.

In some embodiments, the P3 amino acid in the tripeptide is selected from the group consisting of alanine, D-alanine, D-leucine, glutamic acid, L-leucine, O-allyl tyrosine, phenylalanine, proline, threonine, and valine, the P2 amino acid in the tripeptide is selected from the group consisting of α-aminoisobutyric acid, alanine, D-leucine, glutamic acid, glutamine, glycine, leucine, proline, serine, and valine, and the P1 amino acid in the tripeptide is selected from the group consisting of alanine, aspartic acid, citrulline, gamma-carboxy-glutamic acid, glutamic acid, glutamine, leucine, and lysine, wherein —P3-P2-P1- is not -Glu-Val-Cit- or -Asp-Val-Cit-. In some embodiments of any of the variations provided herein, —P3-P2-P1- is not -Glu-Val-Cit- or -Asp-Val-Cit-.

In some embodiments of tripeptides, the P3 amino acid is in the D-amino acid configuration, one of the P2 or P1 amino acid has an aliphatic side chain with hydrophobicity no greater than that of leucine (e.g., no greater than that of valine), and the other of the P2 or P1 amino acid is a polar amino acid or is negatively charged (e.g., at plasma physiological pH). In some embodiments, the P3 amino acid is in the D-amino acid configuration, the P2 amino acid has an aliphatic side chain with hydrophobicity no greater than that of leucine (e.g., no greater than that of valine), and the P1 amino acid is a polar amino acid or is negatively charged (e.g., at plasma physiological pH). In some embodiments, the P3 amino acid is in the D-amino acid configuration, the P1 amino acid has an aliphatic side chain with hydrophobicity no greater than that of leucine (e.g., no greater than that of valine), and the P2 amino acid is a polar amino acid or is negatively charged (e.g., at plasma physiological pH). In some embodiments, —P3-P2-P1- is selected from the group consisting of -D-Leu-Ala-Asp-, -D-Leu-Ala-Glu-, -D-Ala-Ala-Asp-, and -D-Ala-Ala-Glu-. In some embodiments, —P3-P2-P1- is selected from the group consisting of -D-Leu-Asp-Ala-, -D-Leu-Glu-Ala-, -D-Ala-Asp-Ala-, and -D-Ala-Glu-Ala-.

In other particularly preferred embodiments —P2-P1- is selected from the group consisting of -Ala-Glu-, -Leu-Glu-, -Ala-Met(O)— and -Leu-Met(O)— with the side chains of both amino acids in the L-stereochemical configuration. In some embodiment, —P2-P1- is selected from the group consisting of -Ala-Ala-, -Ala-Asp-, -Ala-Cit-, -Ala-(γ-carboxsy-glutamic acid)-, -Ala-Glu-, -Ala-Gln-, -Ala-Leu-, -Ala-Lys-, -Ala-Met(O)—, -Ala-selenomethionine-, -D-Leu-Glu-, -Leu-Glu-, -Glu-Ala-, -Glu-Cit-, -Glu-Leu-, -Gly-Glu-, -Leu-Cit-, -Leu-Glu-, -Leu-Lys-, -Leu-Met(O)—, -(naphthylalanine)-Lys-, -Pro-Cit-, -Ser-Asp-, -Ser-Glu-, -Val-Cit-, and -Val-Gln-. In some embodiments, —P2-P1- is -Ala-Glu-. In some embodiments, —P2-P1- is -Ala-Asp-.

In some embodiments, —P3-P2- is selected from the group consisting of -Ala-Ser-, -Ala-Ala-, -Leu-Ala-, -Leu-Glu-, -Leu-Gly-, -Leu-Leu-, Leu-Ser-, -Leu-Val-, -Glu-Ala-, -Glu-Leu-, -Glu-Pro-, -Glu-Val-, -Lys-Leu-, —(O-allyl tyrosine)-Leu-, —(O-allyl tyrosine)-Pro-, -Phe-Ser-, -Pro-Leu-, -Pro-(naphthylalanine)-, and -Thr-Glu-. In some embodiments, —P3-P2- is selected from the group consisting of -Ala-Ser-, -D-Ala-Ala-, -D-Leu-Ala-, -D-Leu-Glu-, -D-Leu-Gly-, -D-Leu-Leu-, D-Leu-Ser-, -D-Leu-Val-, -Glu-Ala-, -Glu-Leu-, -Glu-Pro-, -Glu-Val-, L-Leu-Ala-, -Lys-Leu-, —(O-allyl tyrosine)-D-Leu-, —(O-allyl tyrosine)-Pro-, -Phe-Ser-, -Pro-Leu-, -Pro-(naphthylalanine)-, and -Thr-Glu-. In some embodiments, —P3-P2- is -D-Leu-Ala- or -L-Leu-Ala-. In some embodiments, —P3-P2- is -D-Leu-Ala-. In some embodiments, —P3-P2- is -D-Ala-Ala-.

In some embodiments, —P3-P2-P1- is selected from the group consisting of -Ala-Ser-Asp-, -Ala-Ser-Glu-, -Ala-Ala-Cit-, -Ala-Ala-Glu-, -Leu-Ala-Ala-, -Leu-Ala-Asp-, -Leu-Ala-Cit-, -Leu-Ala-(γ-carboxy-glutamic acid)-, -Leu-Ala-Glu-, -Leu-Ala-Gln-, -Leu-Ala-Leu-, -Leu-Ala-Lys-, -Leu-Ala-Met(O)—, -Leu-Ala-(selenomethionine)-, -Leu-Glu-Ala-, -Leu-Glu-Cit-, -Leu-Gly-Glu-, -Leu-Leu-Cit-, -Leu-Leu-Glu-, -Leu-Leu-Lys-, -Leu-Leu-Met(O)—, Leu-Ser-Glu-, -Leu-Val-Gln-, -Glu-Ala-Leu-, -Glu-Leu-Cit-, -Glu-Pro-Cit-, -Lys-Leu-Cit-, —(O-allyl tyrosine)-Leu-Glu-, —(O-allyl tyrosine)-Pro-Cit-, -Phe-Ser-Glu-, -Pro-Leu-Glu-, -Pro-(naphthylalanine)-Lys-, and -Thr-Glu-Leu-. In some embodiments, —P3-P2-P1- is selected from the group consisting of -Ala-Ser-Asp-, -Ala-Ser-Glu-, -D-Ala-Ala-Cit-, -D-Ala-Ala-Glu-, -D-Leu-Ala-Ala-, -D-Leu-Ala-Asp-, -D-Leu-Ala-Cit-, -D-Leu-Ala-(γ-carboxy-glutamic acid)-, -D-Leu-Ala-Glu-, -D-Leu-Ala-Gln-, -D-Leu-Ala-Leu-, -D-Leu-Ala-Lys-, -D-Leu-Ala-Met(O)—, -D-Leu-Ala-(selenomethionine)-, -D-Leu-Glu-Ala-, -D-Leu-Glu-Cit-, -D-Leu-Gly-Glu-, -D-Leu-Leu-Cit-, -D-Leu-Leu-Glu-, -D-Leu-Leu-Lys-, -D-Leu-Leu-Met(O)—, -D-Leu-Ser-Glu-, -D-Leu-Val-Gln-, -Glu-Ala-Leu-, -Glu-Leu-Cit-, -Glu-Pro-Cit-, -L-Leu-Ala-Glu-, -Lys-Leu-Cit-, —(O-allyl tyrosine)-D-Leu-Glu-, —(O-allyl tyrosine)-Pro-Cit-, -Phe-Ser-Glu-, -Pro-Leu-Glu-, -Pro-(naphthylalanine)-Lys-, and -Thr-Glu-Leu-. In some embodiments, —P3-P2-P1- is selected from the group consisting of Ala-Cit-Cit-, -Cit-Cit-Cit-, -Cit-Glu-Cit-, -Cit-Glu-Glu-, -D-Leu-Ala-Glu-, -D-Leu-Ala-Lys-, -D-Leu-Cit-Glu-, -D-Leu-Glu-Lys-, -D-Leu-Leu-Cit-, -D-Leu-Leu-Glu-, -D-Leu-Leu-Lys-, -D-Leu-Leu-Met(O)—, -D-Leu-Phe-Glu-, -Glu-Ala-Glu-, -Glu-Ala-Met(O)—, -Glu-Glu-Cit-, -Leu-(naphthylalanine)-Lys-, -Lys-Glu-Met(O)—, -Pro-Ala-Cit-, -Pro-Ala-Glu-, -Pro-Cit-Cit-, -Pro-Cit-Glu-, -Pro-Glu-Ala-, -Pro-Glu-Cit-, -Pro-Glu-Glu-, -Pro-Glu-Lys-, -Pro-Lys-Glu-, -Pro-(naphthylalanine)-Lys-, and -Thr-Cit-Cit-.

It is understood that the Peptide Cleavable Unit (W) of a Ligand Drug Conjugate is a peptide sequence that can contain more than three amino acids. In peptide sequences containing four or more amino acids, the tripeptide described herein is any three contiguous amino acids within the sequence (i.e., the tripeptide can occupy any three adjacent positions of the sequence). Therefore, the embodiments described herein for P1, P2, and P3 can be applied to amino acids of any positions corresponding to three contiguous amino acids of the Peptide Cleavable Unit (W). For example, if the tripeptide that is recognized by the intracellular protease is located at positions —P6-P5-P4-, embodiments for P3 described herein apply to P6, embodiments for P2 described herein apply to P5, and embodiments for P1 described herein apply to P4. In another example, if the tripeptide that is recognized by the intracellular protease is located at positions —P4-P3-P2-, embodiments for P3 described herein apply to P4, embodiments for P2 described herein apply to P3, and embodiments for P1 described herein apply to P2. It is further understood that for a Peptide Cleavable Unit (W) in which the tripeptide is located at positions other than —P3-P2-P1-, the P1 amino acid of the Peptide Cleavable Unit (W) is an amino acid that is amenable to cleavage, for example by endopeptidase action. In some embodiments P1 amino acid is not in D-configuration. In some embodiments, the C-terminal amino acid is γ-carboxy-glutamic acid. In some embodiments, wherein the Peptide Cleavable Unit contains four or more amino acids, the amino acid(s) extrinsic to the tripeptide do not increase the overall hydrophobicity of the peptide sequence. In some embodiments, when the Peptide Cleavable Unit contains amino acid(s) in addition to the tripeptide, the additional amino acid(s) do not contain hydrophobic residues (e.g., residues more hydrophobic than leucine or residues more hydrophobic than valine).

The hydrophobicity of a given compound, including relative hydrophobicities of different compounds, can be assessed experimentally or computationally by methods known in the art. Hydrophobicity can be assessed, for example, by determination of a partition coefficient P, which may be determined experimentally and expressed as log P, or which can be determined computationally and expressed as clogP. Values of clogP can be computed using various types of commercially available software, such as ChemDraw or DataWarrior. Such methods may be used to assess the hydrophobicity of an amino acid or to assess the relative hydrophobicities of different amino acids. Such methods may also be used to assess the hydrophobicity of a Drug-Linker Compound as described herein or to assess the relative hydrophobicities of different Drug-Linker Compounds.

In some embodiments, provided are Ligand-Drug Conjugates (e.g., ADCs) that are less active than the comparator Ligand Drug Conjugate (e.g., dipeptide ADC containing -val-cit-), either in vivo or in vitro, but are also significantly less toxic. Without being bound by theory, the Ligand-Drug Conjugate is not required to be as active because the therapeutic window will still be increased if it is less active and less toxic. Exemplary compound exhibiting this effect may include Compounds 38 and 39 herein with AIB in position P2.

In still other particularly preferred embodiments the tripeptide has the structure of:

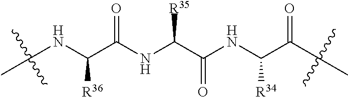

or a salt thereof, in particular a pharmaceutical acceptable salt, wherein the wavy line at the nitrogen atom of the tripeptide N-terminal amino acid, which is indicated as P3 in the afore-described Drug Linker compounds and drug linker moieties of Ligand Drug Conjugates derived therefrom, indicates the site of covalent attachment as an amide bond to the P4 amino acid residue when W is comprised of a tetrapeptide in which the selectivity conferring tripeptide is the C-terminal component of the tetrapeptide, or to A′ or L_{R}/L_{R}′ when W consists of the tripeptide and subscript a′ is 1 or 0, respectively, and the wavy line at the tripeptide's C-terminal amino acid residue, which is indicated as P1 in the afore-described Drug Linker compounds and drug linker moieties of Ligand Drug Conjugates derived therefrom, is the site of covalent attachment to the P-1 residue, when W is comprised of a tetrapeptide in which the selectivity conferring tripeptide is the N-terminal component of the tetrapeptide or to —Y_{y}-D when W consists of the tripeptide; and
wherein R³⁶, in the R stereochemical configuration, is —CH(CH₃)₂, R³⁵ is —CH(CH₃)₂, or —CH₃, and R³⁴ is —CH₂SH, —CH₂CH₂CH₂CH₂NH₂, —CH(OH)CH₃ or —CH₂CH₂CO₂H.

In more particular preferred drug linker moieties and Drug Linker compounds, R³⁶ is —CH(CH₃)₂ in the R stereochemical configuration and R³⁴ is —CH₂CH₂CO₂H. In especially preferred embodiments R³⁶ is —CH(CH₃)₂ in the R stereochemical configuration; and R³⁵ is —CH₃ and R³⁴ is —CH₂CH₂CO₂H, both of which are in the S stereochemical configuration as shown.

In some embodiments, the normal tissue homogenate is from bone marrow and the tumor tissue homogenate is from the tumor of a xenograft model of the same species, wherein greater selectivity for proteolysis by tumor tissue homogenate over the normal tissue homogenate is in comparison to a comparator Conjugate having a val-cit dipeptide Cleavable Unit. In some embodiments greater selectivity for tumor tissue over normal tissue by an Antibody Drug Conjugate in which the Peptide Cleavable Unit is comprised of the selectivity conferring tripeptide is shown in a xenograft model by substantial retention of the tumor growth profile obtained from administering an Antibody Drug Conjugate in which the Peptide Cleavable Unit is val-cit and with administration of the corresponding tripeptide-based non-binding control Conjugate showing reduced non-target mediated cytoxicity to normal bone marrow when compared to the corresponding dipeptide-based non-binding control, wherein that cytoxicity to normal cells is responsible for an adverse event associated with administering the dipeptide-based ADC at its maximum tolerated dose. In some embodiments, the normal tissue is bone marrow, liver, kidney, esophageal, breast, or corneal.

In some of those embodiments reduced non-target mediated cytoxicity is observed from histology of normal tissue (e.g., bone marrow, liver, kidney, esophageal, breast, or corneal tissue) from the same or different rodent species as used in the xenograft model on administering a non-binding control conjugate corresponding to the targeting tripeptide-based Antibody Drug Conjugate by showing reduced loss of nuclei staining of mononuclear cells in comparison to that from administration of the dipeptide-based non-binding control, so as to provide an improved therapeutic window for the tripeptide-based ADC. In some embodiments, the normal tissue is bone marrow. In a preferred embodiment mouse is used in the xenograft study and bone marrow is from rat, because rat is more sensitive to MMAE toxicity than mouse. In other embodiments the improvement in tolerability is shown by reduction in neutrophil and/or reticulocyte loss and/or from more rapid rebound from that loss.

### 2.2.4 Stretcher Units

In the above and following embodiments, a primary linker within a drug linker moiety of a Ligand Drug Conjugate may exemplify the general formula of -M²-A(BU)—[HE]-A_{O}-B—, -M²-A(BU)—[HE]-A′_{a′}-, -M²-A-[HE]-A_{O}-B—, -M²-A-[HE]-A′_{a′}, -M³-A(BU)—[HE]-A_{O}-B— or -M³-A(BU)—[HE]-A′_{a′}-, and a primary linker of a Drug Linker compound, which can be used to prepare a Ligand Drug Conjugate, may exemplify the general formula of M¹-A(BU)—[HE]-A_{O}-B—, M¹-A(BU)—[HE]-A′_{a′}-, M¹-A-[HE]-A_{O}-B—, or M¹-A-[HE]-A′_{a′}-, wherein BU is an acyclic or cyclic Basic Unit; [HE] when present is—preferably —C(═O)—, which is provided by a first optional Stretcher Unit (A) that is present; M² is succinimide moiety; M³ is succinic acid amide moiety and M¹ is a maleimide moiety, wherein A represents either a single discreet unit or a first subunit of A, which is sometimes indicated as A₁ when A_{O} is present as a second subunit of A, which is sometimes indicated as A₂, wherein A/A₂ is covalently attached to A′ in those primary linkers with no Branching Unit (B) and in which subscript a′ is 1 so that A′ becomes a subunit of A, or is covalently attached to W when subscript a′ is 0, or is covalently attached to B in those primary linkers containing a Branching Unit.

When either A_{O} or A′ is present in any one those embodiments, that subunit of a first Stretcher Unit (A) is indicated as A₂ to signify it as a subunit of A, wherein preferably A_{O}/A′ correspond independently in structure to an optionally substituted amine-containing acid (e.g., an amino acid) residue, wherein the residue of the carboxylic acid terminus of the amine-containing acid is covalently attached to B in those primary linkers in which that component is present, or to A′, if present as A₂, or to W in those primary linkers in which B and A′ are absent, wherein said covalent attachment is through an amide functional group and the residue of the amine terminus is covalently attached to the remainder of A. If B is present and A_{O} is absent, A is a single discreet unit that is bonded to B, and if B is absent and A is a single discreet unit then A is bonded to W through [HE], which is provided by A, wherein [HE] is —C(═O)—.

In some of those embodiments, A_{O}/A′ has or is comprised of the formula of -L^{P}(PEG)-, wherein L^{P} is a Parallel Connector Unit and PEG is a PEG Unit. In those embodiments, the PEG Unit contains a total of 2 to 36 ethyleneoxy monomer units and L^{P} is an amine-containing acid residue, preferably an amino acid residue, covalently attached within LU of a drug linker moiety of a Ligand Drug Conjugate compound or LU′ of a Drug Linker compound through amide functional groups. In preferred embodiments, the PEG Unit contains a total of 4 to 24 contiguous ethyleneoxy monomer units.

In other of those embodiments, A_{O}/A′ is an amine-containing acid residue having the structure of formula 3a, formula 4a or formula 5a:

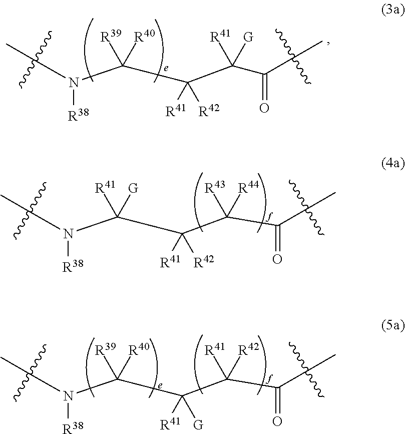

wherein the wavy line adjacent to the nitrogen atom indicates the site of covalent attachment to the remainder of A, and the wavy line adjacent to the carbonyl carbon atom indicates the site of covalent attachment to B if B is present or to A′/W when B is absent; subscripts e and f are independently 0 or 1; and
G is hydrogen, —OH, —OR^{PR}, —CO₂H, —CO₂R^{PR} or an optionally substituted C₁-C₆alkyl, wherein the optional substituent when present is selected from the group consisting of —OH, —OR^{PR}, —CO₂H, and —CO₂R^{PR}; and wherein R^{PR} is a suitable protecting group, or
G is N(R^{PR})(R^{PR}) or an optionally substituted C₁-C₆ alkyl, wherein the optional substituent when present is N(R^{PR})(R^{PR}), wherein R^{PR} are independently a protecting group or R^{PR} together form a suitable protecting group, or
G is —N(R⁴⁵)(R⁴⁶), or an optionally substituted C₁-C₆ alkyl, wherein the optional substituent when present is —N(R⁴⁵)(R⁴⁶), wherein one of R⁴⁵ and R⁴⁶ is hydrogen or R^{PR}, wherein R^{PR} is a suitable protecting group, and the other is hydrogen or optionally substituted C₁-C₆ alkyl;
R³⁸ is hydrogen or optionally substituted C₁-C₆ alkyl; and
R³⁹-R⁴⁴ are independently selected from the group consisting of hydrogen, optionally substituted C₁-C₆ alkyl, optionally substituted C₆-C₂₀ aryl, and optionally substituted C₅-C₂₀ heteroaryl, or
R³⁹, R⁴⁰ together with the carbon atom to which both are attached define a C₃-C₆ carbocyclo, and R⁴¹-R⁴⁴ are as defined herein,
or R⁴³, R⁴⁴ together with the carbon atom to which both are attached define a C₃-C₆ carbocyclo, and R³⁹-R⁴² are as defined herein,
or R⁴⁰ and R⁴¹, or R⁴⁰ and R⁴³, or R⁴¹ and R⁴³ to together with the carbon atom or heteroatom to which both are attached and the atoms intervening between those carbon atoms and/or heteroatoms define a C₅-C₆ carbocyclo or a C₅-C₆ heterocyclo, and R³⁹, R⁴⁴ and the remainder of R⁴⁰-R⁴³ are as defined herein,
or A_{O}/A′ is an α-amino or β-amino acid residue, wherein the nitrogen atom of the α-amino residue is covalently attached to the remainder of A, and the carbonyl carbon atom of its carboxylic acid residue is covalently attached to B if B is present or to W when B is absent, wherein both attachments are preferably through amide functional groups.

### 2.2.5 Spacer Units

A Spacer Unit is a component of a secondary linker (L_{O}) of Drug Linker Compound or a Linker Unit in a drug linker moiety of a Ligand Drug Conjugate compound represented by the structure of:

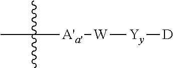

in which subscript y is 1 or 2, indicating the presence of one or two Spacer Unit, so that Y_{y} is Y or —Y—Y′—, wherein subscript a is 0 or 1, A′ is an optional first Stretcher Unit, which becomes a component of the primary linker (L_{R}/L_{R}′) as a subunit of a first optional Stretcher Unit (A) that is present when subscript a′ is 1 are there is no Branching Unit (B) in L_{R}/L_{R}′; W is a Peptide Cleavable Unit of formula —[Pₙ] . . . [P3]-[P2]-[P1]- or [Pₙ] . . . [P3]-[P2]-[P1]-[P-1]-, wherein subscript n ranges from 0 to 12 (e.g., 0-10, 3-12 or 3-10) and Pₙ . . . P3, P2, P1, P-1 are amino acid residues wherein the P1, P2 and P3 are the tripeptide amino acid residues conferring selectivity for protease cleavage by tumor tissue homogenate over normal tissue homogenate as described herein and/or which alters the biodistribution of a Ligand Drug Conjugate so that the Conjugate whose Peptide Cleavable Unit is comprised of the P3-P2-P1 tripeptide favors the tumor tissue in comparison to the normal tissue when compared to the biodistribution of a comparator peptide in which the Peptide Cleavable Unit is the dipeptide val-cit.

When W does not contain a P-1 residue, proteolytic action on L_{O} releases a drug linker fragment of formula —Y-D, when subscript y is 1, or —Y—Y′-D, when subscript y is 2, wherein Y is a first Spacer Unit and Y′ is a second Spacer Unit, whereupon the Spacer Units in those fragments undergo self-immolation to complete release of D as free drug. When W does contain a P-1 residue, proteolytic action on L_{O} releases a first drug linker fragment of formula [P-1]-Y-D or [P-1]-Y—Y′-D. However, for convenience the P-1 residue will be associated with the sequence in SEQ IDs describing such Peptide Cleavable Units. Completing release of free drug then requires exopeptidase action to remove the [P-1]amino acid residue to provide either Y-D or —Y—Y′-D as a second drug linker fragment similarly to when W does not contain a P-1 residue. The —Y—Y′-D linker fragment then proceeds to a third drug linker fragment of formula Y′-D. In either variant, Y-D or Y′-D spontaneously decomposes to complete release of D as free drug.

A self-immolative Spacer Unit (Y) covalently bonded to P1 or P-1 of a peptide Cleavage Unit (W) is comprised or consists of a self-immolating moiety as defined herein so that enzymatic processing of W activates the self-immolative moiety of Y for its self-destruction thus initiating release of the Drug Unit as free Drug. In those aspects in which subscript y is 1, the self-immolative moiety of Y is directly attached to an optionally substituted heteroatom of the Drug Unit. As previously discussed when subscript y is 2, then Y_{y} is —Y—Y′— wherein Y is a first self-immolative Spacer covalently attached to the Peptide Cleavable Unit (W) and Y′ is second self-immolative Spacer Unit, which in some aspects is a carbamate functional group shared between Y and D. In other aspects Y′ is a methylene carbamate unit. In either aspect Y_{y} is bonded to the Drug Unit (D) such that spontaneous self-destruction of the first self-immolative Spacer Unit Y initiated by endopeptidase action on the amide bond covalently attaching W to Y or exopeptidase action on the amide bond of [P-1]-D releases Y′-D, which then spontaneously decomposes to complete release of D as free drug.

In some embodiments Y contains a PAB or PAB-related self-immolative moiety bonded to -D or —Y′-D, in which subscript y is 1 or 2, respectively, which have a central arylene or heteroarylene substituted by a masked electron donating group (EDG) and a benzylic carbon bonded to D through a shared heteroatom or functional group, or bonded to D indirectly through an intervening second Spacer Unit (Y′), wherein the masked EDG and benzylic carbon substituents are ortho or para to each other (i.e., 1,2 or 1,4 substitution pattern). In those embodiments the second Spacer Unit (Y′) is capable of self-immolation or spontaneous decomposition or is absent.

Exemplary structures of self-immolative Spacer Units having a PAB or PAB-related self-immolative moiety in which the central (hetero)arylene has the requisite 1,2 or 1,4 substitution pattern that allows for 1,4- or 1,6-fragmentation for release D or [P-1]-D, when subscript y is 1, or —Y′-D, or —[P-1]-Y′-D in which subscript y is 2, wherein Y′ is capable of self-immolation or spontaneous decomposition, are represented by:

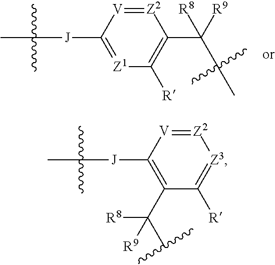

wherein the wavy line adjacent to J indicates the site of covalent attachment to P1 if the selectivity conferring tripeptide is directly attached —Y′-D or to P-1 if the selectivity conferring tripeptide is indirectly attached —Y′-D through that amino acid residue, and the other wavy line indicates the site of covalent attachment to —Y′-D, wherein J is a heteroatom, optionally substituted where permitted (i.e., optionally substituted —NH—), Y′ is an optional second Spacer Unit, D is a Drug Unit, wherein when Y‘ is absent Y’ is replaced by a heteroatom from D so that D becomes D′, which is the remainder of the Drug Unit; and
wherein V, Z¹, Z², Z³ are independently ═N or ═C(R²⁴)—, wherein each R²⁴ is independently selected from the group consisting of hydrogen and optionally substituted C₁-C₁₂ alkyl, optionally substituted C₂-C₁₂ alkenyl, optionally substituted C₂-C₁₂ alkynyl, optionally substituted C₆-C₂₀ aryl, optionally substituted (C₆-C₂₀ aryl)-C₁-C₆ alkyl-, optionally substituted C₅-C₂₀ heteroaryl and optionally substituted (C₅-C₂₀ heteroaryl)-C₁-C₆ alkyl-, and halogen and an electron withdrawing group; R′ is hydrogen or optionally substituted C₁-C₁₂ alkyl, optionally substituted C₂-C₁₂ alkenyl, optionally substituted C₂-C₁₂ alkynyl, optionally substituted C₆-C₂₀ aryl, optionally substituted (C₆-C₂₀ aryl)-C₁-C₆ alkyl-, optionally substituted C₅-C₂₀ heteroaryl, or optionally substituted C₅-C₂₀ heteroaryl)-C₁-C₆ alkyl-, or an electron donating group; and R⁸ and R⁹ are independently selected from the group consisting of hydrogen, optionally substituted C₁-C₁₂ alkyl, optionally substituted C₂-C₁₂ alkenyl, optionally substituted C₂-C₁₂ alkynyl, optionally substituted C₆-C₂₀ aryl and optionally substituted C₅-C₂₀ heteroaryl, or both R⁸ and R⁹ together with the carbon atom to which they are attached define a C₃-C₈ carbocyclo. In preferred embodiments, one or more of V, Z¹, Z² or one or more of V, Z², Z³ is ═CH—. In other preferred embodiments R′ is hydrogen or an electron donating group, including C₁-C₆ ethers such as —OCH₃ and —OCH₂CH₃, or one of R⁸, R⁹ is hydrogen and the other is hydrogen or C₁-C₄ alkyl. In more preferred embodiments two or more of V, Z¹ and Z² are ═CH— or two or more of V, Z² and Z³ are ═CH—. In other more preferred embodiments R⁸, R⁹ and R′ are each hydrogen.

Intracellular cleavage of the bond to J or the amide bond between P1 and P-1 results in release of Y′-D or —[P-1]-Y′-D, respectively, wherein —[P-1]-Y′-D is convertible to —Y′-D by exopeptidase activity of an intracellular protease of a targeted cell.

In some preferred embodiments, —Y_{y}-D in which subscript y is 2 has the structure of —Y—Y′-D is as follows:

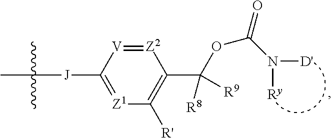

wherein —N(R^{y})D′ represents D, wherein D′ is the remainder of D, and wherein the dotted line indicates optional cyclization of R^{y} to D, wherein R^{y} is optionally substituted C₁-C₆ alkyl in absence of cyclization to D′ or optionally substituted C₁-C₆ alkylene when cyclized to D′; -J- is an optionally substituted heteroatom where permitted, including 0, S and optionally substituted —NH—, wherein J, a functional group comprised of J, or P-1 is bonded to P1, as indicated by the adjacent wavy line, of the tripeptide that confers selectivity for intracellular proteolysis over proteolysis by freely circulating proteases and selectivity for proteolysis by tumor tissue homogenate over proteolysis by normal tissue homogenate and/or selective biodistribution to tumor tissue over biodistribution to normal tissue, wherein cleavage of that bond initiates release of D as a secondary amine-containing biologically active compound from a compound of a Ligand Drug Conjugate composition and wherein the remaining variable groups are as defined above. Those variables are selected so that reactivity of J when released from processing of Peptide Cleavable Unit W within the targeted cells is balanced with the pKa of Y′-D or D eliminated from the PAB or PAB-type self-immolative moiety and the stability of the quinone-methide type intermediate resulting from that elimination.

In those embodiments, the intervening moiety between D and the benzylic carbon of the PAB or PAB-related self-immolative moiety of Spacer Unit Y represents Y′ in —C(R⁸)(R⁹)—Y′-D so that a carbamate functional group is shared between Y and D. In such embodiments fragmentation of the Spacer Unit Y with expulsion of Y′-D is followed by loss of CO₂ for release of D as biologically active compound having a primary or secondary amine whose nitrogen atom was bonded to the secondary linker comprised of the PAB or PAB-related self-immolative moiety.

In other preferred embodiments, —Y_{y}-D having a PAB or PAB-type moiety bound to —Y′-D or -D has the structure of:

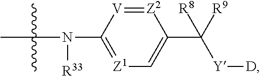

wherein the wavy line adjacent to the nitrogen atom indicates the point of covalent attachment to P-1 or the tripeptide of W that confers selectivity for intracellular proteolysis over proteolysis by freely circulating proteases and proteolysis by tumor tissue homogenate over proteolysis by normal tissue homogenate, wherein that bond is susceptible to the intracellular proteolysis, Y′ is an optional Spacer Unit that when absent is replaced with a phenolic oxygen atom or a sulfur atom from D, and when present is a carbamate functional group the nitrogen atom of which is from D; R³³ is hydrogen or optionally substituted C₁-C₆ alkyl, in particular hydrogen or C₁-C₄ alkyl, preferably hydrogen, —CH₃ or —CH₂CH₃, more preferably hydrogen. In more preferred embodiments, V, Z¹ and Z² are each ═CH— and R³³ is hydrogen.

In particularly preferred embodiments-Y_{y}-D has the structure of:

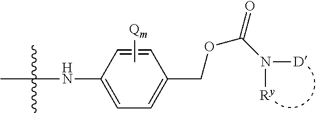

wherein —N(R^{y})D′ has its previous meaning and the wavy line indicates covalent attachment to P1; Q is —C₁-C₈alkyl, —O—(C₁-C₈ alkyl), or other electron donating group, -halogen, -nitro or -cyano or other electron withdrawing group (preferably, Q is —C₁-C₈ alkyl, —O—(C₁-C₈ alkyl), halogen, nitro or cyano); and subscript m is an integer ranging from 0-4 (i.e., the central arylene has no other substituents or 1-4 other substituents). In preferred embodiments subscript m is 0, 1 or 2 and each Q is an independently selected electron donating group.

In especially preferred embodiments, —Y_{y}— has the structure of:

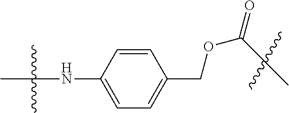 [SMILES: *Nc1ccc(COC(*)=O)cc1]

respectively, wherein the wavy line adjacent to the carbonyl carbon atom indicates the site of covalent attachment to an oxygen or sulfur atom of D to form a carbonate or thiocarbamate functional group that is shared between D and Y wherein that shared functional group is Y′, or to a secondary nitrogen atom to form a carbamate that is shared between D and Y, wherein that shared functional group is Y′, and the wavy line adjacent to the nitrogen atom indicates the site of covalent attachment as an amide bond to the carboxylic acid residue of P1.

Other structures of general formula —Y—Y′— in which Y is a self-immolative Spacer Unit are other than a PAB or PAB-type self-immolative Spacer Unit are illustrated in the following drug linker moieties.

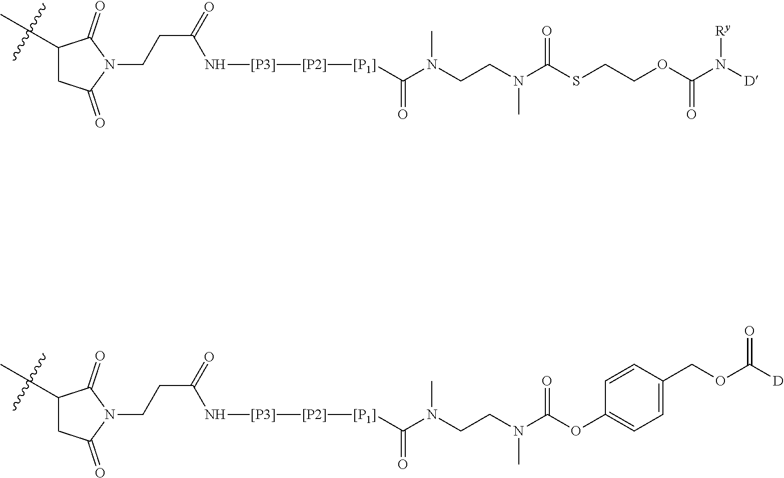

Without being bound by theory, the sequential self-immolation of Y in which Y is a PAB self-immolative Spacer Unit and Y′ is a carbamate functional group is illustrated for the secondary linker of Ligand Drug Conjugates and Drug Linker compounds having a tripeptide Peptide Cleavable Unit are as follows:

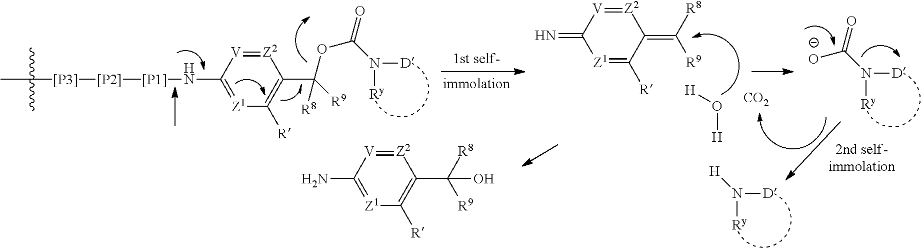

### 2.2.5 Drug Linkers

In general, a drug linker moiety of Formula 1A has the structure of:

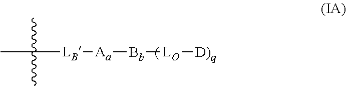

wherein the wavy line indicates covalent attachment of L_{B} to a Ligand Unit, A is a first optional Stretcher Unit; subscript a is 0 or 1 indicating the absence or presence of A, B is an optional Branching Unit; subscript b is 0 or 1 indicating the absence or presence of B, respectively, provided that subscript b is 1 when subscript q ranges from 2 to 4 and
L_{O} is a secondary linker having the formula of:

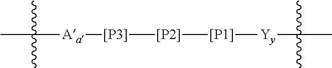

or a salt thereof, in particular a pharmaceutically acceptable salt, wherein A′ is a second optional Stretcher Unit, subscript a′ is 0 or 1 indicating the absence or presence of A′, respectively, Y is an optional Spacer Unit, subscript y is 0, 1 or 2 indicating the absence or presence of 1 or 2 Spacer Units, respectively, and P1, P2 and P3 are amino acid residues that together provide selectivity for proteolysis by a homogenate of tumor tissue over proteolysis by a homogenate of normal tissue, and/or together provide for preferred biodistribution of a Formula 1 Conjugate into tumor tissue in comparison to normal tissue, wherein cytotoxicity of the free drug released from the Conjugate towards the normal tissue is responsible at least in part for an adverse event typically associated with administration of a therapeutically effective amount of a comparator dipeptide-based Conjugate, wherein proteolytic cleavage occurs at the covalent bond between P1 and Y if subscript y is 1 or 2, or at the covalent bond between P1 and D if subscript y is 0 or
L_{O} is a secondary linker having the formula of:

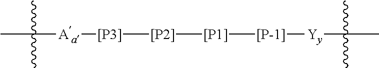

or a salt thereof, in particular a pharmaceutically acceptable salt, wherein A′, a′, Y, and y retain their previous meanings and P1, P2 and P3 are amino acid residues, optionally with the P-1 amino acid, that together provide selectivity for proteolysis by tumor tissue homogenate over proteolysis by normal tissue homogenate, and/or together provide for preferred biodistribution of the Formula 1 Conjugate into tumor tissue in comparison to normal tissue, wherein cytotoxicity of the free drug released from the Conjugate towards the normal tissue is responsible at least in part for an adverse event typically associated with administration of a therapeutically effective amount of a comparator dipeptide-based Conjugate, wherein proteolytic cleavage occurs at the covalent bond between P1 and P-1 to release a linker fragment having the structure of [P-1]-Y_{y}-D, or
L_{O} is a secondary linker having the formula of:

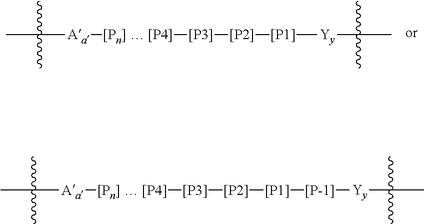

or a salt thereof, in particular a pharmaceutically acceptable salt, wherein A′, a′, Y, and y retain their previous meanings and P-1 and P1, P2, P3 . . . Pₙ are amino acid residues, wherein subscript n ranges from 0 to 12 (e.g., 0-10, 3-12 or 3-10) and P1, P2 and P3, optionally with P-1, together provide selectivity for proteolysis by tumor tissue homogenate over proteolysis by normal tissue homogenate and/or together provide for preferred biodistribution of the Formula 1 Conjugate prepared from the Drug Linker compound into tumor tissue in comparison to normal tissue, wherein cytotoxicity of the free drug released from the Conjugate towards the normal tissue is responsible at least in part for an adverse event typically associated with administration of a therapeutically effective amount of a comparator dipeptide-base Conjugate, wherein proteolytic cleavage occurs at the covalent bond between P1 and Y_{y}-D or between and P1 and P-1 to release a linker fragment having the structure of Y_{y}-D or [P-1]-Y_{y}-D, respectively, in which the later subsequently undergoes exopeptidase cleavage to release the linker fragment having the structure of Y_{y}-D. In both instances the Y_{y}-D linker fragment undergoes spontaneous decomposition to complete release of D as free drug.

The additional P4, P5 . . . Pₙ amino acid residues are selected so as to not alter the cleavage site that provides the —Y_{y}-D or —[P-1]-Y_{y}-D fragment, but instead are selected to retain a desired physiochemical and/or pharmokinetic property to the Ligand Drug Conjugate provided primarily by the P1, P2 and P3 amino acid residues, such as increased biodistribution of the Conjugate into tumor tissue, which is at the detriment for normal tissue distribution or to enhance that physiochemical and/or pharmokinetic property in comparison to a comparator dipeptide-based Conjugate.

In either one of those embodiments of L_{O} if subscript q is 1, then subscript b is 0 so that B is absent and A′ becomes an optional subunit of A and if subscript q is 2, 3 or 4, then subscript b is 1 so that B is present, A′ remains a component of L_{O} as shown and an optional subunit of A is indicated as A_{O}.

In some embodiments, in addition to improving global selectivity and/or improving biodistribution favoring tumor-associated proteases in comparison to that of normal tissue, the P1, P2 and P3 amino acid residues also reduce aggregation of a Conjugate that incorporates an amino acid sequence comprised of these amino acids in comparison to a dipeptide comparator conjugate. In some of those embodiments in which the Drug Unit is that of MMAE the drug linker moieties of the comparator Conjugate have the formula of mc-vc-PABC-MMAE.

In preferred embodiments of -L_{SS} and -L_{S}-containing drug linker moieties of a Formula 1A Ligand Drug Conjugate compound, the L_{SS} and L_{S} moieties contain a heterocyclo cyclic Basic Unit. Exemplary drug linker moieties in which subscript q is 1 and having those primary linkers in which the Peptide Cleavable Unit is a tripeptide are represented by the structures of Formula 1B, Formula 1C and Formula 1D:

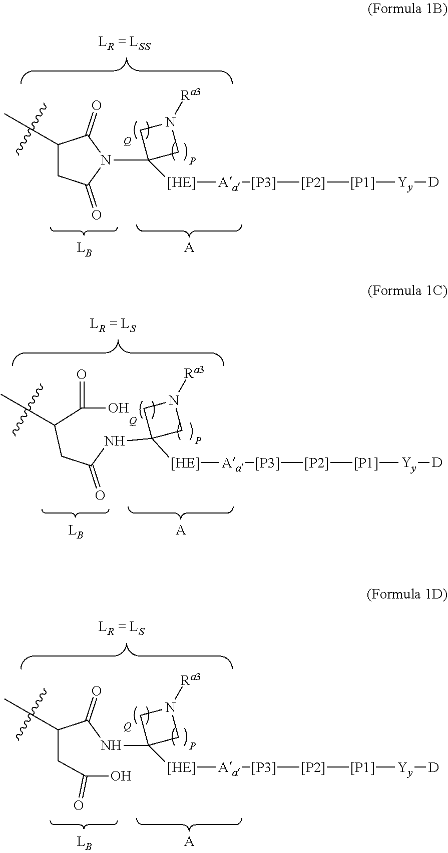

or a salt thereof, in particular a pharmaceutical acceptable salt, wherein HE is an optional Hydrolysis Enhancing Unit; A′ is an subunit, when present, of a first Stretcher Unit (A); subscript a′ is 0 or 1, indicating the absence or presence of A′, respectively; subscript P is 1 or 2; subscript Q ranges from 1 to 6, preferably subscript Q is 1 or 2, more preferably subscript Q has the same value as subscript P; and wherein R^{a3} is —H, optionally substituted C₁-C₆ alkyl, optionally substituted —C₁-C₄ alkylene-(C₆-C₁₀ aryl), or —R^{PEG1}—O—(CH₂CH₂O)₁₋₃₆—R^{PEG2}, wherein R^{PEG1} is C₁-C₄ alkylene, R^{PEG2} is —H or C₁-C₄ alkylene, wherein the basic nitrogen bonded to R^{a3} is optionally protonated in a salt form, preferably in a pharmaceutically acceptable salt form, or R^{a3} is a nitrogen protecting group such as a suitable acid-labile protecting group; the wavy line indicated covalent binding to a sulfur atom of a Ligand Unit; P1, P2 and P3 are as previously defined for any one of the embodiments of Peptide Cleavable Units; and the remaining variable groups are as described for any one of the embodiments of a drug linker moiety of Formula 1A.

In other preferred embodiments of -L_{SS} and -L_{S}-containing drug linker moieties of Formula 1A of a Ligand Drug Conjugate compound, the L_{SS} and L_{S} moieties contain a acyclic cyclic Basic Unit. Exemplary drug linker moieties having those primary linkers in which the Peptide Cleavable Unit is a dipeptide are represented by the structures of Formula 1E, Formula 1F and Formula 1G:

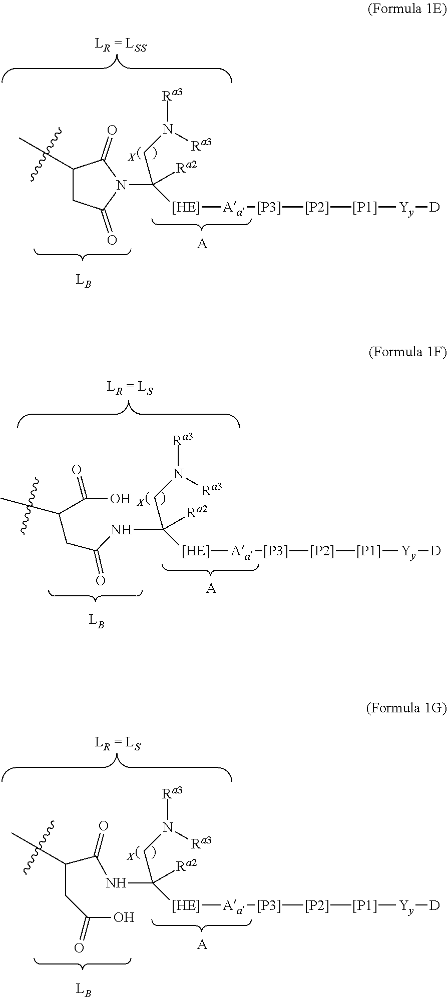

or a salt thereof, in particular a pharmaceutical acceptable salt, wherein HE is an optional Hydrolysis Enhancing Unit; A′ is an subunit, when present, of a first Stretcher Unit (A); subscript a′ is 0 or 1, indicating the absence or presence of A′, respectively; subscript x is 1 or 2; R^{a2} is —H, optionally substituted C₁-C₆ alkyl, —CH₃ or —CH₂CH₃; R^{a3}, at each instance, is independently a nitrogen protecting group, —H or optionally substituted C₁-C₆ alkyl, preferably —H, an acid-labile protecting group, —CH₃ or —CH₂CH₃, or both R^{a3} together with the nitrogen to which they are attached define a nitrogen protecting group or an azetidinyl, pyrrolidinyl or piperidinyl heterocyclyl, in which a basic primary, secondary or tertiary amine so defined is optionally protonated in a salt form, preferably a pharmaceutically acceptable salt form; the wavy line indicated covalent binding to a sulfur atom of a Ligand Unit; P1, P2 and P3 are as previously defined for any one of the embodiments of Peptide Cleavable Units and the remaining variable groups are as described for any one of the embodiments of a drug linker moiety of Formula 1A.

In other preferred embodiments, a primary linker does not have a Basic Unit. Exemplary drug linker moieties having that primary linker in which the Peptide Cleavable Unit is a tripeptide are represented by the structures of Formula 1H, Formula 1J and Formula 1K:

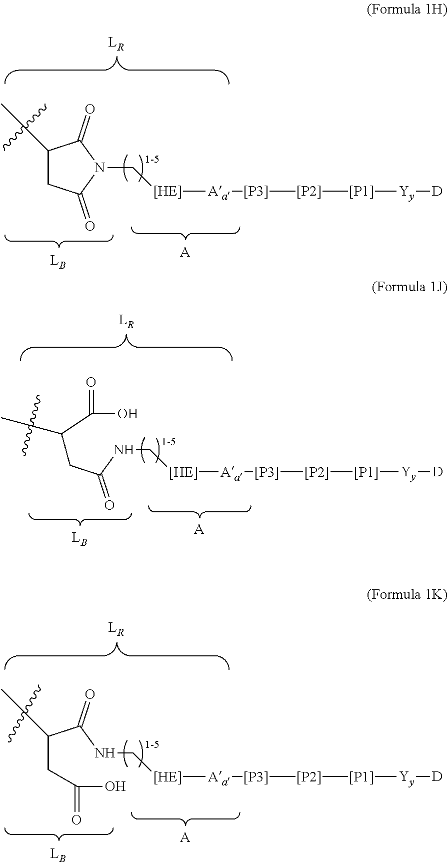

or a salt thereof, in particular a pharmaceutical acceptable salt, wherein HE is an optional Hydrolysis Enhancing Unit; A′ is a subunit (A₂), when present, of a first Stretcher Unit (A); subscript a′ is 0 or 1, indicating the absence or presence of A′; the wavy line indicates covalent binding to a sulfur atom of a Ligand Unit; P1, P2 and P3 are as previously defined for any one of the embodiments of Peptide Cleavable Units and the remaining variable groups are as described for any one of the embodiments of a drug linker moiety of Formula 1A.

In more preferred embodiments in which there is a heterocyclo cyclic Basic Unit in the Linker Unit, a majority of Ligand Drug Conjugate compounds in a Ligand Drug Conjugate composition have drug linker moieties represented by the structures of:

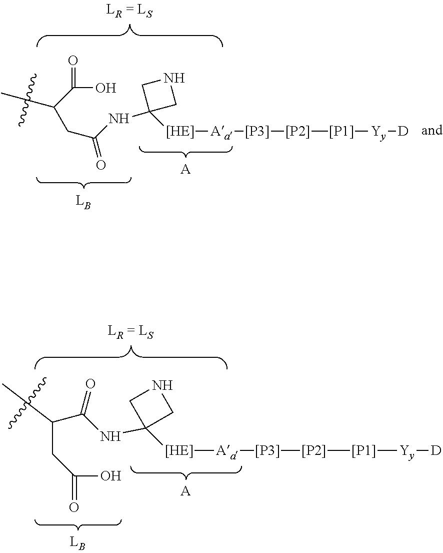

optionally in a salt form, in particular in pharmaceutical acceptable salt form, and in more preferred embodiments in which there is an acyclic Basic Unit in the Linker Unit, a majority of Ligand Drug Conjugate compounds in a Ligand Drug Conjugate composition have drug linker moieties represented by the structures of:

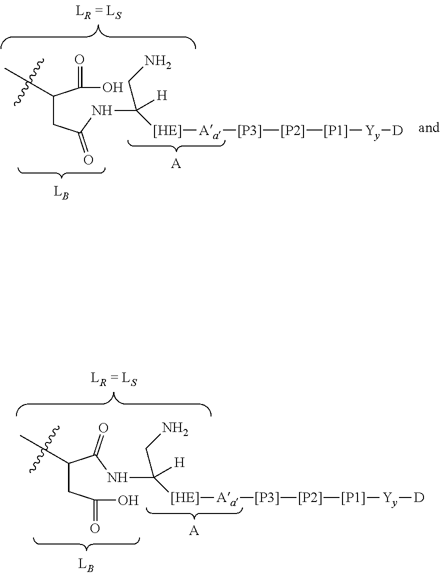

optionally in salt form, in particular in pharmaceutical acceptable salt form, wherein the variable groups of the L_{SS} and L_{S}-containing drug linker moieties are as previously described for drug linker moieties having a acyclic or heterocyclo cyclic Basic Unit,
and in other more preferred embodiments in which there is no Basic Unit in the Linker Unit, the predominate Ligand Drug Conjugate compound in a Ligand Drug Conjugate composition has drug linker moieties represented by the structure of Formula 1H, wherein the variable groups are as previously described for drug linker moieties of that formula.

In any one of the preceding drug linker moieties, HE is preferably present as —C(═O) and/or subscript y is 1 or 2, indicating the presence of one or two self-immolative Spacer Units, respectively.

In particularly preferred embodiments the —[P3]-[P2]-[P1]tripetide in in any one of the above drug linker moieties is, D-Leu-Leu-Met(O) or D-Leu-Ala-Glu, wherein Met(O) is methionine in which its sulfur atom is oxidized to a sulfoxide.

In especially preferred embodiments in which there is a heterocyclo cyclic Basic Unit in the Linker Unit, a majority of Ligand Drug Conjugate compounds in a Ligand Drug Conjugate composition have drug linker moieties represented by the structure of:

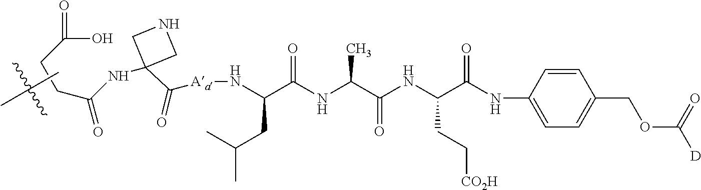

and salts thereof, in particular pharmaceutically acceptable salts, wherein the wavy line indicates covalent attachment to a sulfur atom from a Ligand Unit; subscript a′ is 0 or 1, indicating the absence or presence of A, respectively, wherein A′ is an amine-containing acid residue of formula 3a, 4a or 5a as described herein for a second optional Stretcher Unit or a subunit of a first optional Stretcher Unit, or A′ is an α-amino acid or β-amino acid residue; and D is a cytotoxic drug having a secondary amino group as the site of attachment to the Linker Unit of the drug linker moiety.

In other especially preferred embodiments in which there is a acyclic Basic Unit in the Linker Unit, a majority of Ligand Drug Conjugate compounds in a Ligand Drug Conjugate composition have drug linker moieties represented by the structure of:

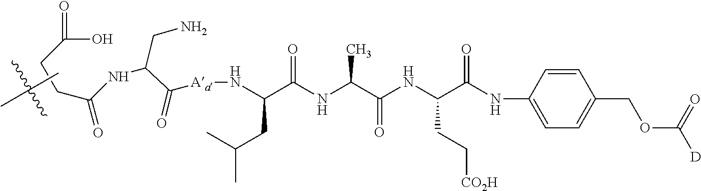

and salts thereof, in particular pharmaceutically acceptable salts, wherein the variable groups are as previously described for drug linker moieties having a cyclic Basic Unit.

In other especially preferred embodiments in which there is no Basic Unit, the predominate Ligand Drug Conjugate compound in a Ligand Drug Conjugate composition has drug linker moieties represented by the structure of:

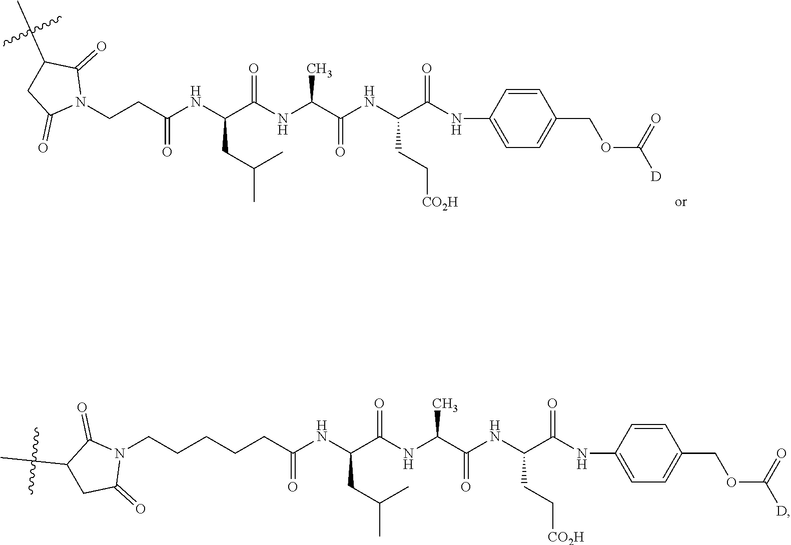

or a salt thereof, in particular a pharmaceutically acceptable salt, wherein the variable groups are as previously described for drug linker moieties having a cyclic Basic Unit. In those embodiments in which no BU is present, a Ligand Drug Conjugate composition comprised of either predominate Ligand Drug Conjugate compound is optionally further comprised of Ligand Drug Conjugate compounds in which the succinimide ring is in hydrolyzed form.

### 2.2.6 Auristatin Drug Units

An auristatin Drug Unit of a Ligand Drug Conjugate compound or Drug Linker compound incorporates an auristatin drug through covalent attachment of a Linker Unit of the Conjugate or Drug Linker compound to the secondary amine of an auristatin free drug having structure of D_{E} or D_{F} as follows:

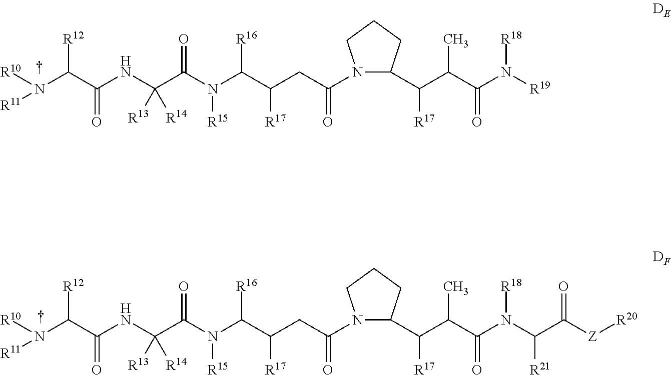

wherein the dagger indicates the site of covalent attachment of the nitrogen atom that provides a carbamate functional group, wherein —OC(═O)— of that functional group is Y′ on incorporation of the auristatin drug compound as -D into any one of the drug linker moieties of a Ligand Drug Conjugate compound or into any one of the Drug Linker compounds as described herein, so that for either type of compound subscript y is 2; and
one R¹⁰ and R¹¹ is hydrogen and the other is C₁-C₈ alkyl; R¹² is hydrogen, C₁-C₈ alkyl, C₃-C₈ carbocyclyl, C₆-C₂₄ aryl, —X¹—C₆-C₂₄ aryl, —X¹—(C₃-C₈ carbocyclyl), C₃-C₈ heterocyclyl or —X¹—(C₃-C₈ heterocyclyl); R¹³ is hydrogen, C₁-C₈alkyl, C₃-C₈ carbocyclyl, C₆-C₂₄ aryl, —X¹— C₆-C₂₄ aryl, —X¹—(C₃-C₈ carbocyclyl), C₃-C₈ heterocyclyl and —X¹—(C₃-C₈ heterocyclyl); R¹⁴ is hydrogen or methyl, or R¹³ and R¹⁴ taken together with the carbon to which they are attached comprise a spiro C₃-C₈ carbocyclo; R¹⁵ is hydrogen or C₁-C₈ alkyl; R¹⁶ is hydrogen, C₁-C₈ alkyl, C₃-C₈ carbocyclyl, C₆-C₂₄ aryl, —C₆-C₂₄—X¹-aryl, —X¹—(C₃-C₈ carbocyclyl), C₃-C₈ heterocyclyl and —X¹—(C₃-C₈ heterocyclyl); R¹⁷ independently are hydrogen, —OH, C₁-C₈ alkyl, C₃-C₈ carbocyclyl and O—(C₁-C₈ alkyl); R¹⁸ is hydrogen or optionally substituted C₁-C₈alkyl; R¹⁹ is —C(R^{19A})₂—C(R^{19A})₂-C₆-C₂₄ aryl, —C(R^{19A})₂—C(R^{19A})₂—(C₃-C₈ heterocyclyl) or —C(R^{19A})₂—C(R^{19A})₂—(C₃-C₈ carbocyclyl), wherein C₆-C₂₄ aryl and C₃-C₈ heterocyclyl are optionally substituted; R^{19A} independently are hydrogen, optionally substituted C₁-C₈ alkyl, —OH or optionally substituted —O—C₁-C₈ alkyl; R²⁰ is hydrogen or optionally substituted C₁-C₂₀ alkyl, optionally substituted C₆-C₂₄ aryl or optionally substituted C₃-C₈ heterocyclyl, or —(R⁴⁷O)ₘ—R⁴⁸, or —(R⁴⁷O)ₘ—CH(R⁴⁹)₂; R²¹ is optionally substituted —C₁-C₈ alkylene-(C₆-C₂₄ aryl) or optionally substituted —C₁-C₈ alkylene-(C₅-C₂₄ heteroaryl), or C₁-C₈ hydroxylalkyl, or optionally substituted C₃-C₈ heterocyclyl; Z is O, S, NH, or NR⁴⁶; R⁴⁶ is optionally substituted C₁-C₈ alkyl; subscript m is an integer ranging from 1-1000; R⁴⁷ is C₂-C₈ alkyl; R⁴⁸ is hydrogen or C₁-C₈ alkyl; R⁴⁹ independently are —COOH, —(CH₂)ₙ—N(R⁵⁰)₂, —(CH₂)ₙ—SO₃H, or —(CH₂)ₙ—SO₃—C₁-C₈alkyl; R⁵⁰ independently are C₁-C₈ alkyl, or —(CH₂)ₙ—COOH; subscript n is an integer ranging from 0 to 6; and X¹ is C₁-C₁₀ alkylene.

In some embodiments the auristatin drug compound has the structure of Formula D_{E-1}, Formula D_{E-2} or Formula D_{F-1}:

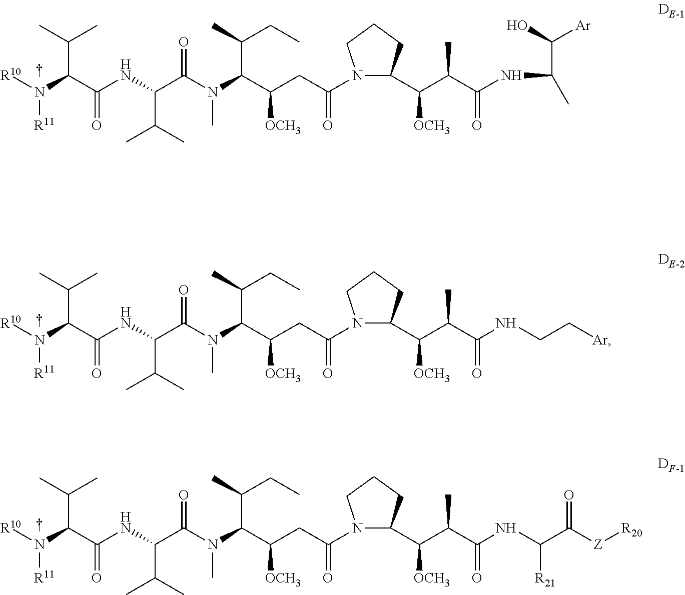

wherein Ar in Formula D_{E-1} or Formula D_{E-2} is C₆-C₁₀ aryl or C₅-C₁₀ heteroaryl, and in Formula D_{F-1}, Z is —O—, or —NH—; R²⁰ is hydrogen or optionally substituted C₁-C₆ alkyl, optionally substituted C₆-C₁₀ aryl or optionally substituted C₅-C₁₀ heteroaryl; and R²¹ is optionally substituted C₁-C₆ alkyl, optionally substituted —C₁-C₆ alkylene-(C₆-C₁₀ aryl) or optionally substituted —C₁-C₆ alkylene-(C₅-C₁₀ heteroaryl).

In some embodiments of Formula D_{E}, D_{F}, D_{E-1}, D_{E-2} or D_{F-1}, one of R¹⁰ and R¹¹ is hydrogen and the other is methyl.

In some embodiments of Formula D_{E-1} or D_{E-2}, Ar is phenyl or 2-pyridyl.

In some embodiments of Formula D_{F-1}, R²¹ is X¹—S—R^{21a} or X¹—Ar, wherein X¹ is C₁-C₆ alkylene, R^{21a} is C₁-C₄ alkyl and Ar is phenyl or C₅-C₆ heteroaryl and/or —Z— is —O— and R²⁰ is C₁-C₄ alkyl or Z is —NH— and R²⁰ is phenyl or C₅-C₆ heteroaryl.

In preferred embodiments the auristatin drug compound has the structure of Formula D_{F/E-3}:

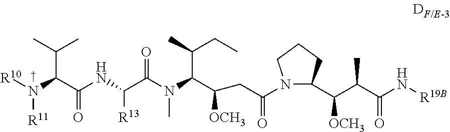

wherein one of R¹⁰ and R¹¹ is hydrogen and the other is methyl; R¹³ is isopropyl or —CH₂—CH(CH₃)₂; and R^{19B} is —CH(CH₃)—CH(OH)-Ph, —CH(CO₂H)—CH(OH)—CH₃, —CH(CO₂H)—CH₂Ph, —CH(CH₂Ph)-2-thiazolyl, —CH(CH₂Ph)-2-pyridyl, —CH(CH₂-p-Cl-Ph), —CH(CO₂Me)-CH₂Ph, —CH(CO₂Me)-CH₂CH₂SCH₃, —CH(CH₂CH₂SCH₃)C(═O)NH-quinol-3-yl, —CH(CH₂Ph)C(═O)NH-p-Cl-Ph, or R^{19B} has the structure of

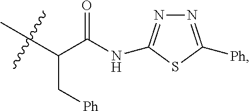 [SMILES: *C(Cc1ccccc1)C(=O)Nc1nnc(-c2ccccc2)s1]

wherein the wavy line indicates covalent attachment to the remainder of the auristatin compound.

In more preferred embodiments the auristatin drug compound incorporated into -D is monomethylauristatin E (MMAE) or monomethylauristatin F (MMAF).

In some embodiments, the Ligand-Drug Conjugate composition is represented by the structure of:

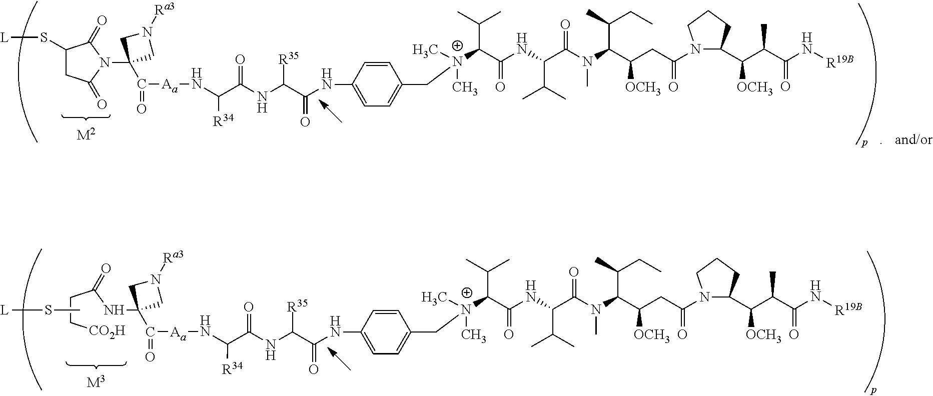

wherein subscript a is 1, so that A is present, wherein A is an α-amino acid or β-amino acid residue; R^{a3} is —H, optionally substituted C₁-C₆ alkyl, optionally substituted -C₁-C₄ alkylene-(C₆-C₁₀ aryl), —R^{PEG1}—O—(CH₂CH₂O)ₙ—R^{PEG2}, wherein R^{PEG1} is C₁-C₄ alkylene, R^{PEG2} is —H or C₁-C₄ alkyl, and subscript n′ ranges from 1 to 36, wherein the basic nitrogen bonded to R^{a3} is optionally protonated; R^{19B} is —CH(CH₃)—CH(OH)-Ph, —CH(CO₂H)—CH(OH)—CH₃, or —CH(CO₂H)—CH₂Ph; R³⁴ is isopropyl and R³⁵ is methyl or —(CH₂)₃NH(C═O)NH₂.

In some embodiments, the Ligand-Drug Conjugate composition is represented by the structure of:

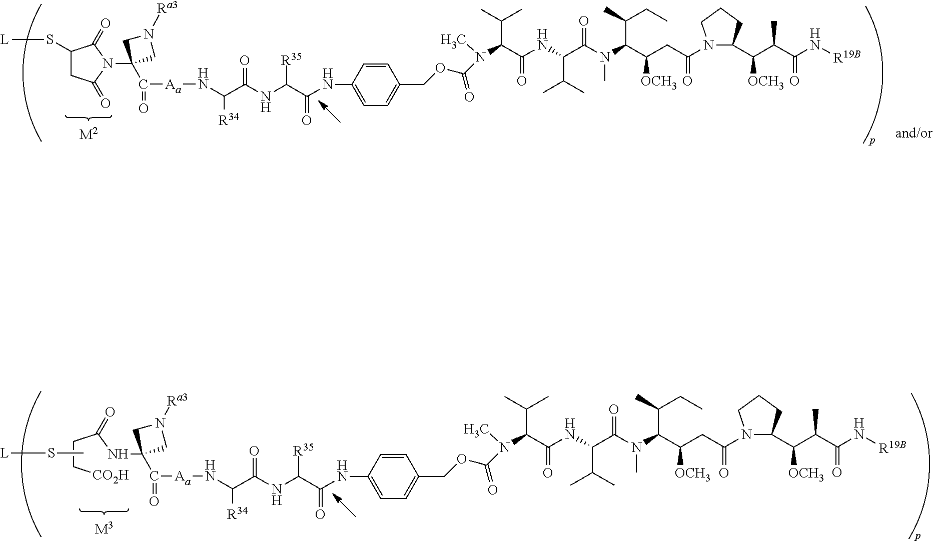

wherein subscript a is 1 so that A is present, wherein A is an α-amino acid or β-amino acid residue; R^{a3} is —H, optionally substituted C₁-C₆ alkyl, optionally substituted —C₁-C₄ alkylene-(C₆-C₁₀ aryl), —R^{PEG1}—O—(CH₂CH₂O)_{n′}—R^{PEG2}; R^{PEG1} is C₁-C₄ alkylene; R^{PEG2} is —H or C₁-C₄ alkyl; subscript n′ ranges from 1 to 36; and wherein the basic nitrogen atom bonded to R^{a3} is optionally protonated; R^{19B} is —CH(CH₃)—CH(OH)-Ph, —CH(CO₂H)—CH(OH)—CH₃, or —CH(CO₂H)—CH₂Ph; R³⁴ is isopropyl; and R³⁵ is methyl or —(CH₂)₃NH(C═O)NH₂.

In some embodiments, the Ligand Drug Conjugate compound is represented by:

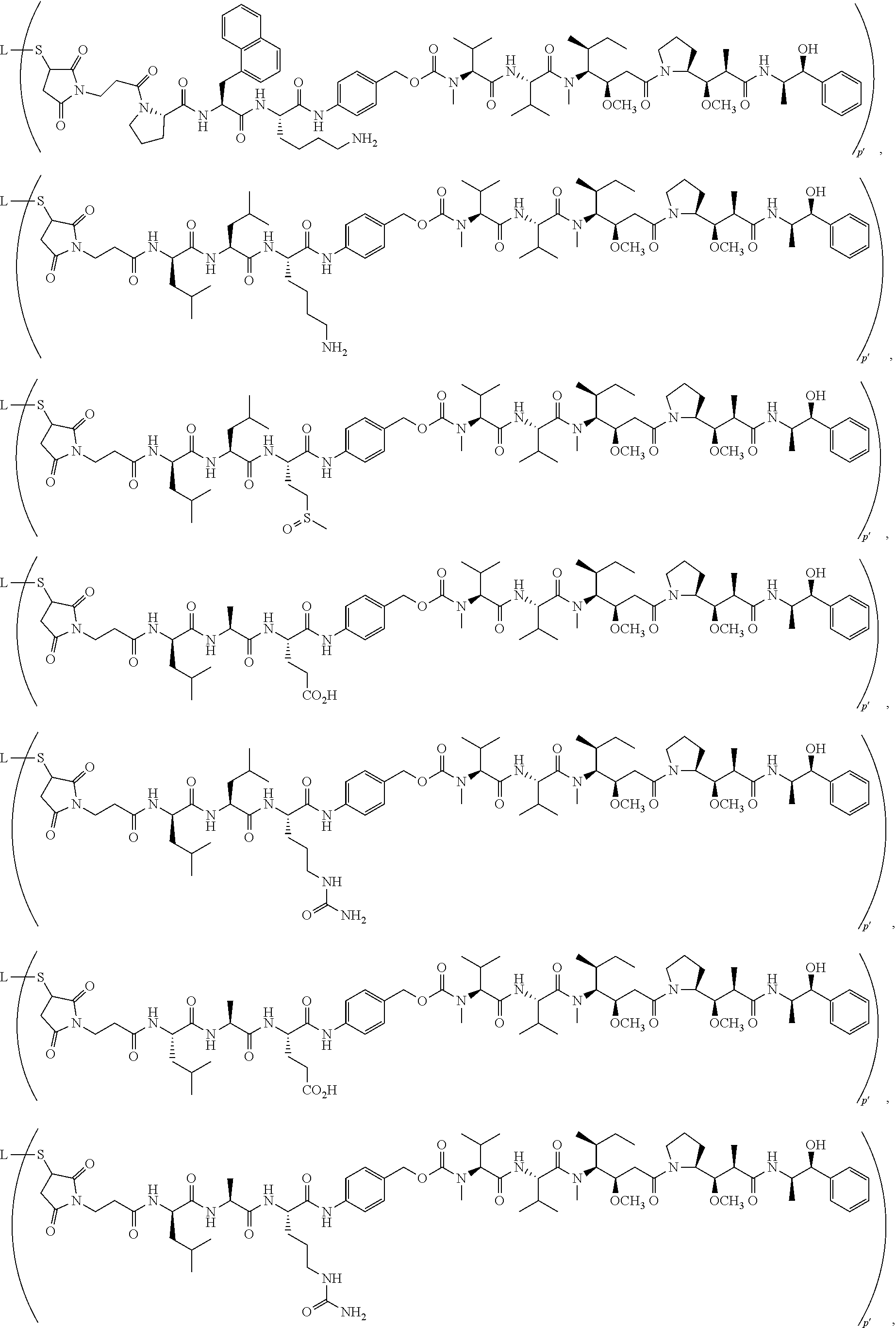

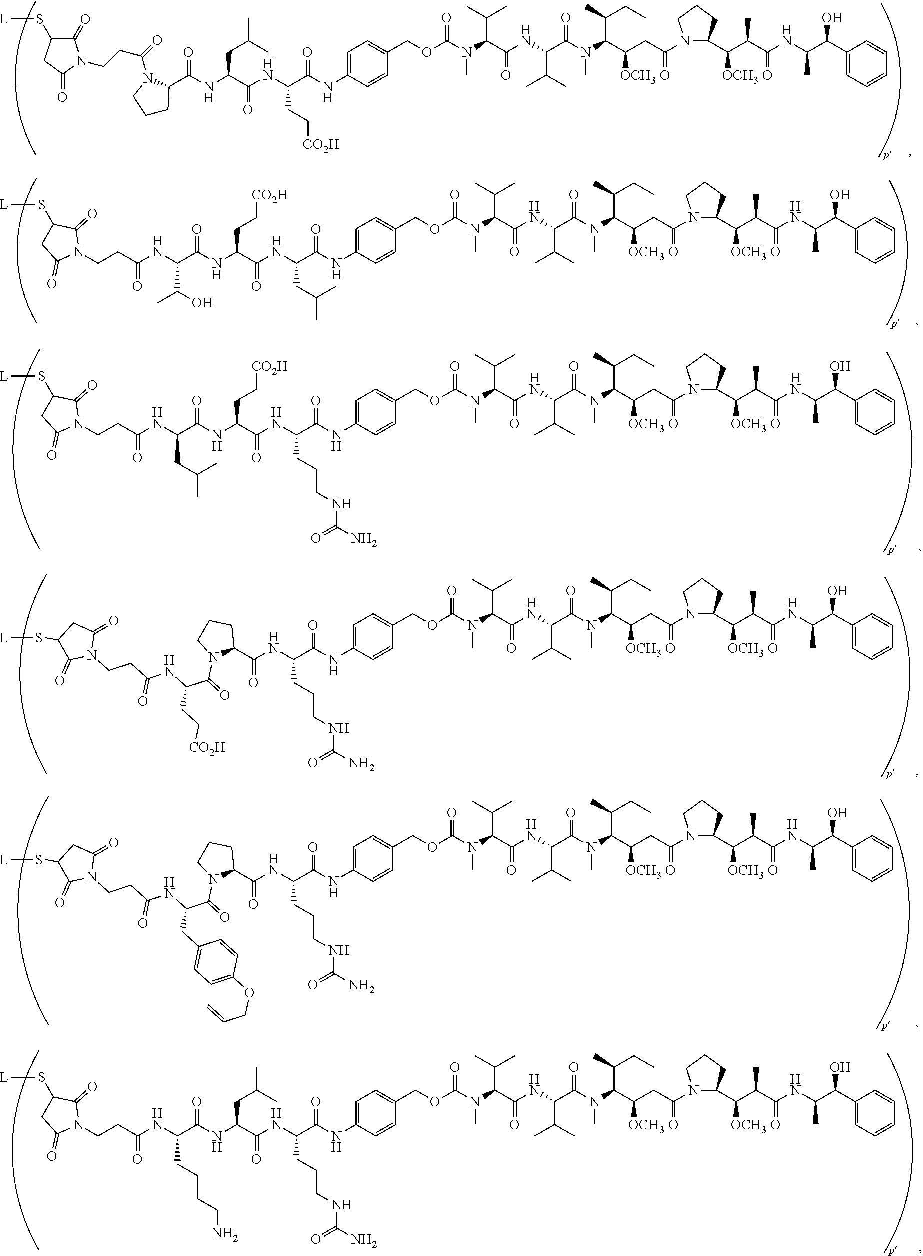

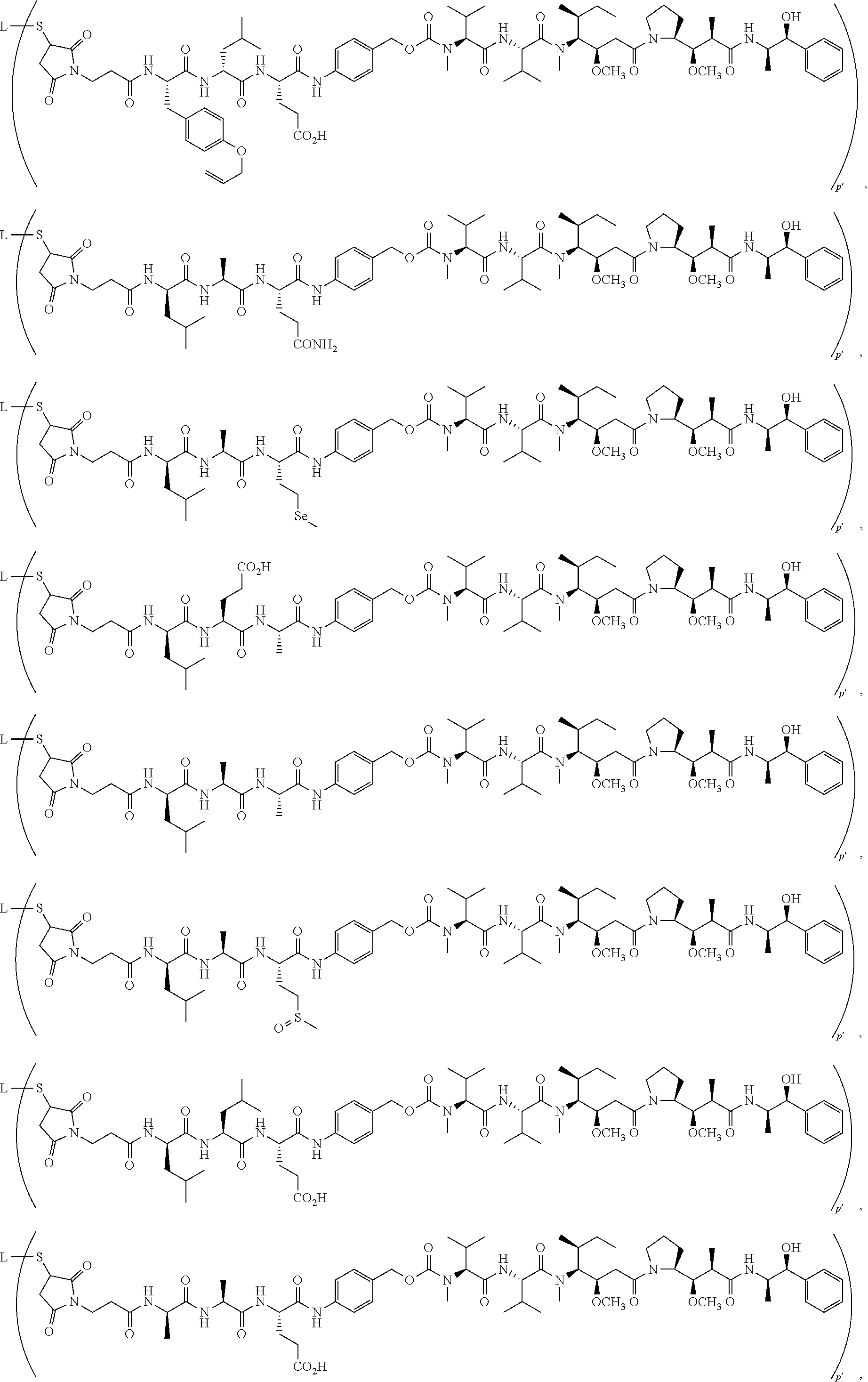

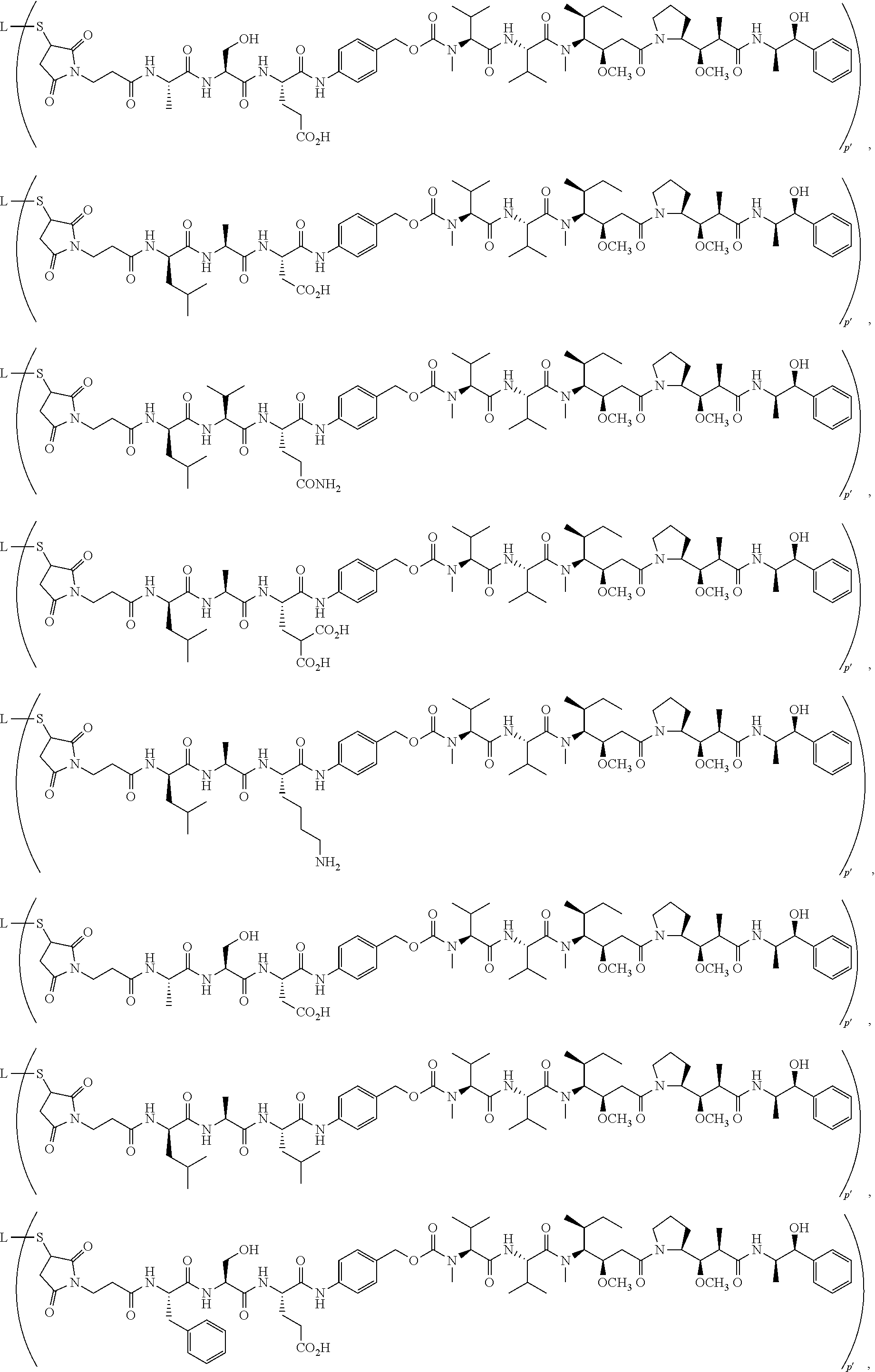

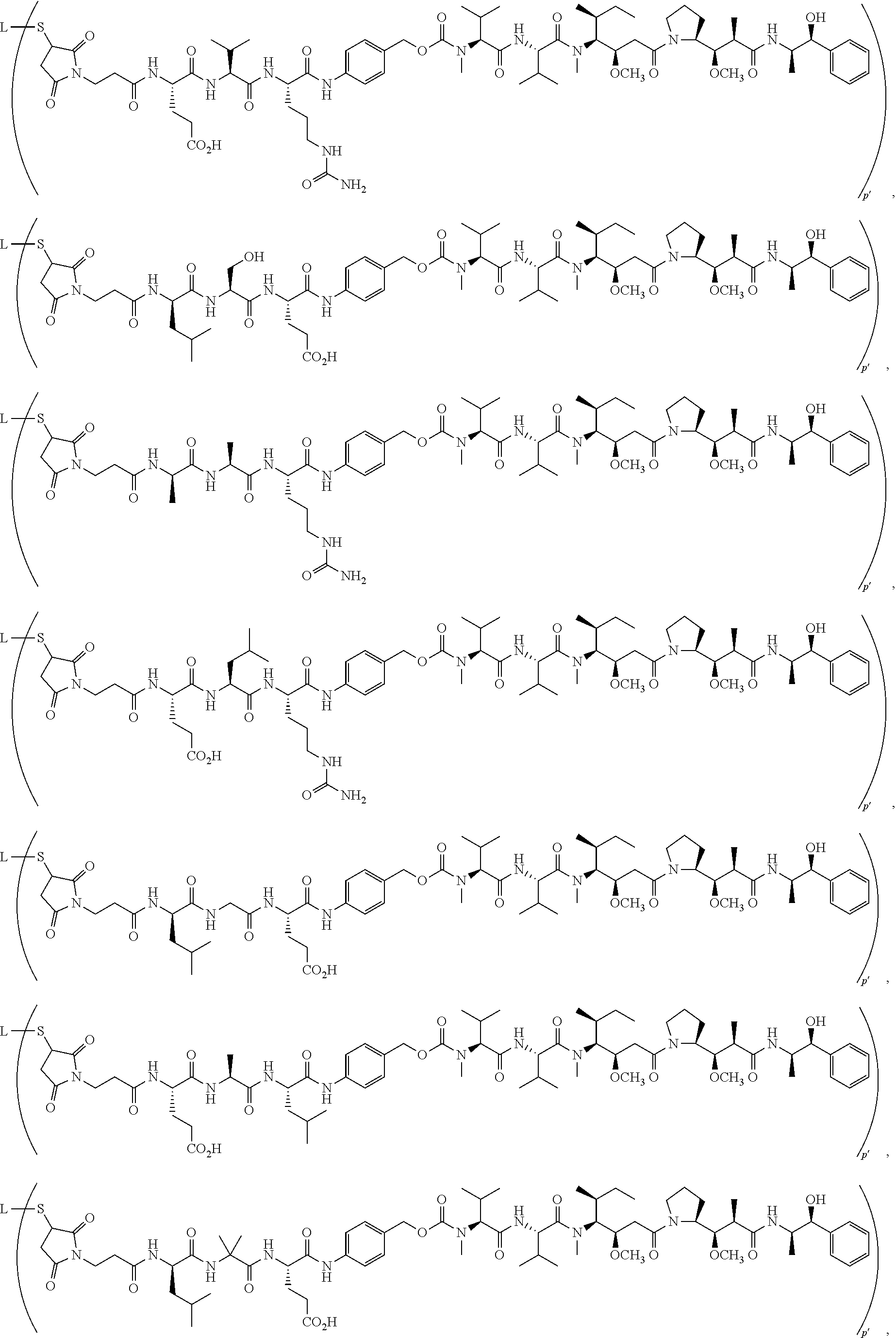

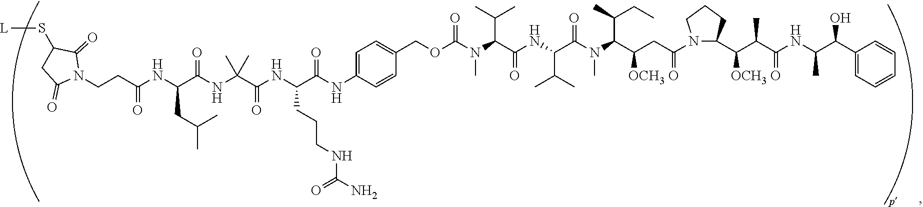

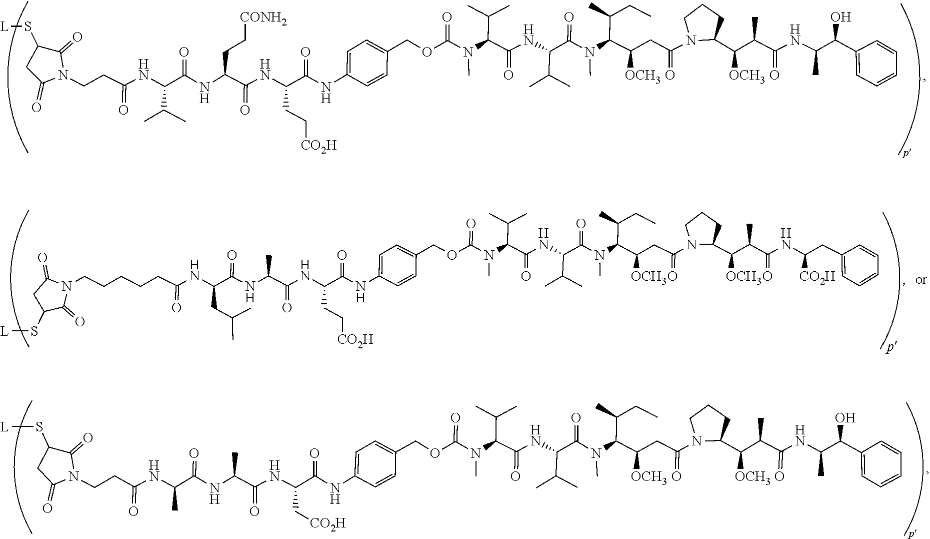

or a salt thereof (e.g., a pharmaceutically acceptable salt thereof), wherein L is a Ligand Unit, and subscript p′ is an integer from 1 to 24. It is understood that where L is an antibody, a sulfur atom S bonded to L in the aforementioned chemical structures represents a sulfur of the side chain of a cysteine residue of the antibody. In some embodiments, the subscript p′ is an integer from 1 to 12, 1 to 10 or 1 to 8 or is 4 or 8. In some embodiments, the subscript p′ is 1, 2, 3, 4, 5, 6, 7, 8, 9, 10, 11, 12, 13, 14, 15, 16, 17, 18, 19, 20, 21, 22, 23, or 24. In some embodiments, the subscript p′ is 2, 4, 6, or 8. In some embodiments, the subscript p′ is 2. In some embodiments, the subscript p′ is 4. In some embodiments, the subscript p′ is 6. In some embodiments, the subscript p′ is 8. Also included are Ligand Drug Conjugate compositions containing any of the Ligand Drug Conjugate compounds listed above wherein p′ is replaced with p as described herein.

### 2.3 Drug Linker Compounds

A Drug Linker compound is represented by the structure of Formula I:

LU′-(D′) (I)
or a salt thereof, wherein LU′ is LU precursor; and D′ represents from 1 to 4 Drug Units, which are preferably identical to each other, wherein the Drug Linker compound is further defined by the structure of Formula IA:

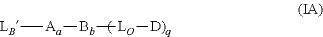

wherein L_{B}′ is an ligand covalent binding moiety precursor; A is a first optional Stretcher Unit; subscript a is 0 or 1 indicating the absence or presence of A, respectively, B is an optional Branching Unit; subscript b is 0 or 1 indicating the absence or presence of B, respectively, provided that subscript b is 1 when subscript q is selected from 2 to 4 and
L_{O} is a secondary linker having the formula of:

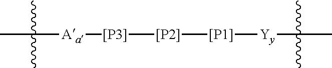

or a salt thereof, in particular a pharmaceutically acceptable salt, wherein A′ is a second optional Stretcher Unit, subscript a′ is 0 or 1 indicating the absence or presence of A′, respectively, Y is an optional Spacer Unit, subscript y is 0, 1 or 2 indicating the absence or presence of 1 or 2 Spacer Units, respectively, and P1, P2 and P3 are amino acid residues that together provide selectivity for proteolysis by a homogenate of tumor tissue over proteolysis by a homogenate of normal tissue, and/or together provide for preferred biodistribution of a Conjugate prepared from the Formula IA Drug Linker compound into tumor tissue in comparison to normal tissue, wherein cytotoxicity of the free drug released from the Conjugate towards the normal tissue is responsible at least in part for an adverse event typically associated with administration of a therapeutically effective amount of a comparator dipeptide-base Conjugate, wherein proteolytic cleavage occurs at the covalent bond between P1 and Y if subscript y is 1 or 2, or at the covalent bond between P1 and D if subscript y is 0 or
L_{O} is a secondary linker having the formula of:

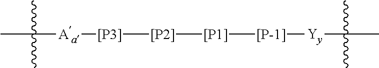

or a salt thereof, in particular a pharmaceutically acceptable salt, wherein A′, a′, Y, and y retain their previous meanings and P1, P2 and P3 are amino acid residues, optionally with the P-1 amino acid, that together provide selectivity for proteolysis by tumor tissue homogenate over proteolysis by normal tissue homogenate, and/or together provide for preferred biodistribution of a Conjugate prepared from the Formula IA Drug Linker compound into tumor tissue in comparison to normal tissue, wherein cytotoxicity of the free drug released from the Conjugate towards the normal tissue is responsible at least in part for an adverse event typically associated with administration of a therapeutically effective amount of a comparator dipeptide-base Conjugate, wherein proteolytic cleavage occurs at the covalent bond between P1 and P-1 to release a linker fragment having the structure of [P-1]-Y_{y}-D, or
L_{O} is a secondary linker having the formula of:

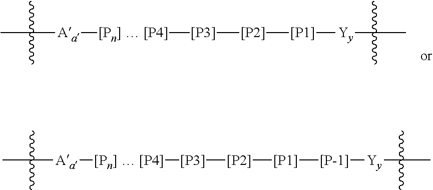

or a salt thereof, in particular a pharmaceutically acceptable salt, wherein A′, a′, Y, and y retain their previous meanings and P-1 and P1, P2, P3 . . . Pₙ are contiguous amino acid residues, wherein subscript n is an integer value providing for up to 12 (e.g., 3-12 or 3-10) of these amino acids and P1, P2 and P3, optionally with P-1, together provide selectivity for proteolysis by tumor tissue homogenate over proteolysis by normal tissue homogenate and/or together provide for preferred biodistribution of a Conjugate prepared from the Drug Linker compound into tumor tissue in comparison to normal tissue, wherein cytotoxicity of the free drug released from the Conjugate towards the normal tissue is responsible at least in part for an adverse event typically associated with administration of a therapeutically effective amount of a comparator dipeptide-base Conjugate, wherein proteolytic cleavage occurs at the covalent bond between P1 and Y_{y}-D or between and P1 and P-1 to release a linker fragment having the structure of Y_{y}-D or [P-1]-Y_{y}-D, respectively, in which the later subsequently undergoes exopeptidase cleavage to release the linker fragment having the structure of Y_{y}-D. In both instances the Y_{y}-D linker fragment undergoes spontaneous decomposition to complete release of D as free drug.

The additional P4, P5 . . . Pₙ amino acid residues are selected so as to not alter the cleavage site that provides the —Y_{y}-D or —[P-1]-Y_{y}-D fragment, but instead are selected to retain a desired physiochemical and/or pharmokinetic property for the Ligand Drug Conjugate that is prepared from the Formula IA Drug Linker compound, wherein the desired physiochemical and/or pharmokinetic property is provided primarily by the P1, P2 and P3 amino acid residues, such as increased biodistribution of the Conjugate into tumor tissue, which is to the detriment of normal tissue distribution, or to enhance that physiochemical and/or pharmokinetic property in comparison to a comparator dipeptide-base Conjugate.

In either one of those embodiments of L_{O} if subscript q is 1, then subscript b is 0 so that B is absent and A′ becomes an optional subunit of A and if subscript q is 2, 3 or 4, then subscript b is 1 so that B is present, A′ remains a component of L_{O} as shown and an optional subunit of A is then indicated as A_{O}.

A Drug Linker compound is particularly useful in preparing a Ligand Drug Conjugate of Formula 1 so that LU′ is a LU precursor for a drug linker moiety of a Ligand Drug Conjugate compound.

In some embodiments L_{B}′-A- of a Drug Linker compound has or is comprised of one of the structures of:

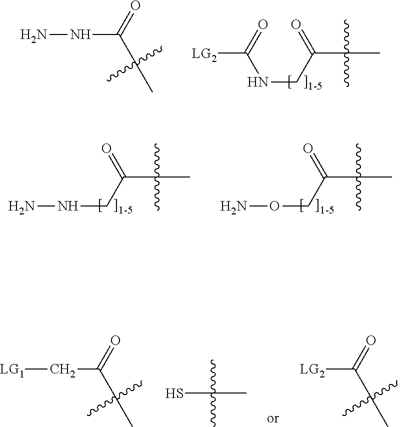

or a salt thereof, wherein LG₁ is a leaving group suitable for nucleophilic displacement by a targeting agent nucleophile; LG₂ is a leaving group suitable for amide bond formation to a targeting agent, or —OH to provide an activateable carboxylic acid suitable for amide bond formation to a targeting agent; and the wavy line indicates the site of covalent attachment to the remainder of the Drug Linker compound structure.

In other embodiments of a Formula IA Drug Linker compound in which subscript q is 1, L_{B}′-A- of has or is comprised of one of the structures of:

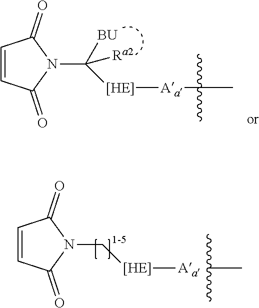

or a salt thereof, wherein A′ is an optional second subunit of A, sometimes indicated as A₂ if that subunit is present; subscript a′ is 0 or 1, indicating the absence or presence of A′, respectively; the wavy line adjacent to A′ indicates the site of covalent attachment to another subunit of A or to the Peptide Cleavable Unit; [HE] is an optional Hydrolysis Enhancing Unit, which is a component provided by A or a first subunit thereof; BU is a Basic Unit; R^{a2} is an optionally substituted C₁-C₁₂ alkyl group; and the dotted curved line indicates optional cyclization so that in the absence of said cyclization, BU is an acyclic Basic Unit having a primary, secondary or tertiary amine functional group as the basic function group of the acyclic Basic Unit, or in the presence of said cyclization BU is a cyclized Basic Unit in which R^{a2} and BU together with the carbon atom to which both are attached, define an optionally substituted spiro C₃-C₂₀ heterocyclo containing a skeletal basic nitrogen atom of a secondary or tertiary amine functional group as the basic function group of the cyclic Basic Unit,
wherein the basic nitrogen atom of the acyclic Basic Unit or cyclic Basic Unit is optionally suitably protected by a nitrogen protecting group, dependent on the degree of substitution of the basic nitrogen atom, or is optionally protonated

In other embodiments in which subscript q is 2, 3 or 4, L_{B}′-A- is comprised of one of the structures of:

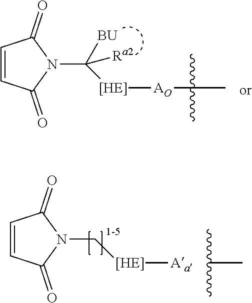

or a salt thereof, wherein the wavy line adjacent to A_{O} indicates the site of covalent attachment to B, A_{O} is an optional subunit of A, sometimes indicated as A₂ if that subunit is present and the remaining variable groups are as defined for Formula IA drug linker compounds in which subscript q is 1.

In some preferred embodiments in which subscript q is 1, L_{B}′-A- of a Drug Linker compound has or is comprised of one of the structures of:

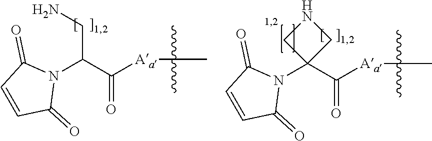

or a salt thereof, in particular as an acid addition salt, wherein A′ and subscript a′ are as previously described. Those L_{B}′-A- structures are exemplary self-stabilizing precursor moieties, sometimes indicated as L_{SS}′, since each is capable of being converted to a L_{SS} moiety of a Ligand Drug Conjugate compound.

In other preferred embodiments L_{B}′-A- of a Drug Linker compound has or is comprised of one of the structures of:

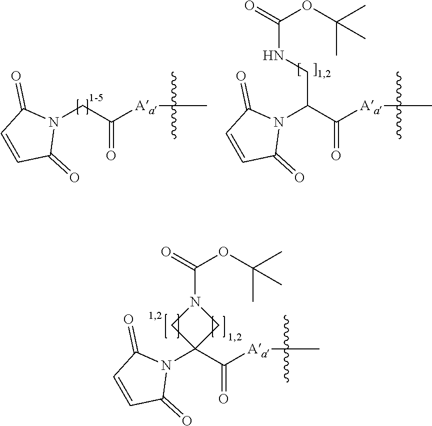

wherein A′ and subscript a′ are as previously described for Formula IA drug linker compounds in which subscript q is 1.

In preferred embodiments of L_{SS}′-containing Drug Linker compounds, the L_{SS}′ moiety contains a heterocyclo cyclic Basic Unit. Exemplary Drug Linker compounds having those primary linkers in which the Peptide Cleavable Unit is a tripeptide is represented by the structure of Formula IB:

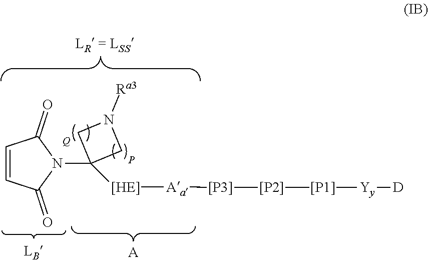

or a salt thereof, wherein HE is an optional Hydrolysis Enhancing Unit; A′ is an subunit, when present, of a first Stretcher Unit (A); subscript a′ is 0 or 1, indicating the absence or presence of A′, respectively; subscript P is 1 or 2; subscript Q ranges from 1 to 6, preferably subscript Q is 1 or 2, more preferably subscript Q has the same value as subscript P; and wherein R^{a3} is —H, optionally substituted C₁-C₆ alkyl, optionally substituted —C₁-C₄ alkylene-(C₆-C₁₀ aryl), or —R^{PEG1}—O—(CH₂CH₂O)₁₋₃₆—R^{PEG2}, wherein R^{PEG1} is C₁-C₄ alkylene, R^{PEG2} is —H or C₁-C₄ alkylene, wherein the basic nitrogen bonded to R^{a3} is optionally protonated in a salt form, preferably in a pharmaceutically acceptable salt form, or Ra is a nitrogen protecting group such as a suitable acid-labile protecting group; P1, P2 and P3 are as previously defined for any one of the embodiments of Peptide Cleavable Units for a drug linker moiety of a Ligand Drug Conjugate compound; and the remaining variable groups are as described for a Drug Linker compound of Formula IA.

In other preferred embodiments of L_{SS}′-containing Drug Linker compounds of Formula IA the L_{SS}′ moiety contains an acyclic cyclic Basic Unit. Exemplary Drug Linker compounds having that primary linker in which the Peptide Cleavable Unit is a dipeptide are represented by the structures of Formula IE:

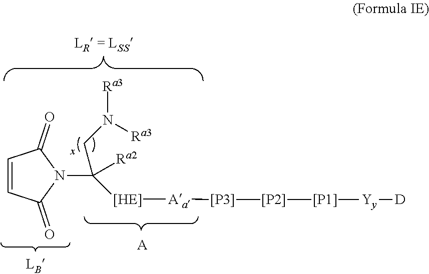

or a salt thereof, wherein HE is an optional Hydrolysis Enhancing Unit; A′ is an subunit, when present, of a first Stretcher Unit (A); subscript a′ is 0 or 1, indicating the absence or presence of A′, respectively; subscript x is 1 or 2; R^{a2} is hydrogen or —CH₃ or —CH₂CH₃; R^{a3}, at each instance, is independently hydrogen, —CH₃ or —CH₂CH₃, or both R^{a3} together with the nitrogen to which they are attached define an azetidinyl, pyrrolidinyl or piperidinyl heterocyclyl, in which a basic primary, secondary or tertiary amine so defined is optionally protonated in a salt form, preferably a pharmaceutically acceptable salt form; P1, P2 and P3 are as previously defined for any one of the embodiments of Peptide Cleavable Units and the remaining variable groups are as described for a Drug Linker compound of Formula IA.

In other preferred embodiments, a primary linker does not have a Basic Unit. Exemplary Drug Linker compounds having that primary linker in which the Peptide Cleavable Unit is a tripeptide are represented by the structure of Formula IH:

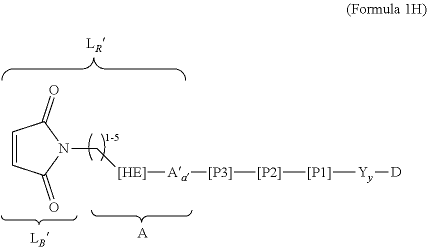

or a salt thereof, wherein HE is an optional Hydrolysis Enhancing Unit; A′ is an subunit, when present, of a first Stretcher Unit (A); subscript a′ is 0 or 1, indicating the absence or presence of A′; P1, P2 and P3 are as previously defined for any one of the embodiments of Peptide Cleavable Units of a drug linker moiety of a Ligand Drug Conjugate compound and the remaining variable groups are as described for any one of the embodiments of a Drug Linker compound of Formula IA.

In more preferred embodiments in which there is a heterocyclo cyclic Basic Unit in the Linker Unit, a Drug Linker compound is represented by the structure of:

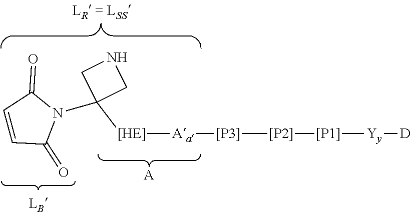

optionally in a salt form, in particular in pharmaceutical acceptable salt form, and in more preferred embodiments in which there is an acyclic Basic Unit in the Linker Unit, a Drug Linker compound is represented by the structure of:

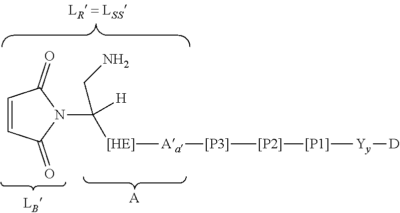

optionally in salt form, wherein the variable groups of the L_{SS}′-containing Drug Linker compound is as previously described for a Drug Linker compound having a acyclic or heterocyclo cyclic Basic Unit.

In any one of the preceding drug linker moieties, HE is preferably present as —C(═O) and/or subscript y is 1 or 2, indicating the presence of one or two self-immolative Spacer Units, respectively.

In particularly preferred embodiments the —[P3]-[P2]-[P1]- tripetide in any one of the above Drug Linker compounds is D-Leu-Leu-Cit, D-Leu-Leu-Lys, D-Leu-Leu-Met(O), D-Leu-Ala-Glu or Pro-Ala(Nap)-Lys, wherein Met(O) is methionine in which its sulfur atom is oxidized to a sulfoxide, Cit is citrulline, and Ala(Nap) is alanine in which its methyl side chain is substituted by napthth-1-yl.

In especially preferred embodiments in which there is a heterocyclo cyclic Basic Unit in the Linker Unit, the Drug Linker compound is represented by the structure of:

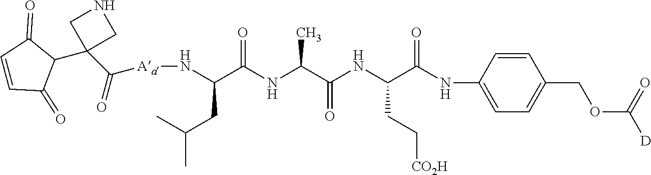

or salt thereof, wherein subscript a′ is 0 or 1, indicating the absence or presence of A′, respectively, wherein A′ is an amine-containing acid residue of formula 3a, 4a or 5a as described herein for a second optional Stretcher Unit or a subunit of a first optional Stretcher Unit, or A′ is an α-amino acid or β-amino acid residue; and D is a cytotoxic drug having a secondary amino group as the site of attachment to the Linker Unit of the drug linker moiety.

In other especially preferred embodiments in which there is an acyclic Basic Unit in the Linker Unit, the Drug linker compound is represented by the structure of:

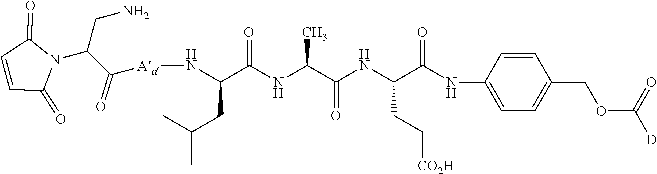

or salt thereof, wherein the variable groups are as previously described for Drug Linker compounds having a cyclic Basic Unit.

In other especially preferred embodiments in which there is no Basic Unit, the Drug Linker compound is represented by the structure of:

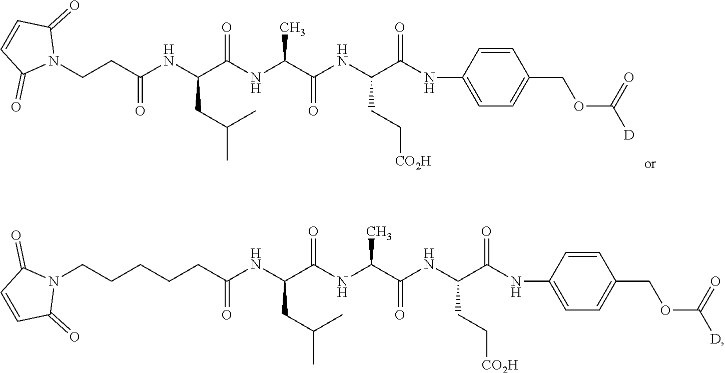

or salt thereof, wherein the variable groups are as previously described for Drug Linker compounds having a cyclic Basic Unit.

In some embodiments, the Drug Linker compound is represented by:

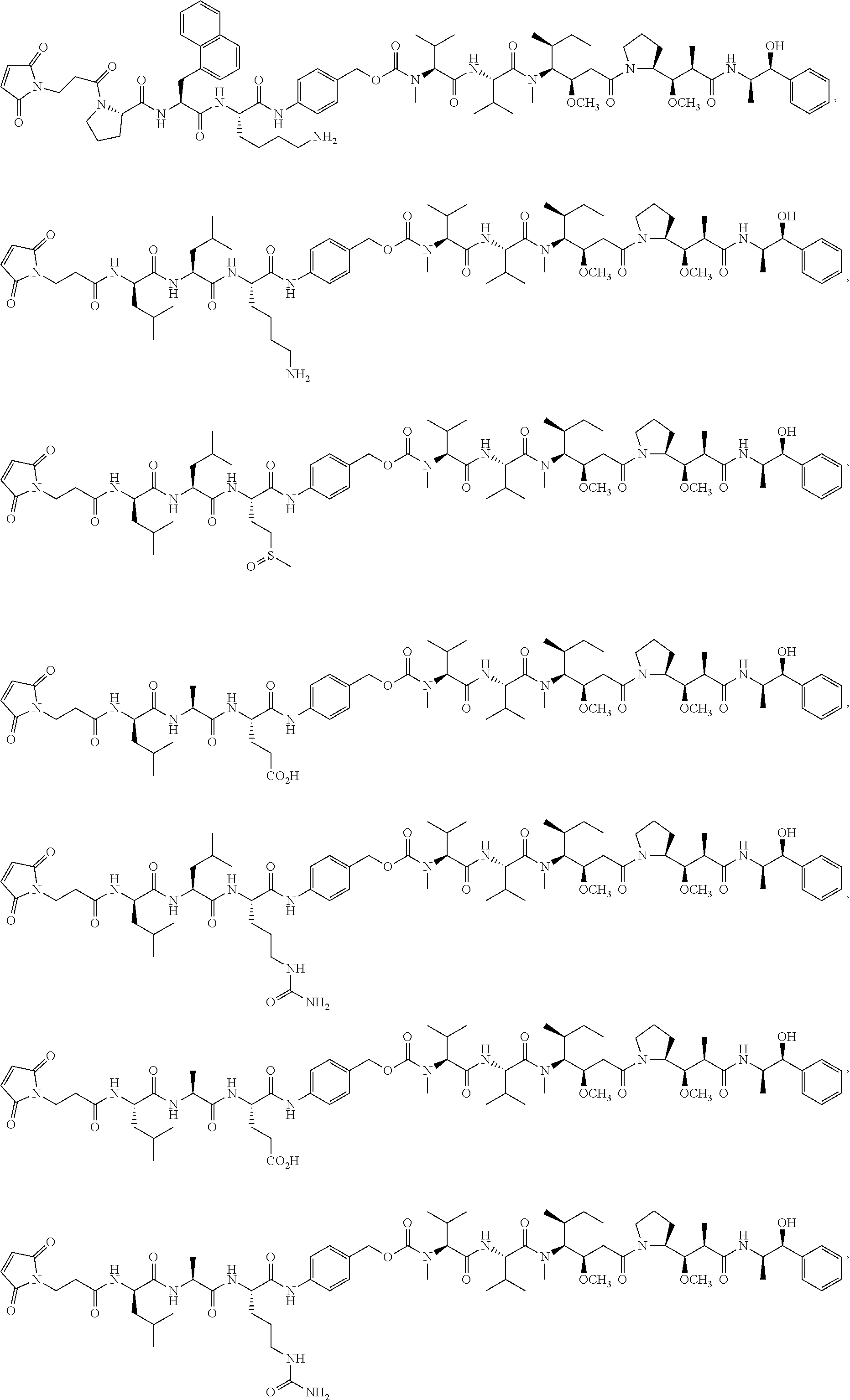

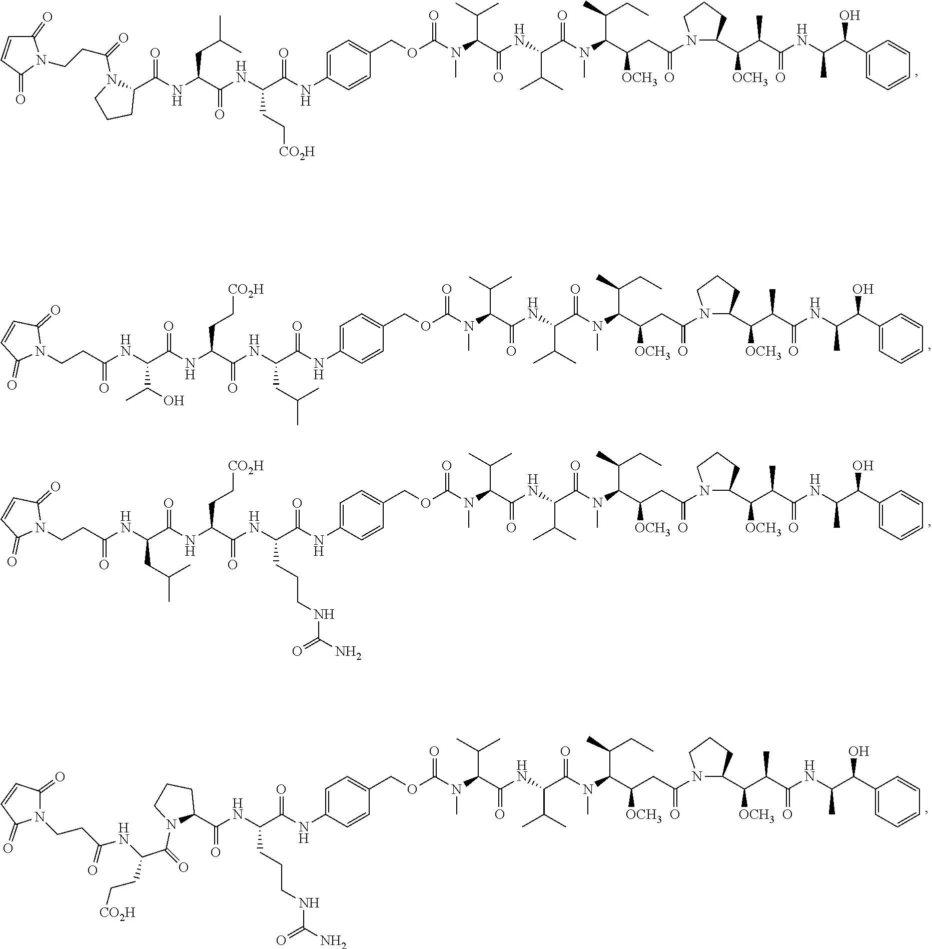

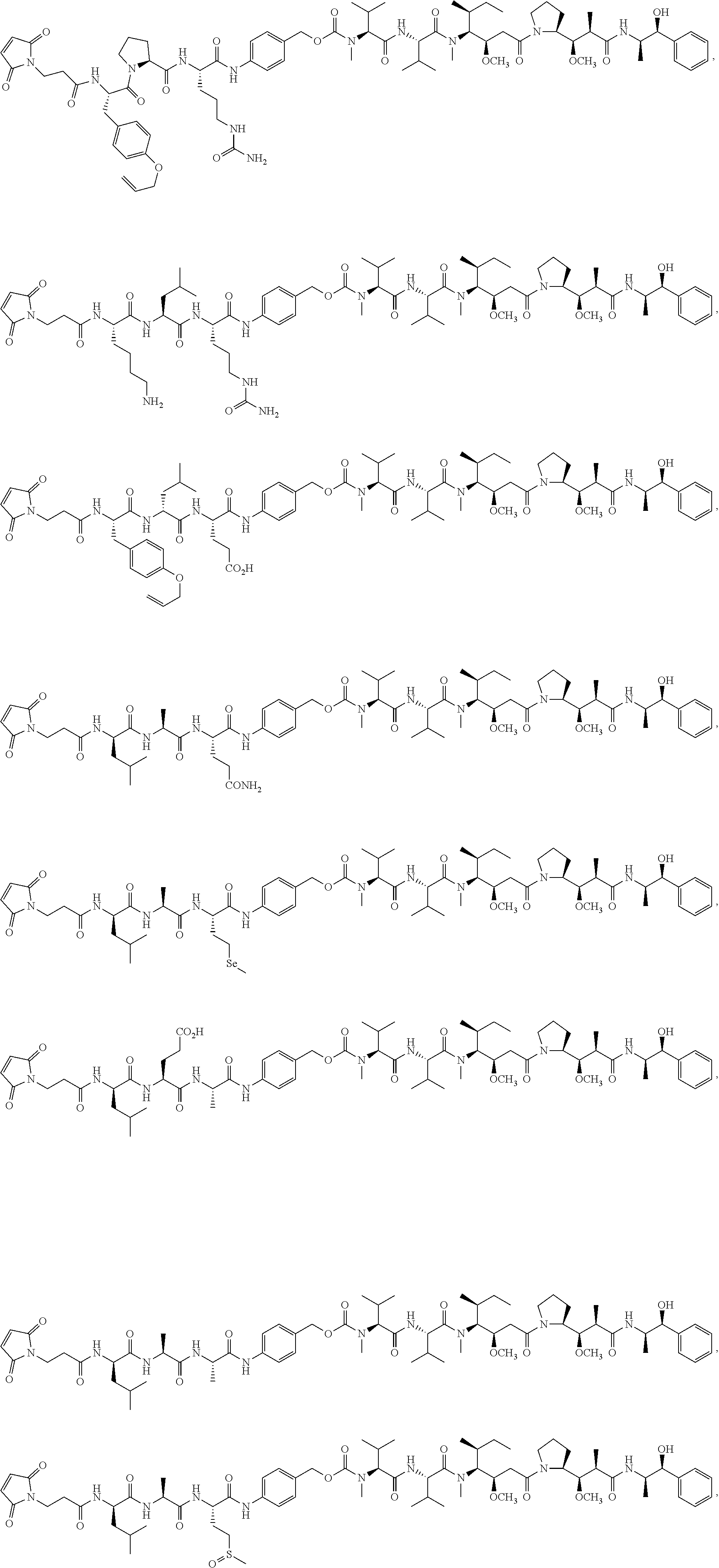

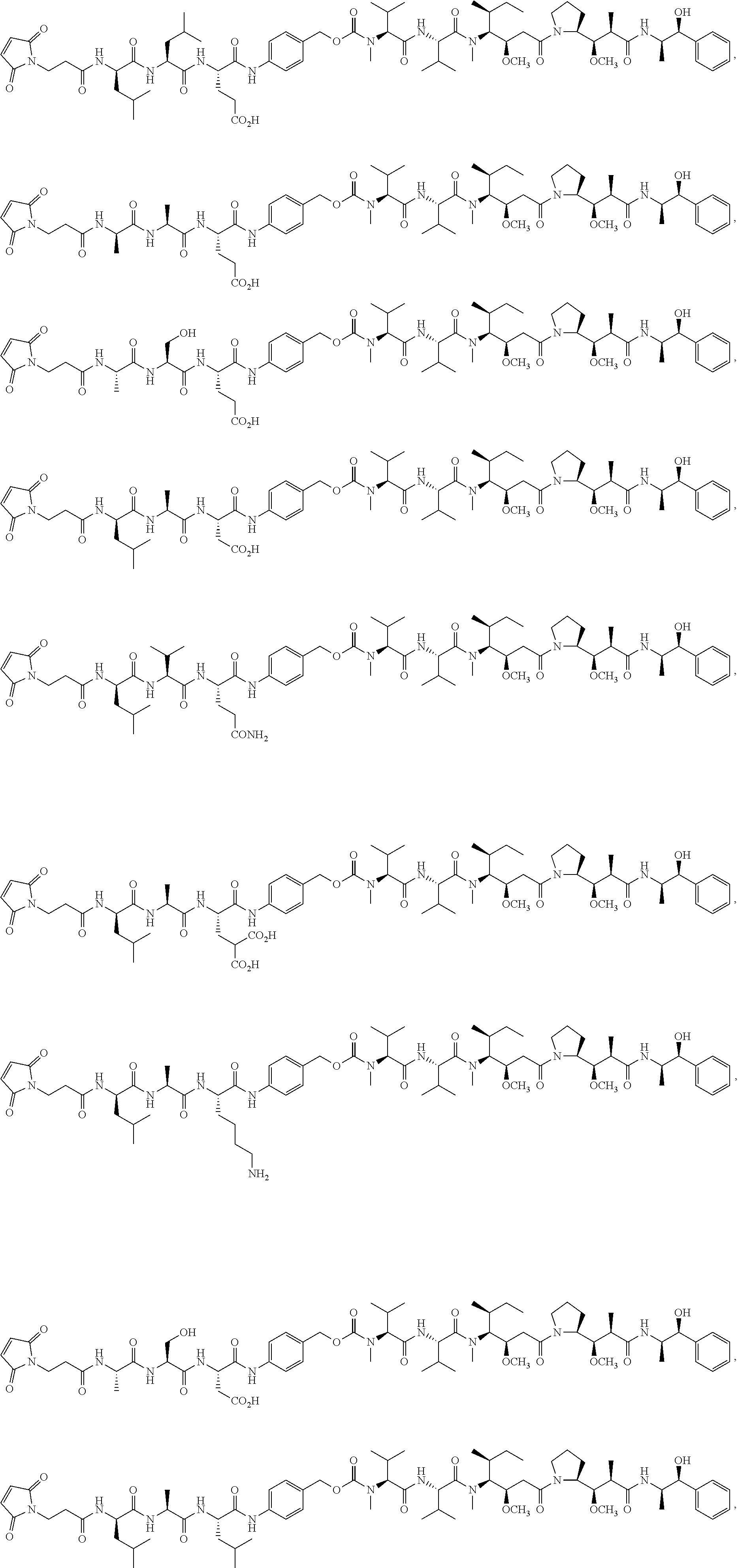

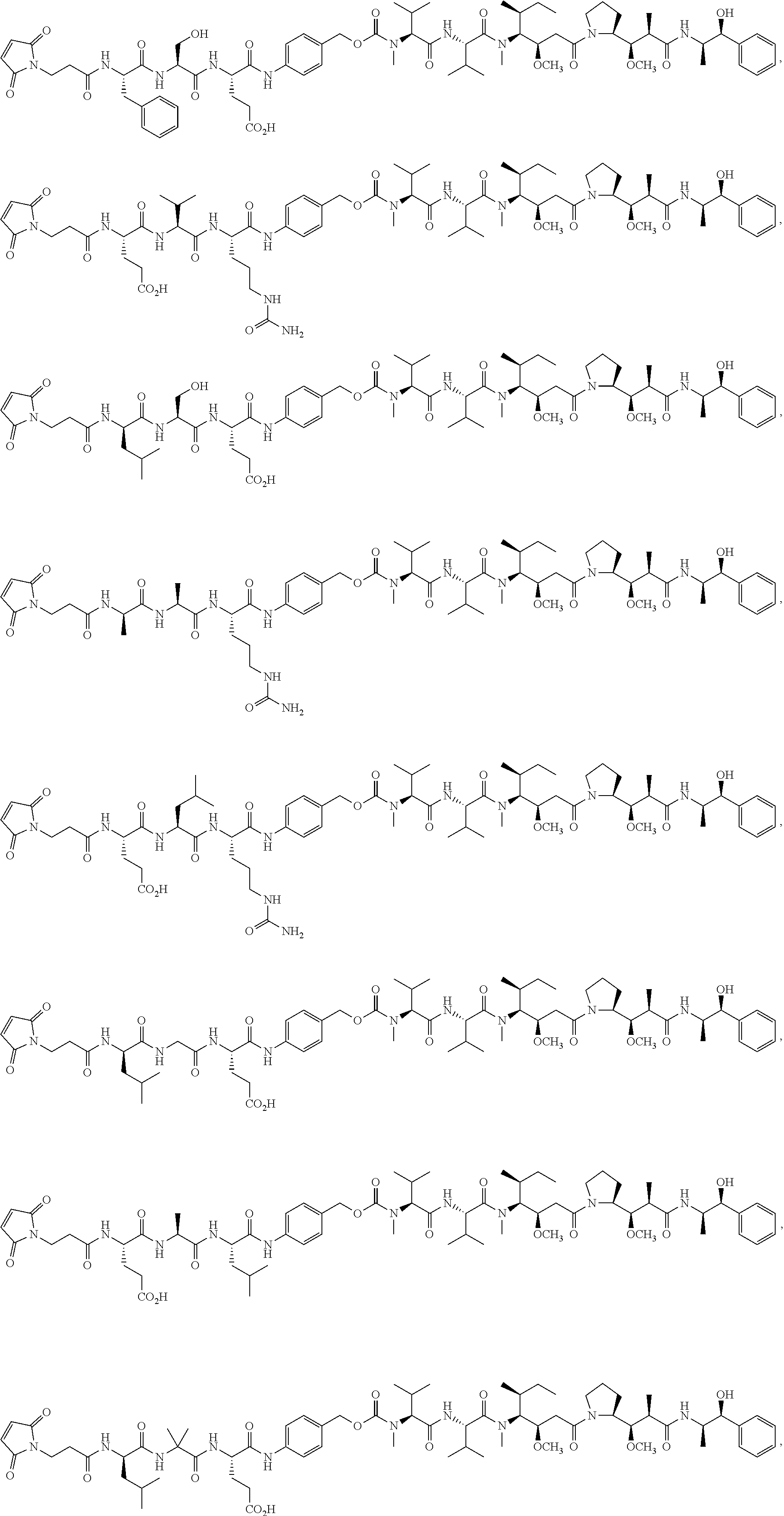

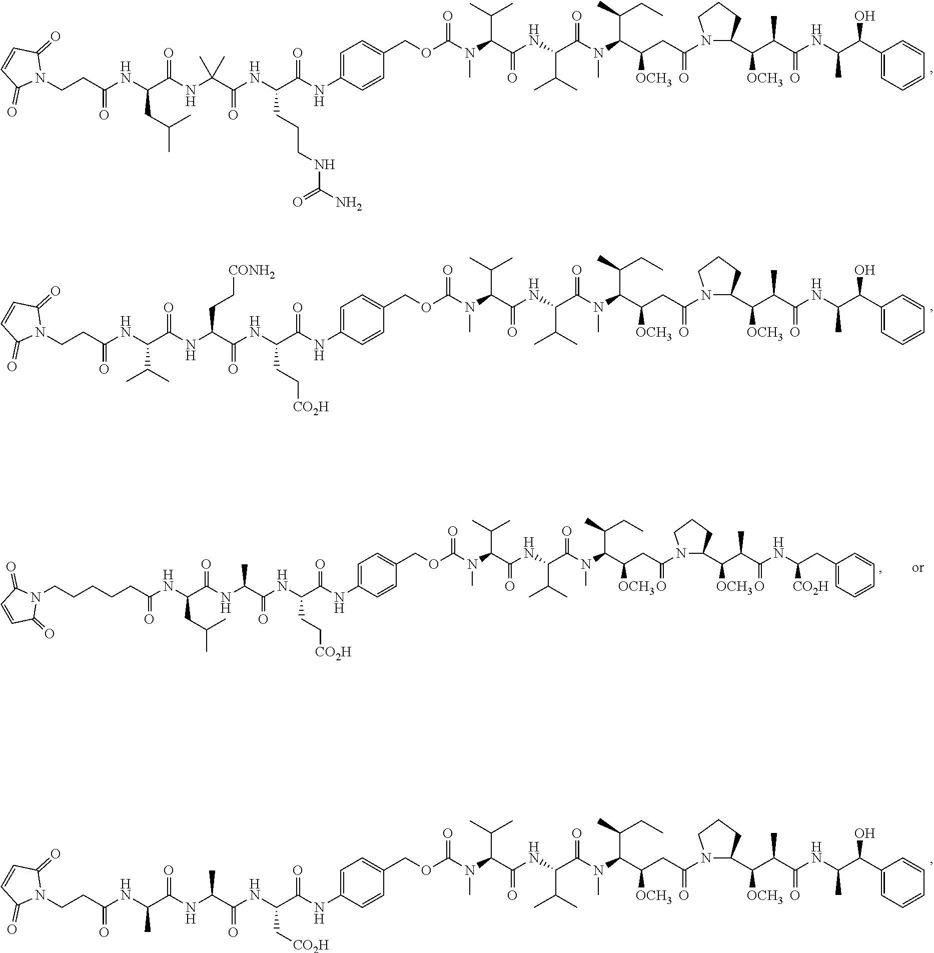

or a salt thereof.

In some embodiments, provided is a Drug Linker Precursor compound represented by the structure:

PG-W—Y_{y}-D
or a salt thereof, wherein W, Y, subscript y, and D retain their previous meanings, and PG is an amine protecting group or hydrogen. In some embodiments, the amine protecting group is Fmoc.

In some embodiments, Drug Linker Precursor compound represented by the structure:

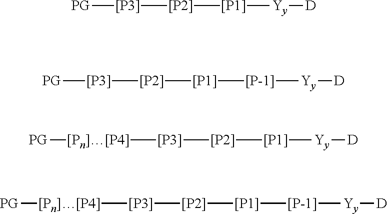

or a salt thereof, wherein P-1, P1, P2, P3 . . . Pₙ, Y, subscript y, and D retain their previous meanings, and PG is an amine protecting group or hydrogen.

In some embodiments, Drug Linker Precursor compound represented by the structure:

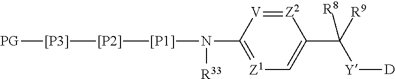

or a salt thereof, wherein P1, P2, P3, R⁸, R⁹, R³³, V, Y′, Z¹, Z², and D retain their previous meanings, and PG is an amine protecting group or hydrogen.

In any of the Drug Linker compounds described herein, the L_{B}′-Aₐ-B_{b}-A′_{a′}-portion can be replaced by PG to form a Drug Linker Precursor compound represented by the structure:

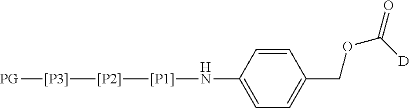

or a salt thereof, wherein P1, P2, P3, and D retain their previous meanings, and PG is an amine protecting group or hydrogen.

It is understood that a Drug Linker Precursor can be further modified with a stretcher unit for attachment to a ligand such as an antibody. In some embodiments, the Drug Linker Precursor may be further reacted with a stretcher unit suitable for attachment to a cysteine residue of an antibody. Suitable stretcher units for attachment to a cysteine residue of an antibody are described herein, including stretcher units comprising an maleimide moiety. In some embodiments, the Drug Linker Precursor may be further reacted with a stretcher unit suitable for attachment to a lysine residue of an antibody. Suitable stretcher units for attachment to a lysine residue of an antibody are described herein, including stretcher units comprising an NHS ester moiety. In some embodiments, the Drug Linker Precursor is an intermediate in the synthesis of Drug Linker compounds.

In any of the embodiments described herein for W, P-1, P1, P2, P3 . . . Pₙ, Y, subscript y, R⁸, R⁹, R³³, V, Y′, Z¹, Z², and D with respect to, for example, Ligand Drug Conjugate (LDC) compounds, Drug Linker compounds, drug linker moieties, Peptide Cleavable Units, Spacer Units, and Drug Units, the embodiments are also applicable for Drug Linker Precursor compounds described herein.

In some embodiments, the Drug Linker Precursor compound is represented by:

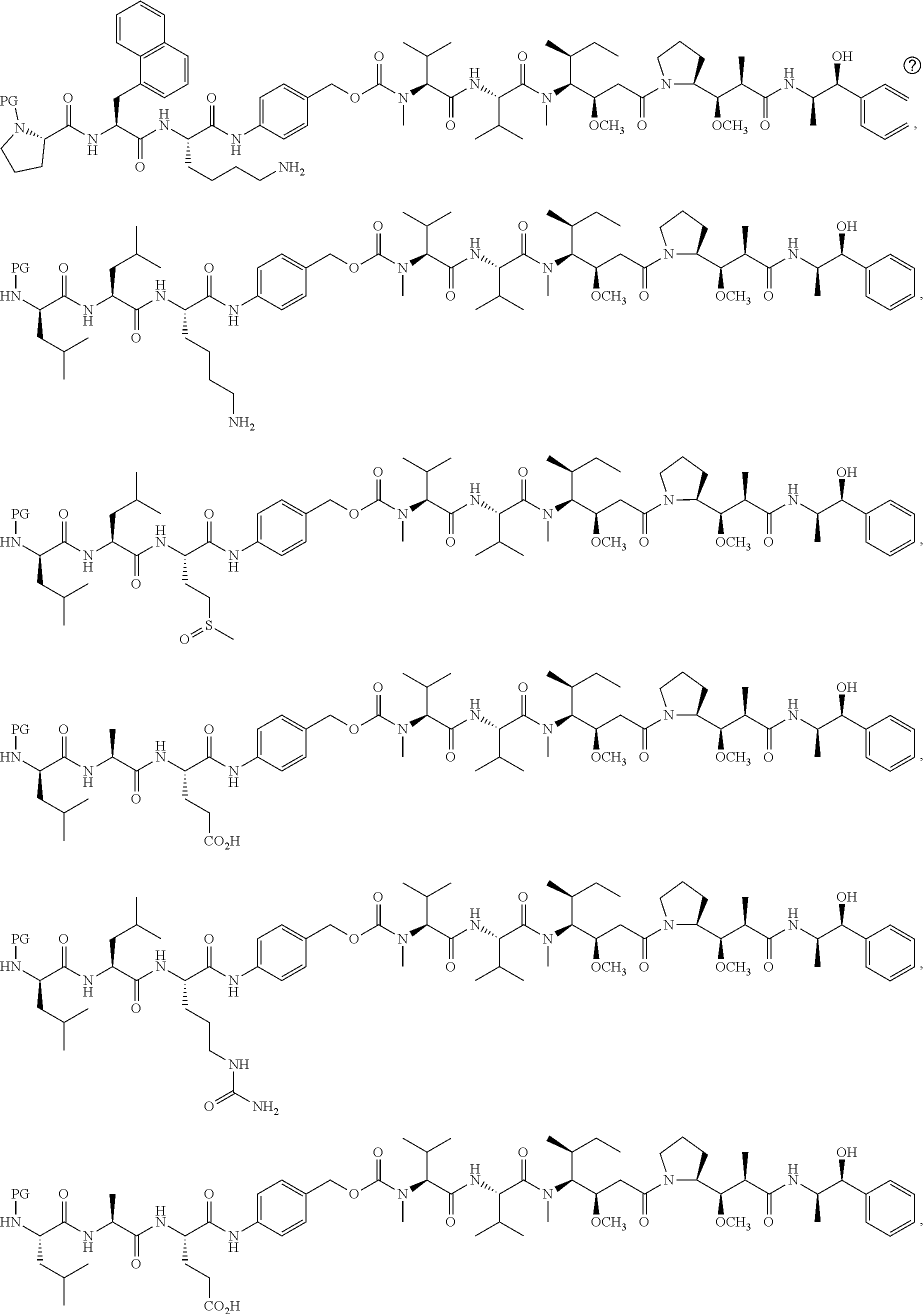

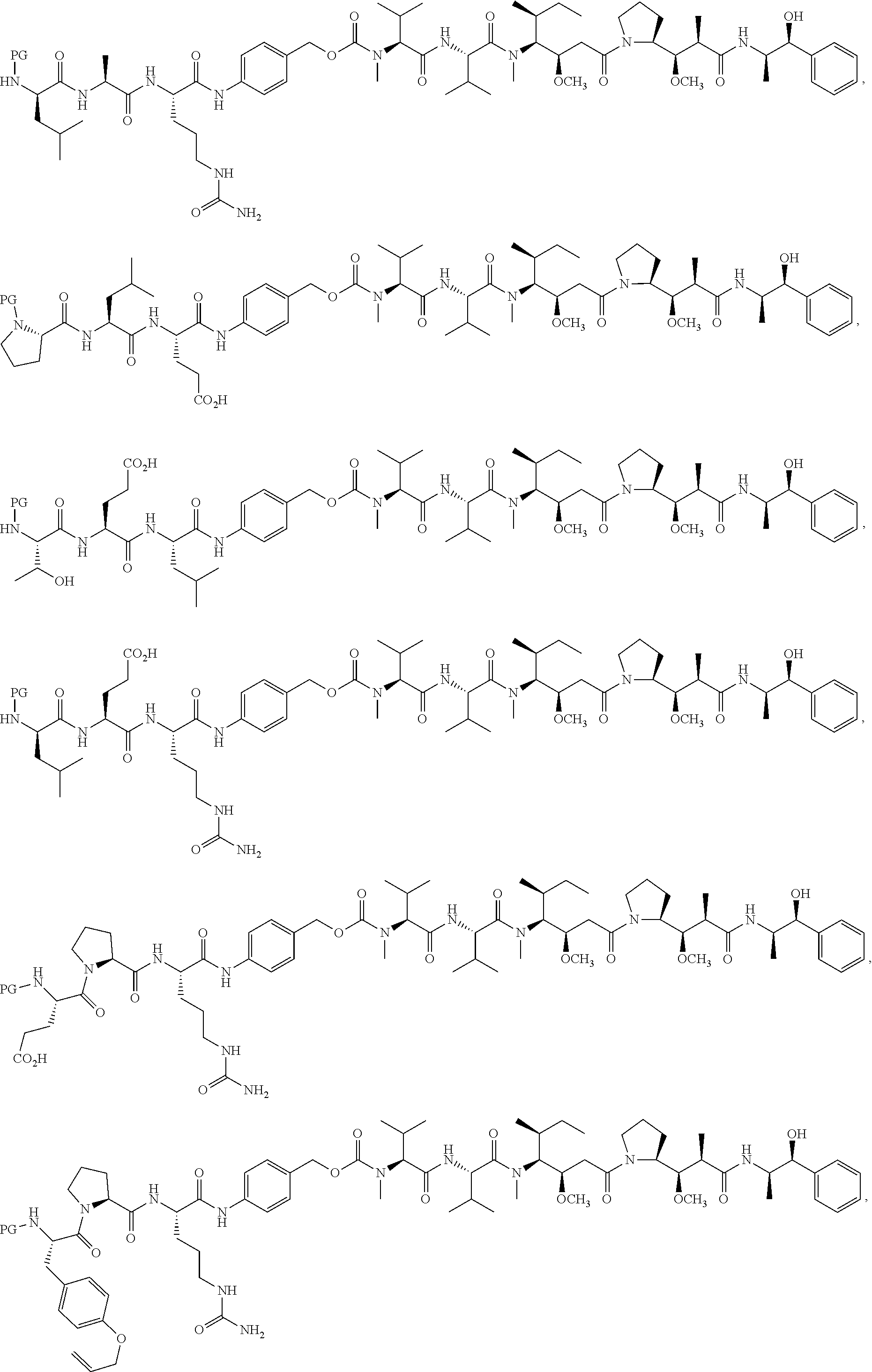

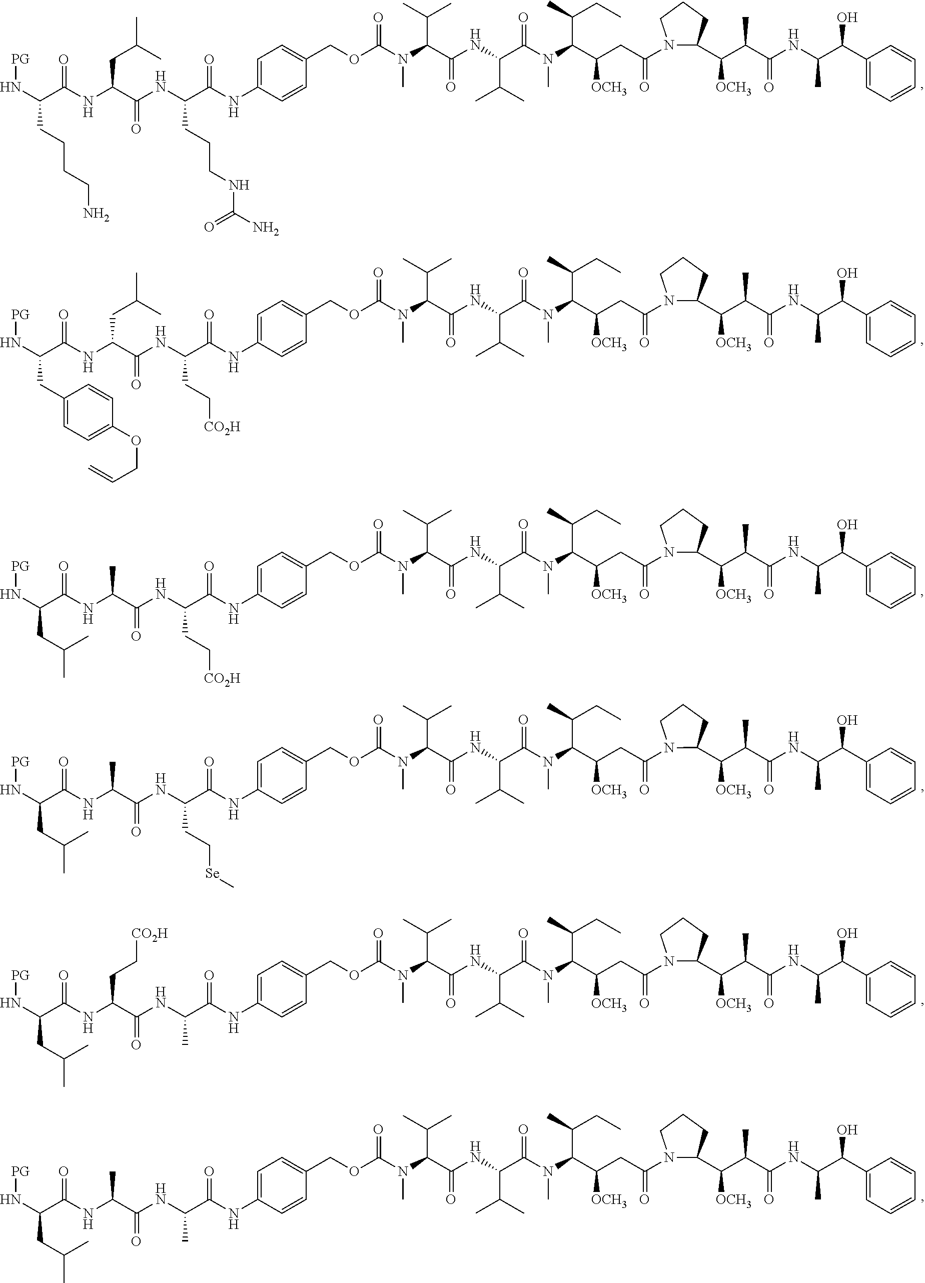

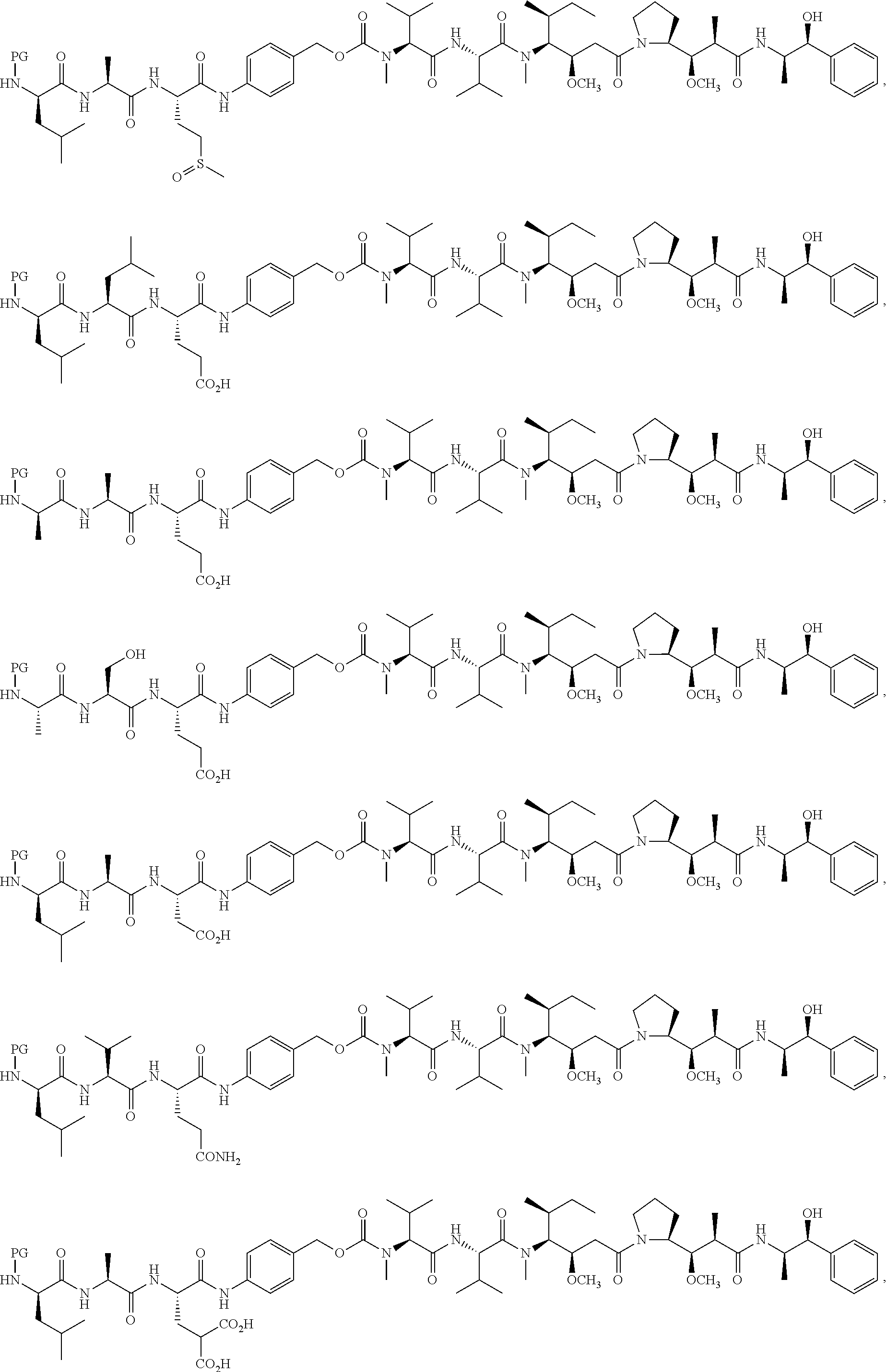

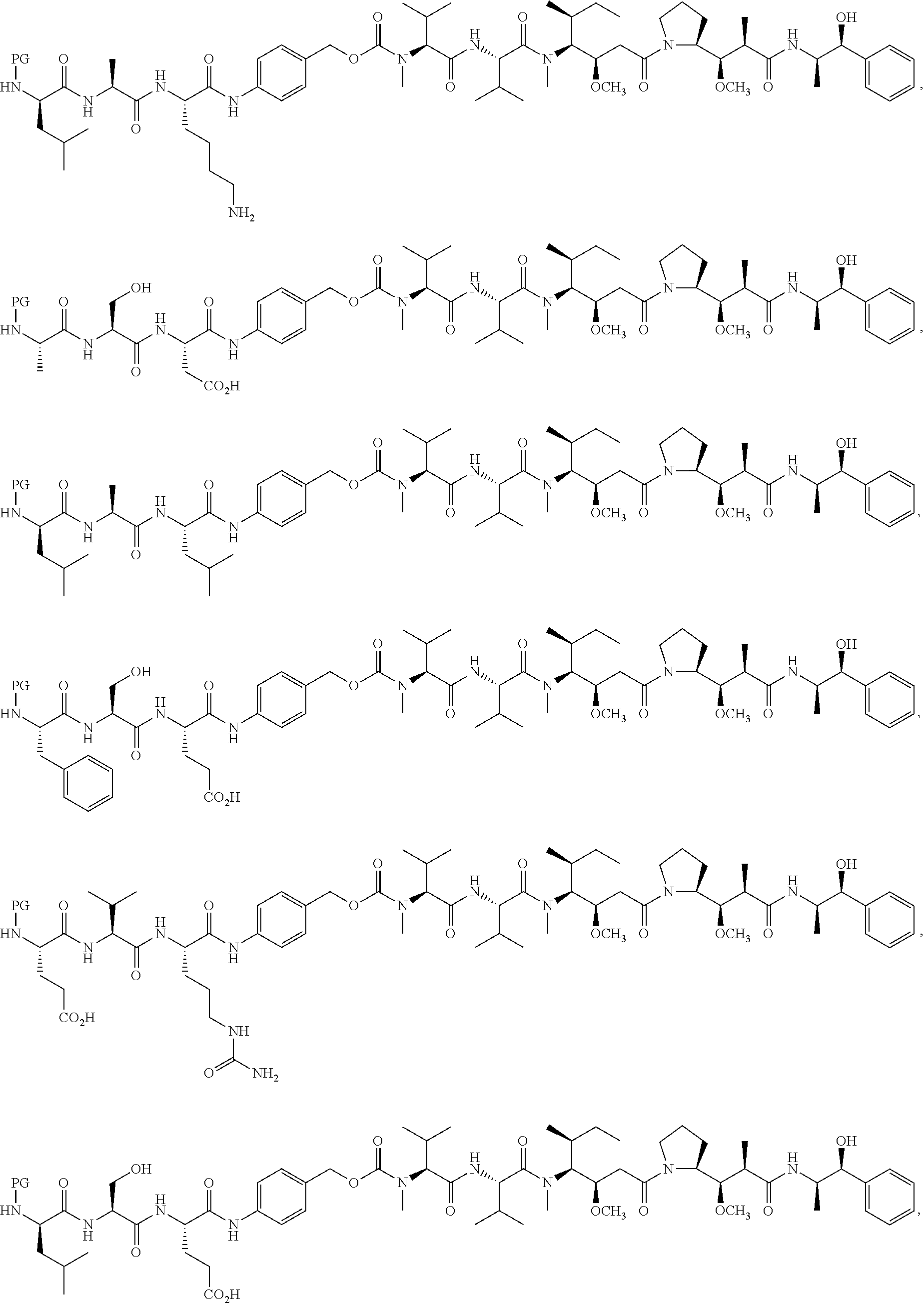

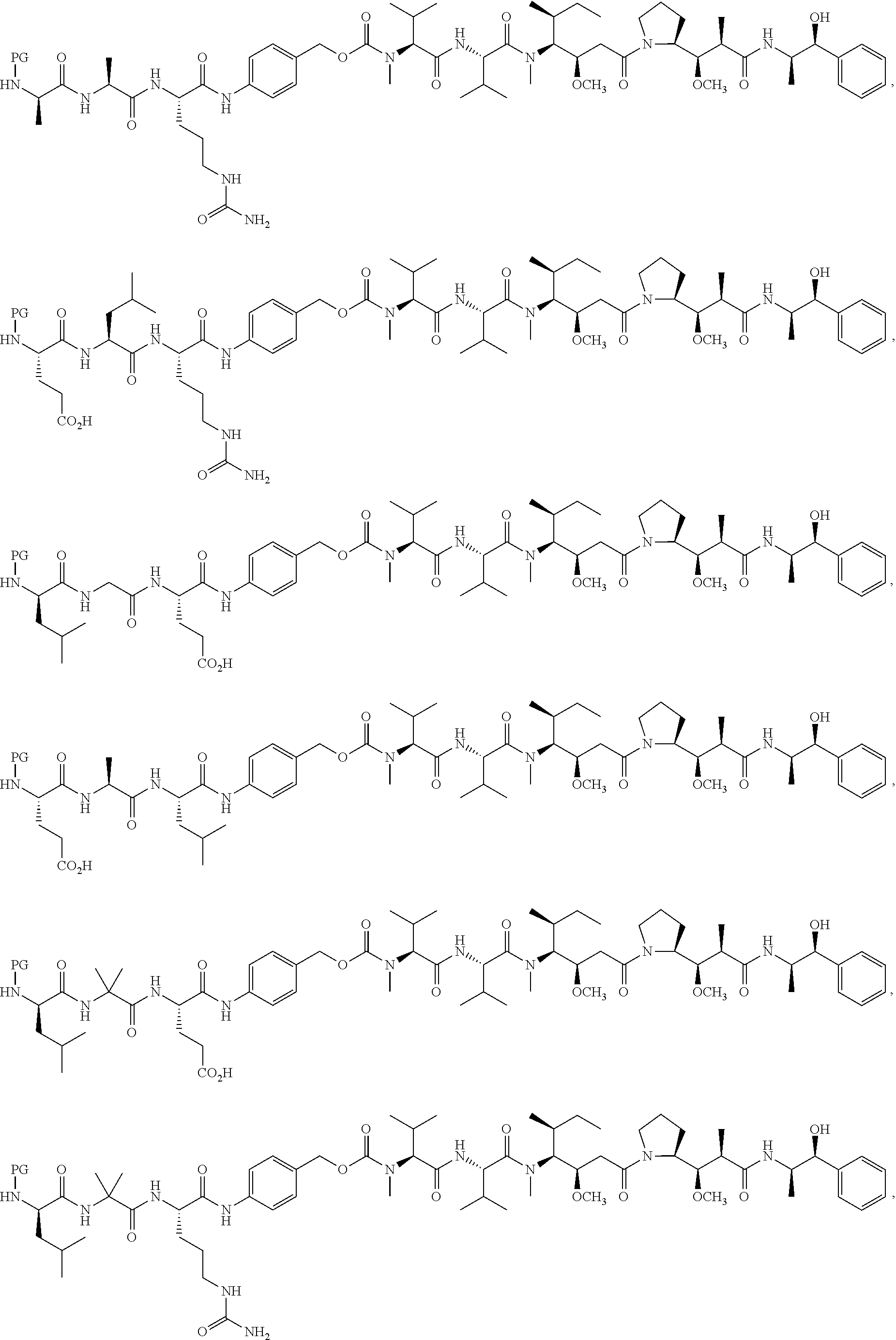

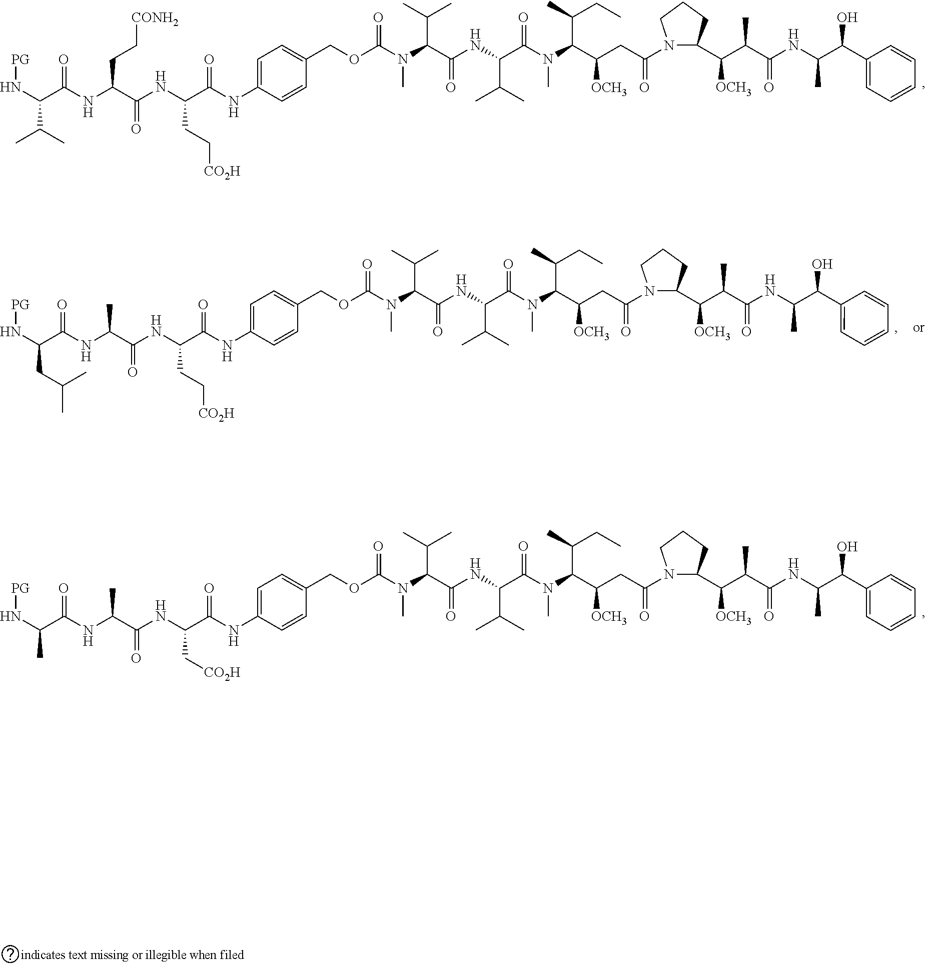

or a salt thereof, wherein PG is an amine protecting group (e.g., Fmoc) or hydrogen.

### 2.4 Linker Compounds

A Linker compound is represented by the structure of Formula IA-L:

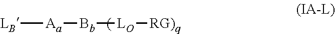

or a salt thereof, wherein L_{B}′, A, subscript a, B, subscript b, L_{O}, and subscript q retain their previous meanings, and RG is a reactive group. In some embodiments, the reactive group is 4-nitrophenoxy or perfluorophenoxy. In some embodiments, the reactive group is 4-nitrophenoxy.

In some embodiments, the Linker compound is represented by the structure of Formula IA-L-1:

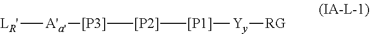

or a salt thereof, wherein L_{R}′, A′, subscript a′, P1, P2, P3, Y, and subscript y retain their previous meanings, and RG is a reactive group.

In some embodiments, the Linker compound is represented by the structure of Formula IA-L-2:

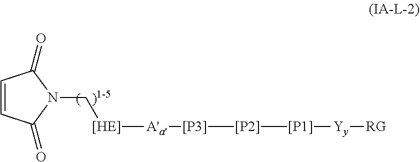

or a salt thereof, wherein HE, A′, subscript a′, P1, P2, P3, Y, and subscript y retain their previous meanings, and RG is a reactive group.

In some embodiments, the Linker compound is represented by the structures of Formula IA-L-3 or Formula IA-L-4:

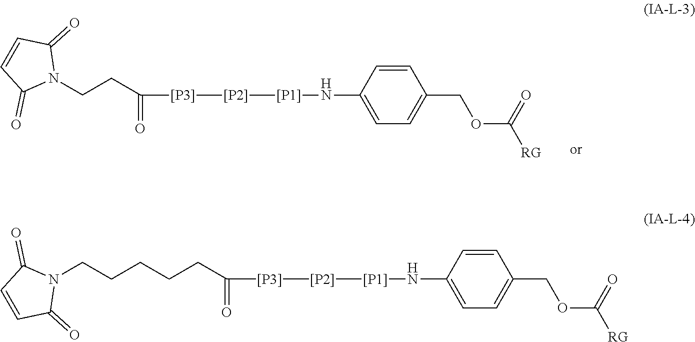

or a salt thereof, wherein P1, P2, and P3 retain their previous meanings, and RG is a reactive group. In some embodiments, RG is perfluorophenoxy. In some embodiments, RG is 4-nitrophenoxy.

In any of the embodiments described herein for L_{B}′, A, subscript a, B, subscript b, L_{O}, subscript q, L_{R}′, A′, subscript a′, P1, P2, P3, Y, subscript y, and HE with respect to Ligand Drug Conjugate (LDC) compounds, primary linkers, secondary linkers, Drug Linker compounds, drug linker moieties, Peptide Cleavable Units, Stretcher Units, and Spacer Units, the embodiments are also applicable for Linker compounds described herein, such as compounds of Formula IA-L, Formula IA-L-1, Formula IA-L-2, Formula IA-L-3, or Formula IA-L-4.

In any of the Drug Linker compounds described herein, the Drug Unit (D) can be replaced by a suitable reactive group (i.e., a group suitable for attachment to the Drug Unit (D)) to form a Linker compound, for example a structure represented by Formula IA-L, Formula IA-L-1, Formula IA-L-2, Formula IA-L-3, or Formula IA-L-4. The reactive group is a group suitable for reacting the linker compound with an auristatin drug compound as described herein (such as MMAE or MMAF) to form a Drug Linker compound.

In some embodiments, the Linker compound is represented by:

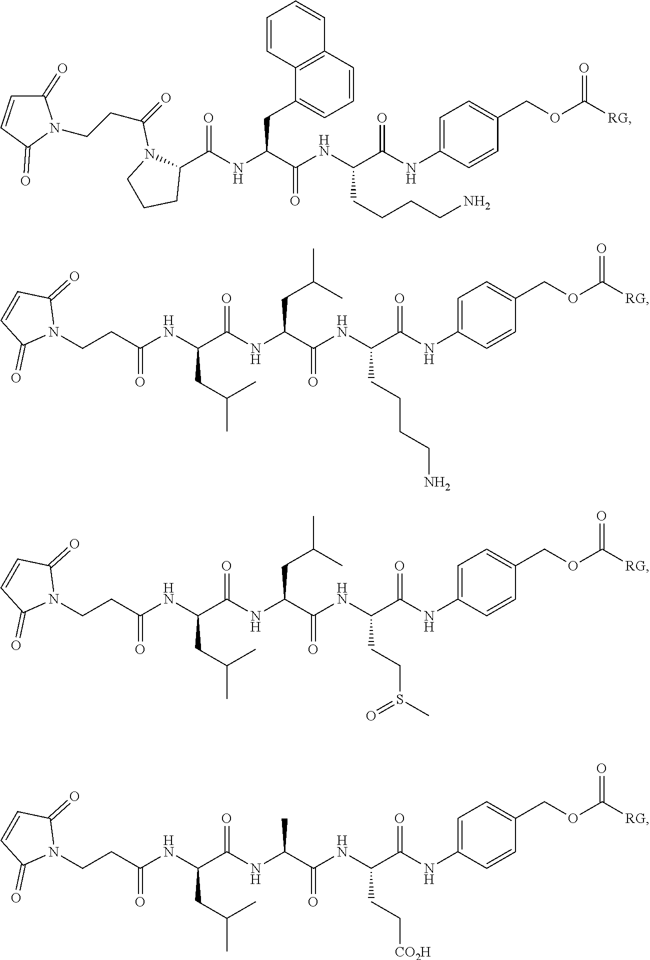

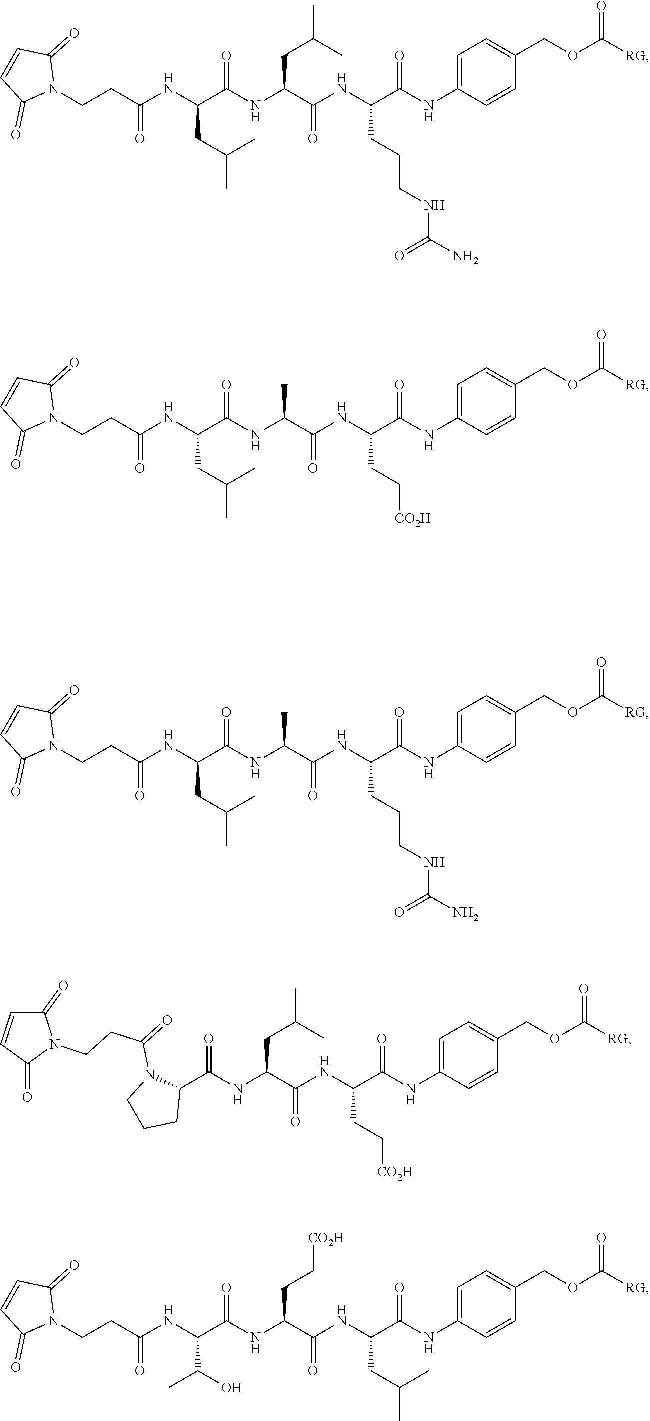

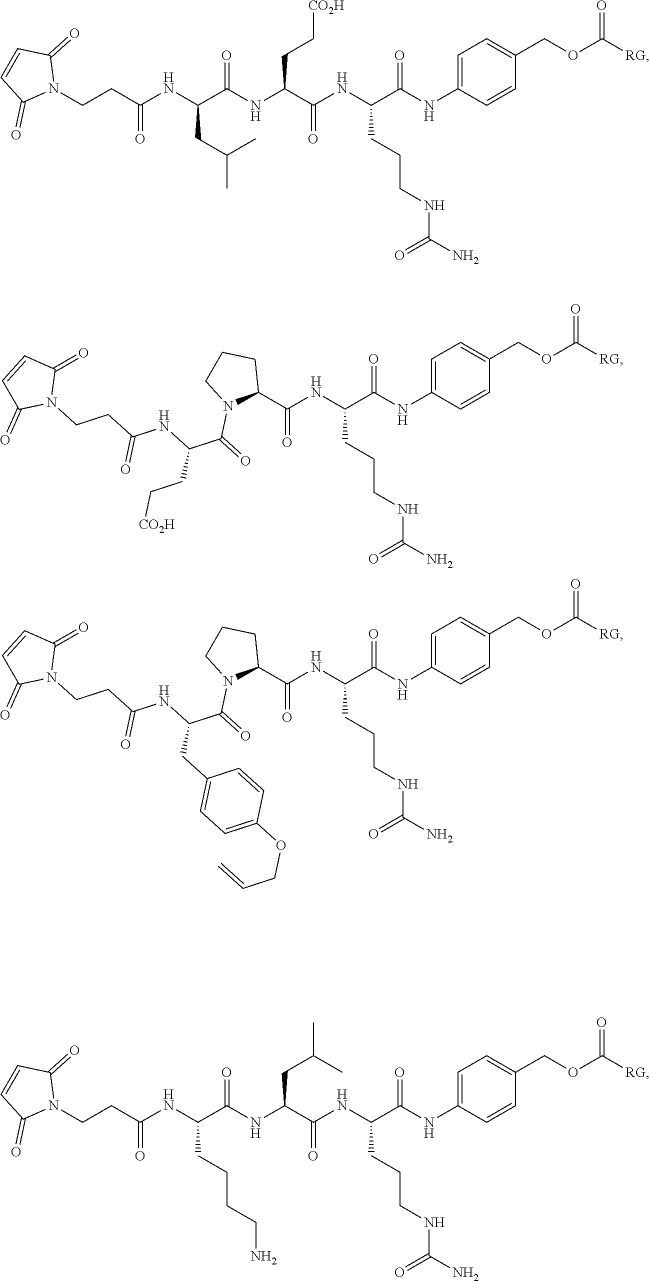

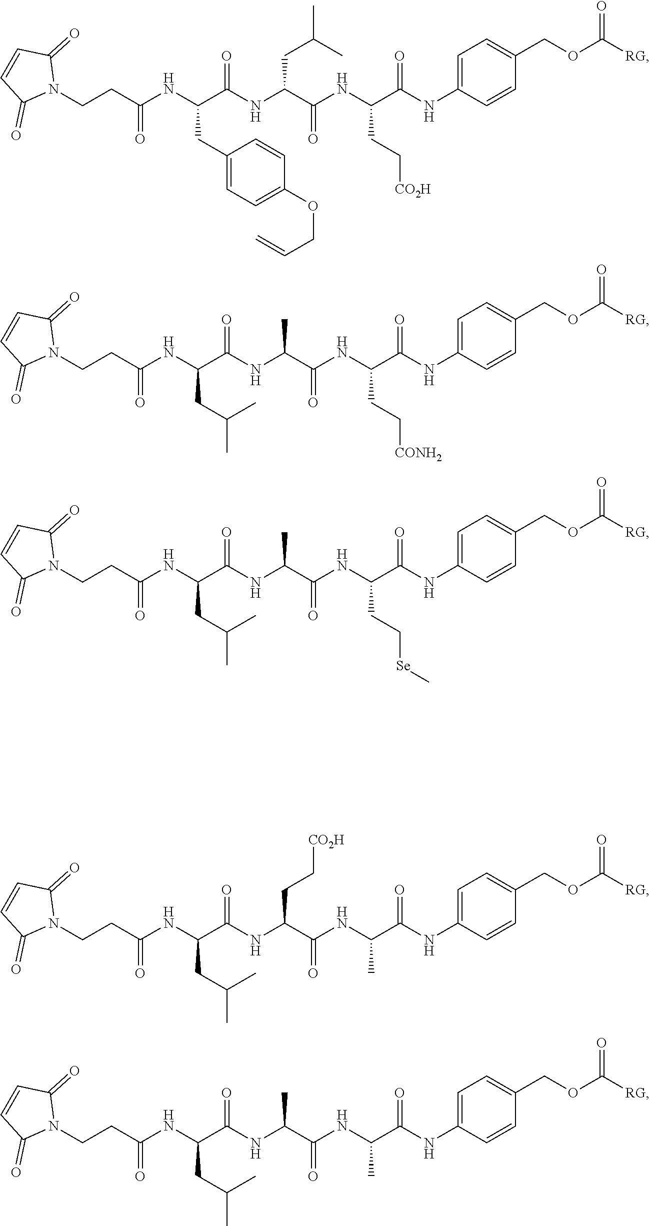

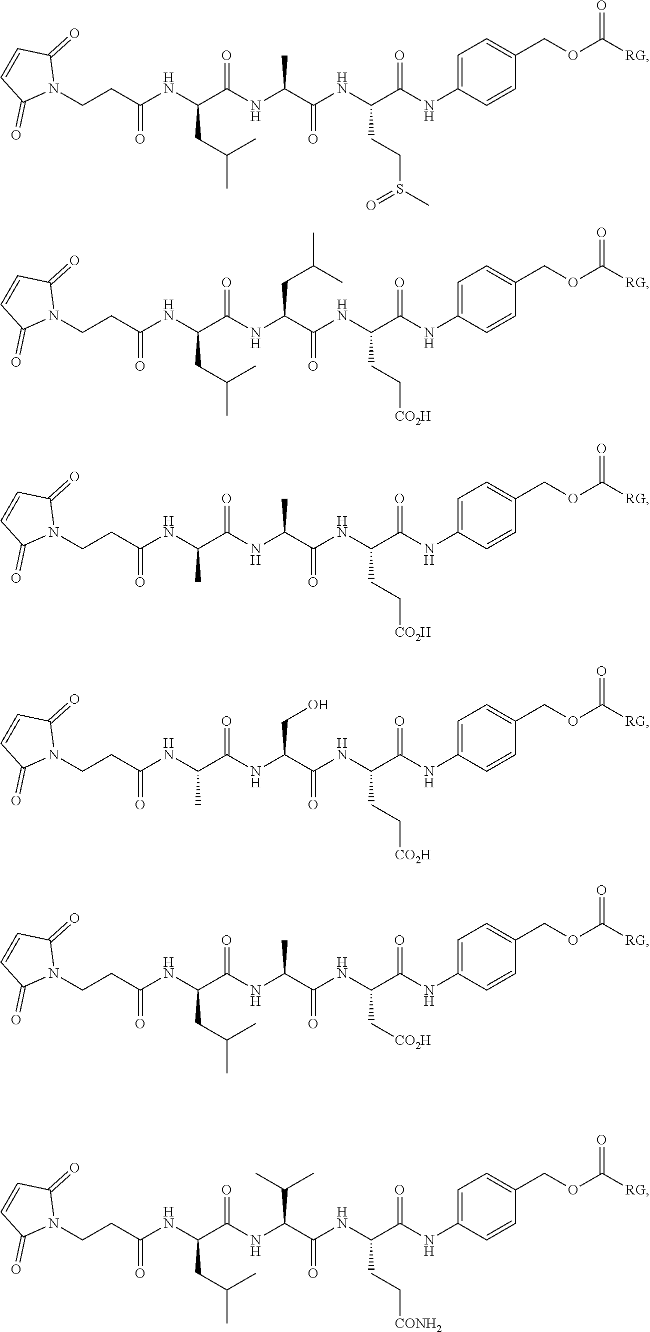

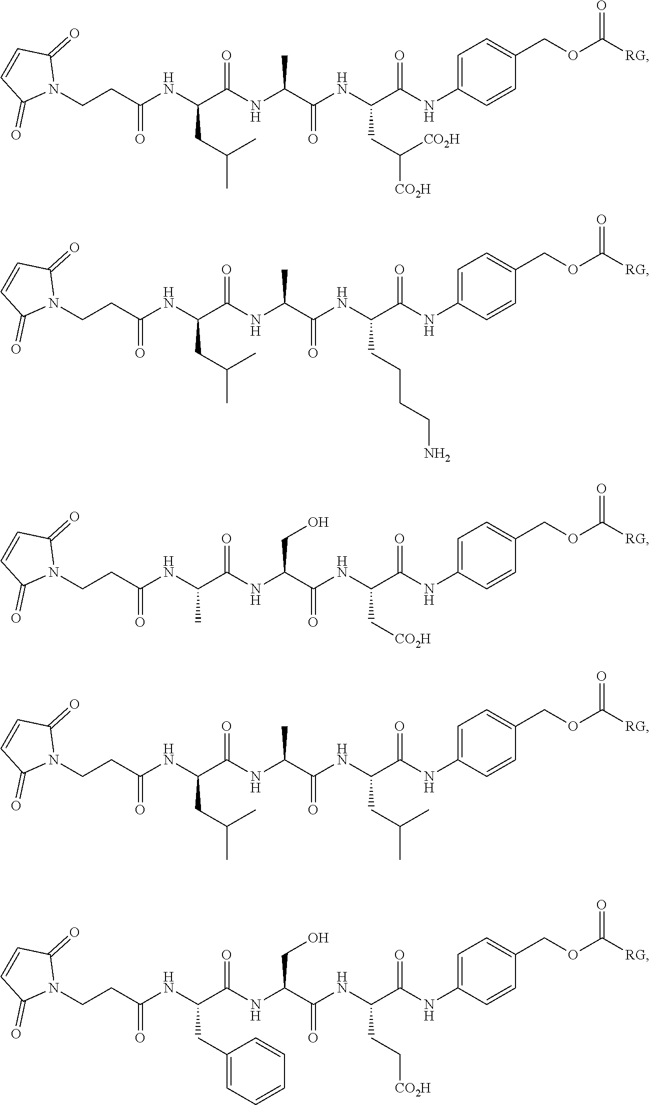

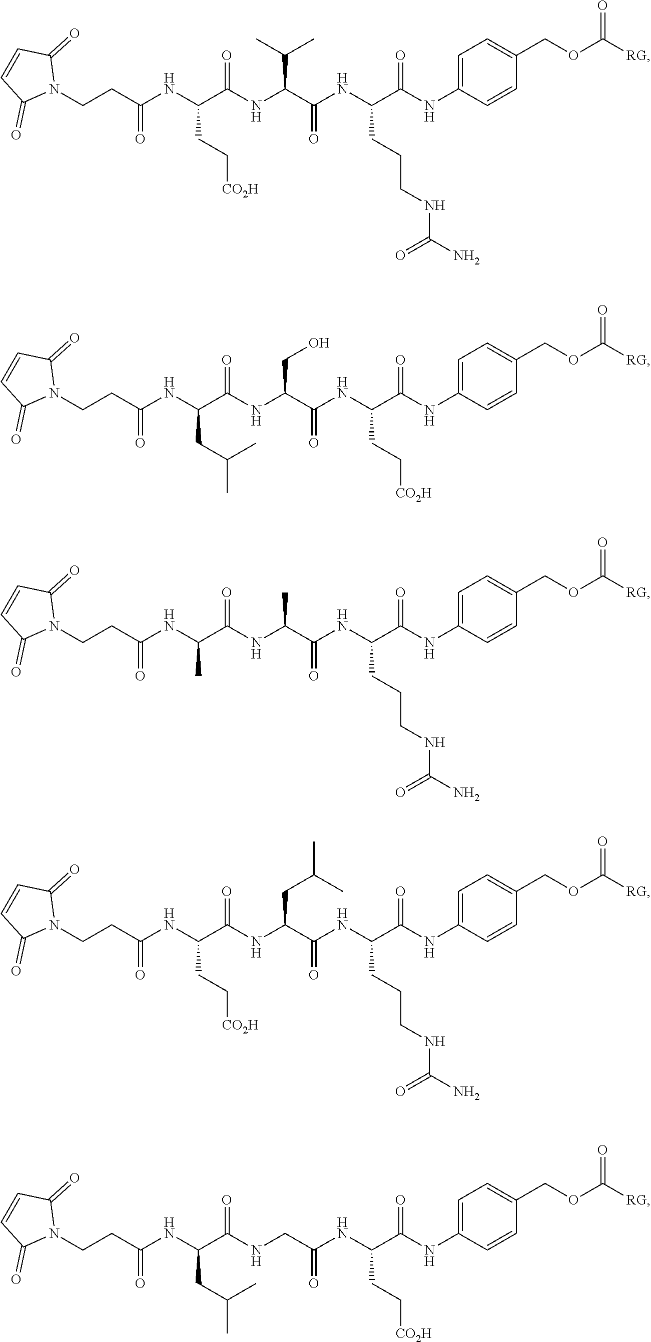

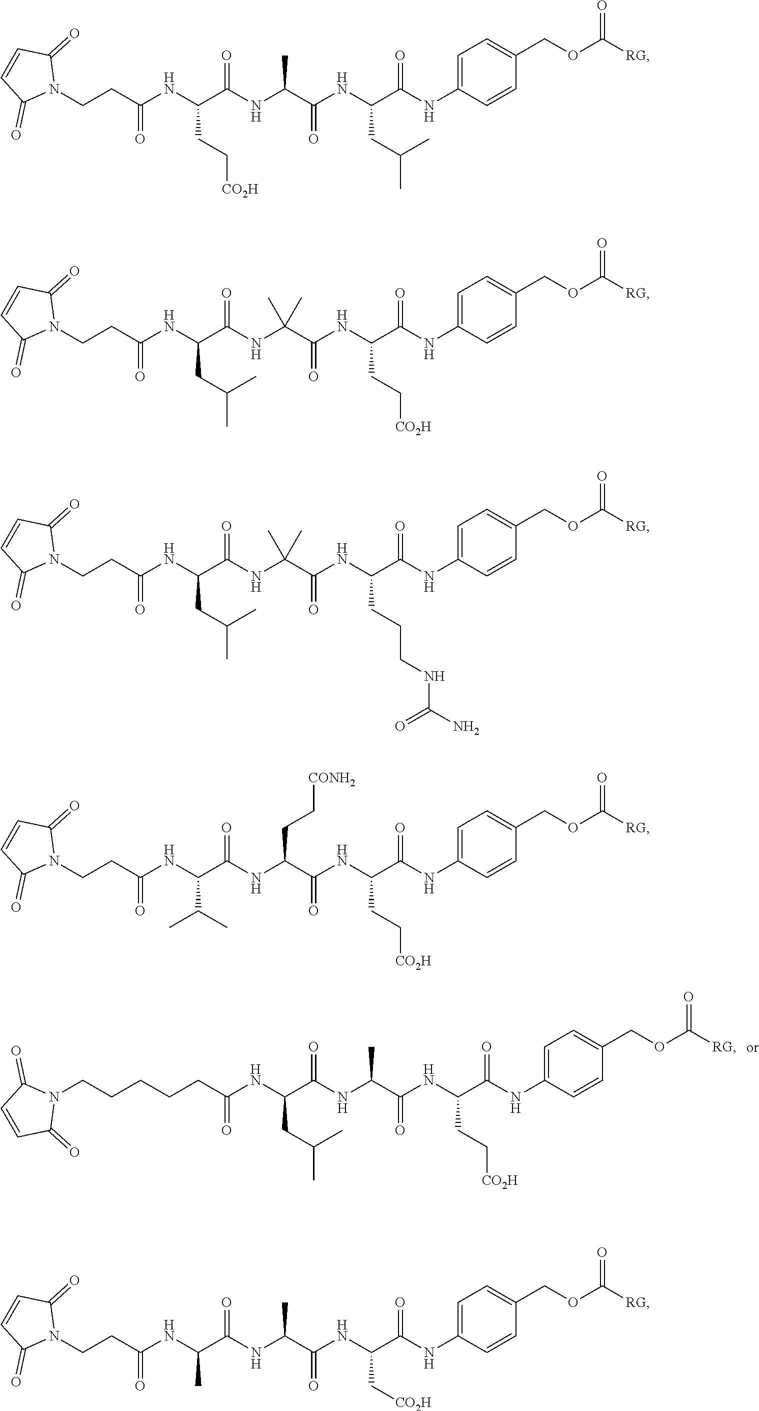

or a salt thereof, wherein RG is a reactive group.

### 3. PHARMACEUTICAL COMPOSITION

The present invention provides pharmaceutical compositions comprising an LDC composition, which is a collection of Ligand Drug Conjugate compounds described herein, and at least one pharmaceutically acceptable excipient such as a pharmaceutically acceptable carrier. The pharmaceutical compositions are in any form that allows for an LDC composition to be administered to a patient for treatment of a disorder associated with expression of the targeted moiety to which the Ligand Unit of the LDC binds. For example, the pharmaceutical compositions can be in the form of a liquid or a lyophilized solid. The preferred route of administration is parenteral. Parenteral administration includes subcutaneous injections, intravenous, intramuscular, and intrasternal injection or infusion techniques. In preferred embodiments, a pharmaceutical composition comprising an LDC composition is administered intravenously in the form of a liquid solution.

Pharmaceutical compositions are formulated so as to allow a Ligand Drug Conjugate compound to be bioavailable upon administration of the Ligand Drug Conjugate composition to a patient in need thereof. Such pharmaceutical compositions can take the form of one or more dosage units, where for example, a lyophilized solid may provide a single dosage unit when reconstituted as a solution or suspension on addition of a suitable liquid carrier.

Materials used in preparing the pharmaceutical compositions are preferably non-toxic in the amounts used. It will be evident to those of ordinary skill in the art that the optimal dosage of the active ingredient(s) in the pharmaceutical composition will depend on a variety of factors. Relevant factors include, without limitation, the type of animal (e.g., human), the particular form of the pharmaceutical composition, the manner of administration, and the LDC composition employed.

The pharmaceutical composition in some embodiments is in the form of a liquid. The liquid is useful for delivery by injection. In a pharmaceutical composition for administration by injection, one or more of a surfactant, preservative, wetting agent, dispersing agent, suspending agent, buffer, stabilizer and isotonic agent is included.

The liquid compositions, whether they are solutions, suspensions or other like form, include one or more pharmaceutically acceptable excipient selected from the group consisting of: sterile diluents such as water for injection, saline solution, preferably physiological saline, Ringer's solution, isotonic sodium chloride, fixed oils such as a synthetic mono or diglyceride, which in some embodiments also serves as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as amino acids, acetates, citrates or phosphates; detergents, such as nonionic surfactants, polyols; and agents for the adjustment of tonicity such as sodium chloride or dextrose. In preferred embodiments a parenteral composition is enclosed in ampoule, a disposable syringe or a multiple-dose vial made of glass, plastic or other material. Physiological saline is an exemplary adjuvant. An injectable pharmaceutical composition is preferably sterile.

The amount of the Conjugate that is effective in the treatment of a particular disorder or condition will depend on the nature of the disorder or condition, and can be determined by standard clinical techniques. In addition, in vitro or in vivo assays are optionally be employed to help identify optimal dosage ranges. The precise dose to be employed in the compositions will also depend on the route of administration, and the seriousness of the disease or disorder, and should be decided according to the judgment of the practitioner and each patient's circumstances.

The pharmaceutical composition comprises an effective amount of an LDC composition such that a suitable dosage will be obtained for administration to a subject in need thereof. Typically, that amount is at least about 0.01% by weight of the pharmaceutical composition.

For intravenous administration, the pharmaceutical composition comprises from about 0.01 to about 100 mg of an LDC composition per kg of the animal's body weight. In a preferred embodiment, the pharmaceutical composition includes from about 1 to about 100 mg of a LDC composition per kg of the animal's body weight. In more preferred embodiments, the amount administered will be in the range from about 0.1 to about 25 mg/kg of body weight of an LDC composition.

Generally, the dosage of an LDC composition administered to a patient is typically about 0.01 mg/kg to about 100 mg/kg of the subject's body weight. In some embodiments, the dosage administered to a patient is between about 0.01 mg/kg to about 15 mg/kg of the subject's body weight. In some embodiments, the dosage administered to a patient is between about 0.1 mg/kg and about 15 mg/kg of the subject's body weight. In some embodiments, the dosage administered to a patient is between about 0.1 mg/kg and about 20 mg/kg of the subject's body weight. In some embodiments, the dosage administered is between about 0.1 mg/kg to about 5 mg/kg or about 0.1 mg/kg to about 10 mg/kg of the subject's body weight. In some embodiments, the dosage administered is between about 1 mg/kg to about 15 mg/kg of the subject's body weight. In some embodiments, the dosage administered is between about 1 mg/kg to about 10 mg/kg of the subject's body weight. In some embodiments, the dosage administered is between about 0.1 to 4 mg/kg, preferably 0.1 to 3.2 mg/kg, or more preferably 0.1 to 2.7 mg/kg of the subject's body weight over a treatment cycle.

An LDC is administered by any convenient route, for example by infusion or bolus injection, by absorption through epithelial or mucocutaneous linings (e.g., oral mucosa, rectal and intestinal mucosa). Administration is systemic or local. Various delivery systems are known, e.g., encapsulation in liposomes, microparticles, microcapsules, capsules, and can be used to administer a compound. In certain embodiments, more than one pharmaceutical composition is administered to a patient.

In one embodiment, a Ligand Drug Conjugate composition is formulated in accordance with routine procedures as a pharmaceutical composition adapted for intravenous administration to animals, particularly human beings. Typically, the carriers or vehicles for intravenous administration are sterile isotonic aqueous buffer solutions. Where necessary, the compositions also include a solubilizing agent. Pharmaceutical compositions for intravenous administration optionally comprise a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where a pharmaceutical composition of a Ligand Drug Conjugate composition is to be administered by infusion, it is preferably dispensed with an infusion bottle containing sterile pharmaceutical grade water or saline. Where the pharmaceutical composition of a Ligand Drug Conjugate composition is administered by injection, an ampoule of sterile water for injection or saline can be provided so that the ingredients can be mixed prior to administration.

The pharmaceutical compositions are generally formulated as sterile, substantially isotonic and in full compliance with all Good Manufacturing Practice (GMP) regulations of the U.S. Food and Drug Administration.

Pharmaceutical compositions of the present invention comprise LDC compositions of the present invention and at least one pharmaceutically acceptable excipient such as pharmaceutically acceptable carrier. In some preferred embodiments, all, or substantially all, or more than 50% of the LDC compounds of the LDC composition in the pharmaceutical composition comprises a hydrolyzed thio-substituted succinimide. In some preferred embodiments, more than 55%, 60%, 65%, 70%, 75%, 80%, 85%, 90%, 91%, 92%, 93%, 94%, 95%, 96%, 97%, 98%, or 99% of the Ligand Drug Conjugates present in the pharmaceutical composition comprises a hydrolyzed thio-substituted succinimide.

### 4. TREATMENT OF HYPER-PROLIFERATING CONDITIONS

The Ligand-Drug Conjugates are useful for inhibiting the multiplication of a tumor cell or cancer cell or causing apoptosis in a tumor or cancer cell. The Ligand-Drug Conjugates are also useful in a variety of settings for the treatment of cancer. Accordingly, The Ligand-Drug Conjugates are used to deliver a drug to a tumor cell or cancer cell. Without being bound by theory, in one embodiment, the Ligand Unit of a Ligand-Drug Conjugate compound binds to or associates with a cell-surface cancer cell-or a tumor cell-associated antigen or receptor, and upon binding, the Ligand-Drug Conjugate compound is taken up (internalized) inside the tumor cell or cancer cell through antigen- or receptor-mediated endocytosis or other internalization mechanism. In another embodiment the antigen is an extracellular matrix protein associated with the tumor cell or cancer cell. Once inside the cell, via an enzymatic proteolysis mechanism, free drug is released within the cell. In an alternative embodiment, the Drug Unit is cleaved from the Ligand-Drug Conjugate compound within the vicinity of the tumor cell or cancer cell, and free drug released as a result subsequently penetrates the cell.

The Ligand-Drug Conjugate compounds provide improved conjugation-specific tumor or cancer drug targeting, thus reducing general toxicity of the drug. That improvement is due to greater selectivity for cleavage of the tripetide-based Linker Unit of the Ligand Drug Conjugate compound within a tumor to effect intracellular or extracellular delivery of free drug to the cancer cells of the tumor compared to cleavage within normal tissue typically associated with an adverse event with administering a comparator Conjugate having a dipeptide-based Linker Units and/or by increasing bioavailability of the Ligand Drug Conjugate compound for the tumor tissue, which decreases the bioavailability to the normal tissue.

In some embodiments, the peptide-based Linker Units also stabilizes the Ligand-Drug Conjugate compounds to enzymatic action by extracellular proteases in blood yet are capable of liberating drug once inside the cell.

In one embodiment, the Ligand Unit binds to the tumor cell or cancer cell.

In another embodiment, the Ligand Unit binds to a tumor cell or cancer cell antigen that is on the surface of the tumor cell or cancer cell.

In another embodiment, the Ligand Unit binds to a tumor cell or cancer cell antigen which is an extracellular matrix protein associated with the tumor cell or cancer cell.

The specificity of the Ligand Unit for a particular tumor cell or cancer cell is an important consideration for determining those tumors or cancers that are most effectively treated. For example, a Ligand Drug Conjugate having a BR96 Ligand Unit can be useful for treating antigen positive carcinomas including those of the lung, breast, colon, ovaries, and pancreas. Ligand-Drug Conjugates having an anti-CD30 or an anti-CD70 binding Ligand unit can be useful for treating hematologic malignancies.

Other particular types of cancers that can be treated with a Ligand Drug Conjugate include, but are not limited to the following solid tumors, blood-borne cancers, acute and chronic leukemias, and lymphomas.

Solid tumors include but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma.

Blood-borne cancers include but are not limited to acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CML”, chronic lymphocytic leukemia “CLL”, hairy cell leukemia, and multiple myeloma.

Acute and chronic leukemias include but are not limited to lymphoblastic, myelogenous, lymphocytic, and myelocytic leukemias.

Lymphomas include but are not limited to Hodgkin's disease, non-Hodgkin's Lymphoma, Multiple myeloma, Waldenström's macroglobulinemia, Heavy chain disease, and Polycythemia vera.

Cancers, including, but not limited to, a tumor, metastasis, or other diseases or disorders characterized by hyper-proliferating cells, are treatable or its progression inhibited in some embodiments by administration of an LDC composition.

In other embodiments, methods for treating cancer are provided, including administering to a patient in need thereof an effective amount of an LDC composition and a chemotherapeutic agent. In one embodiment the cancer to be treated with a chemotherapeutic in combination with an LDC has not been found to be refractory to the chemotherapeutic agent. In another embodiment, the cancer to be treated with a chemotherapeutic in combination with an ADC is refractory to the chemotherapeutic agent. The LDC compositions can be administered to a patient that has also undergone surgery as treatment for the cancer.

In some embodiments, the patient also receives an additional treatment, such as radiation therapy. In a specific embodiment, the Ligand-Drug Conjugate is administered concurrently with the chemotherapeutic agent or with radiation therapy. In another specific embodiment, the chemotherapeutic agent or radiation therapy is administered prior or subsequent to administration of a ligand drug conjugate.

A chemotherapeutic agent is often administered over a series of sessions. Any one or a combination of the chemotherapeutic agents, such a standard of care chemotherapeutic agent(s), is capable of being administered along with a Ligand Drug Conjugate, but it is preferable that the chemotherapeutic agent(s) effect cell killing by a different mechanism than that of free drug released from the Ligand Drug Conjugate compound.

Additionally, methods of treatment of cancer with a Ligand-Drug Conjugate are provided as an alternative to chemotherapy or radiation therapy where the chemotherapy or the radiation therapy has proven or can prove too toxic, e.g., results in unacceptable or unbearable side effects, for the subject being treated. The patient being treated can, optionally, be treated with another cancer treatment such as surgery, radiation therapy or chemotherapy, depending on which treatment is found to be acceptable or bearable.

Also provided is the use of a compound or a composition as detailed herein for the manufacture of a medicament for the treatment of any disease or condition described herein, such as cancer.

Also provided is a compound or a composition as detailed herein for use in medical therapy. Further provided is a compound or a composition as detailed herein for use in treatment of any disease or condition described herein, such as cancer.

Also provided is the use of a compound or a composition as detailed herein for medical therapy. Further provided is the use of a compound or a composition as detailed herein for treatment of any disease or condition described herein, such as cancer.

Further provided is a kit comprising a compound or a composition as detailed herein. In some embodiments, the kit comprises instructions for use according to any of the methods provided herein.

In another aspect, provided is a method of making a compound or a composition as detailed herein.

### ENUMERATED EMBODIMENTS

Embodiment 1. A Ligand Drug Conjugate composition represented by Formula 1:

L-[LU-D′]ₚ (1)
or a salt thereof, in particular a pharmaceutically acceptable salt, wherein
L is a Ligand Unit;
LU is a Linker Unit;
D′ represents from 1 to 4 Drug Units (D) in each drug linker moiety of formula -LU-D′; and
subscript p is a number from 1 to 12, from 1 to 10 or from 1 to 8 or is about 4 or about 8,
wherein the Ligand Unit is from an antibody or an antigen-binding fragment of an antibody that is capable of selective binding to an antigen of tumor tissue for subsequent release of the Drug Unit(s) as free drug,
wherein the drug linker moiety of formula -LU-D′ in each of the Ligand Drug Conjugate compounds of the composition has the structure of Formula 1A:

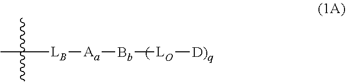

or a salt thereof, in particular a pharmaceutically acceptable salt, P
wherein the wavy line indicates covalent attachment to L;
D is the Drug Unit;
L_{B} is a ligand covalent binding moiety;
A is a first optional Stretcher Unit;
subscript a is 0 or 1, indicating the absence or presence of A, respectively;
B is an optional Branching Unit;
subscript b is 0 or 1, indicating the absence or presence of B, respectively;
L_{O} is a secondary linker moiety, wherein the secondary linker has the formula of;

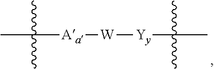

wherein the wavy line adjacent to Y indicates the site of covalent attachment of L_{O} to the Drug Unit and the wavy line adjacent to A′ indicates the site of covalent attachment to the remainder of the drug linker moiety;
A′ is a second optional Stretcher Unit, which in the absence of B becomes a subunit of A,
subscript a′ is 0 or 1, indicating the absence or presence of A′, respectively,
W is a peptide Cleavable Unit, wherein the peptide Cleavable Unit is a contiguous sequence of up to 12 amino acids, wherein the sequence is comprised of a selectivity conferring tripeptide that provides improved selectivity for exposure of tumor tissue over normal tissue to free cytotoxic compound released from the Ligand Drug Conjugate compounds of the composition in comparison to the compounds of a comparator Ligand-Drug Conjugate composition in which the peptide sequence of its peptide Cleavable Unit is the dipeptide -valine-citrulline- or -valine-alanine-;
wherein the tumor and normal tissues are of rodent species and wherein the formula I composition provides said improved exposure selectivity demonstrated by:
retaining efficacy in a tumor xenograft model of the comparator conjugate composition when administered at the same effective amount and dose schedule previously determined for the comparator conjugate composition, and
showing a reduction in plasma concentration of free drug, and/or preservation of normal cells in tissue when administration at the same effective amount and dose schedule as in the tumor xenograft model to a non-tumor bearing rodent in comparison that same administration of the comparator conjugate in which the Ligand Units of both conjugate compositions are replaced by a non-binding antibody,
wherein the normal tissue is of the same tissue type in human and wherein cytotoxicity to cells of that tissue is responsible at least in part to an adverse event in a human subject to whom is administered a therapeutically effective amount of the comparator conjugate composition;
Y is a self-immolative Spacer Unit;
subscript y is 0, 1 or 2 indicating the absence or presence of 1 or 2 of Y, respectively; and
subscript q is an integer ranging from 1 to 4,
provided that subscript q is 1 when subscript b is 0 and subscript q is 2, 3 or 4 when subscript b is 1; and
wherein the Ligand Drug Conjugate compounds of the composition have the structure of Formula 1 in which subscript p is replaced by subscript p′, wherein subscript p′ is an integer from 1 to 12, 1 to 10 or 1 to 8 or is 4 or 8.

Embodiment 2. The Ligand Drug Conjugate composition of embodiment 1, wherein the xenograft model is SCID or nude mouse implanted with HPAF-II, Ramos SK-MEL-5 or SU-DHL-4 cancer cells, in particular nude mouse implanted with HPAF-II cancer cells.

Embodiment 3. The Ligand Drug Conjugate composition of embodiment 1 or 2, wherein the normal tissue is rat bone marrow.

Embodiment 4. The Ligand Drug Conjugate composition of embodiment 1 or 2, wherein the Formula I composition provides said improved exposure selectivity is further demonstrated by an increased ratio of proteolysis of the Formula 1 composition by homogenized tumor xenograft tissue over proteolysis of the comparator conjugate by homogenized normal tissue when incubated under the same conditions in comparison to that ratio for the comparator conjugate.

Embodiment 5. The Ligand Drug Conjugate composition of embodiment 4, wherein the normal tissue is from bone marrow of rat or of human.

Embodiment 6. The Ligand Drug Conjugate composition of any one of embodiments 1-5, wherein the tumor xenograft tissue is from nude mice implanted with HPAF-II cancer cells.

Embodiment 7. The Ligand Drug Conjugate composition of any one of embodiments 1-6, wherein each drug linker moiety has the formula of:

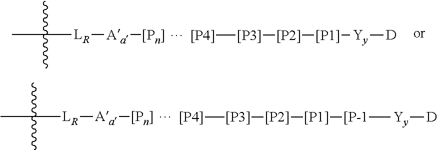

or a salt thereof, in particular a pharmaceutically acceptable salt, wherein
L_{R} is a primary linker of formula -L_{B}-Aₐ-B_{b}—, provided that A′ is a subunit of A so that A′ is a component of L_{R} when subscript a and a′ are each 1 and subscript b is 0; and
each P is an amino acid residue of the contiguous amino acid sequence of the peptide Cleavable Unit and wherein subscript n has an integer value providing for up to 12 of these residues.

Embodiment 8. The Ligand Drug Conjugate composition of any one of embodiments 1-6, wherein each drug linker moiety has the formula of:

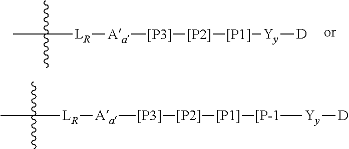

or a salt thereof, in particular a pharmaceutically acceptable salt,
wherein L_{R} is a primary linker of formula -L_{B}-Aₐ-B_{b}—, provided that A′ is a subunit of A so that A′ is a component of L_{R} when subscript a and a′ are each 1 and subscript b is 0; and
wherein each P is an amino acid residue of the contiguous amino acid sequence of the peptide Cleavable Unit.

Embodiment 9. The Ligand Drug Conjugate composition of any one of embodiments 1-6, wherein each drug linker moiety has the formula of:

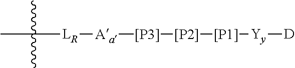

or a salt thereof, in particular a pharmaceutically acceptable salt, wherein
L_{R} is a primary linker of formula -L_{B}-Aₐ-B_{b}—, provided that A′ is a subunit of A so that A′ is a component of L_{R} when subscript a and a′ are each 1 and subscript b is 0;
each P is an amino acid residue of the contiguous amino acid sequence of the peptide Cleavable Unit and wherein subscript n has an integer value providing for up to 12 of these residues; and
P1 is a L-amino acid residue having at physiological pH a negatively charged side chain or a non-positively charged polar side chain.

Embodiment 10. The Ligand Drug Conjugate composition of any one of embodiments 1-9, wherein P1 is a L-amino acid residue selected from the group consisting of glutamic acid, methionine-sulfoxide, aspartic acid, (S)-3-aminopropane-1,1,3-tricarboxylic acid and phospho-threonine.

Embodiment 11. The Ligand Drug Conjugate composition of any one of embodiments 1-6, wherein each drug linker moiety has the formula of:

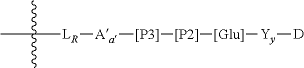

or a salt thereof, in particular a pharmaceutically acceptable salt, wherein
L_{R} is a primary linker of formula -L_{B}-Aₐ-B_{b}—, provided that A′ is a subunit of A so that A′ is a component of L_{R} when subscript a and a′ are each 1 and subscript b is 0; and
each P is an amino acid residue of the contiguous amino acid sequence of the peptide Cleavable Unit.

Embodiment 12. The Ligand Drug Conjugate composition of any one of embodiments 1-11, wherein P2 is a residue of glycine or an L-amino acid, the side chain of which has no more than three contiguous carbon atoms.

Embodiment 13. The Ligand Drug Conjugate composition of any one of embodiments 1-11, wherein the P2 amino acid is L-alanine, L-valine or glycine or an unnatural amino acid, wherein the unnatural amino acid is Abu, Aib, Ala, Gly, Leu, Nva or Pra, wherein Abu, Aib, Nva, and Pra have the structures of:

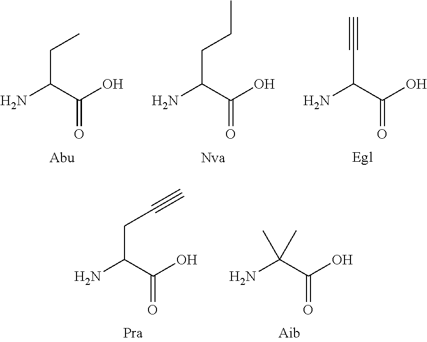

and wherein the side chains of Abu, Nva and Pra are in the same stereochemical configuration of an L-amino acid.

Embodiment 14. The Ligand Drug Conjugate composition of any one of embodiments 1-6, wherein each drug linker moiety has the formula of:

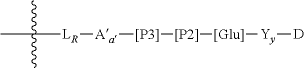

or a salt thereof, in particular a pharmaceutically acceptable salt, wherein
L_{R} is a primary linker of formula -L_{B}-Aₐ-B_{b}—, provided that A′ is a subunit of A so that A′ is a component of L_{R} when subscript a and a′ are each 1 and subscript b is 0; and
P3 is an amino acid residue of the contiguous amino acid sequence of the peptide Cleavable Unit.

Embodiment 15. The Ligand Drug Conjugate composition of any one of embodiments 1-14, wherein P3 is a D-amino acid, the side chain of which is uncharged at physiological pH.

Embodiment 16. The Ligand Drug Conjugate composition of any one of embodiments 1-14 wherein P3 is a D-Leu, L-Leu, L-Cit or L-Pro, preferably D-Leu.

Embodiment 17. The Ligand Drug Conjugate composition of embodiment 1-9, wherein the selectivity conferring tripeptide, —[P3]-[P2]-[P1]-, is -D-Leu-Ala-Glu-, or a salt thereof, in particular a pharmaceutically acceptable salt.

Embodiment 18. The Ligand Drug Conjugate composition of any one of embodiments 1-17, wherein -L_{R}- in the drug linker moieties of each Ligand Drug Conjugate compound has or is comprised of one of the structures of:

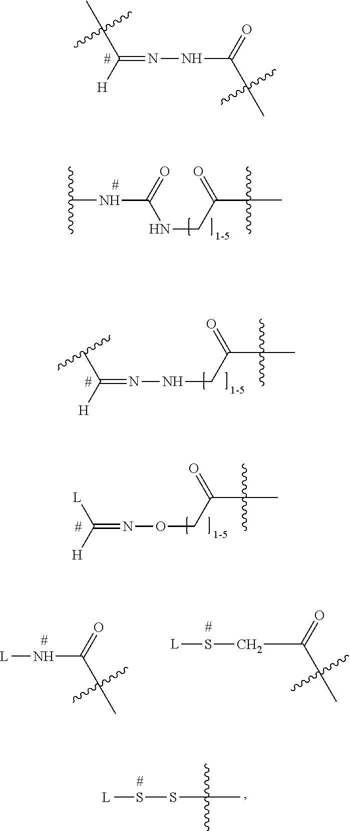

wherein the indicated (#) nitrogen, carbon or sulfur atom is from the Ligand Unit; and wherein the wavy line adjacent thereto indicates the site of covalent attachment to the remainder of the Ligand Unit and the other wavy line indicates the site of covalent attachment to the remainder of one of the drug linker moieties.

Embodiment 19. The Ligand Drug Conjugate composition of any one of embodiments 1-17, wherein subscript q is 1 and L_{R} is -L_{B}-A-,
wherein -L_{B}-A- in the drug linker moieties of each Ligand Drug Conjugate compound predominately has the structure of:

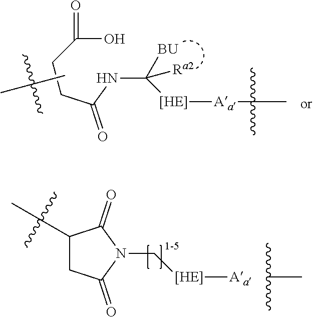

or a salt thereof, in particular a pharmaceutically acceptable salt, wherein
the wavy line adjacent to A′_{a′} indicates the site of covalent attachment to the Peptide Cleavable Unit of one of the drug linker moieties; and the other wavy line indicates the site of covalent attachment to a sulfur atom of the Ligand Unit;
[HE] is a Hydrolysis Enhancing Unit;
BU is a Basic Unit;
R^{a2} is an optionally substituted C₁-C₁₂ alkyl group; and
the dotted curved line indicates optional cyclization so that in the absence of said cyclization, BU is an acyclic Basic Unit having a primary, secondary or tertiary amine functional group as the basic function group of the acyclic Basic Unit, or in the presence of said cyclization BU is a cyclized Basic Unit in which R^{a2} and BU together with the carbon atom to which both are attached, define an optionally substituted spiro C₃-C₂₀ heterocyclo containing a skeletal basic nitrogen atom of a secondary or tertiary amine functional group as the basic function group of the cyclic Basic Unit, wherein the basic nitrogen atom of the acyclic Basic Unit or cyclic Basic Unit is optionally suitably protected by a nitrogen protecting group, dependent on the degree of substitution of the basic nitrogen atom or is optionally protonated.

Embodiment 20. The Ligand Drug Conjugate composition of embodiment 19, wherein -L_{B}-A- in the drug linker moieties of each Ligand Drug Conjugate compound predominately have the structure of:

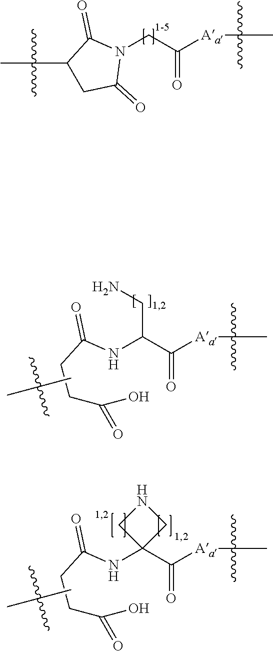

or a salt thereof, in particular a pharmaceutically acceptable salt.

Embodiment 21. The Ligand Drug Conjugate composition of any one of embodiments 1-20, wherein subscript q is 1 and A′ is present as a subunit of A, wherein A′ is comprised of an amine-containing acid residue having the structure of formula (3) or formula (4):

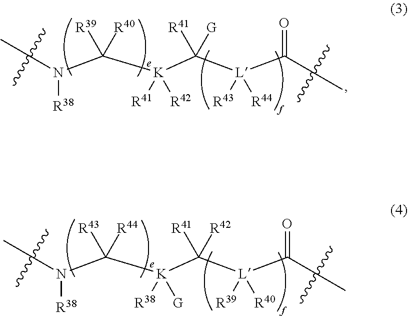

or a salt thereof, in particular a pharmaceutically acceptable salt, wherein
the wavy line adjacent to the nitrogen atom indicates the site of covalent attachment to [HE], wherein [HE] is —C(═O)— and the wavy line adjacent to the carbonyl carbon atom indicates the site of covalent attachment to the remainder of A′ or to the N-terminal amino acid residue of the Peptide Cleavable Unit, wherein both attachments are through amide functional groups;
K and L′ independently are C, N, O or S, provided that when K or L′ is O or S, R⁴¹ and R⁴² to K, R³⁸ and G to K, R⁴³ and R⁴⁴ to L′, and R³⁹ and R⁴⁰ to L′ are absent, and when K or L′ are N, one of R⁴¹ or R⁴² to K and one of R³⁸ or G to K, one of R⁴³ or R⁴⁴ to L′ for each unit of -L′(R⁴³)(R⁴⁴), and one of R³⁹ or R⁴⁰ to L′ for each unit of -L′(R³⁹)(R⁴⁰) are absent, and provided that no two adjacent L′ are independently selected as N, O, or S;
wherein subscripts e and f are independently selected integers that range from 0 to 12, and subscript g is an integer ranging from 1 to 12:
G is hydrogen, optionally substituted C₁-C₆ alkyl, —OH or —CO₂H;
R³⁸ is hydrogen or optionally substituted C₁-C₆ alkyl;
R³⁹-R⁴⁴ are independently selected from the group consisting of hydrogen, optionally substituted C₁-C₆ alkyl and optionally substituted C₅-C₁₀ (hetero)aryl,
or R³⁹ and R⁴⁰ together with the carbon atom to which both are attached, or R⁴¹ and R⁴² together with K to which both are attached when K is a carbon atom, define a C₃-C₆ carbocyclo, and the remainder of R³⁹-R⁴⁴ are as defined herein,
or R⁴³ and R⁴⁴ together with L′ to which both are attached when L′ is a carbon atom define a C₃-C₆ carbocyclo, and R³⁹-R⁴² are as defined herein,
or R⁴⁰ and R⁴¹, or R⁴⁰ and R⁴³, or R⁴¹ and R⁴³ to together with the carbon atom or heteroatom to which both are attached and the optional atoms intervening between those carbon atoms and/or heteroatoms define a C₅-C₆ carbocyclo or a C₅-C₆ heterocyclo, and R³⁹, R⁴⁴ and the remainder of R⁴⁰-R⁴³ are as defined herein,
provided that when K is O or S, R⁴¹ and R⁴² are absent, and when K is N, one of R⁴¹, R⁴² is absent, and when L′ is O or S, R⁴³ and R⁴⁴ are absent, and when L′ is N, one of R⁴³, R⁴⁴ is absent, or
A′ is comprised of an alpha-amino, beta-amino or another amine-containing acid residue, wherein its amino nitrogen atom is covalently attached to the carbonyl carbon atom of HE, and its carboxylic acid carbonyl carbon atom is covalently attached to the remainder of A′ or to N-terminal amino acid of the Peptide Cleavable Unit, wherein both covalent attachments are through amide functional groups.

Embodiment 22. The Ligand Drug Conjugate composition of embodiment 21, wherein A′ is an amine-containing acid residue having the structure of formula 3a, formula 4a or formula 5a:

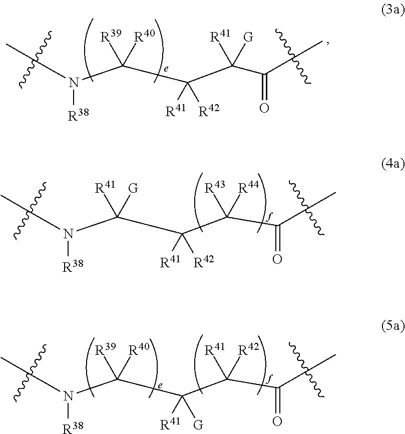

or a salt thereof, in particular a pharmaceutically acceptable salt, wherein
subscripts e and f are independently 0 or 1; and
R³⁸-R⁴⁴ are each hydrogen;
or A′ is an α-amino or β-amino acid residue.

Embodiment 23. The Ligand Drug Conjugate composition of any one of embodiments 1-20, wherein subscript q is 1 and A′ is comprised of a β-amino acid residue or -L^{P}(PEG)-,
wherein PEG is a PEG Unit and L^{P} is Parallel Connector Unit having the structure of Formula L^{P}-1 or L^{P}-2:

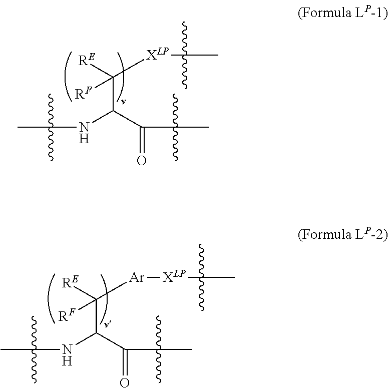

or
wherein -L^{P}(PEG)- or a PEG-containing subunit thereof has the structure of Formula L^{P}-3 or Formula L^{P}-4:

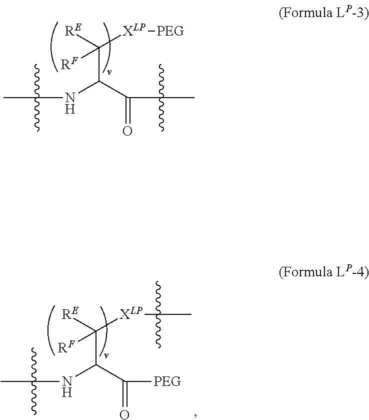

wherein subscript v is an integer ranging from 1 to 4;
subscript v′ is an integer ranging from 0 to 4;
X^{LP} is provided by a natural or un-natural amino acid side chain or is selected from the group consisting of —O—, —NR^{LP}—, —S—, —S(═O)—, —S(═O)₂—, —C(═O)—, —C(═O)N(R^{LP})—, —N(R^{LP})C(═O)N(R^{LP})—, and —N(R^{LP})C(═NR^{LP})N(R^{LP})—, or C₃-C₈ heterocyclo;
wherein each R^{LP} is independently selected from the group consisting of hydrogen and optionally substituted C₁-C₆ alkyl, or two of R^{LP} together along with the carbons atoms to which they are attached, and their intervening atoms define a C₅-C₆ heterocyclo and any remaining R^{LP} are as previously defined;
Ar is a C₆-C₁₀ arylene or a C₅-C₁₀ heteroarylene, each of which is optionally substituted;
each R^{E} and R^{F} is independently selected from the group consisting of —H, optionally substituted C₁-C₆ alkyl, optionally substituted C₂-C₆ alkylene, optionally substituted C₆-C₁₀ arylene or optionally substituted C₅-C₁₀ heteroarylene,
or R^{E} and R^{F} together with the carbon atom to which both are attached defines an optionally substituted spiro C₃-C₆ carbocyclo, or R^{E} and R^{F} from adjacent carbon atoms together with these atoms and any intervening carbon atoms defines an optionally substituted C₅-C₆ carbocyclo with any remaining R^{E} and R^{F} as previously defined;
wherein one of the wavy lines indicate the point of covalent attachment of a PEG Unit and the other two wavy lines indicates covalent attachment of Formula L^{P}-1 or Formula L^{P}-2 within the structure representing the Ligand Drug Conjugate composition, or
L^{P} is Parallel Connector Unit having the structure of a tri-functional amine-containing acid residue or; and
PEG is a PEG Unit.

Embodiment 24. The Ligand Drug Conjugate composition of any one of embodiment 1-20, wherein A′ is comprised of a β-amino acid residue or -L^{P}(PEG)-, wherein PEG is a PEG Unit and L^{P} is Parallel Connector Unit,
wherein the β-amino acid residue has the structure of —NHCH₂CH₂C(═O)—; and
wherein -L^{P}(PEG)- has the structure of:

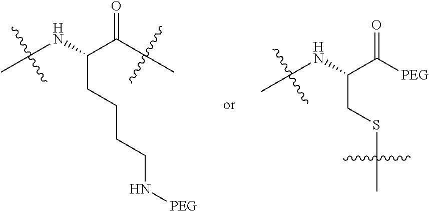

wherein the wavy lines indicate the sites of covalent attachment within the drug linker moiety.

Embodiment 25. The Ligand Drug Conjugate composition of embodiment 23 or 24, wherein the PEG Unit has the structure of:

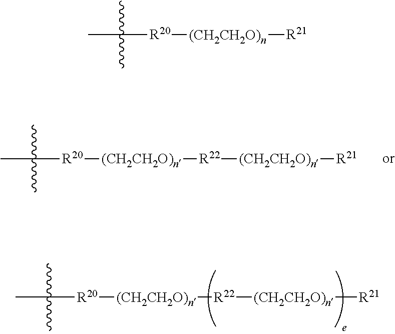

wherein the wavy line indicates the site of covalent attachment to L^{P};
R²⁰ is a PEG Attachment Unit, wherein the PEG Attachment Unit is —C(O)—, —O—, —S—, —S(O)—, —NH—, —C(O)O—, —C(O)C₁-C₁₀ alkyl, —C(O)C₁-C₁₀ alkyl-O—, —C(O)C₁-C₁₀ alkyl-CO₂—, —C(O)C₁-C₁₀alkyl-NH—, —C(O)C₁-C₁₀ alkyl-S—, —C(O)C₁-C₁₀alkyl-C(O)—NH—, —C(O)C₁-C₁₀alkyl-NH—C(O)—, —C₁-C₁₀ alkyl, —C₁-C₁₀alkyl-O—, —C₁-C₁₀alkyl-CO₂—, —C₁-C₁₀alkyl-NH—, —C₁-C₁₀ alkyl-S—, —C₁-C₁₀ alkyl-C(O)—NH—, —C₁-C₁₀ alkyl-NH—C(O)—, —CH₂CH₂SO₂—C₁-C₁₀ alkyl-, —CH₂C(O)—C₁₋₁₀ alkyl-, ═N—(O or N)—C₁-C₁₀ alkyl-O—, ═N—(O or N)—C₁-C₁₀ alkyl-NH—, ═N—(O or N)—C₁-C₁₀ alkyl-CO₂—, ═N—(O or N)—C₁-C₁₀ alkyl-S—,

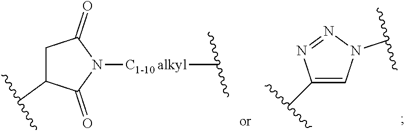

R²¹ is a PEG Capping Unit; wherein the PEG Capping Unit is —C₁-C₁₀ alkyl, —C₂-C₁₀ alkyl-CO₂H, —C₂-C₁₀ alkyl-OH, —C₂-C₁₀ alkyl-NH₂, C₂-C₁₀ alkyl-NH(C₁-C₃ alkyl), or C₂-C₁₀ alkyl-N(C₁-C₃ alkyl)₂;
R²² is an PEG Coupling Unit for coupling multiple PEG subunit chains together, wherein the PEG Coupling Unit is —C₁₋₁₀ alkyl-C(O)—NH—, —C₁₋₁₀ alkyl-NH—C(O)—, —C₂₋₁₀ alkyl-NH—, —C₂-C₁₀ alkyl-O—, —C₁-C₁₀ alkyl-S—, or —C₂-C₁₀ alkyl-NH—;
subscript n is independently selected from 8 to 72, from 10 to 72 or from 12 to 72;
subscript e is selected from 2 to 5; and
each n′ is independently selected from at least 6 to no more than 72, preferably from at least 8 or at least 10 to no more than 36.

Embodiment 26. The Ligand Drug Conjugate composition of any one of embodiments 1-6, wherein a majority of Ligand Drug Conjugate compounds in the Ligand Drug Conjugate composition have drug linker moieties represented by the structures of Formula 1C and Formula 1D:

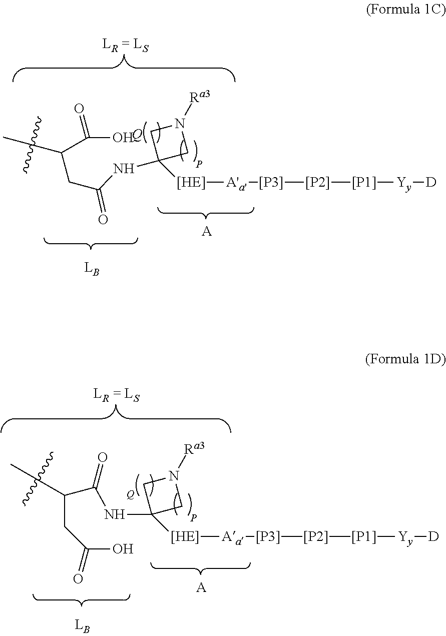

or a salt thereof, in particular a pharmaceutical acceptable salt, wherein
HE is a Hydrolysis Enhancing Unit;
A′ is a subunit, when present, of the indicated first Stretcher Unit (A);
subscript a′ is 0 or 1, indicating the absence or presence of A′, respectively;
subscript P is 1 or 2; and subscript Q ranges from 1 to 6, preferably subscript Q is 1 or 2, more preferably subscript Q has the same value as subscript P;
R^{a3} is —H, optionally substituted C₁-C₆ alkyl, optionally substituted —C₁-C₄ alkylene-(C₆-C₁₀ aryl), or —R^{PEG1}—O—(CH₂CH₂O)₁₋₃₆—R^{PEG2}, wherein R^{PEG1} is C₁-C₄ alkylene and R^{PEG2} is —H or C₁-C₄ alkylene, wherein the basic nitrogen bonded to R^{a3} is optionally protonated in a salt form, preferably in a pharmaceutically acceptable salt form, or
R^{a3} is a nitrogen protecting group such as a suitable acid-labile protecting group;
each P is an amino acid residue of the contiguous amino acid sequence of the peptide Cleavable Unit; and
the wavy line indicates the site of covalent binding to a sulfur atom of the Ligand Unit.

Embodiment 27. The Ligand Drug Conjugate composition of embodiment 1, wherein a majority of Ligand Drug Conjugate compounds in the Ligand Drug Conjugate composition have drug linker moieties represented by the structures of Formula 1F and Formula 1G:

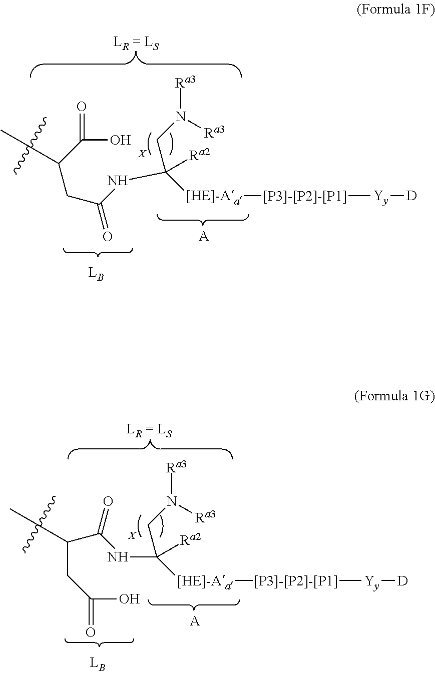

or a salt thereof, in particular a pharmaceutical acceptable salt, wherein
HE is a Hydrolysis Enhancing Unit;
A′ is a subunit, when present, of the indicated first Stretcher Unit (A);
subscript a′ is 0 or 1, indicating the absence or presence of A′, respectively;
subscript x is 1 or 2;
R^{a2} is —H, optionally substituted C₁-C₆ alkyl, —CH₃ or —CH₂CH₃;
R^{a3}, at each instance, is independently a nitrogen protecting group, —H or optionally substituted C₁-C₆ alkyl, preferably —H, an acid-labile protecting group, —CH₃ or —CH₂CH₃,
or both R^{a3} together with the nitrogen to which they are attached define a nitrogen protecting group or an azetidinyl, pyrrolidinyl or piperidinyl heterocyclyl, in which a basic primary, secondary or tertiary amine so defined is optionally protonated in a salt form, preferably a pharmaceutically acceptable salt form; and
the wavy line indicates the site of covalent binding to a sulfur atom of the Ligand Unit.

Embodiment 28. The Ligand Drug Conjugate composition of embodiment 1, wherein the Ligand Drug Conjugate compounds in the Ligand Drug Conjugate composition predominately have drug linker moieties of Formula 1H:

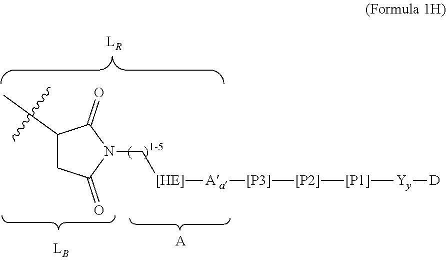

or salts thereof, in particular pharmaceutical acceptable salts, and optionally having a minority of Ligand Drug Conjugate compounds in which one or more of the drug linker moieties in each of such compounds has its the succinimide ring in hydrolyzed form and wherein
HE is a Hydrolysis Enhancing Unit;
A′ is a subunit, when present, of the indicated first Stretcher Unit (A); subscript a′ is 0 or 1, indicating the absence or presence of A′; and
the wavy line indicates the site of covalent binding to a sulfur atom of the Ligand Unit.

Embodiment 29. The Ligand Drug Conjugate composition of embodiment 26, wherein a majority of Ligand Drug Conjugate compounds in the Ligand Drug Conjugate composition have drug linker moieties represented by the structures of:

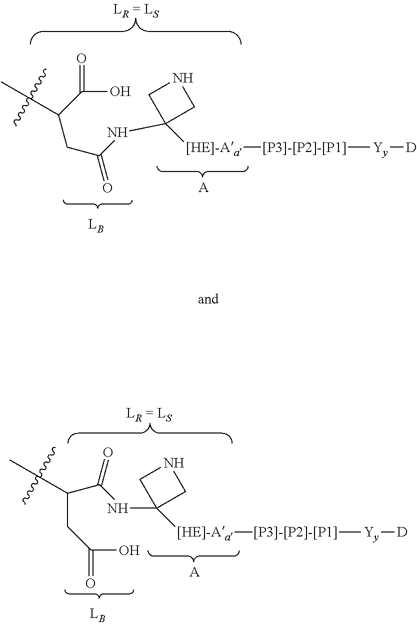

or salts thereof, in particular pharmaceutical acceptable salts.

Embodiment 30. The Ligand Drug Conjugate composition of embodiment 28, wherein a majority of Ligand Drug Conjugate compounds in the Ligand Drug Conjugate composition have drug linker moieties represented by the structures of:

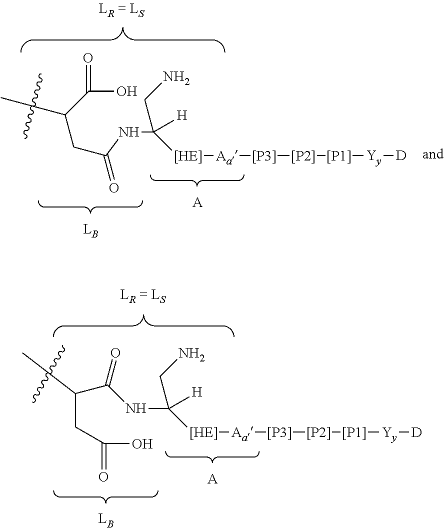

or salts thereof, in particular pharmaceutical acceptable salts.

Embodiment 31. The Ligand Drug Conjugate composition of any one of embodiments 26-30 wherein HE is —C(═O).

Embodiment 32. The Ligand Drug Conjugate composition of any one of embodiments 26-30 wherein HE is —C(═O), subscript a′ is 1 and A′ has the structure of formula 3a, formula 4a or formula 5a of embodiment 17, or A′ is an α-amino acid or (3-amino acid residue.

Embodiment 33. The Ligand Drug Conjugate composition of any one of embodiments 26-32, wherein —[P3]-[P2]-[P1]- is D-Leu-Leu-Met(O), D-Leu-Ala-Glu, L-Leu-Ala-Glu or D-Leu-Ala-Cit wherein Met(O) is methionine in which its sulfur atom is oxidized to a sulfoxide and Cit is citrulline.

Embodiment 34. The Ligand Drug Conjugate composition of any one of embodiments 1-33, wherein —Y_{y}-D has the structure of:

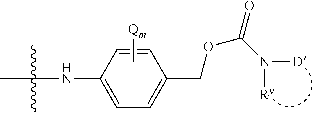

wherein —N(R^{y})D′ represents D, wherein D′ is the remainder of D;
the wavy line indicates the site of covalent attachment to P1 or P-1;
the dotted line indicates optional cyclization of R^{y} to D;
R^{y} is optionally substituted C₁-C₆ alkyl in absence of cyclization to D′ or optionally substituted C₁-C₆ alkylene when cyclized to D′;
each Q is independently —C₁-C₈ alkyl, —O—(C₁-C₈ alkyl), or other electron donating group, -halogen, -nitro or -cyano or other electron withdrawing group, in particular each Q is independently selected from the group consisting of —C₁-C₈ alkyl, —O—(C₁-C₈ alkyl), halogen, nitro and cyano; and
subscript m is 0, 1 or 2, in particular subscript m is 0 or 1 and Q when present is an electron donating group, preferably subscript m is 0.

Embodiment 35. The Ligand Drug Conjugate composition of embodiment 1 wherein predominate drug linker moiety in a majority of Ligand Drug Conjugate compounds of the composition are represented by the structure of:

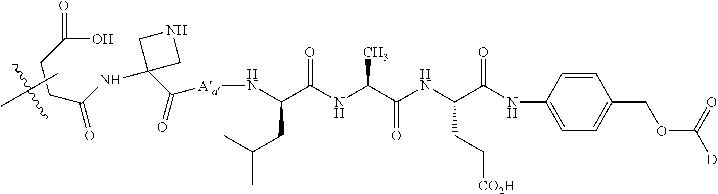

or salts thereof, in particular, pharmaceutically acceptable salts, wherein
the wavy line indicates covalent attachment to a sulfur atom from a Ligand Unit;
subscript a′ is 1, indicating the presence of A′, wherein A′ is an amine-containing acid residue of formula 3a, formula 4a or formula 5a of embodiment 22, or an α-amino acid or β-amino acid residue, in particular —NH—CH₂CH₂—C(═O)—; and
D is a cytotoxic drug having a secondary amino group as the site of attachment to the drug linker moiety.

Embodiment 36. The Ligand Drug Conjugate composition of embodiment 1 wherein predominate drug linker moiety in a majority of Ligand Drug Conjugate compounds of the composition are represented by the structure of:

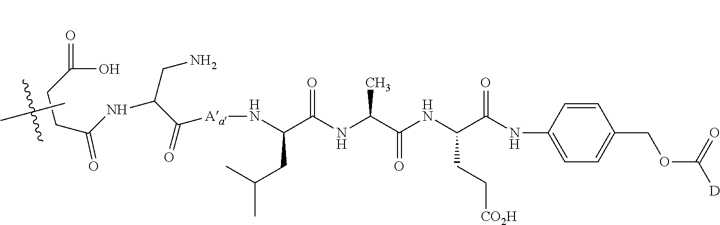

or salts thereof, in particular, pharmaceutically acceptable salts, wherein
the wavy line indicates covalent attachment to a sulfur atom from a Ligand Unit;
subscript a′ is 1, indicating the presence of A, respectively, wherein A′ is an amine-containing acid residue of formula 3a, formula 4a or formula 5a of embodiment 22, or an α-amino acid or β-amino acid residue, in particular —NH—CH₂CH₂—C(═O)—; and
D is a cytotoxic drug having a secondary amino group as the site of attachment to the drug linker moiety.

Embodiment 37. The Ligand Drug Conjugate composition of embodiment 1 wherein predominate drug linker moiety in a majority of Ligand Drug Conjugate compounds of the composition is represented by the structure of:

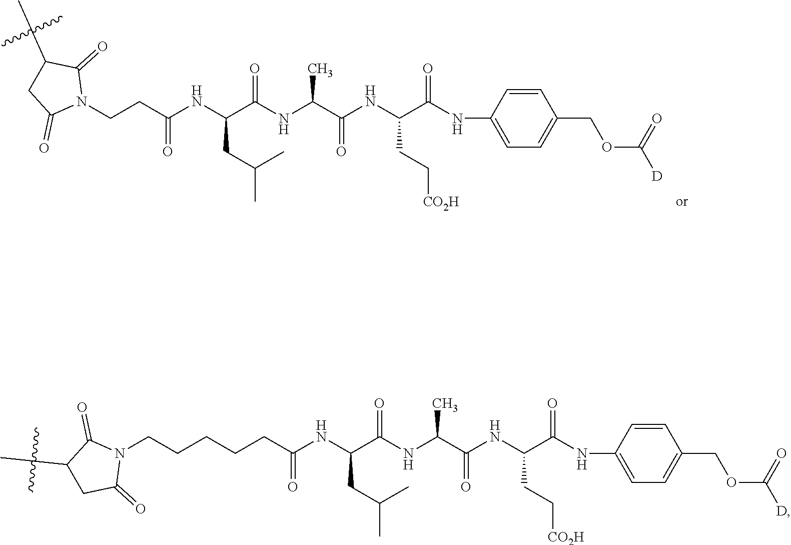

or salts thereof, in particular, pharmaceutically acceptable salts, wherein
the wavy line indicates covalent attachment to a sulfur atom from a Ligand Unit; and
D is a cytotoxic drug having a secondary amino group as the site of attachment to the drug linker moiety.

Embodiment 38. The Ligand Drug Conjugate composition of any one of embodiments 1-37, wherein subscript y′ is 2, and Y_{y} is —Y—Y′—, wherein Y is a first self-immolative Spacer Unit and Y′ is a second self-immolative Spacer Unit having the structure of —OC(═O)— and the cytotoxic drug is a secondary amine-containing auristatin compound wherein the nitrogen atom of the secondary amine is the site of covalent attachment to the carbonyl carbon atom of Y′ through a carbamate functional group shared between D and Y′.

Embodiment 39. The Ligand Drug Conjugate composition of embodiment 38, wherein the secondary amine-containing auristatin compound has the structure of Formula D_{E} or D_{F}:

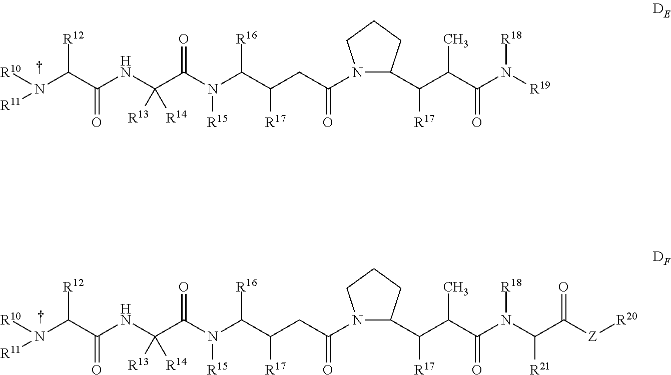

wherein the dagger indicates the site of covalent attachment of the nitrogen atom that provides the carbamate functional group,
one of R¹⁰ and R¹¹ is hydrogen and the other is C₁-C₈ alkyl, preferably one of R¹⁰ and R¹¹ is hydrogen and the other is methyl;
R¹² is hydrogen, C₁-C₈ alkyl, C₃-C₈ carbocyclyl, C₆-C₂₄ aryl, —X¹—C₆-C₂₄ aryl, —X¹—(C₃-C₈ carbocyclyl), C₃-C₈ heterocyclyl or —X¹—(C₃-C₈ heterocyclyl);
R¹³ is hydrogen, C₁-C₈ alkyl, C₃-C₈ carbocyclyl, C₆-C₂₄ aryl, —X¹—C₆-C₂₄ aryl, —X¹—(C₃-C₈ carbocyclyl), C₃-C₈ heterocyclyl or —X¹—(C₃-C₈ heterocyclyl);
R¹⁴ is hydrogen or methyl, or
R¹³ and R¹⁴ taken together with the carbon to which they are attached comprise a spiro C₃-C₈ carbocyclo;
R¹⁵ is hydrogen or C₁-C₈ alkyl;
R¹⁶ is hydrogen, C₁-C₈ alkyl, C₃-C₈ carbocyclyl, C₆-C₂₄ aryl, —C₆-C₂₄—X¹-aryl, —X¹—(C₃-C₈ carbocyclyl), C₃-C₈ heterocyclyl or —X¹—(C₃-C₈ heterocyclyl);
each R¹⁷ independently are hydrogen, —OH, C₁-C₈ alkyl, C₃-C₈ carbocyclyl or O—(C₁-C₈ alkyl);
R¹⁸ is hydrogen or optionally substituted C₁-C₈ alkyl;
R¹⁹ is —C(R^{19A})₂—C(R^{19A})₂—C₆-C₂₄ aryl, —C(R^{19A})₂—C(R^{19A})₂—(C₃-C₈ heterocyclyl) or —C(R^{19A})₂—C(R^{19A})₂—(C₃-C₈ carbocyclyl), wherein C₆-C₂₄ aryl and C₃-C₈ heterocyclyl are optionally substituted;
R^{19A} independently are hydrogen, optionally substituted C₁-C₈ alkyl, —OH or optionally substituted —O—C₁-C₈ alkyl;
R²⁰ is hydrogen or C₁-C₂₀ alkyl, C₆-C₂₄ aryl or C₃-C₈ heterocyclyl, optionally substituted, or —(R⁴⁷O)ₘ—R⁴⁸, or —(R⁴⁷O)ₘ—CH(R⁴⁹)₂;
R²¹ is —C₁-C₈ alkylene-(C₆-C₂₄ aryl) or —C₁-C₈ alkylene-(C₅-C₂₄ heteroaryl), optionally substituted, or C₁-C₈ hydroxylalkyl, or optionally substituted C₃-C₈ heterocyclyl;
Z is O, S, NH, or NR⁴⁶.
R⁴⁶ is optionally substituted C₁-C₈ alkyl; subscript m is an integer ranging from 1-1000;
R⁴⁷ is C₂-C₈ alkylene; R⁴⁸ is hydrogen or C₁-C₈ alkyl;
R⁴⁹ independently are —COOH, —(CH₂)ₙ—N(R⁵⁰)₂, —(CH₂)ₙ—SO₃H, or —(CH₂)ₙ—SO₃—C₁-C₈ alkyl; and
each R⁵⁰ independently are C₁-C₈ alkyl or —(CH₂)ₙ—COOH; subscript n is an integer ranging from 0 to 6; and X¹ is C₁-C₁₀ alkylene.

Embodiment 40. The Ligand Drug Conjugate composition of embodiment 39, wherein the secondary amine-containing auristatin compound has the structure of Formula D_{E}-1, Formula D_{E-2} or Formula D_{F-1}:

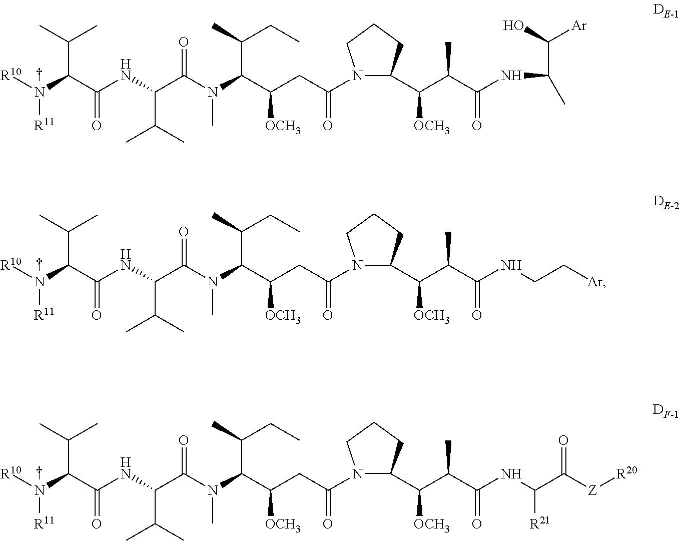

wherein Ar is C₆-C₁₀ aryl or C₅-C₁₀ heteroaryl, preferably Ar is phenyl or 2-pyridyl;
Z is —O— or —NH—; R²⁰ is hydrogen, C₁-C₆ alkyl, C₆-C₁₀ aryl or C₅-C₁₀ heteroaryl, wherein C₁-C₆ alkyl, C₆-C₁₀ aryl and C₅-C₁₀ heteroaryl are optionally substituted; and R²¹ is C₁-C₆ alkyl, —C₁-C₆ alkylene-(C₆-C₁₀ aryl) or —C₁-C₆ alkylene-(C₅-C₁₀ heteroaryl), each of which is optionally substituted.

Embodiment 41. The Ligand Drug Conjugate composition of embodiment 40, wherein the secondary amine-containing auristatin compound has the structure of Formula D_{F-1}
wherein R²¹ is X¹—S—R^{21a} or X¹—Ar, wherein X¹ is C₁-C₆ alkylene, R^{21a} is C₁-C₄ alkyl and Ar is phenyl or C₅-C₆ heteroaryl; and
—Z— is —O— and R²⁰ is C₁-C₄ alkyl, or
—Z— is —NH— and R²⁰ is phenyl or C₅-C₆ heteroaryl.

Embodiment 42. The Ligand Drug Conjugate composition of embodiment 40, wherein the secondary amine-containing auristatin compound has the structure of Formula In preferred embodiments the auristatin drug compound has the structure of Formula D_{F/E-3}:

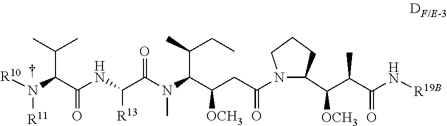

wherein one of R¹⁰ and R¹¹ is hydrogen and the other is methyl;
R¹³ is isopropyl or —CH₂—CH(CH₃)₂; and
R^{19B} is —CH(CH₃)—CH(OH)-Ph, —CH(CO₂H)—CH(OH)—CH₃, —CH(CO₂H)—CH₂Ph, —CH(CH₂Ph)-2-thiazolyl, —CH(CH₂Ph)-2-pyridyl, —CH(CH₂-p-Cl-Ph), —CH(CO₂Me)-CH₂Ph, —CH(CO₂Me)-CH₂CH₂SCH₃, —CH(CH₂CH₂SCH₃)C(═O)NH-quinol-3-yl, —CH(CH₂Ph)C(═O)NH-p-Cl-Ph, or
R^{19B} has the structure of

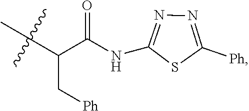 [SMILES: *C(Cc1ccccc1)C(=O)Nc1nnc(-c2ccccc2)s1]

wherein the wavy line indicates covalent attachment to the remainder of the auristatin compound.

Embodiment 43. The Ligand Drug Conjugate composition of embodiment 40 wherein the secondary amine-containing auristatin compound is monomethylauristatin E (MMAE) or monomethylauristatin F (MMAF).

Embodiment 44. The Ligand Drug Conjugate composition of embodiment 1, wherein subscript q is 1 and a majority of Ligand Drug Conjugate compounds in the Ligand Drug Conjugate composition have drug linker moieties represented by the structures of Formula 1C-MMAE and Formula 1D-MMAE:

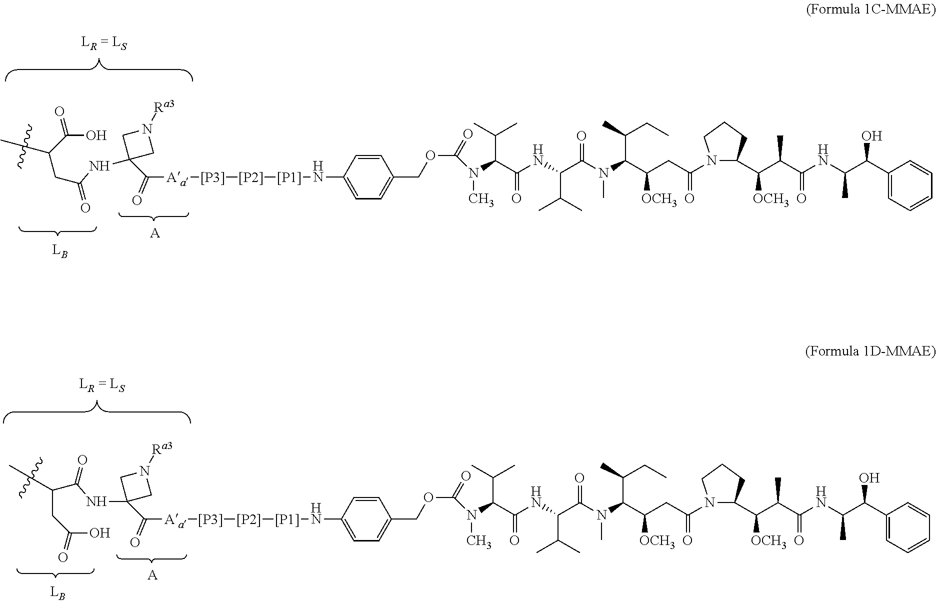

or salts thereof, in particular a pharmaceutical acceptable salts, wherein
A′ is a subunit, when present, of the indicated first Stretcher Unit (A) having the structure of formula 3a, formula 4a or formula 5a of embodiment 22, or an α-amino acid or β-amino acid residue, in particular —NH—CH₂CH₂—C(═O)—;
R^{a3} is —H, optionally substituted C₁-C₆ alkyl, optionally substituted —C₁-C₄ alkylene-(C₆-C₁₀ aryl), or —R^{PEG1}—O—(CH₂CH₂O)₁₋₃₆—R^{PEG2}, wherein R^{PEG1} is C₁-C₄ alkylene, R^{PEG2} is —H or C₁-C₄ alkylene, and wherein the basic nitrogen bonded to R^{a3} is optionally protonated in a salt form, preferably in a pharmaceutically acceptable salt form, or
R^{a3} is a nitrogen protecting group such as a suitable acid-labile protecting group; and
the wavy line indicates the site of covalent binding to a sulfur atom of the Ligand Unit.

Embodiment 45. The Ligand Drug Conjugate composition of embodiment 1, wherein subscript q is 1 and a majority of Ligand Drug Conjugate compounds in the Ligand Drug Conjugate composition have drug linker moieties represented by the structures of Formula 1F-MMAE and Formula 1G-MMAE:

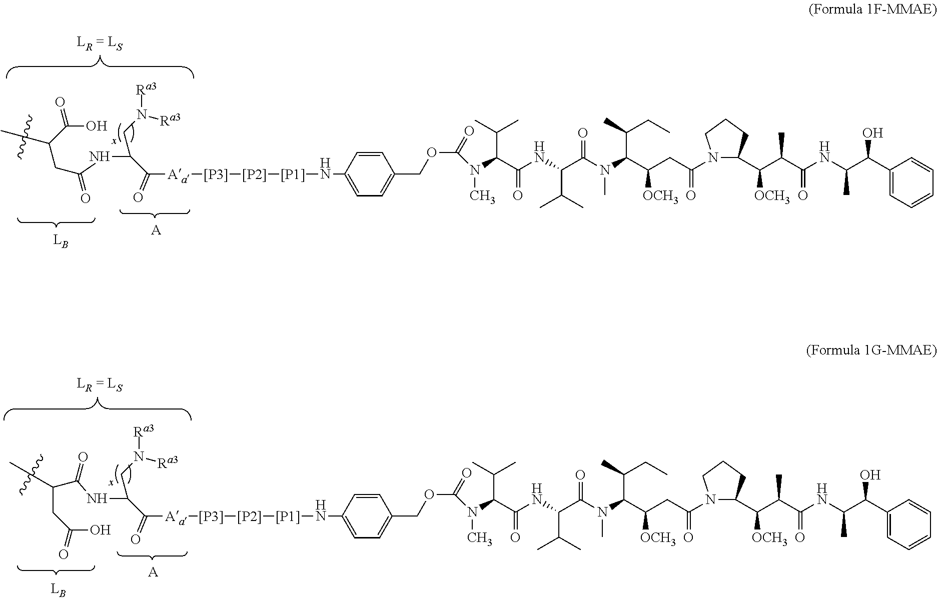

or salt thereof, in particular a pharmaceutical acceptable salts, wherein
A′ is a subunit, when present, of the indicated first Stretcher Unit (A) having the structure of formula 3a, formula 4a or formula 5a of embodiment 22, or an α-amino acid or β-amino acid residue, in particular —NH—CH₂CH₂—C(═O)—;
subscript x is 1 or 2;
R^{a3}, at each instance, is independently a nitrogen protecting group, —H or optionally substituted C₁-C₆ alkyl, preferably —H, an acid-labile protecting group, —CH₃ or —CH₂CH₃,
or both R^{a3} together with the nitrogen to which they are attached define a nitrogen protecting group or an azetidinyl, pyrrolidinyl or piperidinyl heterocyclyl, in which a basic primary, secondary or tertiary amine so defined is optionally protonated in a salt form, preferably a pharmaceutically acceptable salt form; and the wavy line indicates the site of covalent binding to a sulfur atom of the Ligand Unit.

Embodiment 46. The Ligand Drug Conjugate composition of embodiment 1, wherein subscript q is 1 and the Ligand Drug Conjugate compounds in the Ligand Drug Conjugate composition predominately have drug linker moieties of Formula 1H-MMAE:

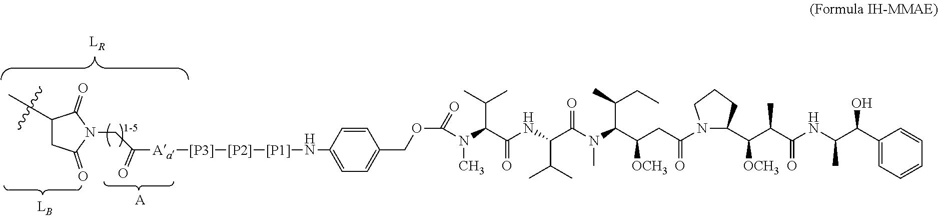

or a salt thereof, in particular a pharmaceutical acceptable salt, and optionally having a minority of Ligand Drug Conjugate compounds in which one or more of the drug linker moieties in each of such compounds has its the succinimide ring in hydrolyzed form and wherein
A′ is a subunit, when present, of the indicated first Stretcher Unit (A) having the structure of formula 3a, formula 4a or formula 5a of embodiment 22, or an α-amino acid or β-amino acid residue, in particular —NH—CH₂CH₂—C(═O)—;
subscript a′ is 0 or 1, indicating the absence or presence of A′; and
the wavy line indicates the site of covalent binding to a sulfur atom of the Ligand Unit.

Embodiment 47. The Ligand Drug Conjugate composition of embodiment 44, 45 or 46, wherein P1 is L-Glu or L-Asp, P2 is L-Val or L-Ala and P3 is L-Leu or D-Leu.

Embodiment 48. The Ligand Drug Conjugate composition of embodiment 1, wherein subscript q is 1 and wherein the predominate drug linker moiety in a majority of Ligand Drug Conjugate compounds of the composition is represented by the structure of:

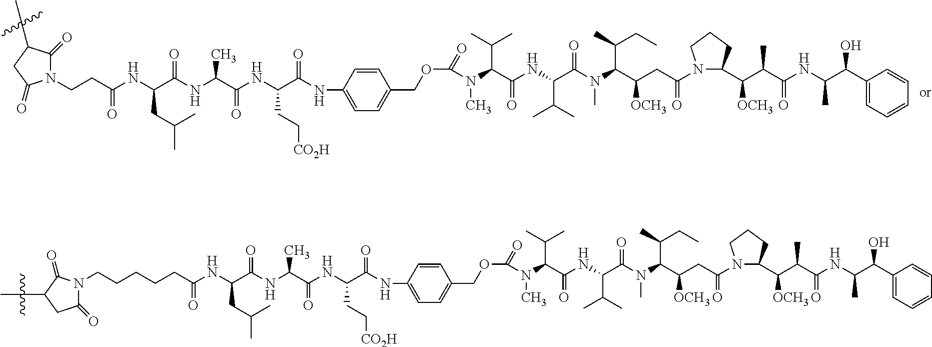 [SMILES: *C1CC(=O)N(CCC(=O)N[C@H](CC(C)C)C(=O)N[C@@H](C)C(=O)N[C@@H](CCC(=O)O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C1=O ; *C1CC(=O)N(CCCCCC(=O)N[C@H](CC(C)C)C(=O)N[C@@H](C)C(=O)N[C@@H](CCC(=O)O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C1=O]

or a salt thereof, in particular a pharmaceutical acceptable salt, and optionally having a minority of Ligand Drug Conjugate compounds in which one or more of the drug linker moieties in each of such compounds has its the succinimide ring in hydrolyzed form

Embodiment 49. The Ligand Drug Conjugate composition of any one of embodiments 1-48, wherein L is an antibody Ligand Unit of an intact antibody or an antigen-binding fragment thereof.

Embodiment 50. The Ligand Drug Conjugate composition of embodiment 49, wherein the intact antibody or fragment thereof is capable of selectively binding to a cancer cell antigen.

Embodiment 51. The Ligand Drug Conjugate composition of embodiment 49, wherein the intact antibody is a chimeric, humanized or human antibody, wherein the antibody is capable of selectively binding to a cancer cell antigen or the antibody is a non-binding control antibody thereby defining a non-binding control Conjugate composition.

Embodiment 52. The Ligand Drug Conjugate composition of any one of embodiments 1-51, wherein subscript p ranges from about 2 to about 12, or from about 2 to about 10, or from about 2 to about 8, in particular subscript p is about 2, about 4 or about 8.

Embodiment 53. A pharmaceutically acceptable formulation, wherein the formulation comprises an effective amount of a Ligand Drug Conjugate composition or an equivalent amount of a non-binding control Conjugate of any one of embodiments 1 to 36 and at least one pharmaceutically acceptable excipient.

Embodiment 54. The pharmaceutically acceptable formulation of embodiment 53, wherein the least one pharmaceutically acceptable excipient is a liquid carrier that provides a liquid formulation, wherein the liquid formulation is suitable for lyophilization or administration to a subject in need thereof and.

Embodiment 55. The pharmaceutically acceptable formulation of embodiment 53, wherein the formulation is a solid from lyophilization or a liquid formulation of embodiment 54, wherein the at least one excipient of the solid formulation is a lyoprotectant.

Embodiment 56. A Drug Linker compound of Formula IA:

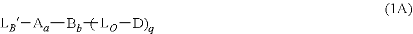

or a salt thereof, wherein
D is a Drug Unit;
L_{B}′ is a ligand covalent binding precursor moiety;
A is a first optional Stretcher Unit;
subscript a is 0 or 1, indicating the absence or presence of A, respectively;
B is an optional Branching Unit;
subscript b is 0 or 1, indicating the absence or presence of B, respectively;
L_{O} is a secondary linker moiety, wherein the secondary linker has the formula of;

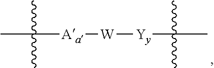

wherein the wavy line adjacent to Y indicates the site of covalent attachment of L_{O} to the Drug Unit and the wavy line adjacent to A′ indicates the site of covalent attachment to the remainder of the Drug Linker compound;
A′ is a second optional Stretcher Unit, which in the absence of B becomes a subunit of A;
subscript a′ is 0 or 1, indicating the absence or presence of A′, respectively,
W is a peptide Cleavable Unit, wherein the peptide Cleavable Unit is a contiguous sequence of up to 12 amino acids, wherein the sequence is comprised of a selectivity conferring tripeptide whose N-terminus provides an amide linkage that is selectively cleavable by a homogenate of tumor tissue to release free drug in comparison to a homogenate of normal tissue, and/or provides improved bioavailability to tumor tissue of a Ligand Drug Conjugate compound of Formula 1 of embodiment 1 in which the Drug Linker compound becomes a drug linker moiety of the Conjugate compound to the detriment of bioavailability to normal tissue in comparison to a comparator Ligand-Drug Conjugate in which the peptide sequence of its peptide Cleavable Unit is the dipeptide -valine-citrulline-;
wherein the tumor and normal tissues are of the same species and wherein an adverse event associated with release of free drug from the comparator Ligand-Drug Conjugate when administered in an effective amount to a subject in need thereof is due to its toxicity towards cells of the normal tissue.
Y is a self-immolative Spacer Unit;
subscript y is 0, 1 or 2 indicating the absence or presence of 1 or 2 of Y, respectively; and
subscript q is an integer ranging from 1 to 4,
provided that subscript q is 1 when subscript b is 0 and subscript q is 2, 3 or 4 when subscript b is 1.

Embodiment 57. The Drug Linker compound of embodiment 56, wherein the Drug Linker compound has the formula of:

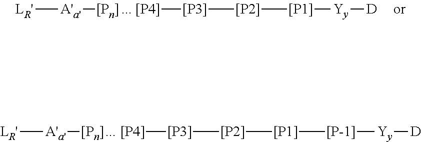

or a salt thereof, wherein
L_{R}′ is a primary linker of formula L_{B}′-Aₐ-B_{b}—, provided that A′ is a subunit of A so that A′ is a component of L_{R}′ when subscript a and a′ are each 1 and subscript b is 0; and
each P is an amino acid residue of the contiguous amino acid sequence of the peptide Cleavable Unit and wherein subscript n has an integer value providing for up to 12 of these residues,
wherein —[P3]-[P2]-[P1]- of the sequence is the selectivity conferring tripeptide.

Embodiment 58. The Drug Linker compound of embodiment 57, wherein the Drug Linker compound has the formula of:

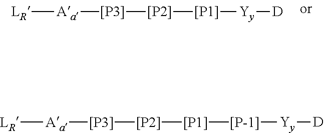

or a salt thereof,
wherein L_{R}′ is a primary linker of formula L_{B}′-Aₐ-B_{b}—, provided that A′ is a subunit of A so that A′ is a component of L_{R}′ when subscript a and a′ are each 1 and subscript b is 0; and
wherein each P is an amino acid residue of the contiguous amino acid sequence of the peptide Cleavable Unit, wherein —[P3]-[P2]-[P1]- of the sequence is the selectivity conferring tripeptide.

Embodiment 59. The Drug Linker compound of embodiment 58, wherein the Drug Linker compound has the formula of:

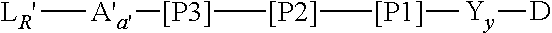

or a salt thereof, wherein P1 is a L-amino acid residue having at physiological pH a negatively charged side chain or a non-positively charged polar side chain.

Embodiment 60. The Drug Linker compound of any one of embodiments 56-59, wherein P1 is a L-amino acid residue selected from the group consisting of glutamic acid, methionine-sulfoxide, aspartic acid, (S)-3-aminopropane-1,1,3-tricarboxylic acid and phospho-threonine.

Embodiment 61. The Drug Linker compound of embodiment 56, wherein the Drug Linker compound has the formula of:

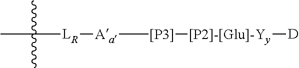

or a salt thereof, in particular a pharmaceutically acceptable salt, wherein each P is an amino acid residue of the contiguous amino acid sequence of the peptide Cleavable Unit.

Embodiment 62. The Drug Linker compound of any one of embodiments 56-61, wherein P2 is a residue of glycine or an L-amino acid, the side chain of which has no more than three contiguous carbon atoms.

Embodiment 63. The Drug Linker compound of embodiment 62, wherein the P2 amino acid is L-alanine, L-valine or glycine or an unnatural amino acid, wherein the unnatural amino acid is Abu, Aib, Ala, Gly, Leu, Nva or Pra which have the structures of:

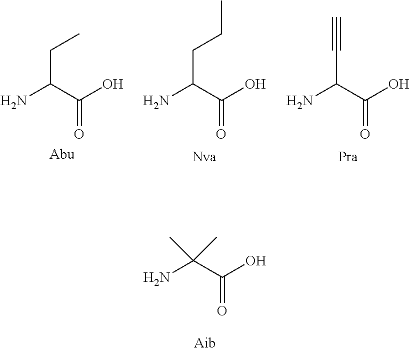

wherein the side chains of Abu, Nva and Pra are in the same stereochemical configuration of an L-amino acid.

Embodiment 64. The Drug Linker compound of embodiment 63, wherein the Drug Linker compound has the formula of:

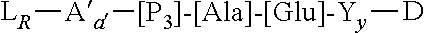

or a salt thereof, wherein P3 is an amino acid residue of the contiguous amino acid sequence of the peptide Cleavable Unit.

Embodiment 65. The Drug Linker compound of any one of embodiments 56-64 wherein P3 is a D-amino acid, the side chain of which is uncharged at physiological pH.

Embodiment 66. The Drug Linker compound of any one of embodiments 56-64 wherein P3 is a D-Leu, L-Leu, L-Cit or L-Pro, preferably D-Leu.

Embodiment 67. The Drug Linker compound of embodiment 66, wherein —[P3]-[P2]-[P1]- is -D-Leu-Ala-Glu-, or a salt thereof, in particular a pharmaceutically acceptable salt.

Embodiment 68. The Drug Linker compound of any one of embodiments 56-67, wherein L_{B}′ is a maleimide moiety capable of reacting with a thiol functional group of a targeting moiety to form a thio-substituted succinimide moiety.

Embodiment 69. The Drug Linker compound of any one of embodiments 56-67, wherein L_{B}′-A- has or is comprised of one of the structures of:

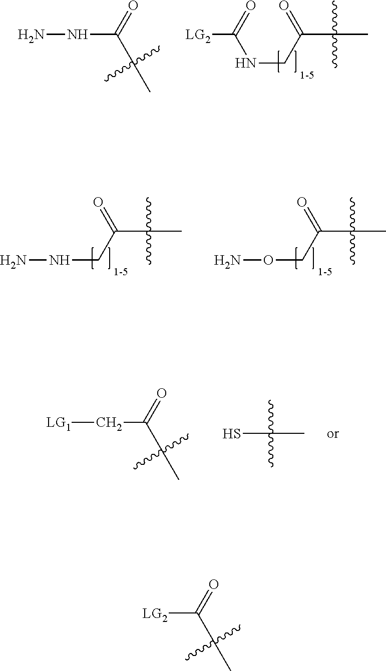

or a salt thereof, wherein
LG₁ is a leaving group suitable for nucleophillic displacement by a targeting agent nucleophile;
LG₂ is a leaving group suitable for amide bond formation to a targeting agent, or —OH to provide an activateable carboxylic acid suitable for amide bond formation to a targeting agent; and
the wavy line indicates the site of covalent attachment to the remainder of the Drug Linker compound structure.

Embodiment 70. The Drug Linker compound of embodiment 69, wherein subscript q is 1 and L_{B}′-A- has the structure of:

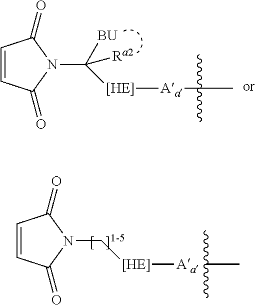

or a salt thereof, wherein
the wavy line adjacent to A′_{a′} indicates the site of covalent attachments to the Peptide Cleavable Unit;
[HE] is an optional Hydrolysis Enhancing Unit, which is a component provided by A or a first subunit thereof;
BU is a Basic Unit;
R^{a2} is an optionally substituted C₁-C₁₂ alkyl group; and
the dotted curved line indicates optional cyclization so that in the absence of said cyclization, BU is an acyclic Basic Unit having a primary, secondary or tertiary amine functional group as the basic function group of the acyclic Basic Unit, or in the presence of said cyclization BU is a cyclized Basic Unit in which R^{a2} and BU together with the carbon atom to which both are attached, define an optionally substituted spiro C₃-C₂₀ heterocyclo containing a skeletal basic nitrogen atom of a secondary or tertiary amine functional group as the basic function group of the cyclic Basic Unit,
wherein the basic nitrogen atom of the acyclic Basic Unit or cyclic Basic Unit is optionally suitably protected by a nitrogen protecting group, dependent on the degree of substitution of the basic nitrogen atom or is optionally protonated as an acid addition salt.

Embodiment 71. The Drug Linker compound of embodiment 70, wherein L_{B}′-A- has the structure of:

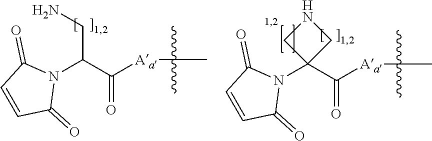

or a salt thereof, in particular as an acid addition salt, or wherein L_{B}′-A- has the structure of:

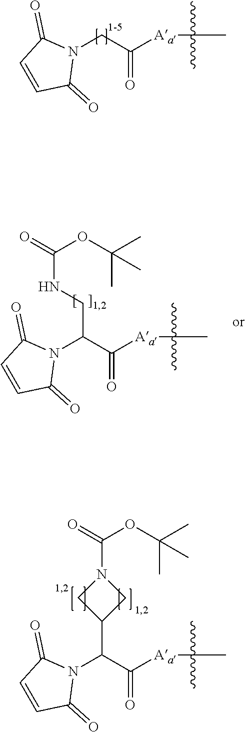

Embodiment 72. The Drug Linker compound of any one of embodiments 56-71, wherein subscript q is 1 and A′ is present as a subunit of A, wherein A′ is comprised of an amine-containing acid residue having the structure of formula (3) or formula (4):

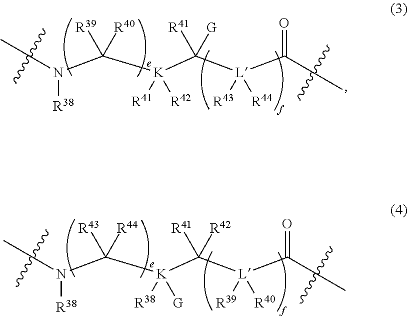

or a salt thereof, wherein
the wavy line adjacent to the nitrogen atom indicates the site of covalent attachment to [HE], wherein [HE] is —C(═O)— and the wavy line adjacent to the carbonyl carbon atom indicates the site of covalent attachment to the remainder of A′ or to the N-terminal amino acid residue of the Peptide Cleavable Unit, wherein both attachments are through amide functional groups;
K and L′ independently are C, N, O or S, provided that when K or L′ is O or S, R⁴¹ and R⁴² to K or R⁴³ and R⁴⁴ to L′ are absent, and when K or L′ are N, one of R⁴¹, R⁴² to K or one of R⁴², R⁴³ to L′ are absent, and provided that no two adjacent L′ are independently selected as N, O, or S;
wherein subscripts e and f are independently selected integers that range from 0 to 12, and subscript g is an integer ranging from 1 to 12:
G is hydrogen, optionally substituted C₁-C₆ alkyl, —OH or —CO₂H;
R³⁸ is hydrogen or optionally substituted C₁-C₆ alkyl;
R³⁹-R⁴⁴ are independently selected from the group consisting of hydrogen, optionally substituted C₁-C₆ alkyl and optionally substituted C₅-C₁₀ (hetero)aryl, or
R³⁹, R⁴⁰ together with the carbon atom to which both are attached, or R⁴¹, R⁴² together with K to which both are attached when K is a carbon atom, define a C₃-C₆ carbocyclo, and R⁴¹-R⁴⁴ are as defined herein,
or R⁴³, R⁴⁴ together with L′ to which both are attached when L′ is a carbon atom define a C₃-C₆ carbocyclo, and R³⁹-R⁴² are as defined herein,
or R⁴⁰ and R⁴¹, or R⁴⁰ and R⁴³, or R⁴¹ and R⁴³ to together with the carbon atom or heteroatom to which both are attached and the atoms intervening between those carbon atoms and/or heteroatoms define a C₅-C₆ carbocyclo or a C₅-C₆ heterocyclo, and R³⁹, R⁴⁴ and the remainder of R⁴⁰-R⁴³ are as defined herein,
provided that when K is O or S, R⁴¹ and R⁴² are absent, and when K is N, one of R⁴¹, R⁴² is absent, and when L′ is O or S, R⁴³ and R⁴⁴ are absent, and when L′ is N, one of R⁴³, R⁴⁴ is absent, or
A′ is comprised of an alpha-amino, beta-amino or another amine-containing acid residue, wherein its amino nitrogen atom is covalently attached to the carbonyl carbon atom of HE, and its carboxylic acid carbonyl carbon atom is covalently attached to the remainder of A′ or to the N-terminal amino acid of the Peptide Cleavable Unit, wherein both covalent attachments are through amide functional groups.

Embodiment 73. The Drug Linker compound of embodiment 72, wherein A′ is an amine-containing acid residue having the structure of formula 3a, formula 4a or formula 5a:

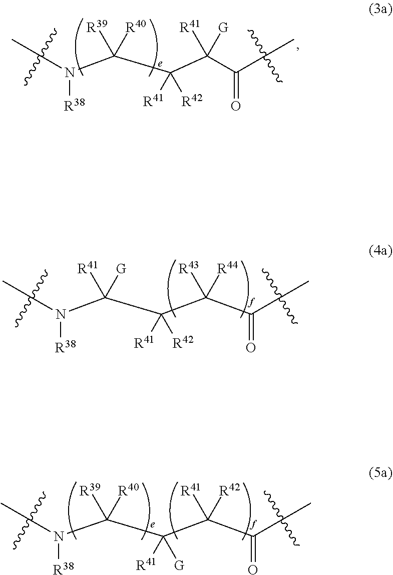

or a salt thereof, wherein
subscripts e and f are independently 0 or 1; and
R³⁸-R⁴⁴ are each hydrogen;
or A′ is an α-amino or β-amino acid residue.

Embodiment 74. The Drug Linker compound of any one of embodiments 56-71, wherein subscript q is 1 and A′ is comprised of a β-amino acid residue or -L^{P}(PEG)-,
wherein L^{P} is Parallel Connector Unit having the structure of Formula L^{P}-1 or L^{P}-2:

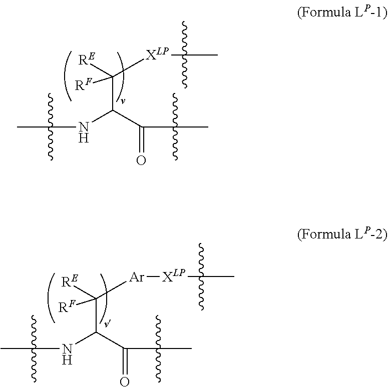

or
wherein -L^{P}(PEG)- or a PEG-containing subunit thereof has the structure of Formula L^{P}-3 or Formula L^{P}-4:

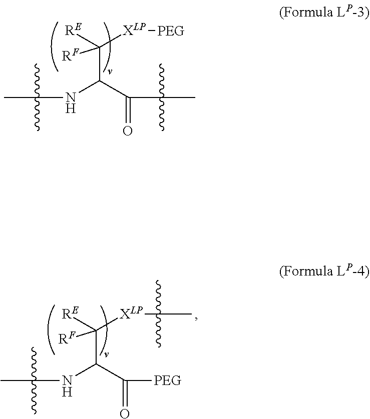

wherein subscript v is an integer ranging from 1 to 4;
subscript v′ is an integer ranging from 0 to 4;
X^{LP} is provided by a natural or un-natural amino acid side chain or is selected from the group consisting of —O—, —NR^{LP}—, —S—, —S(═O)—, —S(═O)₂—, —C(═O)—, —C(═O)N(R^{LP})—, —N(R^{LP})C(═O)N(R^{LP})—, and —N(R^{LP})C(═NR^{LP})N(R^{LP})—, or C₃-C₈ heterocyclo;
wherein each R^{LP} is independently selected from the group consisting of hydrogen and optionally substituted C₁-C₆ alkyl, or two of R^{LP} together along with the carbons atoms to which they are attached, and their intervening atoms define a C₅-C₆ heterocyclo and any remaining R^{LP} are as previously defined;
Ar is a C₆-C₁₀ arylene or a C₅-C₁₀ heteroarylene, optionally substituted;
each R^{E} and R^{F} is independently selected from the group consisting of —H, optionally substituted C₁-C₆ alkyl, optionally substituted C₂-C₆ alkylene, optionally substituted C₆-C₁₀ arylene or optionally substituted C₅-C₁₀ heteroarylene,
or R^{E} and R^{F} together with the carbon atom to which both are attached defines an optionally substituted spiro C₃-C₆ carbocyclo, or R^{E} and R^{F} from adjacent carbon atoms together with these atoms and any intervening carbon atoms defines an optionally substituted C₅-C₆ carbocyclo with any remaining R^{E} and R^{F} as previously defined;
wherein one of the wavy lines indicate the point of covalent attachment of a PEG Unit and the other two wavy lines indicates covalent attachment of Formula L^{P}-1 or Formula L^{P}-2 within the structure representing the Drug Linker Compound, or
L^{P} is Parallel Connector Unit having the structure of a tri-functional amine-containing acid residue or; and
PEG is a PEG Unit.

Embodiment 75. The Drug Linker compound of embodiment 74, wherein A′ is comprised of a β-amino acid residue or -L^{P}(PEG)-,
wherein the β-amino acid residue has the structure of —NHCH₂CH₂C(═O)—; and
wherein -L^{P}(PEG)- has the structure of:

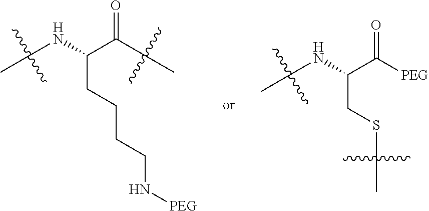

wherein the wavy lines indicate the sites of covalent attachment within the drug linker moiety.

Embodiment 76. The Drug Linker compound of embodiment 74 or 75, wherein the PEG Unit has the structure of:

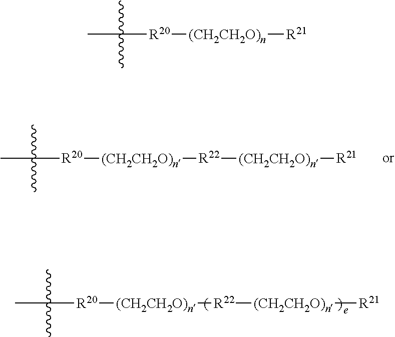

wherein the wavy line indicates the site of covalent attachment to L^{P};
R²⁰ is a PEG Attachment Unit, wherein the PEG Attachment Unit is —C(O)—, —O—, —S—, —S(O)—, —NH—, —C(O)O—, —C(O)C₁-C₁₀ alkyl, —C(O)C₁-C₁₀ alkyl-O—, —C(O)C₁-C₁₀ alkyl-CO₂—, —C(O)C₁-C₁₀alkyl-NH—, —C(O)C₁-C₁₀ alkyl-S—, —C(O)C₁-C₁₀alkyl-C(O)—NH—, —C(O)C₁-C₁₀alkyl-NH—C(O)—, —C₁-C₁₀ alkyl, —C₁-C₁₀alkyl-O—, —C₁-C₁₀alkyl-CO₂—, —C₁-C₁₀alkyl-NH—, —C₁-C₁₀ alkyl-S—, —C₁-C₁₀ alkyl-C(O)—NH—, —C₁-C₁₀ alkyl-NH—C(O)—, —CH₂CH₂SO₂—C₁-C₁₀ alkyl-, —CH₂C(O)—C₁₋₁₀ alkyl-, ═N—(O or N)—C₁-C₁₀ alkyl-O—, ═N—(O or N)—C₁-C₁₀ alkyl-NH—, ═N—(O or N)—C₁-C₁₀ alkyl-CO₂—, ═N—(O or N)—C₁-C₁₀ alkyl-S—,

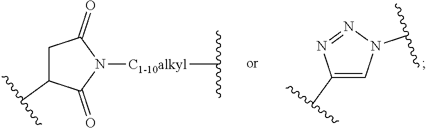

R²¹ is a PEG Capping Unit; wherein the PEG Capping Unit is —C₁-C₁₀ alkyl, —C₂-C₁₀ alkyl-CO₂H, —C₂-C₁₀ alkyl-OH, —C₂-C₁₀ alkyl-NH₂, C₂-C₁₀ alkyl-NH(C₁-C₃ alkyl), or C₂-C₁₀ alkyl-N(C₁-C₃ alkyl)₂;
R²² is an PEG Coupling Unit for coupling multiple PEG subunit chains together, wherein the PEG Coupling Unit is —C₁₋₁₀ alkyl-C(O)—NH—, —C₁₋₁₀ alkyl-NH—C(O)—, —C₂₋₁₀ alkyl-NH—, —C₂-C₁₀ alkyl-O—, —C₁-C₁₀ alkyl-S—, or —C₂-C₁₀ alkyl-NH—;
subscript n is independently selected from 8 to 72, from 10 to 72 or from 12 to 72;
subscript e is selected from 2 to 5; and
each n′ is independently selected from at least 6 to no more than 72, preferably from at least 8 or at least 10 to no more than 36.

Embodiment 77. The Drug Linker compound of embodiment 56, wherein the Drug Linker compound has the structure of Formula IC:

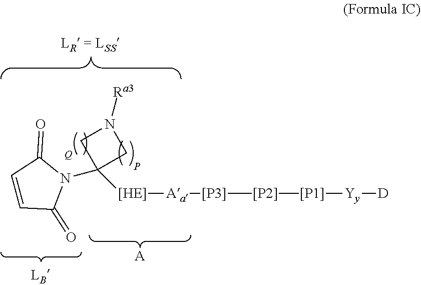

or a salt thereof, wherein
HE is a Hydrolysis Enhancing Unit;
A′ is an subunit, when present, of the indicated first Stretcher Unit (A);
subscript a′ is 0 or 1, indicating the absence or presence of A′, respectively;
subscript P is 1 or 2; and subscript Q ranges from 1 to 6, preferably subscript Q is 1 or 2, more preferably subscript Q has the same value as subscript P;
R^{a3} is —H, optionally substituted C₁-C₆ alkyl, optionally substituted —C₁-C₄ alkylene-(C₆-C₁₀ aryl), or —R^{PEG1}—O—(CH₂CH₂O)₁₋₃₆—R^{PEG2}, wherein R^{PEG1} is C₁-C₄ alkylene and R^{PEG2} is —H or C₁-C₄ alkylene, wherein the basic nitrogen bonded to R^{a3} is protonated in a salt form, or
R^{a3} is a suitable nitrogen protecting group, preferably a suitable acid-labile protecting group; and
each P is an amino acid residue of the contiguous amino acid sequence of the peptide Cleavable Unit.

Embodiment 78. The Drug Linker compound of embodiment 56, wherein the Drug Linker compound has the structure of Formula IF:

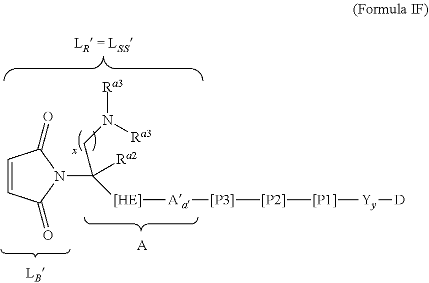

or a salt thereof, wherein
HE is a Hydrolysis Enhancing Unit;
A′ is an subunit, when present, of the indicated first Stretcher Unit (A);
subscript a′ is 0 or 1, indicating the absence or presence of A′, respectively;
subscript x is 1 or 2;
R^{a2} is —H, optionally substituted C₁-C₆ alkyl, —CH₃ or —CH₂CH₃;
R^{a3}, at each instance, is independently a suitable nitrogen protecting group, —H or optionally substituted C₁-C₆ alkyl, preferably —H, a suitable acid-labile protecting group, —CH₃ or —CH₂CH₃, provided that the nitrogen atom to which both R^{a3} are bound is protonated in salt form when neither R^{a3} is a nitrogen protecting group,
or both R^{a3} together with the nitrogen to which they are attached define a nitrogen protecting group or an azetidinyl, pyrrolidinyl or piperidinyl heterocyclyl, in which a basic primary, secondary or tertiary amine so defined is protonated in a salt form.

Embodiment 79. The Drug Linker compound of embodiment 78, wherein the Drug Linker compound has the structure of Formula IH:

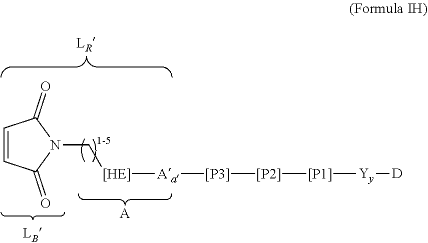

or salt thereof,
HE is a Hydrolysis Enhancing Unit; and
A′ is a subunit, when present, of the indicated first Stretcher Unit (A); subscript a′ is 0 or 1, indicating the absence or presence of A′.

Embodiment 80. The Drug Linker compound of embodiment 77, wherein the Drug Linker compound the structure of:

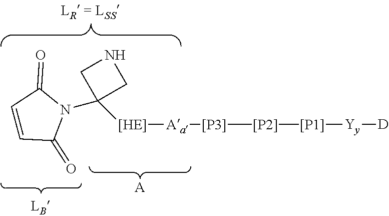

or a salt thereof, wherein the nitrogen atom of the 4-membered heterocyclo of L_{SS}′ is protonated in salt form.

Embodiment 81. The Drug Linker compound of embodiment 56, wherein the Drug Linker compound has the structure of:

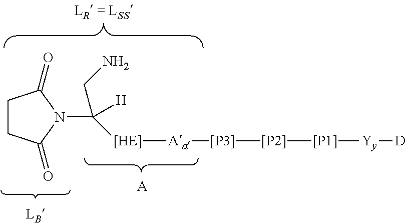

or salt thereof, wherein the primary amine of L_{SS}′ is protonated in salt form.

Embodiment 82. The Drug Linker compound of any one of embodiments 77-81 wherein HE is —C(═O).

Embodiment 83. The Drug Linker compound of any one of embodiments 77-81 wherein HE is —C(═O), subscript a′ is 1 and A′ has the structure of formula 3a, formula 4a or formula 5a of embodiment 73, or A′ is an α-amino acid or β-amino acid residue.

Embodiment 84. The Drug Linker compound of any one of embodiments 77-83, wherein —[P3]-[P2]-[P1]- is D-Leu-Leu-Cit, D-Leu-Leu-Lys, D-Leu-Leu-Met(O), Cit-Ala(Nap)-Thr, D-Leu-Ala-Glu or Pro-Ala(Nap)-Lys, wherein Met(O) is methionine in which its sulfur atom is oxidized to a sulfoxide and Ala(Nap) is alanine in which its methyl side chain is substituted by napthth-1-yl.

Embodiment 85. The Drug Linker compound of any one of embodiments 56-84, wherein —Y_{y}-D has the structure of:

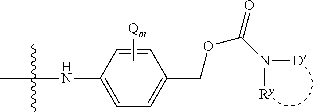

wherein —N(R^{y})D′ represents D, wherein D′ is the remainder of D;
the wavy line indicates the site of covalent attachment to P1 or P-1;
the dotted line indicates optional cyclization of R^{y} to D′;
R^{y} is optionally substituted C₁-C₆ alkyl in absence of cyclization to D′ or optionally substituted C₁-C₆ alkylene when cyclized to D′;
each Q is independently —C₁-C₈ alkyl, —O—(C₁-C₈ alkyl), or other electron donating group, -halogen, -nitro or -cyano or other electron withdrawing group, in particular each Q is independently selected from the group consisting of —C₁-C₈ alkyl, —O—(C₁-C₈ alkyl), halogen, nitro and cyano; and
subscript m is 0, 1 or 2, in particular subscript m is 0 or 1 and Q when present is an electron donating group, preferably subscript m is 0.

Embodiment 86. The Drug Linker compound of embodiment 56, wherein the Drug Linker compound has the structure of:

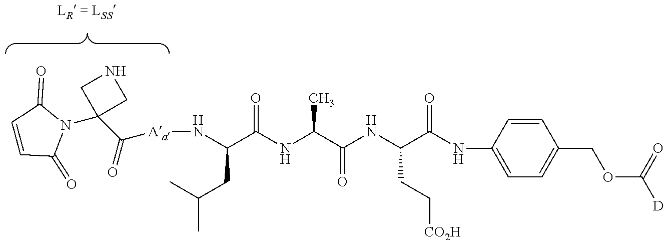

or a salt thereof, wherein
subscript a′ is 1, indicating the presence of A′, wherein A′ is an amine-containing acid residue of formula 3a, formula 4a or formula 5a of embodiment 73, or an α-amino acid or β-amino acid residue, in particular —NH—CH₂CH₂—C(═O)—; and
D is a cytotoxic drug having a secondary amino group as the site of attachment to the Linker Unit of the Drug Linker compound,
wherein the nitrogen atom of the heterocyclo of L_{SS}′ is protonated in salt form.

Embodiment 87. The Drug Linker compound of embodiment 56 wherein the Drug Linker compound has the structure of:

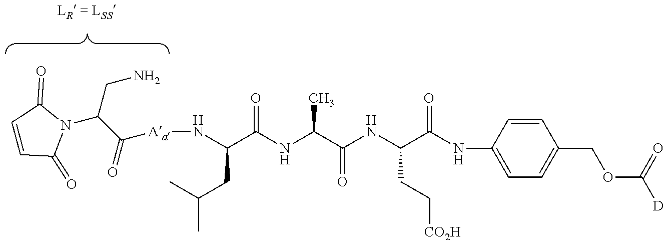

or a salt thereof, wherein
subscript a′ is 1, indicating the presence of A′, wherein A′ is an amine-containing acid residue of formula 3a, formula 4a or formula 5a of embodiment 73, or an α-amino acid or β-amino acid residue, in particular —NH—CH₂CH₂—C(═O)—; and
D is a cytotoxic drug having a secondary amino group as the site of attachment to the Linker Unit of the Drug Linker compound,
wherein the primary amine of L_{SS}′ is protonated in salt form.

Embodiment 88. The Drug Linker compound of embodiment 56 wherein the Drug Linker compound has the structure of:

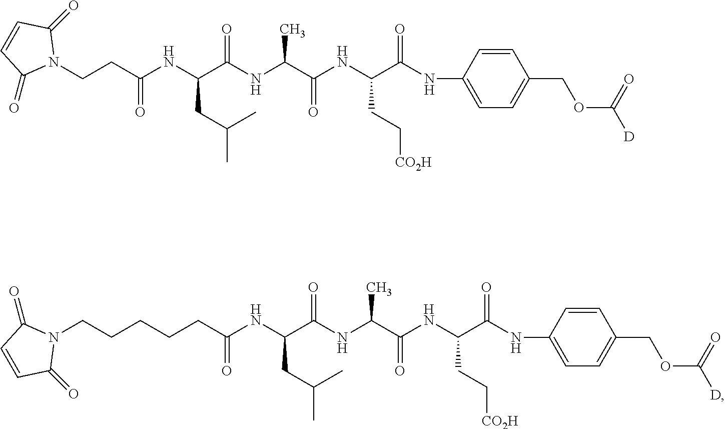

or a salt thereof, wherein
D is a cytotoxic drug having a secondary amino group as the site of attachment to the Linker Unit of the Drug Linker compound.

Embodiment 89. The Drug Linker compound of any one of embodiments 56-88 wherein subscript y′ is 2, wherein Y of —Y—Y′— is a first self-immolative Spacer Unit and Y′ is a second self-immolative Spacer Unit having the structure of —OC(═O)— and the cytotoxic drug is a secondary amine-containing auristatin compound wherein the nitrogen atom of the secondary amine is the site of covalent attachment to the carbonyl carbon atom of Y′ through a carbamate functional group shared between D and Y′.

Embodiment 90. The Drug Linker compound of embodiment 89, wherein the secondary amine-containing auristatin compound has the structure of Formula D_{E} or D_{F}:

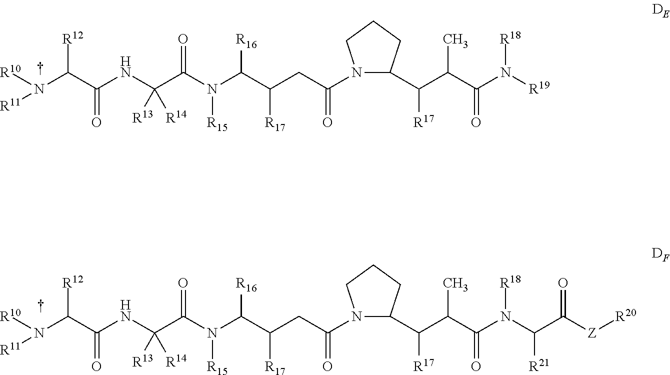

wherein the dagger indicates the site of covalent attachment of the nitrogen atom that provides the carbamate functional group,
one of R¹⁰ and R¹¹ is hydrogen and the other is C₁-C₈ alkyl, preferably one of R¹⁰ and R¹¹ is hydrogen and the other is methyl;
R¹² is hydrogen, C₁-C₈ alkyl, C₃-C₈ carbocyclyl, C₆-C₂₄ aryl, —X¹—C₆-C₂₄ aryl, —X¹—(C₃-C₈ carbocyclyl), C₃-C₈ heterocyclyl or —X¹—(C₃-C₈ heterocyclyl);
R¹³ is hydrogen, C₁-C₈ alkyl, C₃-C₈ carbocyclyl, C₆-C₂₄ aryl, —X¹—C₆-C₂₄ aryl, —X¹—(C₃-C₈ carbocyclyl), C₃-C₈ heterocyclyl and —X¹—(C₃-C₈ heterocyclyl);
R¹⁴ is hydrogen or methyl, or
R¹³ and R¹⁴ taken together with the carbon to which they are attached comprise a spiro C₃-C₈ carbocyclo;
R¹⁵ is hydrogen or C₁-C₈ alkyl;
R¹⁶ is hydrogen, C₁-C₈ alkyl, C₃-C₈ carbocyclyl, C₆-C₂₄ aryl, —C₆-C₂₄—X¹-aryl, —X¹—(C₃-C₈ carbocyclyl), C₃-C₈ heterocyclyl and —X¹—(C₃-C₈ heterocyclyl);
R¹⁷ independently are hydrogen, —OH, C₁-C₈ alkyl, C₃-C₈ carbocyclyl and O—(C₁-C₈ alkyl);
R¹⁸ is hydrogen or optionally substituted C₁-C₈ alkyl;
R¹⁹ is —C(R^{19A})₂—C(R^{19A})₂—C₆-C₂₄ aryl, —C(R^{19A})₂—C(R^{19A})₂—(C₃-C₈ heterocyclyl) or —C(R^{19A})₂—C(R^{19A})₂—(C₃-C₈ carbocyclyl), wherein C₆-C₂₄ aryl and C₃-C₈ heterocyclyl are optionally substituted;
R^{19A} independently are hydrogen, optionally substituted C₁-C₈ alkyl, —OH or optionally substituted —O—C₁-C₈ alkyl;
R²⁰ is hydrogen or C₁-C₂₀ alkyl, C₆-C₂₄ aryl or C₃-C₈ heterocyclyl, optionally substituted, or —(R⁴⁷O)ₘ—R⁴⁸, or —(R⁴⁷O)ₘ—CH(R⁴⁹)₂;
R²¹ is —C₁-C₈ alkylene-(C₆-C₂₄ aryl) or —C₁-C₈ alkylene-(C₅-C₂₄ heteroaryl), optionally substituted, or C₁-C₈ hydroxylalkyl, or optionally substituted C₃-C₈ heterocyclyl;
Z is O, S, NH, or NR⁴⁶.
R⁴⁶ is optionally substituted C₁-C₈ alkyl; subscript m is an integer ranging from 1-1000;
R⁴⁷ is C₂-C₈ alkyl; R⁴⁸ is hydrogen or C₁-C₈ alkyl;
R⁴⁹ independently are —COOH, —(CH₂)ₙ—N(R⁵⁰)₂, —(CH₂)ₙ—SO₃H, or —(CH₂)ₙ—SO₃—C₁-C₈ alkyl; and
R⁵⁰ independently are C₁-C₈ alkyl, or —(CH₂)ₙ—COOH; subscript n is an integer ranging from 0 to 6; and X¹ is C₁-C₁₀ alkylene.

Embodiment 91. The Drug Linker compound of embodiment 90 wherein the secondary amine-containing auristatin compound has the structure of Formula D_{E-1}, Formula D_{E-2} or Formula D_{F-1}:

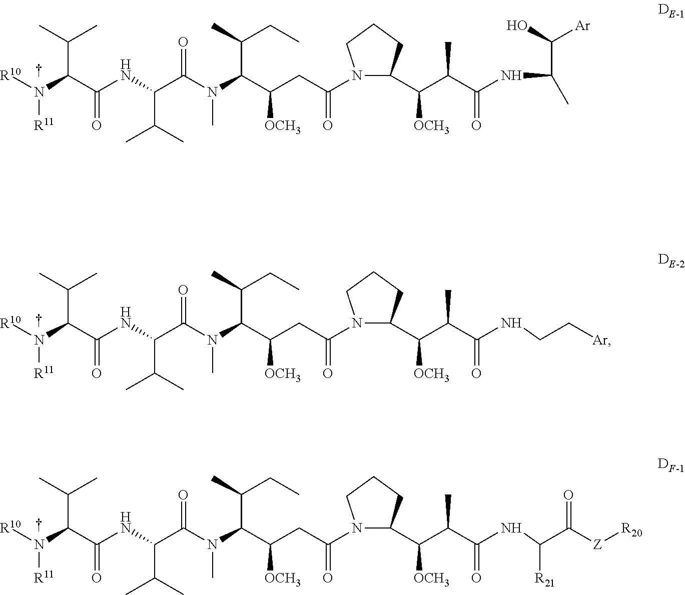

wherein Ar is C₆-C₁₀ aryl or C₅-C₁₀ heteroaryl, preferably Ar is phenyl or 2-pyridyl;
Z is —O—, or —NH—; R²⁰ is hydrogen or C₁-C₆ alkyl, C₆-C₁₀ aryl or C₅-C₁₀ heteroaryl, optionally substituted; and R²¹ is C₁-C₆ alkyl, —C₁-C₆ alkylene-(C₆-C₁₀ aryl) or —C₁-C₆ alkylene-(C₅-C₁₀ heteroaryl), optionally substituted.

Embodiment 92. The Drug Linker compound of embodiment 91, wherein the secondary amine-containing auristatin compound has the structure of Formula D_{F-1}
wherein R²¹ is X¹—S—R^{21a} or X¹—Ar, wherein X¹ is C₁-C₆ alkylene, R^{21a} is C₁-C₄ alkyl and Ar is phenyl or C₅-C₆ heteroaryl; and
—Z— is —O— and R²⁰ is C₁-C₄ alkyl, or
—Z— is —NH— and R²⁰ is phenyl or C₅-C₆ heteroaryl.

Embodiment 93. The Drug Linker compound of embodiment 91 wherein the secondary amine-containing auristatin compound has the structure of Formula In preferred embodiments the auristatin drug compound has the structure of Formula D_{F/E-3}:

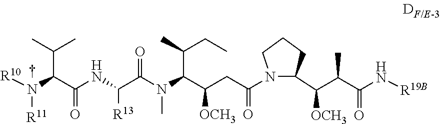

wherein one of R¹⁰ and R¹¹ is hydrogen and the other is methyl;
R¹³ is isopropyl or —CH₂—CH(CH₃)₂; and
R^{19B} is —CH(CH₃)—CH(OH)-Ph, —CH(CO₂H)—CH(OH)—CH₃, —CH(CO₂H)—CH₂Ph, —CH(CH₂Ph)-2-thiazolyl, —CH(CH₂Ph)-2-pyridyl, —CH(CH₂-p-Cl-Ph), —CH(CO₂Me)-CH₂Ph, —CH(CO₂Me)-CH₂CH₂SCH₃, —CH(CH₂CH₂SCH₃)C(═O)NH-quinol-3-yl, —CH(CH₂Ph)C(═O)NH-p-Cl-Ph, or
R^{19B} has the structure of

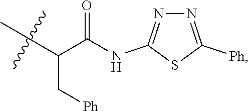 [SMILES: *C(Cc1ccccc1)C(=O)Nc1nnc(-c2ccccc2)s1]

wherein the wavy line indicates covalent attachment to the remainder of the auristatin compound.

Embodiment 94. The Drug Linker compound of embodiment 91 wherein the secondary amine-containing auristatin compound is monomethylauristatin E (MMAE) or monomethylauristatin F (MMAF).

Embodiment 95. The Drug Linker compound of embodiment 56, wherein the Drug Linker compound has the structure of Formula IC-MMAE:

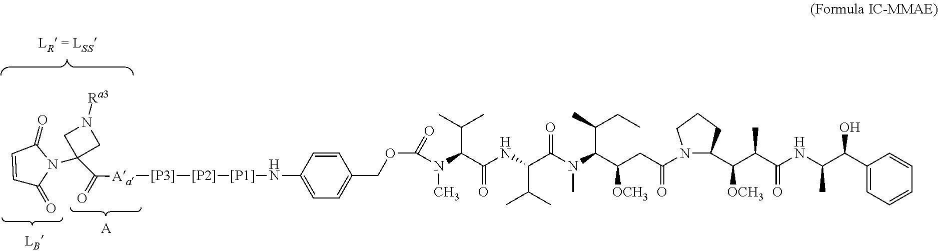

or salts thereof, in particular a pharmaceutical acceptable salts, wherein
A′ is a subunit, when present, of the indicated first Stretcher Unit (A) having the structure of formula 3a, formula 4a or formula 5a of embodiment 73, or an α-amino acid or β-amino acid residue, in particular —NH—CH₂CH₂—C(═O)—;
R^{a3} is —H, optionally substituted C₁-C₆ alkyl, optionally substituted —C₁-C₄ alkylene-(C₆-C₁₀ aryl), or —R^{PEG1}—O—(CH₂CH₂O)₁₋₃₆—R^{PEG2}, wherein R^{PEG1} is C₁-C₄ alkylene, R^{PEG2} is —H or C₁-C₄ alkylene, and wherein the basic nitrogen bonded to R^{a3} is protonated in a salt form, or
R^{a3} is a suitable nitrogen protecting group, preferably a suitable acid-labile protecting group.

Embodiment 96. The Drug Linker compound of embodiment 56, wherein the Drug Linker compound has the structure of Formula IF-MMAE:

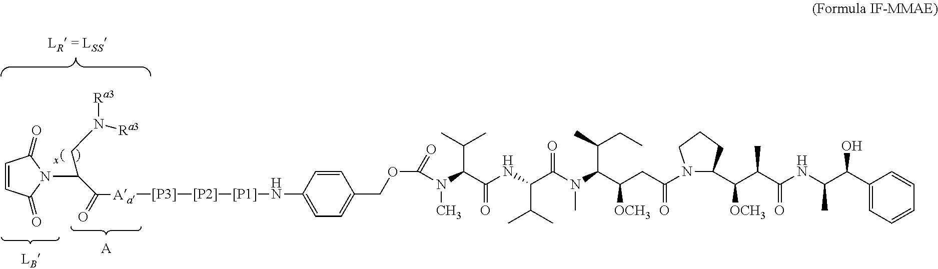

or a salt thereof, wherein
A′ is a subunit, when present, of the indicated first Stretcher Unit (A) having the structure of formula 3a, formula 4a or formula 5a of embodiment 73, or an α-amino acid or β-amino acid residue, in particular —NH—CH₂CH₂—C(═O)—;
subscript x is 1 or 2;
R^{a3}, at each instance, is independently a suitable nitrogen protecting group, —H or optionally substituted C₁-C₆ alkyl, preferably —H, a suitable acid-labile protecting group, —CH₃ or —CH₂CH₃, provided that the nitrogen atom to which both R^{a3} are bound is protonated in salt form when neither R^{a3} is a nitrogen protecting group,
or both R^{a3} together with the nitrogen to which they are attached define a nitrogen protecting group or an azetidinyl, pyrrolidinyl or piperidinyl heterocyclyl, in which a basic primary, secondary or tertiary amine so defined is protonated in a salt form.

Embodiment 97. The Drug Linker compound of embodiment 56, wherein the Drug Linker compound has the structure of Formula IH-MMAE:

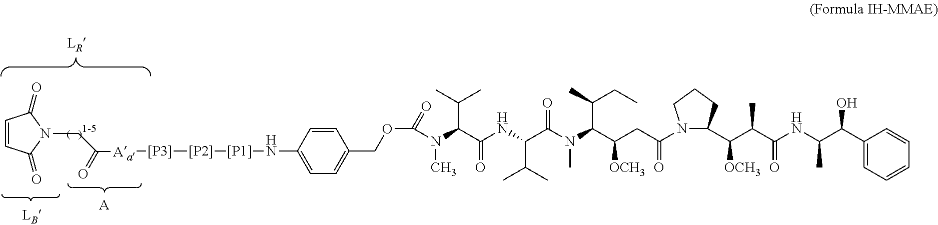

or a salt thereof, wherein
A′ is a subunit, when present, of the indicated first Stretcher Unit (A) having the structure of formula 3a, formula 4a or formula 5a of embodiment 73, or an α-amino acid or β-amino acid residue, in particular —NH—CH₂CH₂—C(═O)—;
subscript a′ is 0 or 1, indicating the absence or presence of A′.

Embodiment 98. The Drug Linker compound of embodiment 95, 96 or 97, wherein P1 is L-Glu or L-Asp, P2 is L-Val or L-Ala and P3 is L-Leu or D-Leu.

Embodiment 98. The Drug Linker compound of embodiment 956, 96 or 97, Drug Linker compound has the structure of:

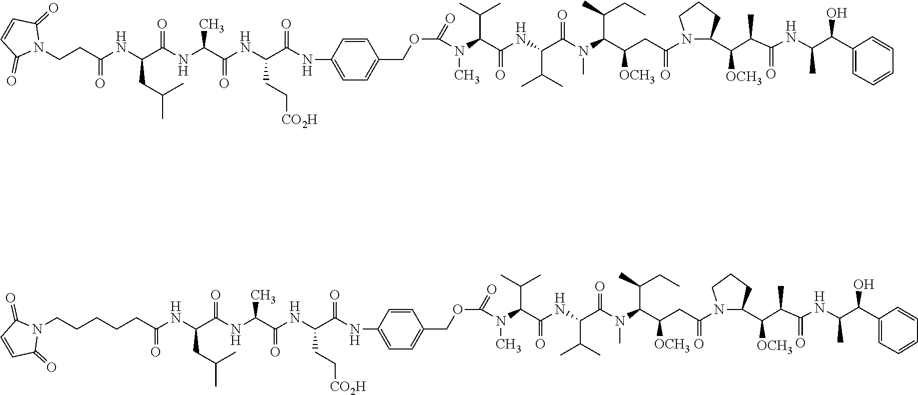 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCC(=O)O)NC(=O)[C@H](C)NC(=O)[C@@H](CC(C)C)NC(=O)CCN2C(=O)C=CC2=O)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCC(=O)O)NC(=O)[C@H](C)NC(=O)[C@@H](CC(C)C)NC(=O)CCCCCN2C(=O)C=CC2=O)cc1)C(C)C]

or a salt thereof.

### EXAMPLES

General Information. All commercially available anhydrous solvents were used without further purification. The UPLC-MS system used for characterizing the tripeptide-based Drug Linker compounds consisted of a Waters SQ mass detector interfaced to an Acquity Ultra Performance LC equipped with an Acquity UPLC BEH C18, 130 Å, 1.7 μm, 2.1×50 mm, reverse phase column or Waters Cortecs UPLC C18, 90 Å, 1.6 μm, 2.1×50 mm. The acidic mobile phase (0.1% formic acid) consisted of a gradient of 3% acetonitrile/97% water to 100% acetonitrile (flow rate=0.5 mL/min). UPLC-MS system 2 consisted of a Waters Xevo G2 ToF mass spectrometer interfaced to a Waters Acquity H-Class Ultra Performance LC equipped with an Acquity UPLC BEH C18 2.1×50 mm, 1.7 μm reverse phase column. Preparative HPLC was carried out on a Waters 2545 Binary Gradient Module with a Waters 2998 Photodiode Array Detector or a Teledyne ISCO ACCQPrep HP150. The tripeptide-based Drug Linker compounds were purified over a C12 Phenomenex Synergi™ 4 μm Max-RP 80 Å, LC column 250 mm of appropriate diameter eluting with 0.1% trifluoroacetic acid in water (solvent A) and 0.1% trifluoroacetic acid in acetonitrile (solvent B). The purification methods generally consisted of linear gradients of solvent A to solvent B, ramping from 90% aqueous solvent A to 10% solvent A. The flow rate was set according to the column requirements with monitoring at 220 nm. NMR spectral data were collected on a Varian Mercury 400 MHz spectrometer. Chemical shifts (6) are given in ppm relative to TMS. Coupling constants (J) are reported in hertz.

In vitro cytotoxicity. The cytotoxicity of a tripeptide-based Antibody Drug Conjugate was measured by a cell proliferation assay employing the protocol described in Promega Corp. Technical Bulletin TB288; and Mendoza et al., 2002, Cancer Res. 62:5485-5488), the methods of which are specifically incorporated by reference herein. Briefly, an aliquot of 40 μl of cell culture containing about 400 cells in medium is deposited in each well of a 384-well, opaque-walled plate. A 10 μL aliquot of free drug or Ligand-Drug Conjugate is added to the experimental wells and incubated for 96 h and are then equilibrated to room temperature for approximately 30 minutes whereupon a volume of CellTiter-Glo™ reagent equal to the volume of cell culture medium present in each well is added. The contents are mixed for 2 minutes on an orbital shaker to induce cell lysis and the plate is incubated at room temperature for 10 minutes to stabilize the luminescence signal for recordation.

Fluorescence assay. To a 384 well plate was added a mixture of tumor or normal tissue homogenate and citrate buffer (100 mM, pH 4.5; 9 μL) followed by a fluorescently labelled library compound (1 μL; dissolved in 50% MeCN). The reaction was incubated at 37° C. and fluorescence (330 nm excitation, 450 nm emission) was detected several times over a 6-hr. period. The fluorescence fold change was determined by dividing the fluorescence value at each time point with the background fluorescence when no homogenate was added.

Conjugation. Antibody was partially reduced using the appropriate equivalents of TCEP according to the procedure, which is specifically incorporated by reference herein, of US 2005/0238649. Briefly, the antibody in phosphate buffered saline with 2 mM EDTA, pH 7.4, was treated with 2.1 eq. TCEP and then incubated at 37° C. for about 45 minutes. The thiol/Ab value was checked by reacting the reduced antibody with compound 1 and using hydrophobic interaction chromatography to determine the loading.

The tripeptide-based auristatin Drug-Linker compounds were conjugated to the partially reduced antibody using the method, which is specifically incorporated by reference herein, of US 2005/0238649. Briefly, Drug-Linker compound (50% excess) in DMSO, was added to the reduced antibody in PBS with EDTA along with additional DMSO for a total reaction co-solvent of 10-20%. After 30 minutes at ambient temperature, an excess of QuadraSil MP™ was added to the mixture to quench all unreacted maleimide groups. The resulting Antibody Drug Conjugate was then purified, and buffer exchanged by desalting using Sephadex G25 resin into PBS buffer and kept at −80° C. until further use. The protein concentration of the resulting ADC composition was determined at 280 nm. The drug-antibody ratio (DAR) of the conjugate was determined by hydrophobic interaction chromatography (HIC).

In vivo cytotoxicity. Cancer cells were implanted into mice. After the tumor reached a volume of 100 mm³, ADC prepared from reduced antibody and tripeptide-based Drug linker compound was administered via an intraperitoneal injection. Tumor size was then measured twice a week until the end of the study.

Tissue Homogenization. Normal tissue or tumor tissue from mouse xenografts were suspended in buffer (50 mM Tris, 150 mM KCl, pH 7.0) and added to a tube containing Matrix D lysing beads (mpbio). The tissue was homogenized with a Precellys™ 24 homogenizer. The homogenized sample was centrifuged at 1000×g for 10 min and the resulting supernatant was collected then frozen at −80° C. until further use.

Toxicity Determinations. Each tripeptide-based Drug Linker compound was reacted with a reduced non-binding antibody to provide a non-binding control ADC and injected i.v. into female Sprague Dawley rats at various concentrations. Animals were euthanized on day 4 or 28 post dose.

### Example 1: Preparation of p-azido-benzyl alcohol (Az-PABA)

To a round bottomed flask was added p-amino-benzyl alcohol (100 mol %) suspended in 5 M HCl (5 mL per g PABA). The flask was cooled to 4° C. followed by the dropwise addition of aqueous NaNO₂ (150 mol %; 20 mL per gram PABA). NaN₃ was then added and the reaction was warmed to room temperature and incubated for 16 h. The reaction was diluted in saturated NaHCO₃ and extracted with EtOAc. The extract was dried with MgSO₄ and concentrated. The product was purified using an EtOAc/hexanes gradient (6%-42% EtOAc) with a SNAP-KP-Sil Biotage column yielding the title compound as an orange material (90% yield). ¹H-NMR (d₆-DMSO) δ 7.38-7.35 (C═CH, d, 2H), 7.11-7.07 (C═CH, d, 2H), 5.25-5.22 (OH, m, 1H), 4.50-4.46 (CH2, d, 2H)

### Example 2: Preparation of p-azido-benzyl bromide

To a round bottomed flask was added Az-PABA (100 mol %) dissolved in chloroform under a nitrogen atmosphere. To the solution was added PBr₃ (120 mol %) dropwise. The reaction was incubated for 2 h at which point it was diluted with CHCl₃ and washed with 1 M HCl followed by brine. The extract was dried with MgSO₄ and concentrated. The product was purified using an EtOAc/hexanes gradient (6%-42% EtOAc) with a SNAP-KP-Sil Biotage column to provide the title compound in 75% yield.

### Example 3: Preparation of methyl (2-(7-hydroxy-2-oxo-2H-chromen-4-yl)acetyl)glycinate (HO-Coum-Gly-OMe)

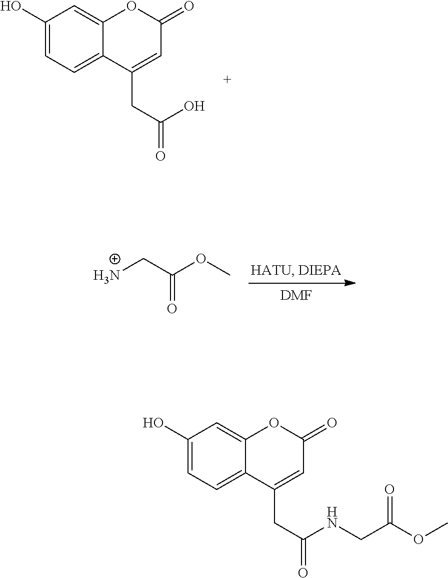

To a scintillation vial was added H-Gly-OMe (300 mol %) dissolved in DMF and DIPEA (350 mol %). To this vial was added 2-(6-hydroxy-2-oxo-2H-chromen-4-yl)acetic acid (100 mol %). DMF was then added until both reagents were fully dissolved. HATU (110 mol %) was then added followed by DIPEA (110 mol %) and the reaction was stirred for 45 min. At that time, the reaction was diluted in EtOAc and washed with 200 mM HCl. The aqueous layer was back extracted 3× with EtOAc. The combined organics were washed with brine, dried with MgSO₄, and concentrated to provide the title compound, which was purified in 80% yield from boiling isopropyl alcohol.

### Example 4: Preparation of methyl (2-(7-((4-azidobenzyl)oxy)-2-oxo-2H-chromen-4-yl)acetyl)glycinate (Az-PABE-Coum-Gly-OMe)

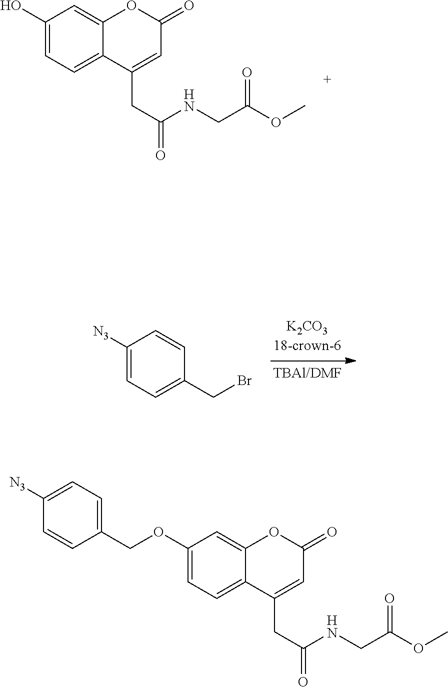 [SMILES: COC(=O)CNC(=O)Cc1cc(=O)oc2cc(O)ccc12 ; [N-]=[N+]=Nc1ccc(CBr)cc1 ; COC(=O)CNC(=O)Cc1cc(=O)oc2cc(OCc3ccc(N=[N+]=[N-])cc3)ccc12]

To a round bottomed flask was added HO-Coum-Gly-OMe (300 mol %), K₂CO₃ (150 mol %), and 18-crown-6 ether (200 mol %) suspended in DMF. After 15 min of vigorous stirring, Az-PAB-Br (100 mol %), prepared according to example 2, was added slowly in 4 separate aliquots. To the resulting solution was added tetrabutylammonium iodide (15 mol %), which was then stirred for 16 h. At that point the reaction was diluted into EtOAc and washed with 200 mM HCl and brine. The separated organic layer was dried with MgSO₄ and concentrated to provide the title compound as crude material that was used without further purification.

### Example 5: Preparation of (2-(7-((4-azidobenzyl)oxy)-2-oxo-2H-chromen-4-yl)acetyl)glycine (Az-PABE-Coum-Gly-OH)

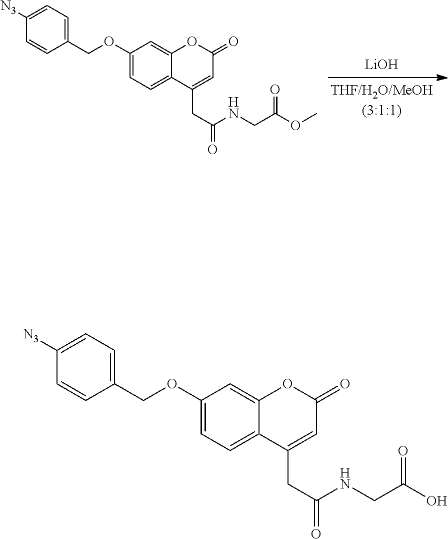 [SMILES: COC(=O)CNC(=O)Cc1cc(=O)oc2cc(OCc3ccc(N=[N+]=[N-])cc3)ccc12 ; "LiOH" ; [N-]=[N+]=Nc1ccc(COc2ccc3c(CC(=O)NCC(=O)O)cc(=O)oc3c2)cc1]

To a round bottomed flask was added crude Az-PABE-Coum-Gly-OMe (100 mol %) in THE (20 mL per 500 mg). To the vial was added MeOH (6 mL per 500 mg) and H₂O (6 mL per 500 mg). At that time LiOH (200 mol %) was added and the reaction was stirred for 1 h whereupon the reaction was diluted with EtOAc and washed twice with 200 mM HCl. The separated organic layer was dried with MgSO₄ and concentrated to provide the title compound in 88% yield. ¹H-NMR (d₇-DMF) δ 8.80 (NH, t, 1H), 8.03-8.01 (C═CH, d, 1H), 7.80-7.77 (C═CH, d, 2H), 7.38-7.36 (C═CH, d, 2H), 7.25 (C═CH, s, 1H), 7.24-7.20 (C═CH, d, 1H), 6.58 (C═CH, s, 1H), 5.47 (CH2, s, 2H), 4.14 (CH2, d, 2H), 4.08 (CH2, s, 2H).

Example 6: Preparation of P1-PABE-Coum-Gly-OH where P1=Fmoc-Leu-OH, Fmoc-D-Leu-OH, Fmoc-Ala-OH, Fmoc-Met-OH, Fmoc-Pro-OH, Fmoc-Cit-OH, Fmoc-Nal-OH, Fmoc-Tyr(All)-OH, Fmoc-Phe-OH, Fmoc-Lys(Mtt)-OH, Fmoc-Thr(Trt)-OH, Fmoc-Glu(0-2-PhiPr)—OH, wherein Cit is citrulline, and Nal is alanine in which its methyl side chain is substituted by napthth-1-yl.

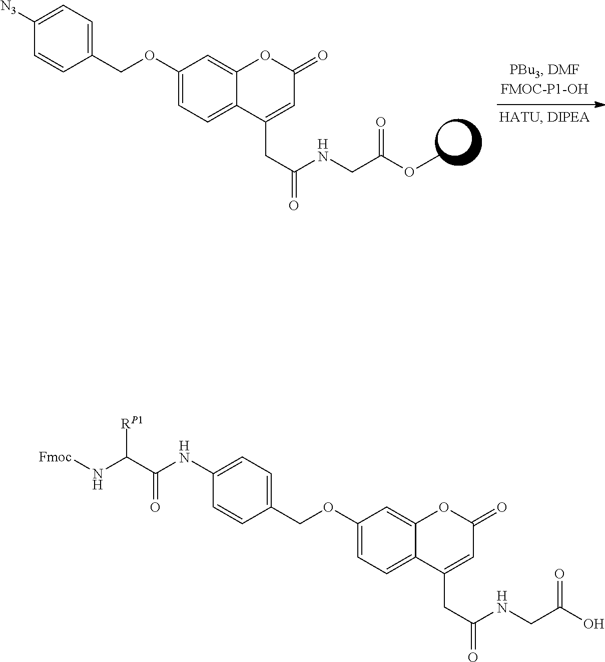

To resin (2-chloro-trityl chloride or rink acid; 100 mol %) swollen in dry DCM was added Az-PABE-Coum-Gly-OH (300 mol %) and DIPEA (310 mol %) dissolved in dry DCM. After mixing for 2 h, the solution was expelled and resin was washed with DCM. To an open round-bottomed flask was added Az-PABE-Coum-Gly-O-linked resin swollen in DMF followed by the addition of PBu₃ (250 mol %) and DIPEA (250 mol %). After mixing for 2 h, the solution was expelled and resin was washed with DMF, DCM, and Et₂O and dried overnight under vacuum. To a vial was added Fmoc-P1-OH (600 mol %) and HATU (600 mol %) dissolved in DMF, followed by DIPEA (800 mol %). The mixture was vortexed for 1 min and then added to the previously synthesized PBu₃ activated Az-PABE-Coum-Gly-O-linked resin (rink acid resin for Fmoc-Lys(Trt)-OH, Fmoc-Thr(Trt)-OH, and Fmoc-Glu(0-2-PhiPr), 2-chloro-trityl resin for all other amino acids) swollen in DMF. After mixing for 2 h, the solution was expelled and resin was washed with DMF and DCM. Fmoc-P1-PABE-Coum-Gly-OH was cleaved from resin using 0.2% TFA in DCM (for rink acid resin) or 5% TFA in DCM (for 2-chloro trityl resin) and purified by RP-HPLC.

### Example 7: Preparation and Screening of a Tripeptide Library

Dipeptide-based Conjugates that have previously been developed were designed to be cleavable by Cathepsin B, which is a lysosomal protease that is upregulated in cancer cells compared to normal cells of the same species. Exemplary comparator dipeptide-based Conjugates have drug linker moieties in which the Drug Unit is a residue of MMAE with one of the following structures.

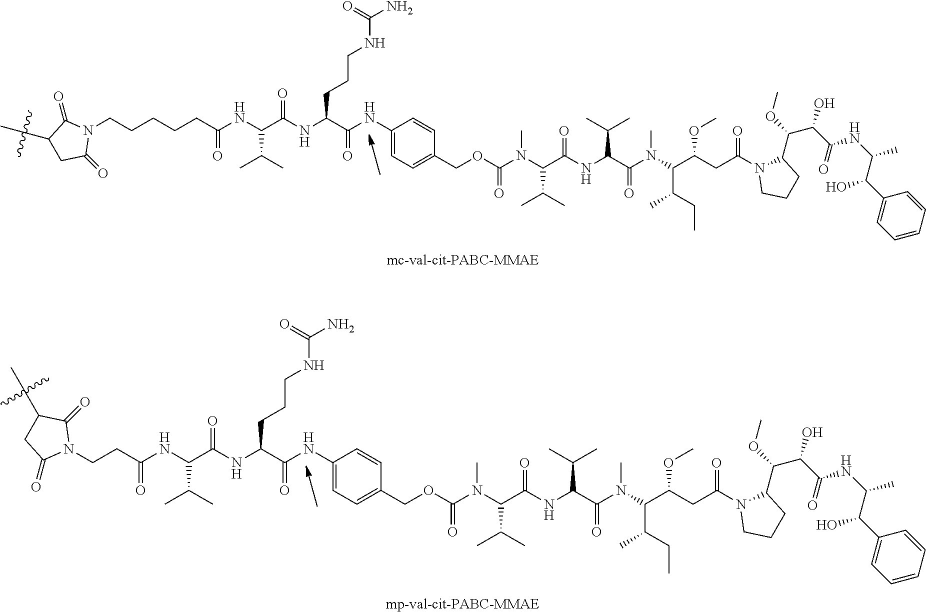

wherein the wavy line indicates the site of covalent attachment to a sulfur atom from the Ligand Unit and the arrow indicates the presumed site of proteolytic cleavage. Although more specific for Cathepsin B, other lysosomal proteases remain capable of that bond cleavage. To discover peptide sequences more specific for proteases upregulated in cancer tissue in comparison to proteases of normal tissue in which unwanted cytotoxicity towards normal cells in that tissue are associated with an adverse event when an effective amount of a comparator Conjugate have the shown dipeptide-based drug linker moieties, a library of fluorescent-quenched tripeptide-containing compounds was synthesized. Members of that library are models for Conjugate drug linker moieties in which a fluorescent tag replaces the Drug Unit and are collectively represented by the following structure:

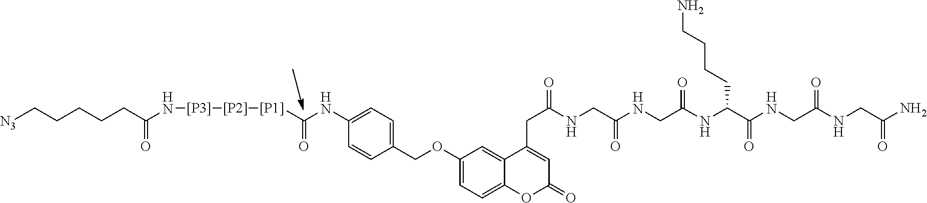

In the above structure, the conjugated coumarin moiety is non-fluorescent. Upon proteolytic cleavage of the indicated amide bond, a free coumarin-containing compound is released, which is now fluorescent. The Gly-Gly-D-Lys-Gly moiety of the free coumarin-containing compound is an artifact of the method in which the library was constructed, which is subsequently described herein. The azide provides a handle for attachment to a Ligand Unit by dipolar cycloaddition of the azide with a suitable alkyne moiety introduced onto the Ligand Unit.

The library was constructed using the non-aromatic hydrophobic amino acids Ala, Leu, Pro and D-Leu, the charged amino acids Glu and Lys, the uncharged hydrophilic amino acids Thr, Met and citrulline, and the hydrophobic aromatic amino acids Phe, Tyr (initially as the alloc-protected amino acid) and Nal (naphthyl-1-yl alanine). Thus, the library contains 1,728 distinct members. When Met is in the P1 position, the methyl sulfide group of its side chain undergoes spontaneous oxidation to the sulfoxide so that the P1 position is occupied by Met(O). When Met is in the P2 or P3 position, a mixture of tripeptides containing Met and Met(O) were obtained.

The library members were synthesized on cellulose support according to the method by Hilpert, K. et al. in “Peptide arrays on cellulose support: SPOT synthesis, a time and cost efficient method for synthesis of large numbers of peptides in a parallel and addressable fashion”, Nature Protocols (2007) 2(6): 1333-1349, the method of which is specifically incorporated by reference herein, with one important modification. That modification uses laser-perforated cellulose paper so that synthesis of each library member occurs with a clearly defined circular disc. After SPOT synthesis, each circular area, which separately contains a discrete library member, is punched out by a multichannel pipette into individual wells of a microtiter plate. The microtiter plate is then placed into an ammonia chamber to cleave the tripeptide containing model compounds from the cellulose discs. The cleaved compounds were then transferred to a fresh microtiter plate after solubilizing each into 50% aqueous acetonitrile. The contents of the wells were then assessed for susceptibility to proteolysis by tumor tissue homogenate in comparison to a comparator peptide-based drug linker compound having the dipeptide val-cit, which is replaced by the —[P1]-[P2]-[P3]- tripeptides in the library of drug linker compounds, by measuring the fluorescence found in each of the wells of the library after contacting with tumor or normal tissue homogenate and dividing it by the fluorescence found for tumor or normal tissue homogenate cleavage of the comparator dipeptide-containing drug linker compound.

The working assumption is that a ratio of tumor tissue to normal tissue proteolysis greater than that obtained for the comparator drug linker compound, which indicates more rapid cleavage in tumor tissue or slower cleavage in normal tissue of the library drug linker compound compared to the dipeptide-containing drug linker compound, will translate to greater selectivity for tumor tissue proteolysis in comparison to proteolysis by normal tissue homogenate of the same species, wherein cytotoxicity towards normal cells of the tissue by a comparator Conjugate having that compound as a dipeptide-based drug linker moiety responsible for an adverse event associated with administration of an effective amount of the comparator Conjugate to a subject in need thereof. The skilled person will understand that such a correlation may not hold for every library member and that the increase in proteolysis observed for the tripeptide-containing drug linker compound is not simply due to the tripeptide being a superior recognition site for Cathepsin B but instead is due at least in part to improved reactivity towards other proteases that are also upregulated in tumor tissue.

FMOC chemistry was used to prepare the Gly-D-Lys-Gly-Gly moiety, covalently attached to the cellulose solid support, in which the cellulose hydroxyl groups of the laser-perforated cellulose paper were first modified as glycine esters. The FMOC group was then removed to provide a free amine, which was confirmed by a pH sensitive indicator. The next amino acid was added, and the process was repeated. For compatibility with a 96-well microtiter plate, the laser perforated discs were 6 mm in diameter to which are added 1 μL aliquots of the FMOC protected amino acid solutions.

FMOC-P1-PABE-Coum-Gly-OH, prepared according to Example 6, was then attached to the free amino group of the NH₂-Gly-Lys-Gly-Gly- residue. The key step in the reaction sequence in Example 6 is reduction of the resin-bound azide intermediate, which provides an iminophosphorane intermediate that is stable enough towards self-immolation in order for the coupling reaction with the first incoming FMOC-amino acid to occur. The P2 and P3 amino acids were then added by standard FMOC chemistry followed by acylation of the free amino group of the deprotected P3 residue to provide the resin-bound library compound, which was cleaved from the resin using an ammonia gas chamber. In Scheme 1, R^{P1}, R^{P2} and R^{P3} are the amino acid side chains of the P1, P2 and P3 amino acid residues, respectively, and X represents the other amino acids in the NH₂-Gly-Gly-D-Lys-Gly-Gly- pentapeptide that tethers the fluorescently labelled tripeptide to the cellulose solid support.

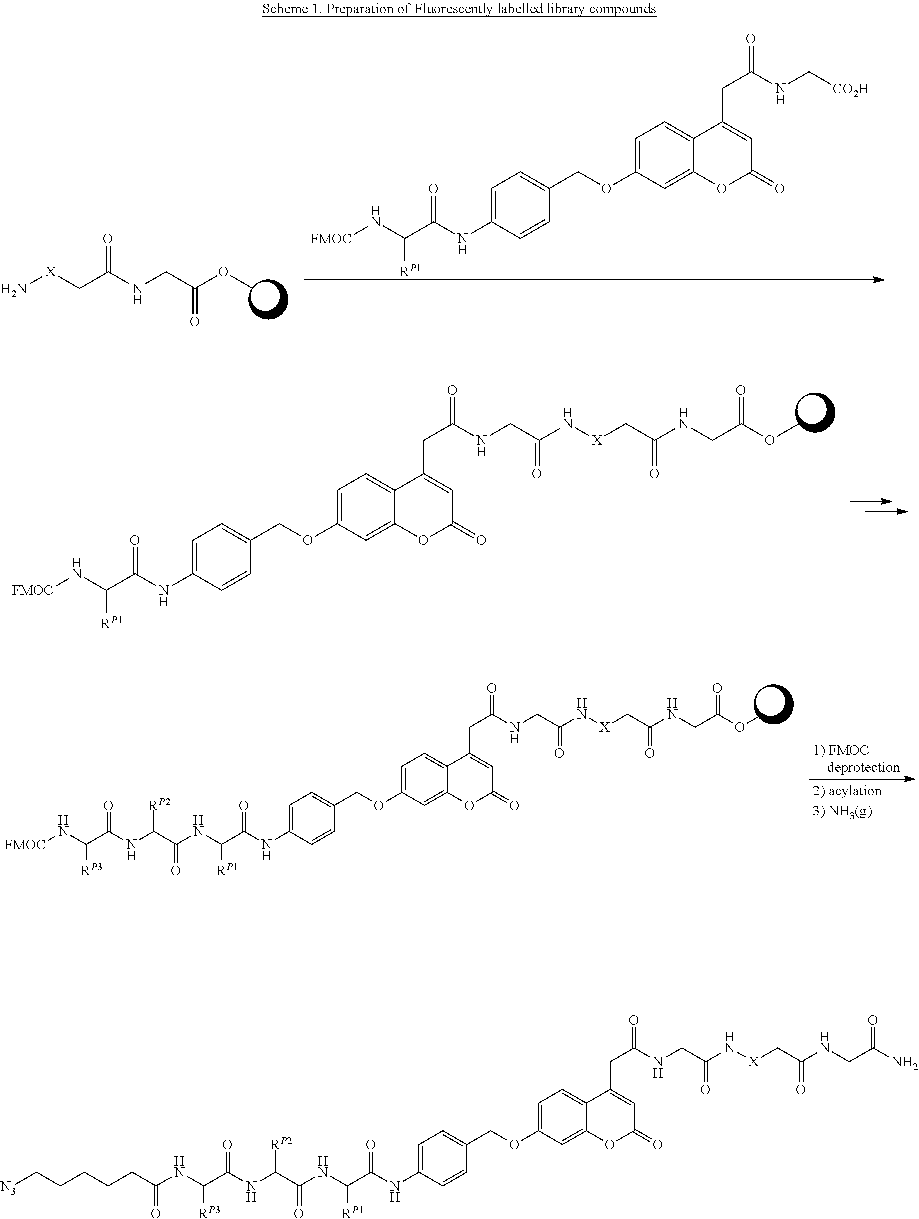

The results of Table 1 A are for the top 20 tripeptide sequences in which the normalized fluorescence ratio from proteolysis by tumor vs normal tissue homogenate is greater than 2.5.

The normalized fluorescence value for tumor homogenate proteolysis is an averaged value for tumor tissue homogenates derived from four mouse xenograft models. Calculation of those normalized values are described following Table A.

The normalized normal tissue fluorescence values are from proteolysis by normal human bone marrow. Human bone marrow was chosen as the normal tissue because it is the site of an adverse event (neutropenia) that has been associated with administering to a human subject in need thereof an effective amount of an Antibody Drug Conjugate having drug linker moieties derived from the Drug Linker compound me-val-cit-PABC-MMAE.

TABLE 1A
Ranking of Tripeptide Library Members by Fluorescence Ratio

| | Normalized | Normalized | Tumor/ |
|---|---|---|---|
| Tripeptide* | Normal Tissue | Tumor Tissue | Normal |
| Pro-Ala-Glu | 0.78 | 2.48 | 3.16 |
| DLeu-Leu-Glu | 1.12 | 3.45 | 3.09 |
| Pro-Ala-Cit | 1.07 | 3.03 | 2.84 |
| Pro-Glu-Cit | 0.85 | 2.35 | 2.77 |
| DLeu-Leu-Cit | 1.37 | 3.70 | 2.70 |
| Pro-Cit-Glu | 0.85 | 2.27 | 2.68 |
| Pro-Cit-Cit | 0.95 | 2.54 | 2.67 |
| Cit-Glu-Cit | 1.21 | 3.16 | 2.61 |
| Pro-Lys-Glu | 0.72 | 1.88 | 2.59 |
| DLeu-Leu-Met(O) | 0.66 | 1.70 | 2.58 |
| Pro-Glu-Glu | 0.73 | 1.87 | 2.58 |
| Pro-Glu-Lys | 0.72 | 1.85 | 2.57 |
| Glu-Glu-Cit | 1.22 | 3.14 | 2.57 |
| DLeu-Phe-Glu | 0.89 | 2.27 | 2.55 |
| DLeu-Leu-Lys | 0.87 | 2.20 | 2.54 |
| Glu-Ala-Glu | 1.05 | 2.66 | 2.53 |
| Ala-Cit-Cit | 1.02 | 2.58 | 2.53 |
| Thr-Cit-Cit | 0.89 | 2.26 | 2.53 |
| Cit-Glu-Glu | 0.97 | 2.44 | 2.53 |
| Cit-Cit-Cit | 1.12 | 2.82 | 2.52 |

*Abbreviations: Cit = citrulline, Met(O) = methionine sulfoxide

A normalized fluorescence value is calculated by dividing the fluorescence value at the final time point (275-315 min) from addition of a tissue homogenate by the fluorescence value when no tissue homogenate was added. That value was then normalized for each peptide in each homogenate by dividing it by the average value for that homogenate. For instance, if one tripeptide had a 2-fold increase when compared to that peptide with no homogenate and the average fold increase with that homogenate was also 2-fold, then the normalized value for that tripeptide in that homogenate was 1. The normalized tumor tissue values of Table 1 were then determined by averaging the normalized fluorescence value for each peptide across all 4 cancer homogenates that were tested. Those tumor homogenates were derived from xenograft models for HPAF-II (Nude mouse), Ramos (SCID mouse), SK-Mel-5 (Nude mouse) and SU-DHL-4 (SCID mouse). The normalized normal tissue values of Table 1 were similarly calculated using homogenized bone marrow. The Tumor/Normal ratios of Table 1A were determined by dividing the normalized tumor tissue values by the normalized normal tissue values.

Given the majority of the tripeptides of Table 1A had an unnatural amino acid or proline in the P3 position and that the P2 position was more variable, three tripeptide sequences were selected that only varied in the P1 position to determine how the position closest to the self-immolative PABC Spacer Unit would alter in vivo selectivity for Ligand Drug Conjugates derived from the Drug Linker compounds containing those tripeptide sequences. Those tripeptides are D-Leu-Leu-Cit, D-Leu-Leu-Met(O) and D-Leu-Leu-Lys.

A new sort was preformed based on tripeptides of Table 1A that exhibited a normalized fluorescence for normal tissue homogenate of less than or equal to 0.7 while having a fluorescence ratio of at least 1.5. The top ten tripeptides from that sort are shown in Table 1B. The top three tripeptide sequences of Table 1B, which are D-Leu-Leu-Met(O), Pro-Nal-Lys, and D-Leu-Ala-Glu, were then selected to determine in vivo selectivity for Ligand Drug Conjugates derived from the Drug Linker compounds containing those tripeptide sequences.

TABLE 1B
Ranking of tripeptide library members by propensity for normal
tissue proteolysis

| | Normalized | Normalized | Tumor/ |
|---|---|---|---|
| Tripeptide* | Normal | Tumor | Normal |
| D-Leu-Leu-Met(O) | 0.66 | 1.70 | 2.58 |
| Pro-Nal-Lys | 0.65 | 1.33 | 2.05 |
| D-Leu-Ala-Glu | 0.69 | 1.30 | 1.87 |
| Pro-Glu-Ala | 0.70 | 1.28 | 1.84 |
| Lys-Glu-Met(O) | 0.70 | 1.16 | 1.66 |
| DLeu-Ala-Lys | 0.68 | 1.16 | 1.70 |
| Leu-Nal-Lys | 0.62 | 1.13 | 1.84 |
| DLeu-Cit-Glu | 0.64 | 1.07 | 1.67 |
| DLeu-Glu-Lys | 0.62 | 1.06 | 1.71 |
| G1u-Ala-Met(O) | 0.65 | 1.06 | 1.63 |

*Abbreviations: Cit = citrulline, Met(O) = methionine sulfoxide, Nal = naphtha-1-yl alanine.

The 5 distinct tripeptide sequences chosen from Tables 1A and 1B were incorporated into Ligand Drug Conjugates in which the Ligand Unit is from an antibody that selectively binds to an internalizable antigen preferentially displayed by cells from a human pancreatic adenocarcinoma cell line and which corresponds in structure to a comparator Conjugate having a non-binding control antibody as the “Ligand Unit” and a dipeptide Cleavable Unit in which the drug linker moieties are mc-val-cit-PABC-MMAE. Those Ligand Drug Conjugates have an average drug loading of 4.

Part B. Preparation of Drug Linker compounds.

Drug Linker compounds in which MMAE is the Drug Unit and which were used for preparing the selected subset of Ligand Drug Conjugates discussed in Part A, are represented by the following structure.

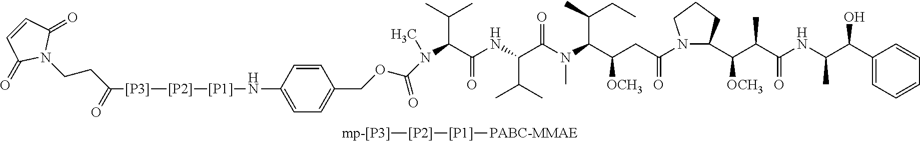

### Example 8: Preparation of Resin-Bound MMAE

Resin-bound MMAE was prepared using DHP HM functionalized resin according to the procedure of Scheme 2A.

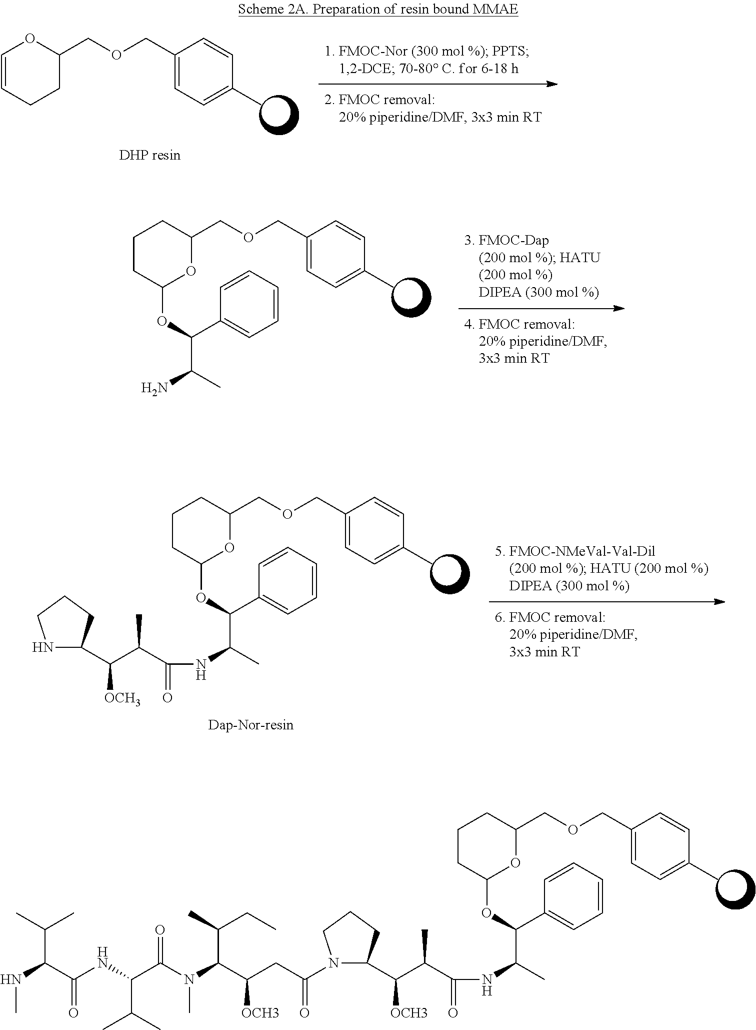 [SMILES: Cc1ccc(COCC2CCC=CO2)cc1 ; Cc1ccc(COCC2CCCC(O[C@@H](c3ccccc3)[C@@H](C)N)O2)cc1 ; CO[C@@H]([C@@H]1CCCN1)[C@@H](C)C(=O)N[C@H](C)[C@@H](OC1CCCC(COCc2ccc(C)cc2)O1)c1ccccc1 ; CC[C@H](C)[C@@H]([C@@H](CC(=O)N1CCC[C@H]1[C@H](OC)[C@@H](C)C(=O)N[C@H](C)[C@@H](OC1CCCC(COCc2ccc(C)cc2)O1)c1ccccc1)OC)N(C)C(=O)[C@@H](NC(=O)[C@@H](NC)C(C)C)C(C)C]

Briefly, to synthesize MMAE on resin, FMOC-Norephedrine and pyridinium p-toluenesulfonate (PPTS) were dissolved in dichloroethane, added to DHP HM functionalized resin, and incubated at 70° C. for 8 h. After deprotection, FMOC-Dap was subsequently activated with HATU and DIPEA and then added to the norepinephrine-resin material. The reaction sequence was repeated with FMOC-N-MeVal-Val-Dil, which after deprotection, provided resin-bound MMAE.

### Example 9: Alternative Preparation of Resin Bound MMAE

An alternative preparation for resin-bound MMAE is shown in Scheme 2B starting from resin bound Dap-Nor of Scheme 2A.

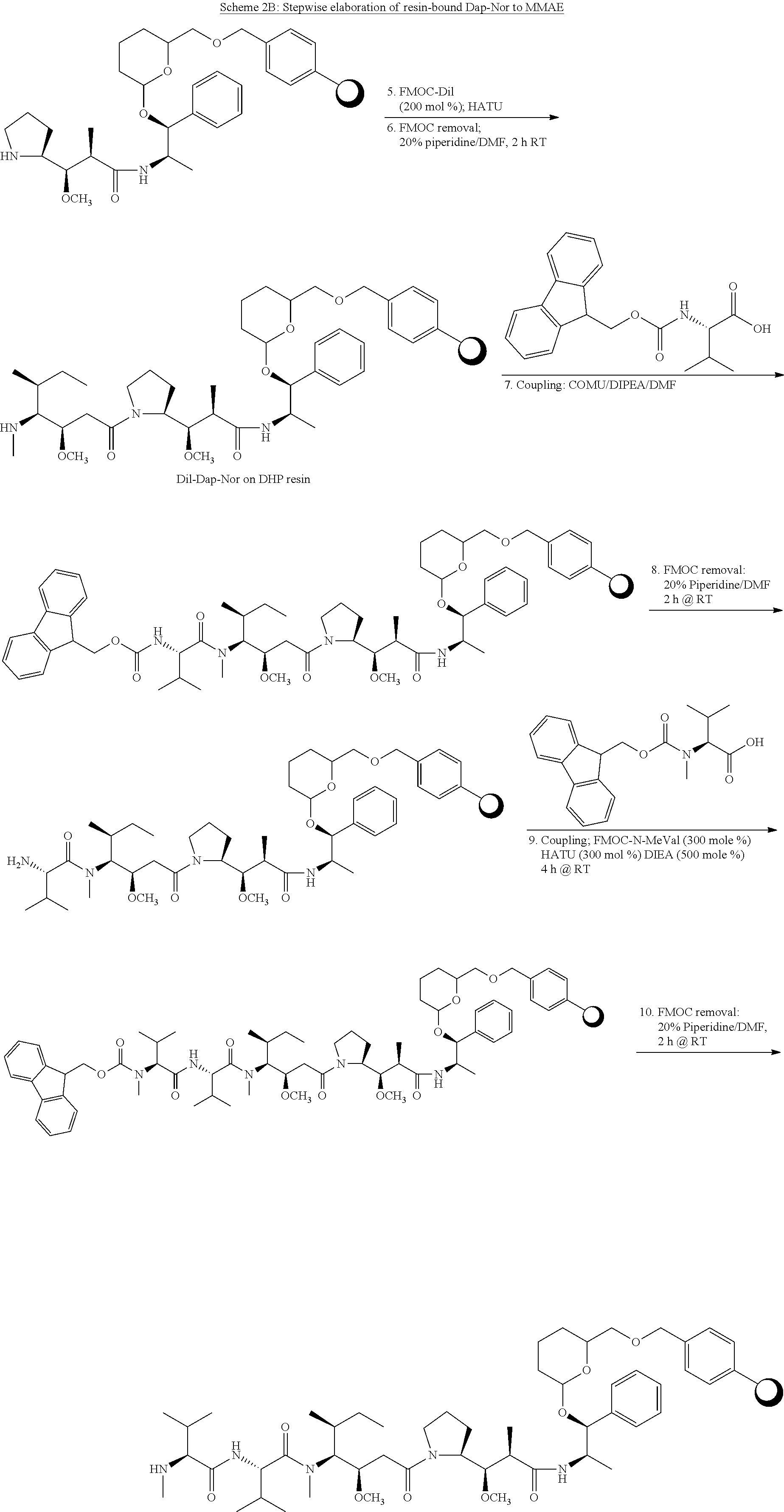

The reaction sequence of Scheme 2B is also useful for preparing radiolabeled MMAE using FMOC-protected [¹⁴C]-valine in step 7. Completion of the Drug Linker compound from resin bound MMAE is shown in Scheme 3.

### Example 10: Preparation of Tripeptide Based MMAE Drug Linker Compounds

Tripeptide-based Drug Linker compounds in which the Drug Unit is derived from MMAE and having tripeptide sequences selected from Tables 1A and 1B were prepared from resin-bound MMAE according to the procedures of Scheme 3 or from MMAE in solution phase according to the procedures of Scheme 3A.

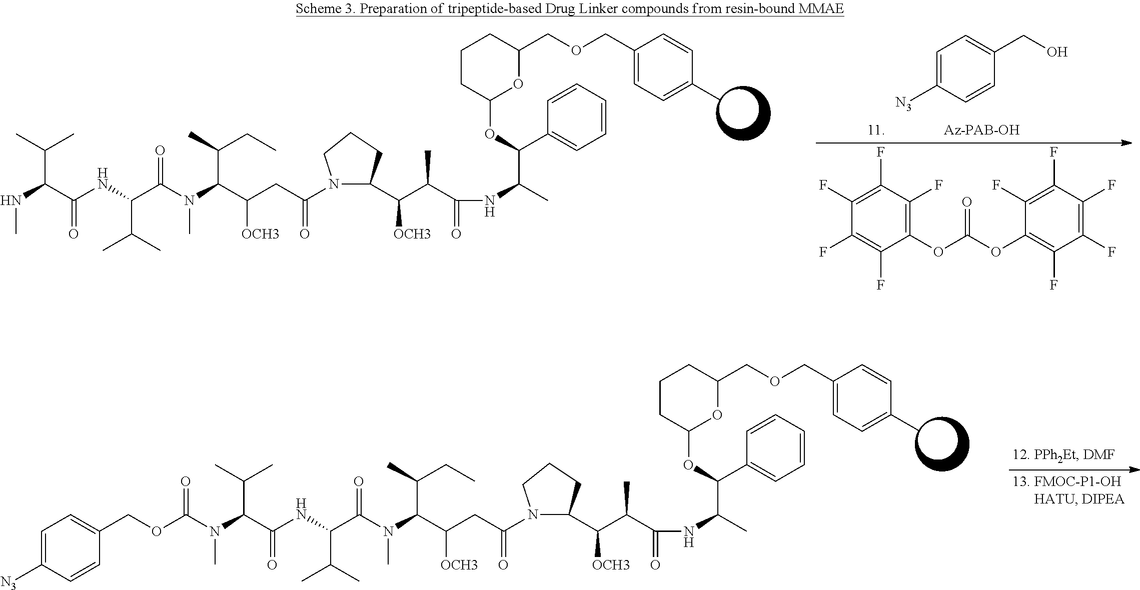 [SMILES: CC[C@H](C)[C@@H](C(CC(=O)N1CCC[C@H]1[C@H](OC)[C@@H](C)C(=O)N[C@H](C)[C@@H](OC1CCCC(COCc2ccc(C)cc2)O1)c1ccccc1)OC)N(C)C(=O)[C@@H](NC(=O)[C@@H](NC)C(C)C)C(C)C ; O=C(Oc1c(F)c(F)c(F)c(F)c1F)Oc1c(F)c(F)c(F)c(F)c1F ; [N-]=[N+]=Nc1ccc(CO)cc1 ; CC[C@H](C)[C@@H](C(CC(=O)N1CCC[C@H]1[C@H](OC)[C@@H](C)C(=O)N[C@H](C)[C@@H](OC1CCCC(COCc2ccc(C)cc2)O1)c1ccccc1)OC)N(C)C(=O)[C@@H](NC(=O)[C@H](C(C)C)N(C)C(=O)OCc1ccc(N=[N+]=[N-])cc1)C(C)C]

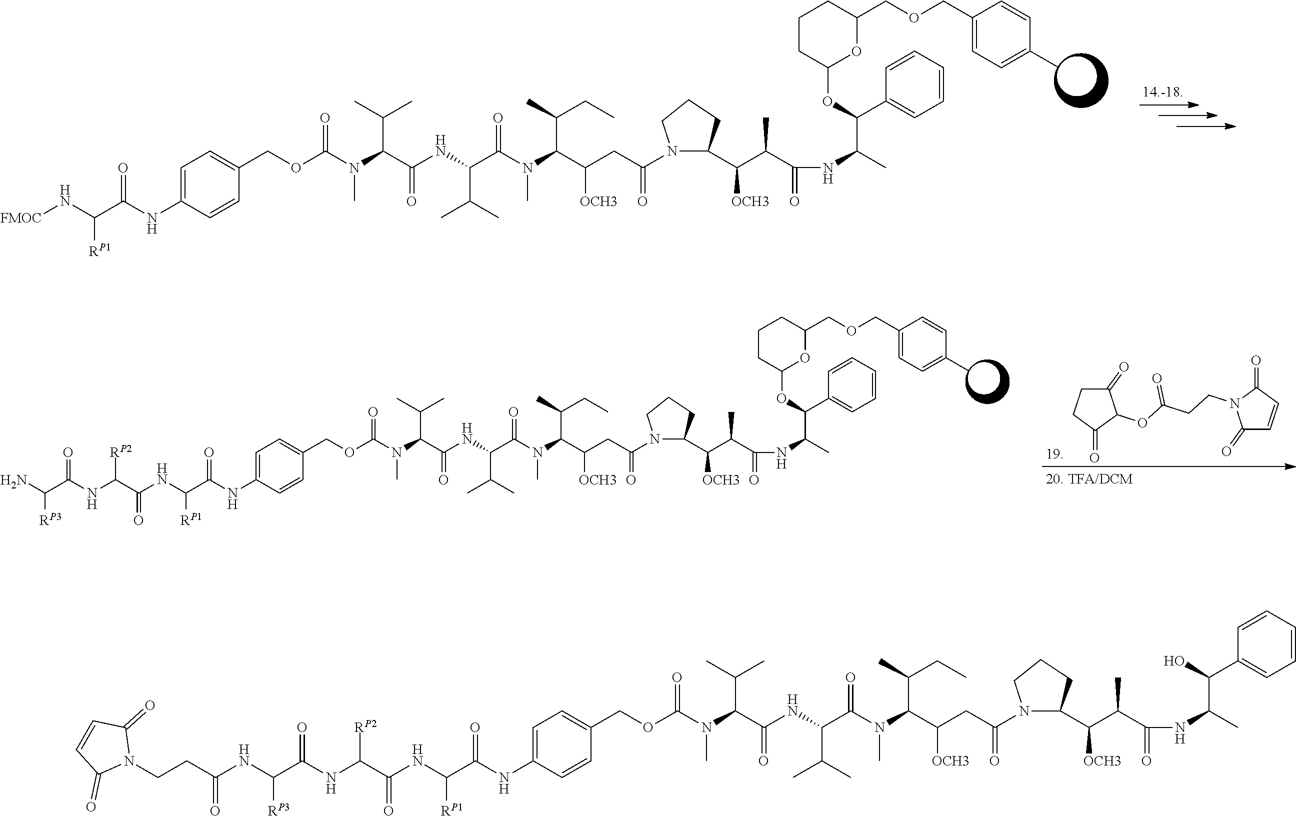

Briefly, Az-PAB-OH, prepared by reaction of NaN₃ with the diazonium salt from p-aminobenzyl alcohol and NaNO₂ in 5M HCl, was reacted with bis(pentafluorophenyl) carbonate and added to MMAE on resin. The azido group of Az-PABC-MMAE was then reduced to the iminophosphorane with PPh₂Et, followed by addition of FMOC-P1. After deprotection, amino acids P2 and P3 were then added through conventional FMOC peptide chemistry followed by reaction of the activated ester 3-(Maleimido)propionic acid N-hydroxysuccinimide ester with the deprotected amine of the terminal P3 amino acid. After cleavage from resin using TFA in DCM, the Drug Linker compound so obtained was purified by reverse phase HPLC.

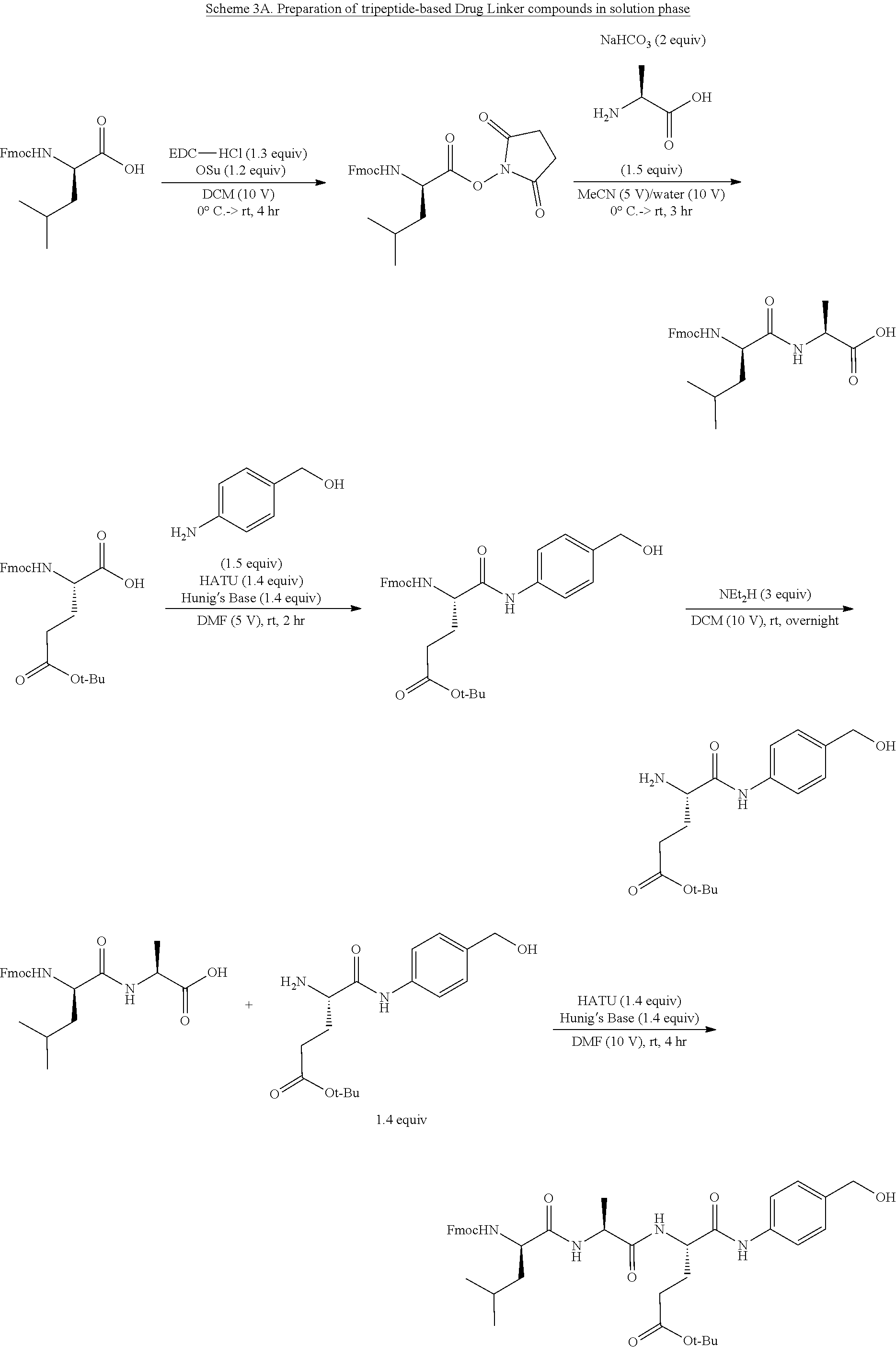

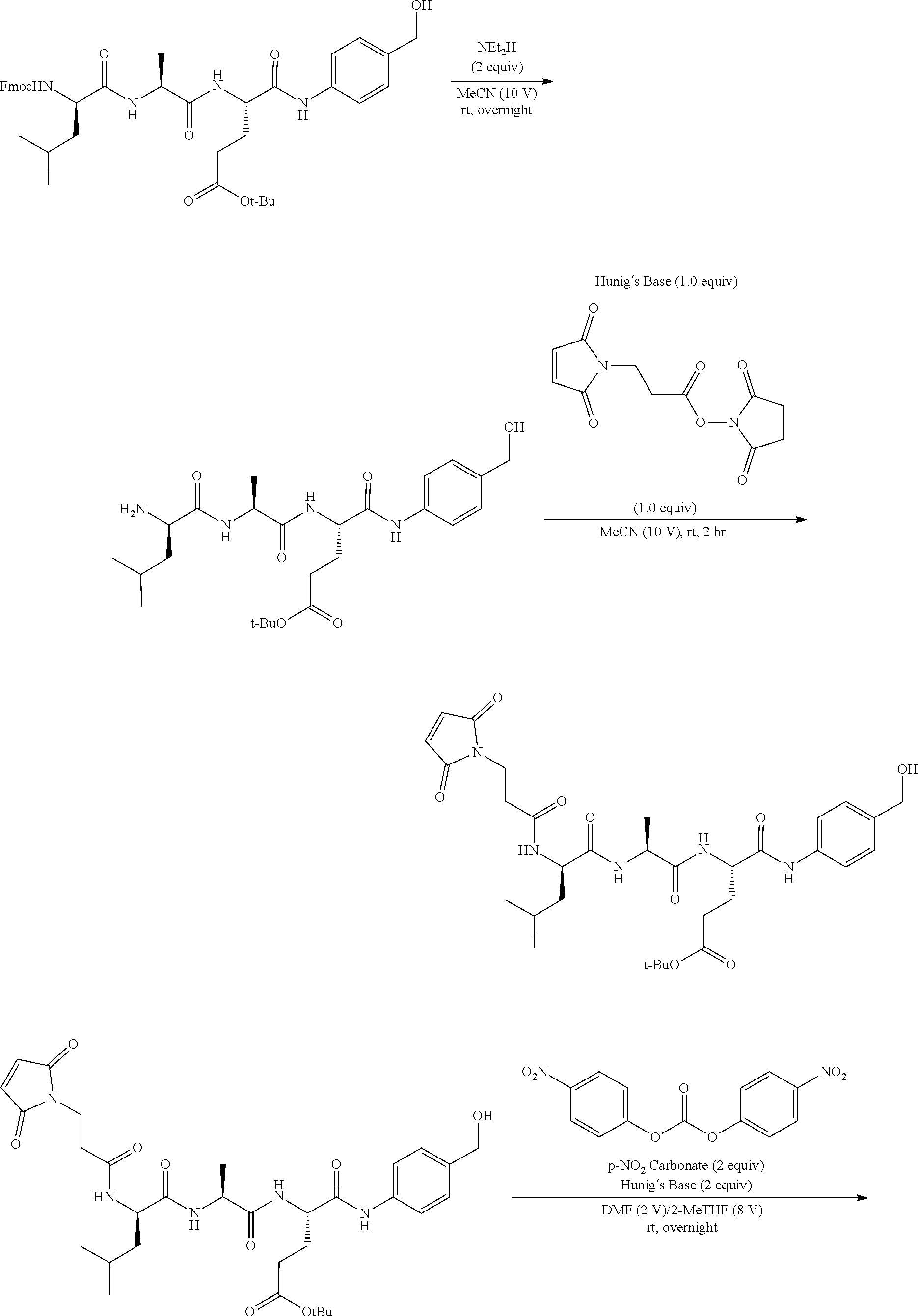

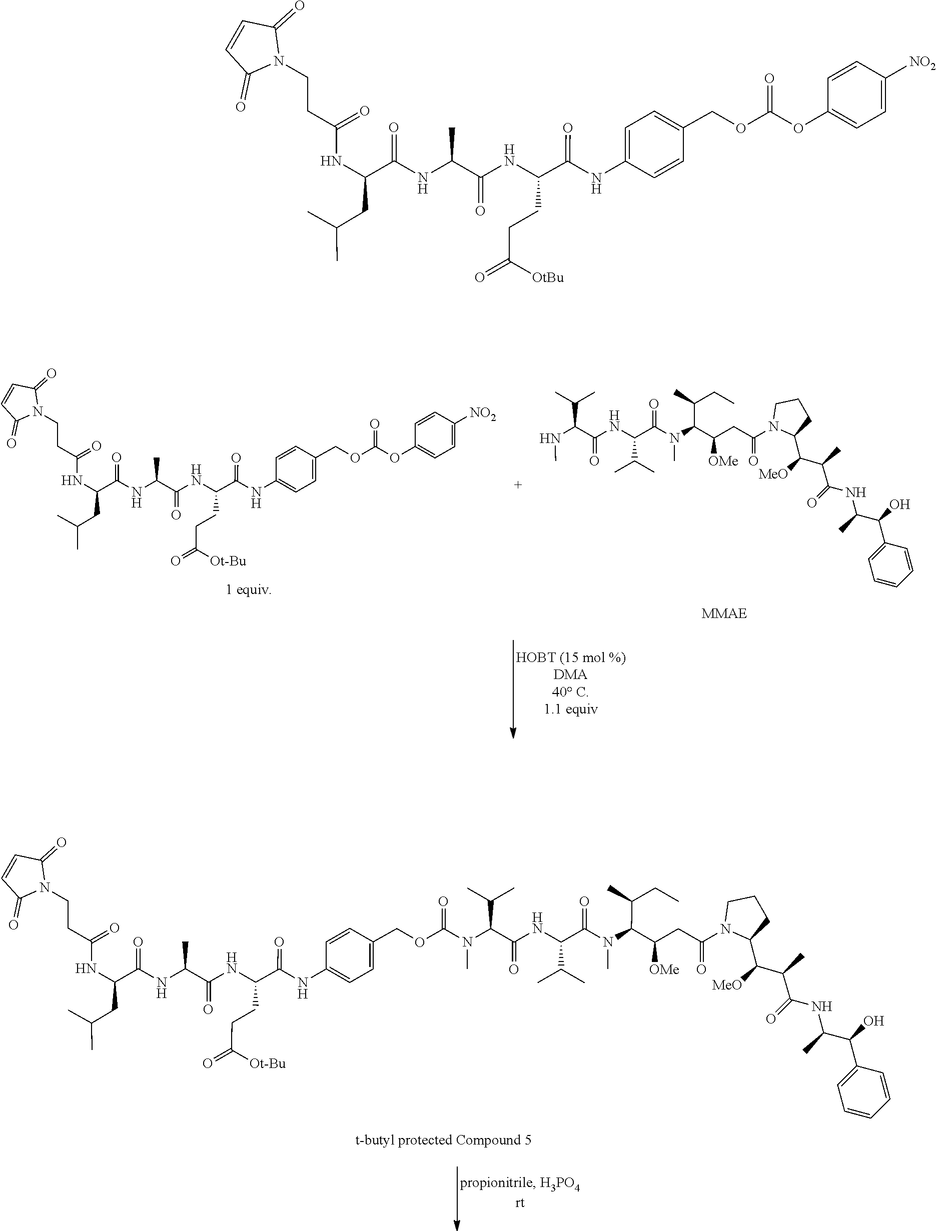

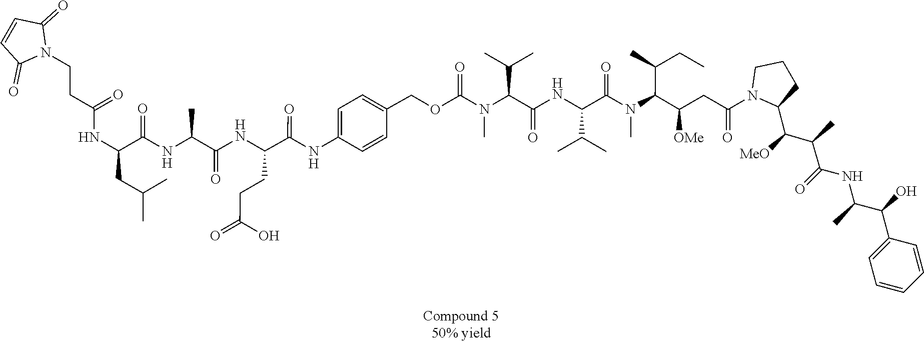 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCC(=O)O)NC(=O)[C@H](C)NC(=O)[C@@H](CC(C)C)NC(=O)CCN2C(=O)C=CC2=O)cc1)C(C)C]

Briefly, (((9H-fluoren-9-yl)methoxy)carbonyl)-D-leucine (1.00 equiv, 50.00 g, 141 mmol) was charged to a 2 L round bottomed flask (RBF) equipped with a magnetic stirbar. Dichloromethane (DCM) (500 ml) was added to the vessel and cooled to 0° C. with stirring followed by addition of ethylcarbodiimide hydrochloride (EDC-HCl) (1.30 ea, 35.26 g, 184 mmol) and N-hydroxysuccinimide (1.20 eq, 19.54 g, 170 mmol) was charged to the reaction. The reaction was stirred at 0° C. for 30 minutes then allowed to warm to rt and stirred at 4 hrs. Upon completion of the reaction water was added to the reaction (500 ml), the organic layer was separated, washed with brine (500 ml) and separated. The DCM solution was evaporated under reduced pressure to give 2,5-dioxopyrrolidin-1-yl (((9H-fluoren-9-yl)methoxy)carbonyl)-D-leucinate as a white foam (65.00 g, 144 mmol, 102% yield). This material was used without further purification.

In the next step, 2,5-dioxopyrrolidin-1-yl (((9H-fluoren-9-yl)methoxy)carbonyl)-D-leucinate (1.00 equiv, 30.0 g, 66.6 mmol) and alanine (1.5 equiv, 8.90 g, 99.9 mmol) were charged to a 1000 ml RBF with a magnetic stirbar. Acetonitrile (150 ml) and water (300 ml) were charged to the vessel and cooled to 0° C. Hunig's base was charged to the reaction in one portion (2.0 equiv, 17.2 g, 133.2 ml). The reaction was stirred at 0° C. for 1 hr then allowed to warm to rt and stirred overnight. Upon completion the solvent was swapped by rotary evaporation to ethyl acetate (EtOAc). The pH was adjusted to pH=2 by addition of 1M HCl. The organic layer was separated and washed with brine. The reaction mixture was concentrated by rotary evaporation to give a white solid (31.29 g). The solid was dissolved in EtOAc (120 ml) in a 1000 ml RBF equipped with a magnetic stirbar. The solid was precipitated by dropwise addition of Heptane (600 ml) over 1 hour. The slurry was stirred overnight. The solid was filtered and washed with Heptane (300 ml) to give a fine white solid. The solid was dried in a vacuum oven overnight at 45° C. to give (((9H-fluoren-9-yl)methoxy)carbonyl)-D-leucyl-L-alanine as a white solid (24.01 g, 85% yield)

(S)-2-((((9H-fluoren-9-yl)methoxy)carbonyl)amino)-5-(tert-butoxy)-5-oxopentanoic acid (50.0 g, 1.00 equiv, 117.5 mmol), (4-aminophenyl)methanol (21.7 g, 1.5 equiv, 176.3 mmol), and HATU (62.9 g, 1.4 equiv, 164.5 mmol) were charged to a 2000 ml RBF equipped with a magnetic stir bar. Dimethyl formamide (DMF) (250 ml) was charged to the vessel and stirred until the solids dissolve. Hunig's base (21.26 g, 1.4 equiv, 164.5 mmol) was charged to the reaction in one portion. The reaction was stirred at rt for two hours. Upon completion water (750 ml) was added by dropwise addition over 30 minutes. The slurry was stirred for an additional 1 hr at rt. The slurry was filtered and washed with water (500 ml) to give an orange solid. The solid was redissolved in DCM (500 ml) and washed with water (500 ml). To this solution in a 2000 ml RBF was added a magnetic stirbar. Diethylamine (25.64 g, 3.0 equiv, 350.54 mmol) was charged to the reaction and stirred at rt overnight (reaction precipitated overnight). Upon completion, Heptane (620 ml) was added to the reaction over 1 hr. The slurry was stirred for 1 hr. The slurry was filtered and washed with Hepante (620 ml) to give a pink solid. The solid was dried in the vacuum oven at 45° C. overnight to give tert-butyl (S)-4-amino-5-((4-(hydroxymethyl)phenyl)amino)-5-oxopentanoate as a brown solid (35.2 g, 98% yield).

(((9H-Fluoren-9-yl)methoxy)carbonyl)-D-leucyl-L-alanine (8.1 g, 1.00 equiv, 19.08 mmol), tert-butyl (S)-4-amino-5-((4-(hydroxymethyl)phenyl)amino)-5-oxopentanoate (8.82 g, 1.5 equiv, 28.62 mmol), and HATU (10.21 g, 1.4 equiv, 26.71 mmol) was charged to a 500 ml RBF. DMF (80 ml) and Hunig's base was charged to the vessel and stirred at rt for 2 hours. Upon completion the reaction was precipitated with dropwise addition of water (160 ml) over 1 hour to give a solid that sticks to the stirbar. The liquid was decanted and the solid was washed with water (80 ml). The solid was reslurried with DCM (80 ml) with heat cycling to get a red solution. The solution was precipitated with dropwise addition of Heptane (80 ml) over 30 minutes. The solid was filtered to give a yellow solid that was washed with heptane (80 ml). The solid was dried in a vacuum oven at 45° C. overnight to give Fmoc-protected tripeptide of D-Leu-Ala-Glu linked to 4-aminobenzyl alcohol a yellow solid (12 g, 88% yield).

For Fmoc-deprotection this tripeptide (1.00 equiv, 26.8 g, 37.49 mmol) was charged to a 400 ml EasyMax Reactor. MeCN (10 V, 270 ml) was charged to the vessel and stirred at 25° C. at 200 rpm (red solution). Diethylamine was added to the reaction in one portion (2.0 equiv, 5.48 g, 74.98 mmol). The reaction was stirred at rt overnight and upon completion the solvent was swapped to 10 V EtOAc by rotary evaporation. The slurry was heated to reflux to give a red solution. The slurry was cooled to 15° C. and stirred overnight. The slurry was filtered and washed with MTBE (3×10 V, 3×270 ml) to give a light brown solid. The solid was dried in a vacuum oven at 40° C. to give tert-butyl (S)-4-((S)-2-((R)-2-amino-4-methylpentanamido)propanamido)-5-((4-(hydroxymethyl)phenyl)amino)-5-oxopentanoate as a pink solid (14.47 g, 78% yield).

Tert-Butyl (S)-4-((S)-2-((R)-2-amino-4-methylpentanamido)propanamido)-5-((4-(hydroxymethyl)phenyl)amino)-5-oxopentanoate (1.00 equiv, 9.51 g, 19.31 mmol) and 2,5-dioxopyrrolidin-1-yl 3-(2,5-dioxo-2,5-dihydro-1H-pyrrol-1-yl)propanoate (1.0 equiv, 5.14 g, 19.31 mmol) were charged to a 200 ml EasyMax Reactor. MeCN (10 V, 100 ml) was added to the reactor and stirred at 25° C. at 200 rpm. Hunig's base (1.0 equiv, 2.50 g, 19.31 mmol) was added to the reaction in one portion. The reaction was stirred at 25° C. at 200 rpm for one hour (red solution). Upon completion solvent was swapped to 10 V EtOAc by rotary evaporation. The product was precipitated with addition of heptane (10 V, 100 ml) over 30 minutes. The slurry was filtered and washed with MTBE (2×10 V, 2×100 ml). The solid was dried in a vacuum oven overnight at 40° C. to give tert-butyl (S)-4-((S)-2-((R)-2-(3-(2,5-dioxo-2,5-dihydro-1H-pyrrol-1-yl)propanamido)-4-methylpentanamido)propanamido)-5-((4-(hydroxymethyl)phenyl)amino)-5-oxopentanoate as a light brown solid (12.38 g, 99% yield)

Tert-butyl (S)-4-((S)-2-((R)-2-(3-(2,5-dioxo-2,5-dihydro-1H-pyrrol-1-yl)propanamido)-4-methylpentanamido)propanamido)-5-((4-(hydroxymethyl)phenyl)amino)-5-oxopentanoate (2.7 g, 1.00 equiv, 4.19 mmol) and 4-nitrophenyl carbonate (2.55 g, 2.0 equiv, 8.39 mmol) was charged to a 100 ml RBF equipped with a magnetic stirbar. DMF (2V, 5 ml) and 2-MeTHF (8V, 20 ml) were charged to the reaction with stirring at rt. Hunig's base was charged to the vessel and stirred at rt for overnight. Upon completion, the reaction was diluted with 10 V 2-MeTHF. The organic layer was successively washed with 20V 5% LiCl, 20V water, and then 10% NaCl. The organic solution was added dropwise to 10V MTBE/10V Heptane over 15 minutes. The slurry was aged for 1 hour with stirring at rt. The slurry was filtered and washed with three times with 5V MTBE/5V heptane. The solid was dried in a vacuum oven at 35° C. overnight to give a pale yellow solid (2.06 g, 61% yield).

The p-nitro carbonate activated tripeptide (1 equiv, 10 mg, 0.01 mmol), MMAE (1.1 equiv, 9.7 mg, 0.01 mmol) and HOBt (0.15 equiv, 29 μl of 10 mg/ml solution in DMA) was charged to a 1 dr vial equipped with a magnetic stir bar. DMA (10 Vol, 200 μl) was charged and the reaction stirred at 40° C. Upon completion, the reaction was cooled to room temperature. Water was added dropwise, until an amorphous solid formed. The solvent was decanted and the solid redissolved in 10 V DCM. The organic solution was washed twice with 20 V HCl (0.5 M) and concentration under vacuum to afford tert-butyl protected compound 5.

The tert-butyl protected compound 5 (1.0 g, 1.00 equiv, 0.72 mmol) was dissolved in 10 mL of propionitrile. 10 mL H₃PO₄ was added to the rxn mixture slowly at rt. Reaction mixture was stirred for 2 h. Upon completion 15 mL water and 10 mL Propionitrile were added. The organic layer was separated and the aqueous layer was extracted with 10 mL Propionitrile. The combined organic layer was washed one more time with 30 mL water. The reaction was concentrated and purified by reverse-phase prep-HPLC to afford Compound 5.

UPLC-MS data for MMAE and MMAF Drug Linker compounds prepared according to the reaction sequences of Scheme 2A, Scheme 3, and Scheme 3A, wherein several compounds have tripeptide sequences selected from Tables 1A and 1B, are shown in Tables 2 and 2A.

UPLC-MS was performed on a Waters single quad detector mass spectrometer interfaced to a Waters Acquity™ UPLC system using the UPLC method (Methods A-D) shown below, wherein Solvent A is 0.1% aqueous formic acid and Solvent B—acetonitrile with 0.1% formic acid.

Method A: Column-Waters Acquity UPLC BEH C18, 130 Å,
1.7 μm, 2.1 × 50 mm, reversed-phase column

| Time | | | |
|---|---|---|---|
| (min) | Flow (mL/min) | A % | B % |
| Initial | 0.5 | 97 | 3 |
| 1.0 | 0.5 | 40 | 60 |
| 1.5 | 0.5 | 5 | 95 |

Method B: Column-Waters CORTECS UPLC C18, 90 Å, 1.6 μm,
2.1 × 50 mm, reversed-phase column

| Time | | | |
|---|---|---|---|
| (min) | Flow (mL/min) | A % | B % |
| Initial | 0.5 | 97 | 3 |
| 1.7 | 0.5 | 40 | 60 |
| 2.0 | 0.5 | 5 | 95 |
| 3.5 | 0.5 | 5 | 95 |
| 3.8 | 0.5 | 97 | 3 |
| 4.0 | 0.5 | 97 | 3 |

Method C: Column-Waters CORTECS UPLC C18, 90 Å, 1.6 μm,
2.1 × 50 mm, reversed-phase column

| Time | | | |
|---|---|---|---|
| (min) | Flow (mL/min) | A % | B % |
| Initial | 0.5 | 97 | 3 |
| 1.5 | 0.5 | 5 | 95 |

Method D: Column-Waters Acquity UPLC BEH C18, 130 Å, 1.7 μm,
2.1 × 50 mm, reversed-phase column

| Time | | | |
|---|---|---|---|
| (min) | Flow (mL/min) | A % | B % |
| Initial | 0.5 | 97 | 3 |
| 1.7 | 0.5 | 40 | 60 |
| 2.0 | 0.5 | 5 | 95 |
| 3.5 | 0.5 | 5 | 95 |
| 3.8 | 0.5 | 97 | 3 |
| 4.0 | 0.5 | 97 | 3 |

TABLE 2
UPLC-MS data for Selected MMAE Drug Linker compounds

| Compound | Tripeptide* | Molecular | MS Calc. | MS | Retention | |
|---|---|---|---|---|---|---|
| # | Sequence | Formula | (M + H)⁺ | found | Time (min.) | Method |
| 2 | Pro-Nal-Lys | C₇₈H₁₀₉N₁₁O₁₅ | 1440.8 | 1441 | 1.31 | A |
| 3 | D-Leu-Leu-Lys | C₇₂H₁₁₃N₁₁O₁₅ | 1372.8 | 1373 | 1.22 | A |
| 4 | D-Leu-Leu-Met(O) | C₇₁H₁₁₀N₁₀O₁₆S | 1391.8 | 1392 | 1.5 | A |
| 5 | D-Leu-Ala-Glu | C₆₈H₁₀₂N₁₀O₁₇ | 1331.7 | 1332 | 1.43 | A |
| 6 | D-Leu-Leu-Cit | C₇₂H₁₁₂N₁₂O₁₆ | 1401.8 | 1402 | 1.48 | A |
| 7 | Leu-Ala-Glu | C₆₈H₁₀₂N₁₀O₁₇ | 1330.7 | 1332.2 | 1.92 | B |
| 8 | D-Leu-Ala-Cit | C₆₉H₁₀₆N₁₂O₁₆ | 1358.8 | 1359.3 | 1.39 | A |
| 9 | Pro-Leu-Glu | C₇₀H₁₀₄N₁₀O₁₇ | 1356.8 | 1357.4 | 1.49 | A |
| 10 | Thr-Glu-Leu | C₆₉H₁₀₄N₁₀O₁₈ | 1360.8 | 1361.4 | 1.44 | A |
| 11 | D-Leu-Glu-Cit | C₇₁H₁₀₈N₁₂O₁₈ | 1416.8 | 1417.4 | 1.35 | A |
| 12 | Glu-Pro-Cit | C₇₀H₁₀₄N₁₂O₁₈ | 1400.8 | 1402.2 | 1.29 | A |
| 13 | Tyr(All)-Pro-Cit | C₇₇H₁₁₀N₁₂O₁₇ | 1474.8 | 1475.9 | 1.46 | A |
| 14 | Lys-Leu-Cit | C₇₂H₁₁₃N₁₃O₁₆ | 1415.8 | 1417.6 | 1.21 | A |
| 15 | Tyr(All)-dLeu-Glu | C₇₇H₁₁₀N₁₀O₁₈ | 1462.8 | 1463.8 | 1.41 | C |
| 16 | D-Leu-Ala-Gln | C₆₈H₁₀₃N₁₁O₁₆ | 1331.6 | 1331.4 | 2.01 | D |
| 17 | D-Leu-Ala-(Se-Met) | C₆₈H₁₀₄N₁₀O₁₅Se | 1381.6 | 1381.4 | 2.22 | D |
| 18 | D-Leu-Glu-Ala | C₆₈H₁₀₂N₁₀O₁₇ | 1332.6 | 1332.2 | 2.08 | D |
| 19 | D-Leu-Ala-Ala | C₆₆H₁₀₀N₁₀O₁₅ | 1274.6 | 1275 | 2.13 | B |
| 20 | D-Leu-Ala-Met(O) | C₆₈H₁₀₄N₁₀O₁₆S | 1350.7 | 1350.1 | 2.07 | B |
| 21 | D-Leu-Leu-Glu | C₇₁H₁₀₈N₁₀O₁₇ | 1374.7 | 1373.9 | 2.26 | B |
| 22 | D-Ala-Ala-Glu | C₆₅H₉₆N₁₀O₁₇ | 1290.5 | 1290.3 | 1.89 | D |
| 23 | Ala-Ser-Glu | C₆₅H₉₆N₁₀O₁₈ | 1306.5 | 1306.4 | 1.88 | B |
| 24 | D-Leu-Ala-Asp | C₆₇H₁₀₀N₁₀O₁₇ | 1318.6 | 1318.5 | 2.09 | B |
| 25 | D-Leu-Val-Gln | C₇₀H₁₀₇N₁₁O₁₆ | 1359.7 | 1359.9 | 2.1 | D |
| 26 | D-Leu-Ala-Gla | C₆₉H₁₀₂N₁₀O₁₉ | 1376.6 | 1376.5 | 2.02 | B |
| 27 | D-Leu-Ala-Lys | C₆₉H₁₀₇N₁₁O₁₅ | 1332.7 | 1331.5 | 1.74 | B |
| 28 | Ala-Ser-Asp | C₆₄H₉₄N₁₀O₁₈ | 1292.5 | 1292.3 | 1.83 | B |
| 29 | D-Leu-Ala-Leu | C₆₉H₁₀₆N₁₀O₁₅ | 1316.7 | 1316.5 | 2.28 | B |
| 30 | Phe-Ser-Glu | C₇₁H₁₀₀N₁₀O₁₈ | 1382.6 | 1382.2 | 2.02 | D |
| 31 | Glu-Val-Cit | C₇₀H₁₀₆N₁₂O₁₈ | 1404.7 | 1418.5 | 1.96 | B |
| 32 | D-Leu-Ser-Glu | C₆₈H₁₀₂N₁₀O₁₈ | 1348.6 | 1349.2 | 2.07 | B |
| 33 | D-Ala-Ala-Cit | C₆₆H₁₀₀N₁₂O₁₆ | 1318.6 | 1318.5 | 1.86 | B |
| 34 | Glu-Leu-Cit | C₇₁H₁₀₈N₁₂O₁₈ | 1418.7 | 1418.5 | 1.96 | B |
| 35 | D-Leu-Gly-Glu | C₆₇H₁₀₀N₁₀O₁₇ | 1318.6 | 1318.5 | 2.02 | B |
| 36 | Glu-Ala-Leu | C₆₈H₁₀₂N₁₀O₁₇ | 1332.6 | 1332.5 | 2.03 | B |
| 38 | D-Leu-Aib-Glu | C₆₉H₁₀₄N₁₀O₁₇ | 1346.7 | 1346.4 | 2.13 | D |
| 39 | D-Leu-Aib-Cit | C₇₀H₁₀₈N₁₂O₁₆ | 1374.7 | 1375.1 | 2.11 | B |
| 40 | Val-Gln-Glu | C₆₉H₁₀₃N₁₁O₁₈ | 1375.6 | 1375.5 | 1.84 | B |

*Abbreviations: Aib = α-aminoisobutyric acid, Cit = citrulline, Met(O) = methionine sulfoxide, Nal = naphthal-1-yl alanine, (Se-Met) = Selenomethionine, Gla = gamma-carboxyglutamate, Tyr(All) = O-allyl tyrosine

TABLE 2A
UPLC-MS data for Selected MMAF Drug Linker compound

| Compound | Tripeptide | Molecular | MS Calc. | MS | Retention | |
|---|---|---|---|---|---|---|
| # | Sequence | Formula | (M + H)⁺ | found | Time (min.) | Method |
| 42 | D-Leu-Ala-Glu | C₇₁H₁₀₆N₁₀O₁₈ | 1389.7 | 1388.6 | 2.16 | B |

Structures of the tripeptide-based Drug Linker compounds 2-36 and 38-40 of Table 2 and compound 42 of Table 2A, as well as the comparator dipeptide-based Drug Linker compound 1, compound 7, and compound 41 are as follows:

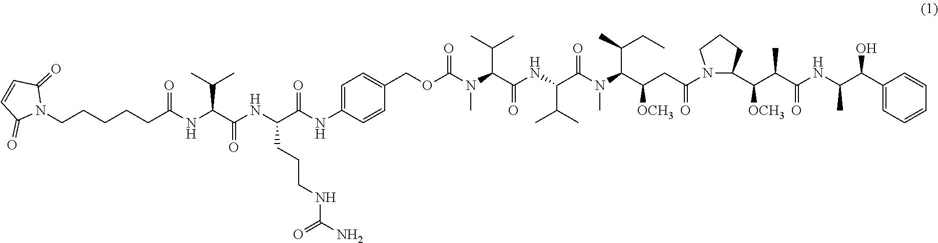 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

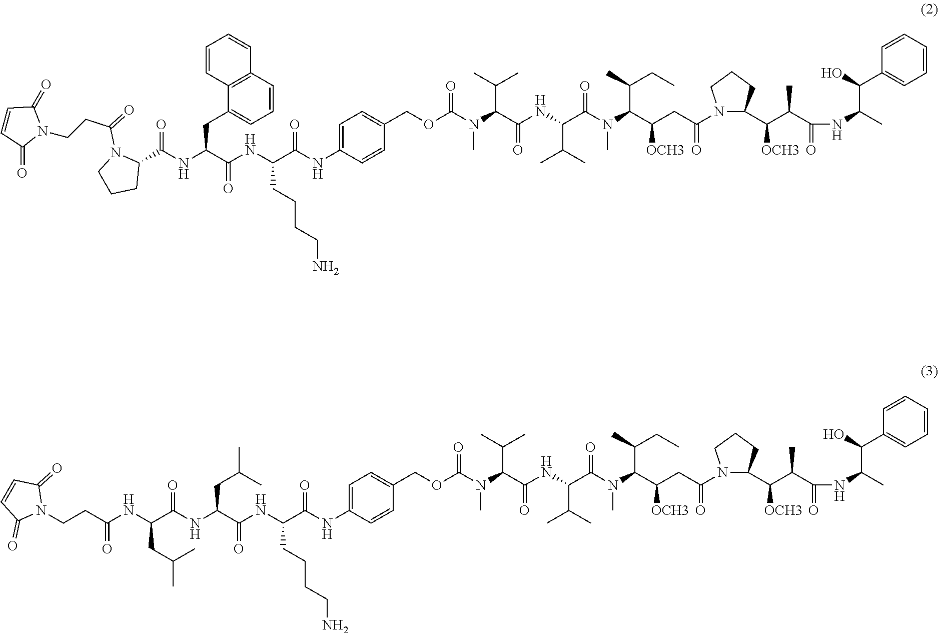 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCCN)NC(=O)[C@H](Cc2cccc3ccccc23)NC(=O)[C@@H]2CCCN2C(=O)CCN2C(=O)C=CC2=O)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCCN)NC(=O)[C@H](CC(C)C)NC(=O)[C@@H](CC(C)C)NC(=O)CCN2C(=O)C=CC2=O)cc1)C(C)C]

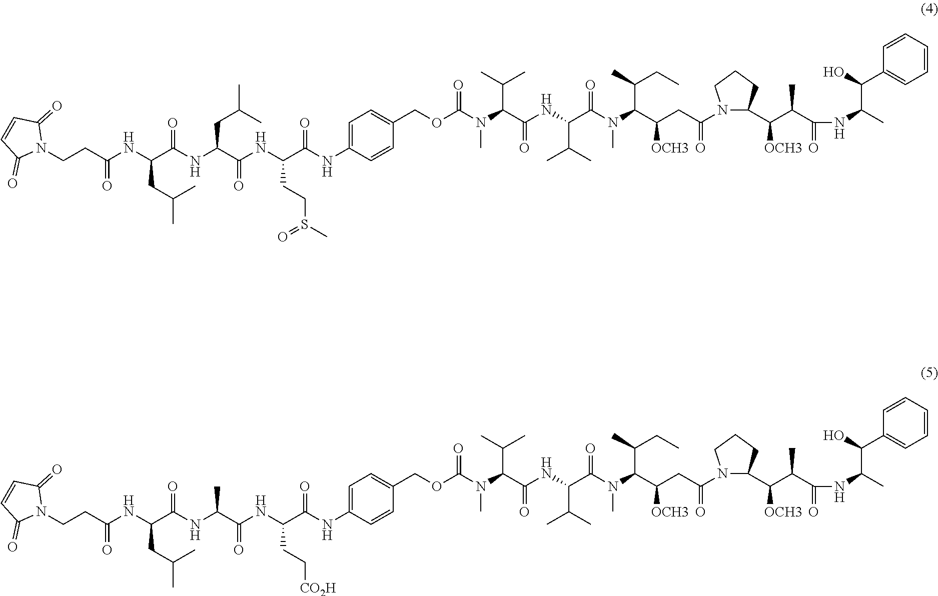 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCS(C)=O)NC(=O)[C@H](CC(C)C)NC(=O)[C@@H](CC(C)C)NC(=O)CCN2C(=O)C=CC2=O)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCC(=O)O)NC(=O)[C@H](C)NC(=O)[C@@H](CC(C)C)NC(=O)CCN2C(=O)C=CC2=O)cc1)C(C)C]

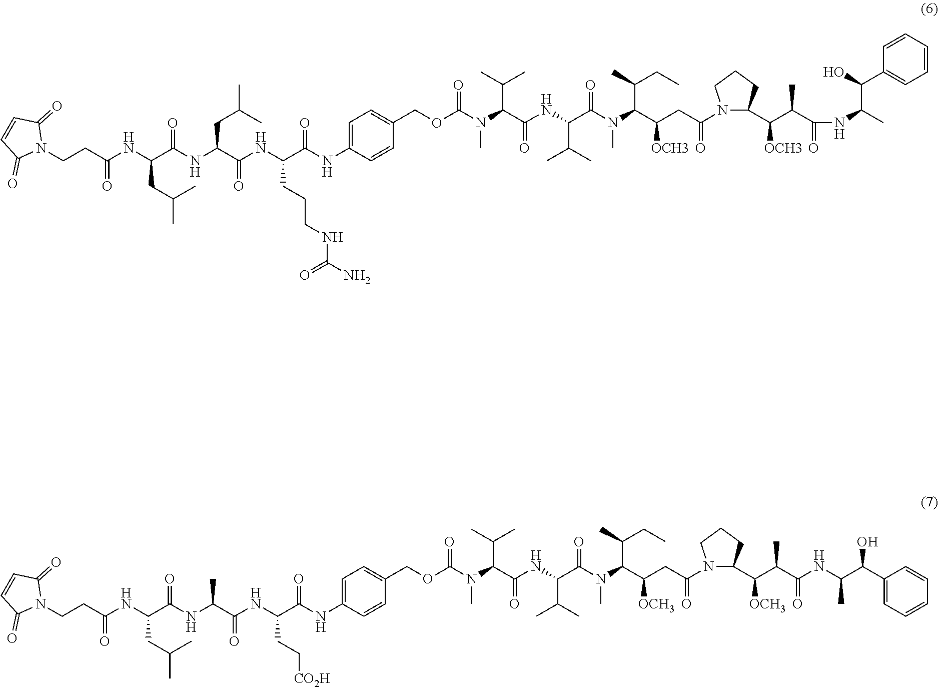 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](CC(C)C)NC(=O)[C@@H](CC(C)C)NC(=O)CCN2C(=O)C=CC2=O)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCC(=O)O)NC(=O)[C@H](C)NC(=O)[C@H](CC(C)C)NC(=O)CCN2C(=O)C=CC2=O)cc1)C(C)C]

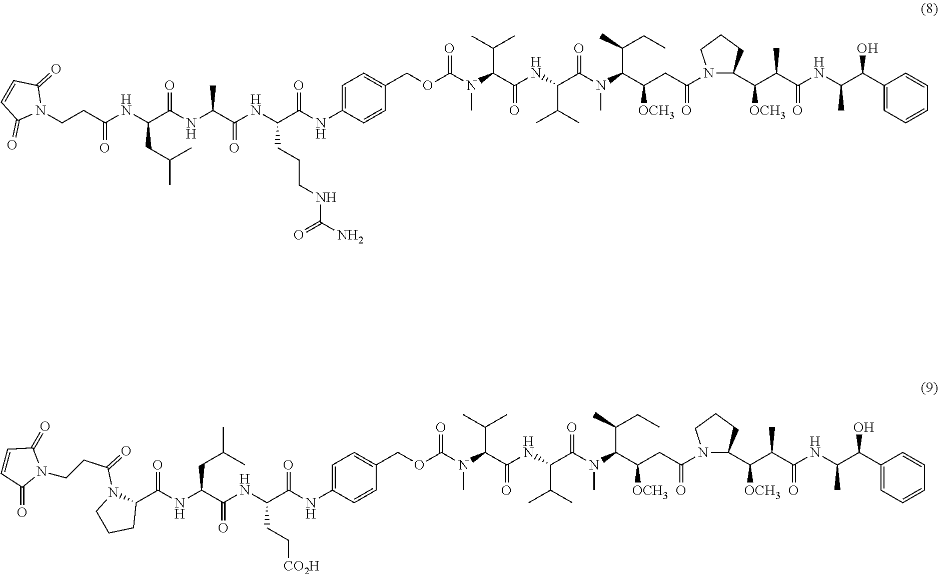 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](C)NC(=O)[C@@H](CC(C)C)NC(=O)CCN2C(=O)C=CC2=O)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCC(=O)O)NC(=O)[C@H](CC(C)C)NC(=O)[C@@H]2CCCN2C(=O)CCN2C(=O)C=CC2=O)cc1)C(C)C]

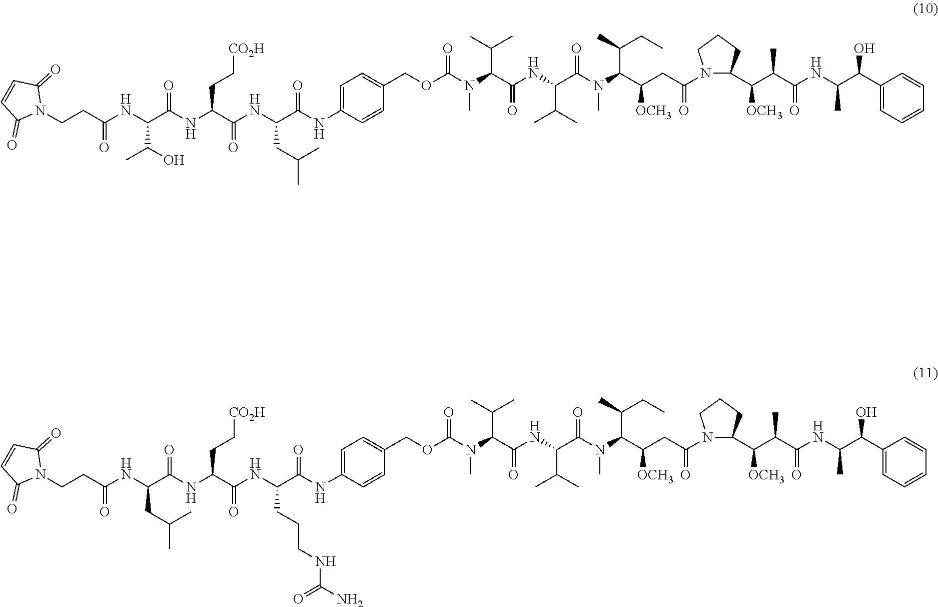 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CC(C)C)NC(=O)[C@H](CCC(=O)O)NC(=O)[C@@H](NC(=O)CCN2C(=O)C=CC2=O)C(C)O)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](CCC(=O)O)NC(=O)[C@@H](CC(C)C)NC(=O)CCN2C(=O)C=CC2=O)cc1)C(C)C]

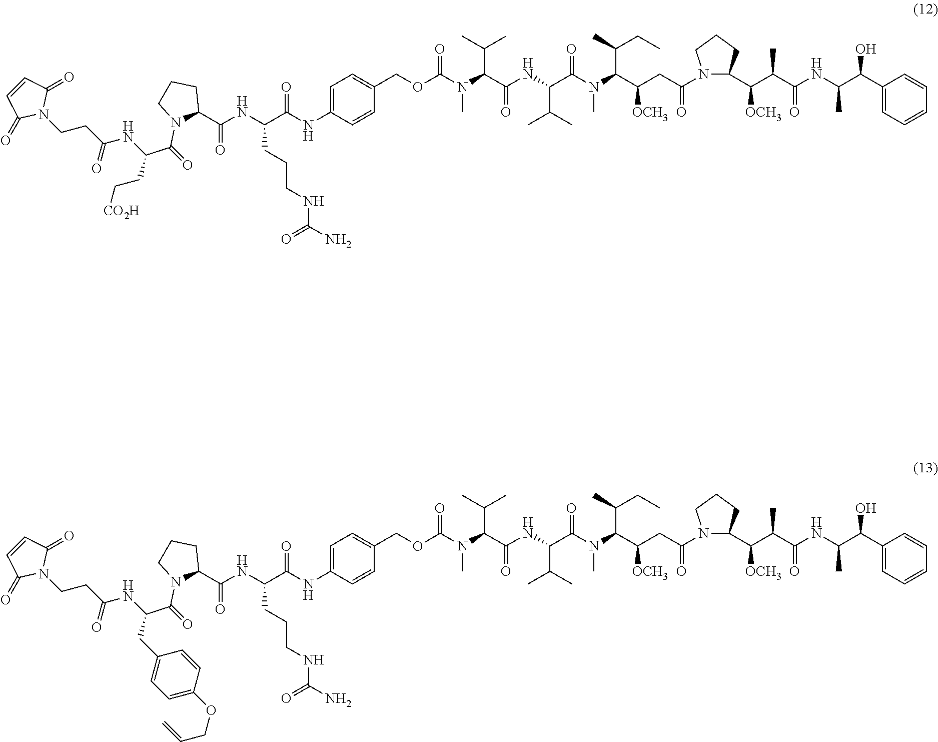 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H]2CCCN2C(=O)[C@H](CCC(=O)O)NC(=O)CCN2C(=O)C=CC2=O)cc1)C(C)C ; C=CCOc1ccc(C[C@H](NC(=O)CCN2C(=O)C=CC2=O)C(=O)N2CCC[C@H]2C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)cc1]

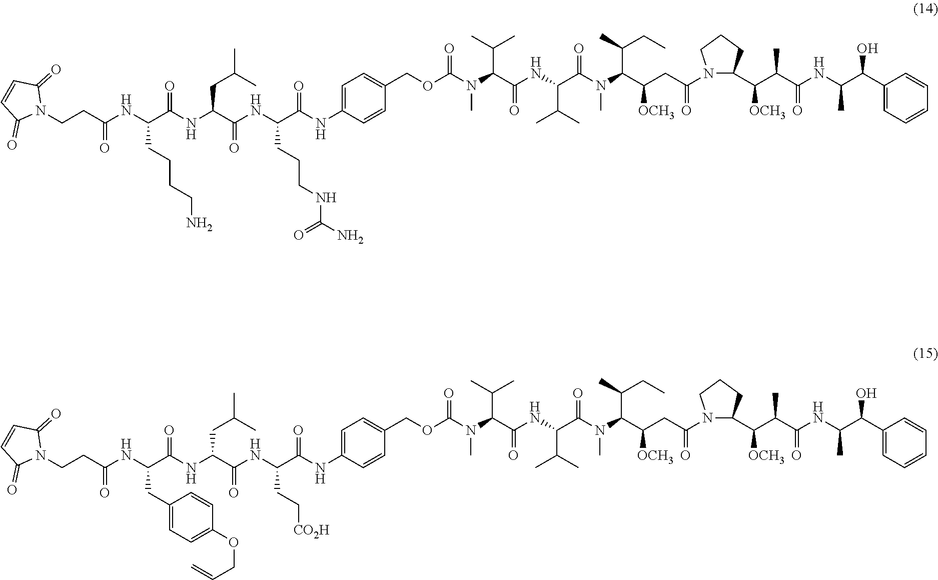 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@H](CC(C)C)NC(=O)[C@H](CCCCN)NC(=O)CCN2C(=O)C=CC2=O)cc1)C(C)C ; C=CCOc1ccc(C[C@H](NC(=O)CCN2C(=O)C=CC2=O)C(=O)N[C@H](CC(C)C)C(=O)N[C@@H](CCC(=O)O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)cc1]

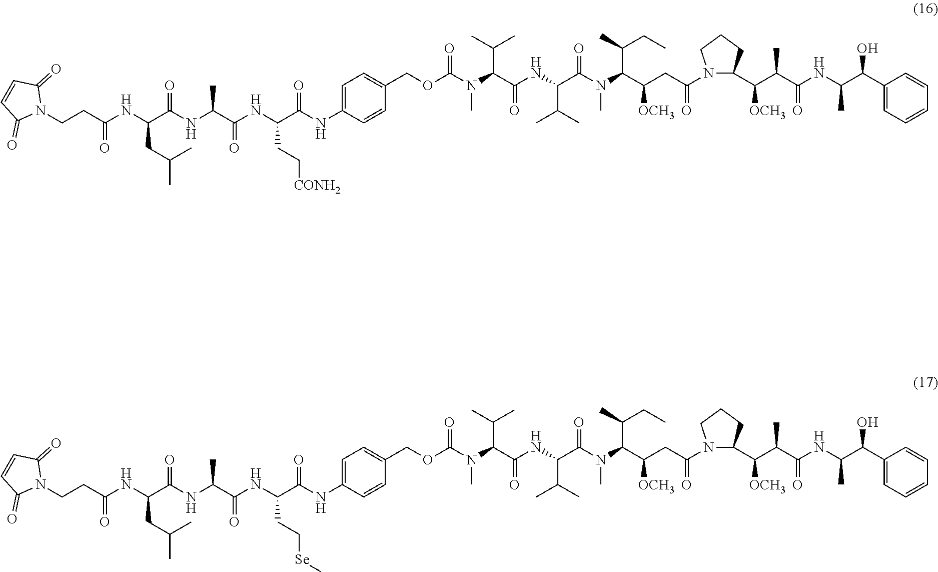 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCC(N)=O)NC(=O)[C@H](C)NC(=O)[C@@H](CC(C)C)NC(=O)CCN2C(=O)C=CC2=O)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CC[Se]C)NC(=O)[C@H](C)NC(=O)[C@@H](CC(C)C)NC(=O)CCN2C(=O)C=CC2=O)cc1)C(C)C]

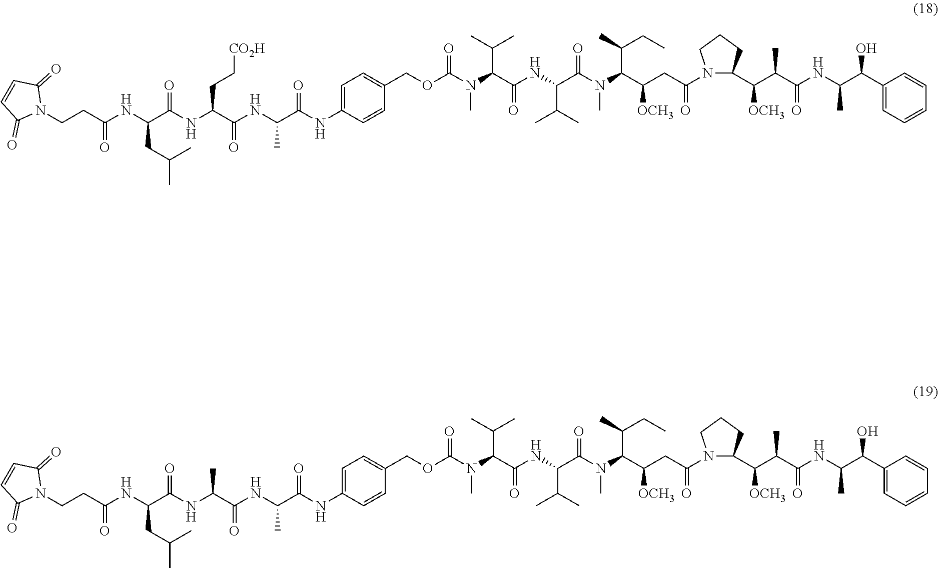 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@H](CCC(=O)O)NC(=O)[C@@H](CC(C)C)NC(=O)CCN2C(=O)C=CC2=O)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@H](C)NC(=O)[C@@H](CC(C)C)NC(=O)CCN2C(=O)C=CC2=O)cc1)C(C)C]

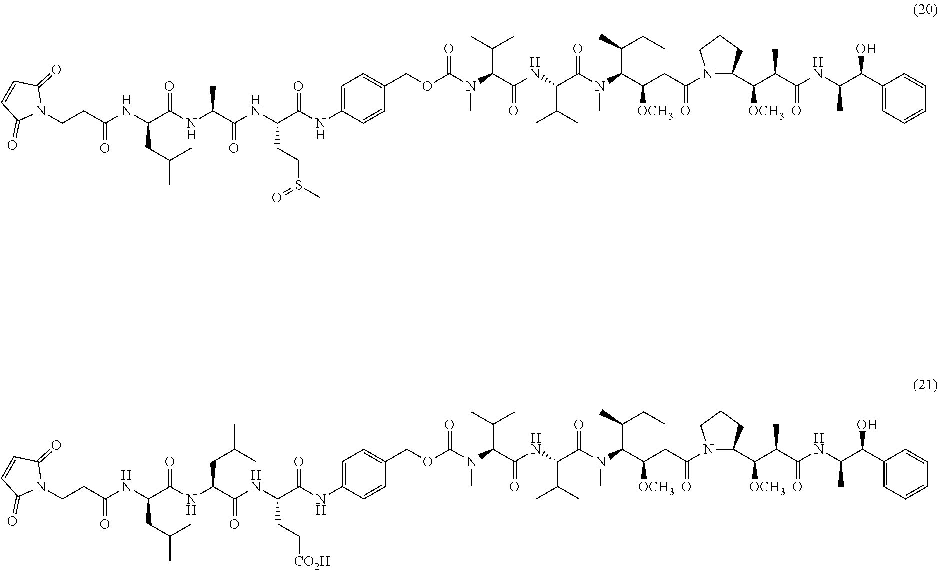 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCS(C)=O)NC(=O)[C@H](C)NC(=O)[C@@H](CC(C)C)NC(=O)CCN2C(=O)C=CC2=O)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCC(=O)O)NC(=O)[C@H](CC(C)C)NC(=O)[C@@H](CC(C)C)NC(=O)CCN2C(=O)C=CC2=O)cc1)C(C)C]

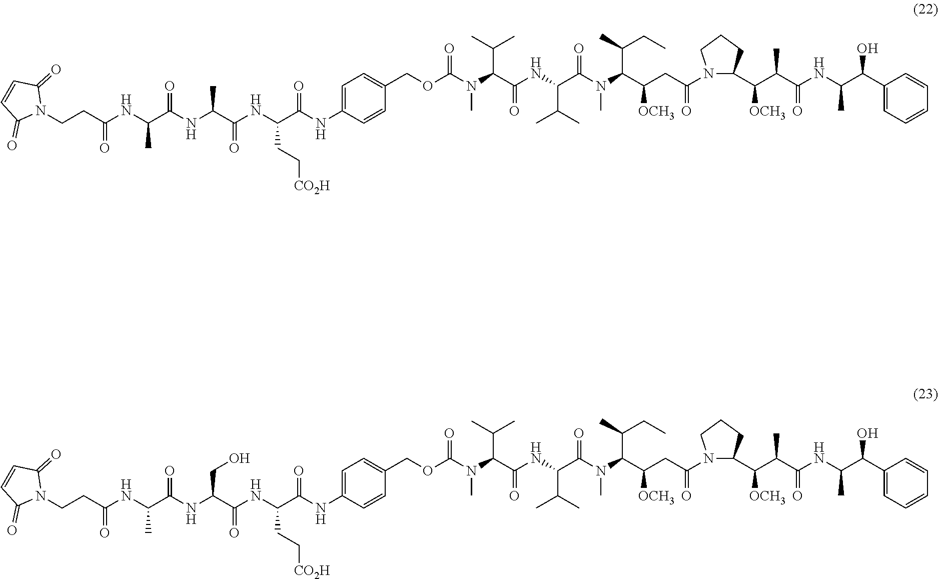 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCC(=O)O)NC(=O)[C@H](C)NC(=O)[C@@H](C)NC(=O)CCN2C(=O)C=CC2=O)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCC(=O)O)NC(=O)[C@H](CO)NC(=O)[C@H](C)NC(=O)CCN2C(=O)C=CC2=O)cc1)C(C)C]

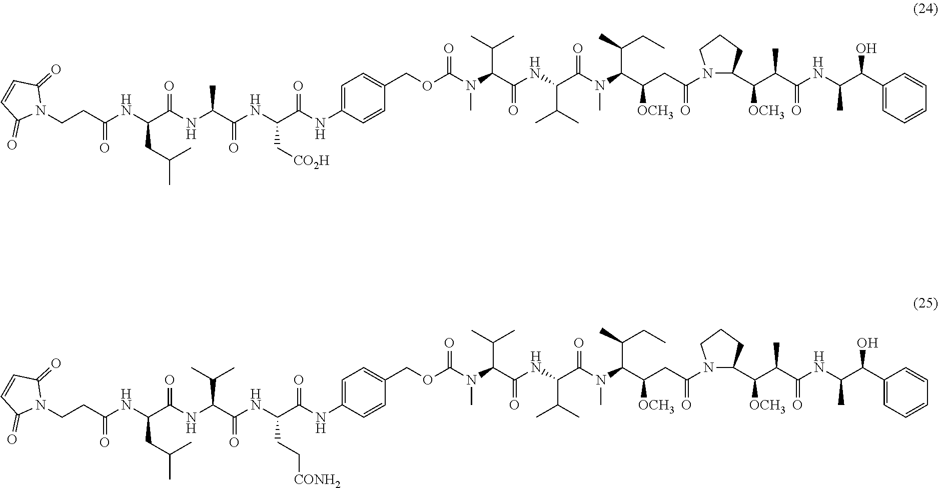 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CC(=O)O)NC(=O)[C@H](C)NC(=O)[C@@H](CC(C)C)NC(=O)CCN2C(=O)C=CC2=O)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCC(N)=O)NC(=O)[C@@H](NC(=O)[C@@H](CC(C)C)NC(=O)CCN2C(=O)C=CC2=O)C(C)C)cc1)C(C)C]

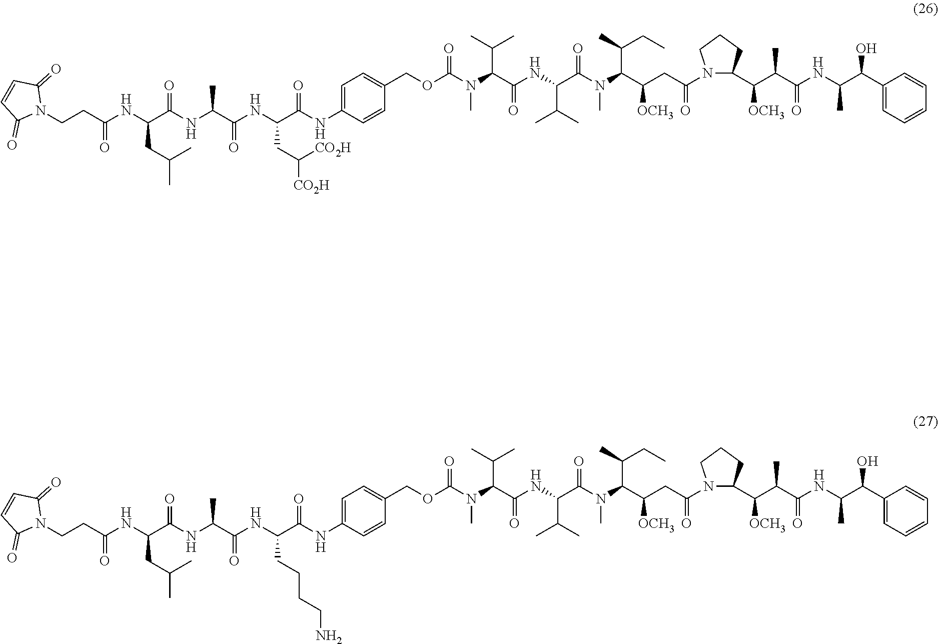 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CC(C(=O)O)C(=O)O)NC(=O)[C@H](C)NC(=O)[C@@H](CC(C)C)NC(=O)CCN2C(=O)C=CC2=O)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCCN)NC(=O)[C@H](C)NC(=O)[C@@H](CC(C)C)NC(=O)CCN2C(=O)C=CC2=O)cc1)C(C)C]

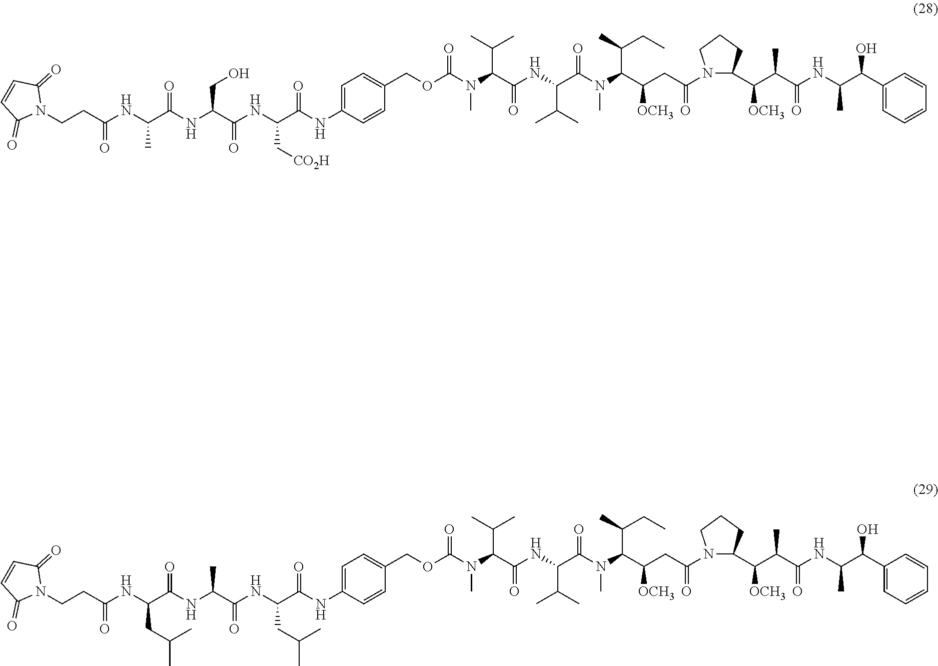 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CC(=O)O)NC(=O)[C@H](CO)NC(=O)[C@H](C)NC(=O)CCN2C(=O)C=CC2=O)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CC(C)C)NC(=O)[C@H](C)NC(=O)[C@@H](CC(C)C)NC(=O)CCN2C(=O)C=CC2=O)cc1)C(C)C]

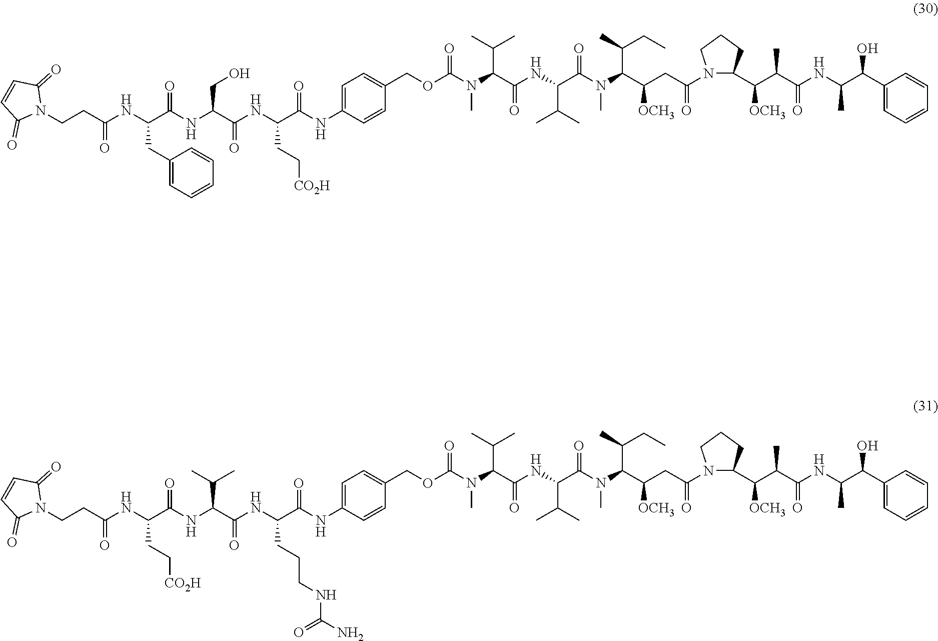 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCC(=O)O)NC(=O)[C@H](CO)NC(=O)[C@H](Cc2ccccc2)NC(=O)CCN2C(=O)C=CC2=O)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)O)NC(=O)CCN2C(=O)C=CC2=O)C(C)C)cc1)C(C)C]

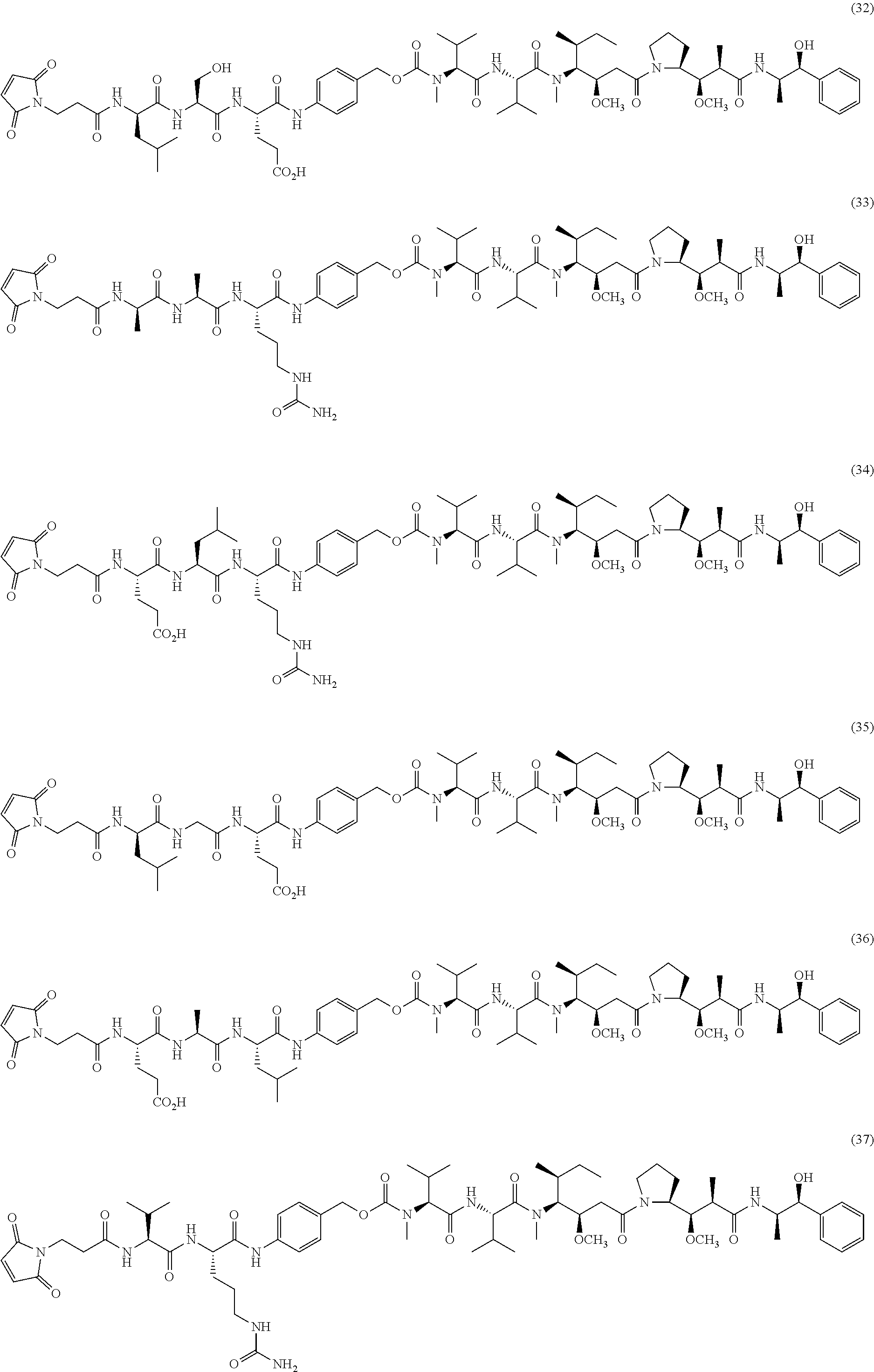

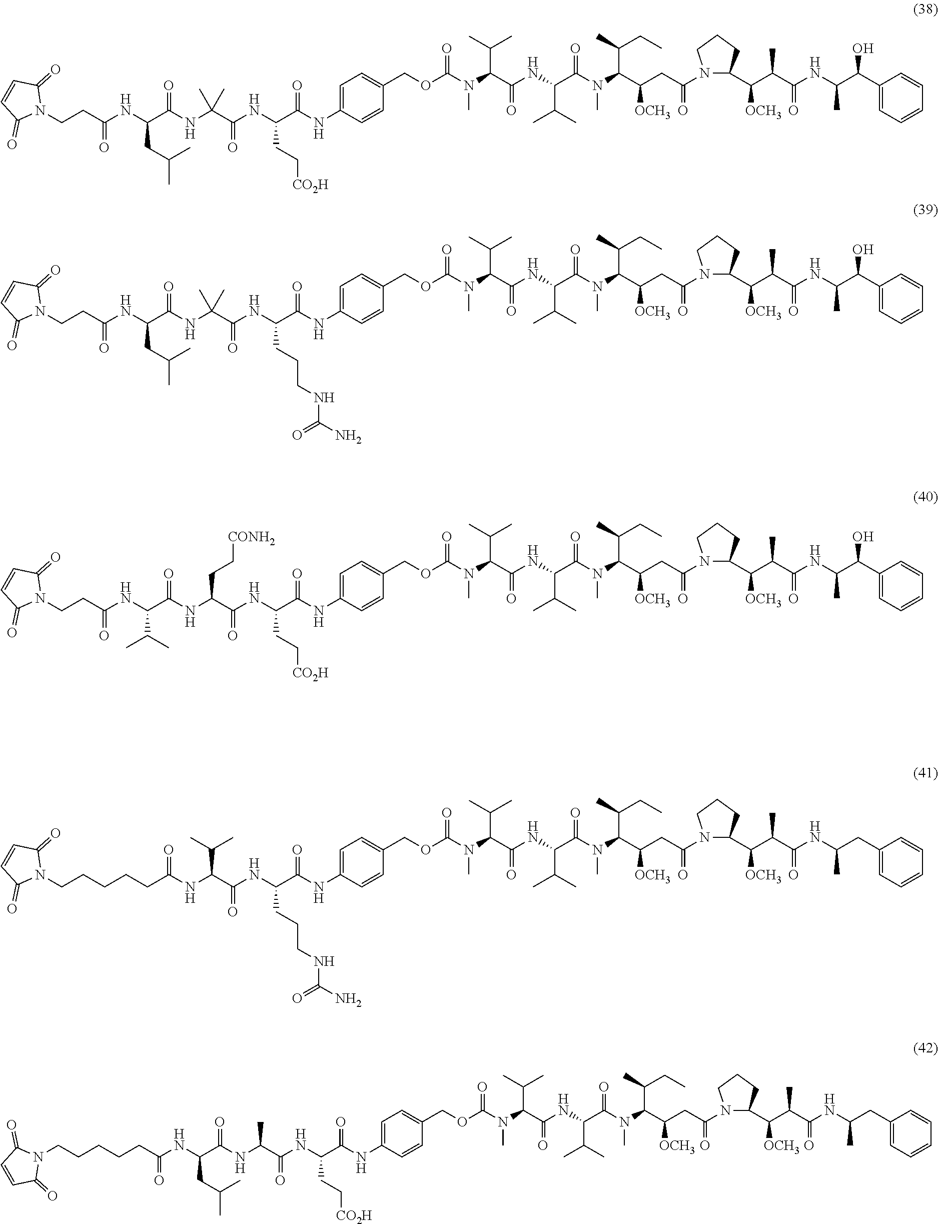

### Example 11: Preparation of Tripeptide Based MMAF Drug Linker Compounds

Drug Linker compounds in which MMAF is the Drug Unit and which may be used for preparing a similar subset of Ligand Drug Conjugates discussed in Part A, are represented by the following structure and were prepared according the reaction sequence of Scheme 4 starting from commercially available L-phenylalanine-2-chlorotrityl ester polymer-bound.

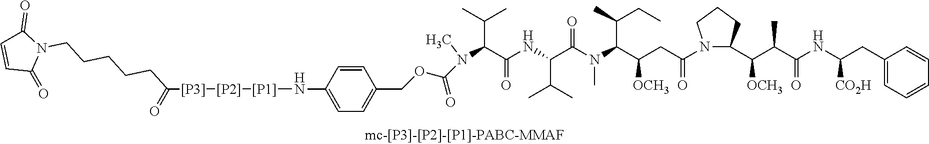

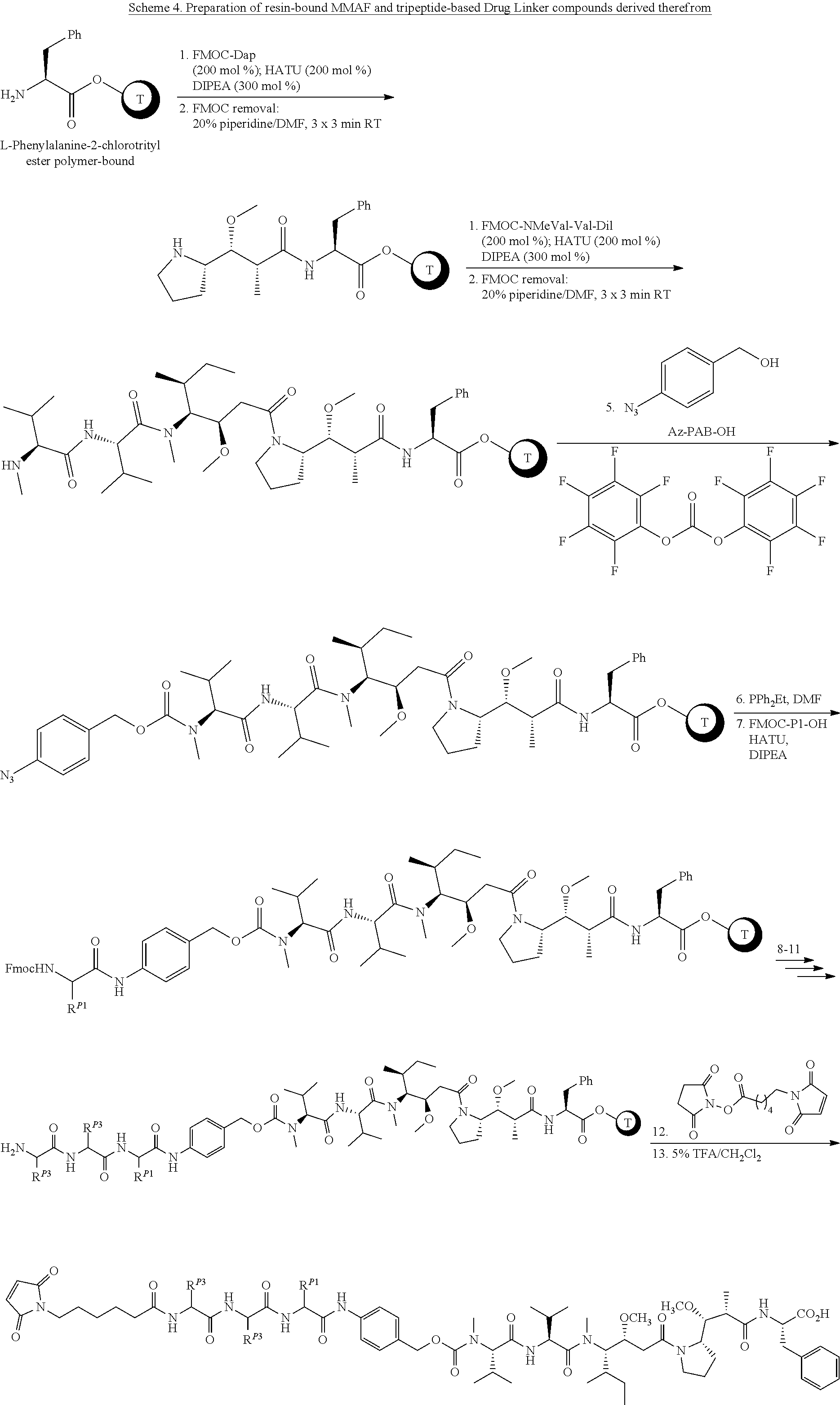

In Schemes 3 and 4, R^{P1}, R^{P2} and R^{P3} are the side chains of the P1, P2 and P3 amino acid residues, respectively.

### Example 12. In Vitro Cytotoxicity of Tripeptide-Based Antibody Drug Conjugates

Antibody Drug Conjugates having a drug antibody ratio (DAR) of about 4 were prepared according to the general procedures from the selected tripeptide based MMAE Drug-Linker compounds of Example 10 and a humanized antibody that selectively binds to an epithelial antigen (Ag1) that is commonly upregulated in various solid tumors including pancreatic, head and neck, lung, and esophageal tumors. Table 3 shows the IC₅₀ values against cells of a pancreatic adenocarcinoma cell line in which the Ag1 antigen is upregulated for the tripeptide-based ADCs (2-6) and for a dipeptide-based comparator conjugate (1) in which -val-cit- replaces the tripeptide Cleavable Unit. Table 3a shows the IC₅₀ values against cells of a HPAFII cell line in which the Ag1 antigen is upregulated for the tripeptide-based ADCs (8-10, 13, 16-21, 30, 31, and 38) and for a dipeptide-based comparator conjugate (1) in which -val-cit- replaces the tripeptide Cleavable Unit. Table 3b shows the IC₅₀ values against cells of a HPAFII cell line in which the Ag1 antigen is upregulated for the tripeptide-based ADCs (7, 15, 22-29, 32-36, 39, and 42) and for a dipeptide-based comparator conjugates (1 and 41) in which -val-cit-replaces the tripeptide Cleavable Unit. The italicized values in Tables 3, 3a, and 3b indicate the percentage of cells remaining after 96 hr incubation at the maximum concentration of drug added. For convenience the numbering for the library members of Tables 2 and 2A are retained for the corresponding Drug linker compounds that are incorporated into the ADC of Tables 3, 3a, and 3b.

TABLE 3
Cytotoxicity of ADCs against pancreatic adenocarcinoma cells

| | Cytotoxicity | |
|---|---|---|
| ADC | IC₅₀ | % |
| Ag1-1 | 54 | 21 |
| Ag1-2 | 36 | 21 |
| Ag1-3 | 22 | 22 |
| Ag1-4 | 35 | 21 |
| Ag1-5 | 62 | 23 |
| Ag1-6 | 30 | 20 |

The results of Table 3 show that the tripeptide-based ADCs (2-6) are equipotent with the comparator dipeptide-based ADC (1) whose tolerability is to be improved by replacing its dipeptide Cleavable Unit with each of the selected tripeptide sequences.

TABLE 3a
Cytotoxicity of ADCs against HPAFII cells

| | Cytotoxicity | |
|---|---|---|
| ADC | IC₅₀ | % |
| Ag1-1 | 39 | 32 |
| Ag1-8 | 279 | 39 |
| Ag1-9 | 85 | 34 |
| Ag1-10 | 37 | 32 |
| Ag1-13 | 69 | 21 |
| Ag1-16 | 83 | 29 |
| Ag1-17 | 57 | 24 |
| Ag1-18 | 81 | 27 |
| Ag1-19 | 29 | 24 |
| Ag1-20 | 70 | 31 |
| Ag1-21 | 75 | 25 |
| Ag1-30 | 144 | 31 |
| Ag1-31 | 47 | 21 |
| Ag1-38 | 484 | 54 |

The results of Table 3a show that several of the tripeptide-based ADCs (e.g. 8 and 30) are less cytotoxic than the comparator dipeptide-based ADC (1), but are similarly efficacious. The results of Table 3a also show that some tripeptide-based ADCs (e.g. 38) are less cytotoxic and efficacious than the comparator dipeptide-based ADC (1), but are less toxic to rat bone marrow, which may still afford an increased therapeutic window compared to comparator dipeptide-based ADC (1).

TABLE 3b
Cytotoxicity of ADCs against HPAFII cells

| | Cytotoxicity | |
|---|---|---|
| ADC | IC₅₀ | % |
| Ag1-1 | 413 | 12 |
| Ag1-7 | 226 | 29 |
| Ag1-15 | 802 | 33 |
| Ag1-22 | 2210 | NA |
| Ag1-23 | 581 | 31 |
| Ag1-24 | 1239 | 32 |
| Ag1-25 | 499 | 26 |
| Ag1-26 | 1725 | 37 |
| Ag1-27 | 99 | 11 |
| Ag1-28 | 768 | 22 |
| Ag1-29 | 261 | 20 |
| Ag1-32 | 318 | 32 |
| Ag1-33 | 279 | 11 |
| Ag1-34 | 158 | 21 |
| Ag1-35 | 859 | 27 |
| Ag1-36 | 160 | 15 |
| Ag1-39 | 2352 | 40 |
| Ag1-41 | 16 | 23 |
| Ag1-42 | 56 | 30 |

The results of Table 3b show that some of the tripeptide-based ADCs (e.g. 22, 24, and, 26) may be less cytotoxic than the comparator ADC (1), but are similarly efficacious.

### Example 13. In Vivo Cancer Cell Cytotoxicity of Tripeptide-Based Antibody Drug Conjugates

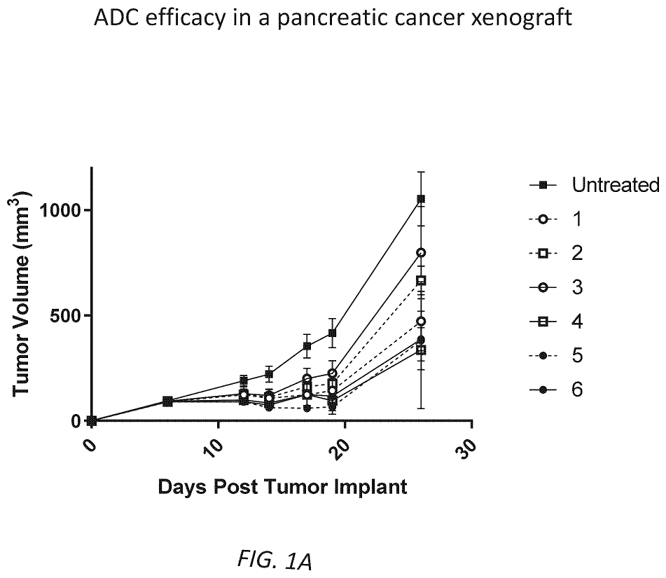

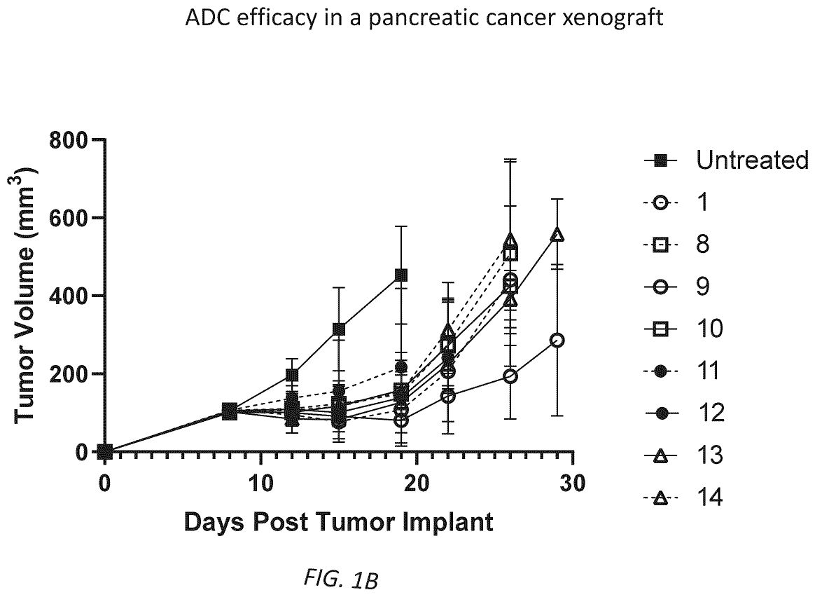

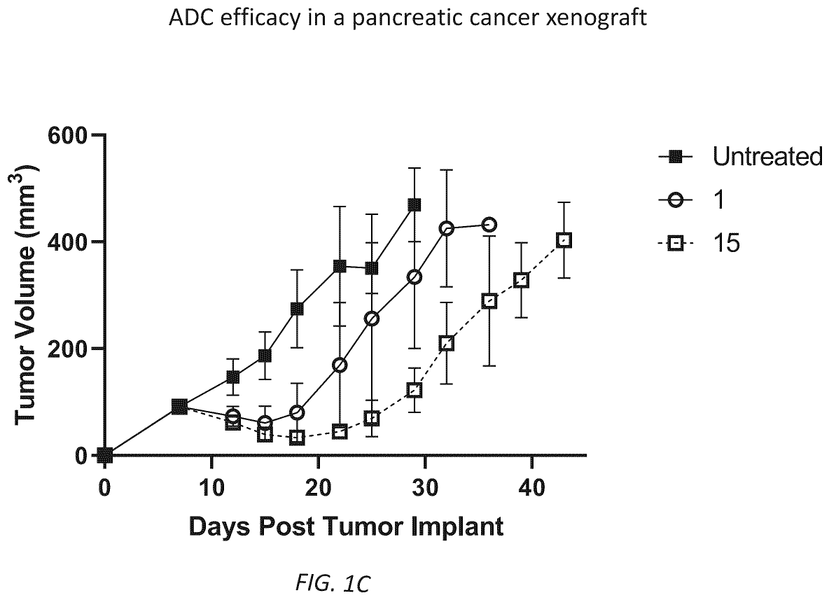

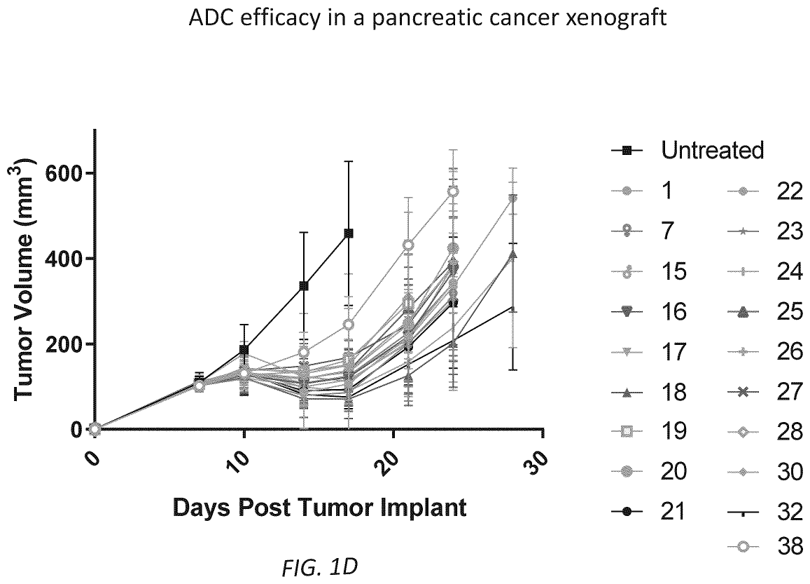

The ADCs of Table 3 were tested in a xenograft model in which cells of the pancreatic adenocarcinoma cell line of Example 12 were implanted in nude mice. Each tripeptide-based ADC was administered at the same subcurative dose (4 mg/Kg) determined for the dipeptide-based comparator conjugate in order to clearly distinguish efficacy differences. As seen in FIG. 1A, most tripeptide-based ADCs are at least as efficacious as the dipeptide-based comparator ADC.

The ADCs of Table 3a were tested in a xenograft model in which cells of the HPAFII cell line of Example 12 are implanted in nude mice. Each tripeptide-based ADC is administered at the same subcurative dose (3 mg/Kg) determined for the dipeptide-based comparator conjugate in order to clearly distinguish efficacy differences. As seen in FIGS. 1B and 1D, most tripeptide-based ADCs are generally at least as efficacious as the dipeptide-based comparator ADC.

The ADCs of Table 3b were tested in a xenograft model in which cells of the HPAFII cell line of Example 12 are implanted in nude mice. Each tripeptide-based ADC was administered at the same subcurative dose (3 mg/Kg), except tripeptide-based ADC Ag1-15 and the comparator dipeptide-based ADC that were both tested at 6 mg/kg (FIG. 1C), determined for the dipeptide-based comparator conjugate in order to clearly distinguish efficacy differences. As seen in FIGS. 1C and 1D, certain tripeptide-based ADCs are at least as efficacious as the dipeptide-based comparator ADC.

### Example 14. In Vivo Bone Marrow Toxicity of Tripeptide-Based Antibody Drug Conjugates

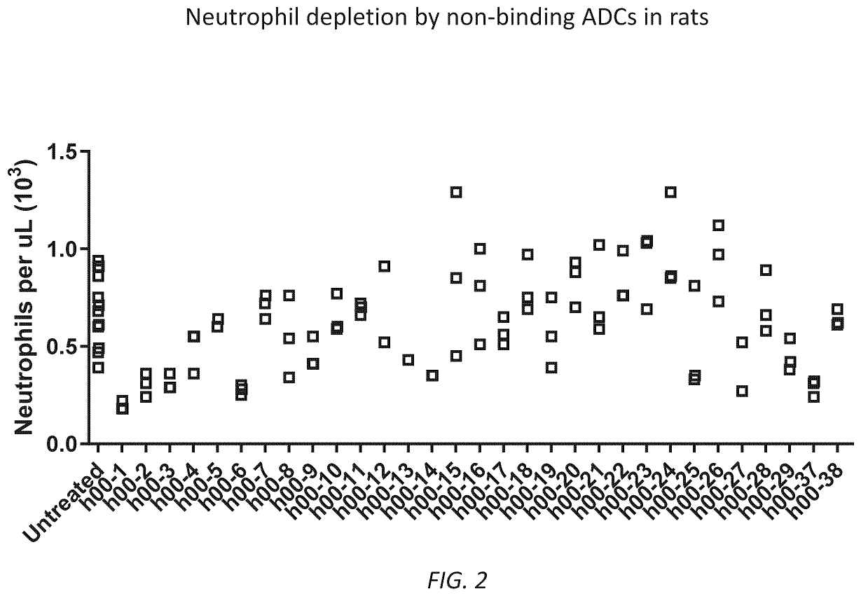

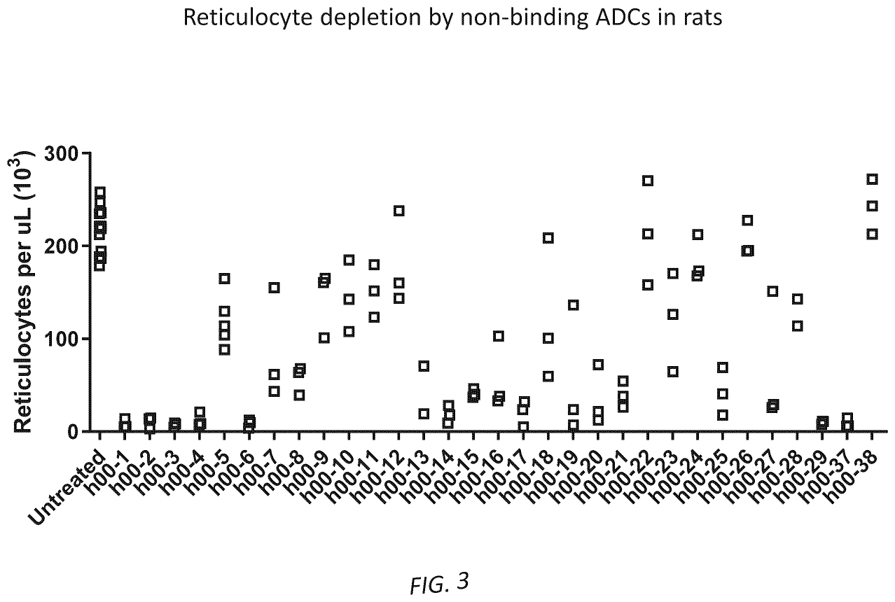

Having shown that ADC efficacy has been at least retained on replacing the dipeptide with most of the selected tripeptide sequences, differences in in vivo cytotoxicity against normal bone marrow tissue was explored by replacing the antibody targeting the Ag1 antigen with a non-binding control (h00) antibody. Each of the resulting non-targeting conjugates were then administered at 10 mg/Kg to rats, whose blood were analyzed at day 5 post-administration for neutrophil and reticulocyte counts as a proxy for bone marrow toxicity compared to sham treated animals. As seen from FIG. 2 some of the tripeptide-based h00 conjugates from Tables 3, 3a, and 3b showed improved neutrophil counts in comparison to the dipeptide-based comparator conjugate (h00-1). With respect to neutrophil counts, the tripeptide-based non-binding conjugates h00-4 and h00-5 showed similar preservation of that bone marrow cell type in comparison to h00-1. However, from the non-binding conjugates analogous to the targeting ADCs of Table 3, only the D-Leu-Ala-Glu non-binding control conjugate (h00-5) corresponding to the tripeptide-based targeting ADC of Table 3 (Ag1-5) exhibited an improved reticulocyte count relative to the comparator conjugate at the tested dose. Many more non-binding conjugates analogous to the targeting ADCs of Tables 3a and 3b exhibited improved preservation of neutrophil counts in comparison to h00-1. Comparisons between FIGS. 2 and 3 seem to indicate that reticulocytes are more sensitive to the MMAE non-binding conjugates than neutrophils, which is believed to be the reason that differences between the other tripeptide-based h00 non-binding conjugates analogous to the targeting ADCs of Table 3 could not be distinguished from each other or from h00-1 at the tested dose. Many more non-binding conjugates analogous to the targeting ADCs of Tables 3a and 3b exhibited improved preservation of reticulocyte counts in comparison to h00-1.

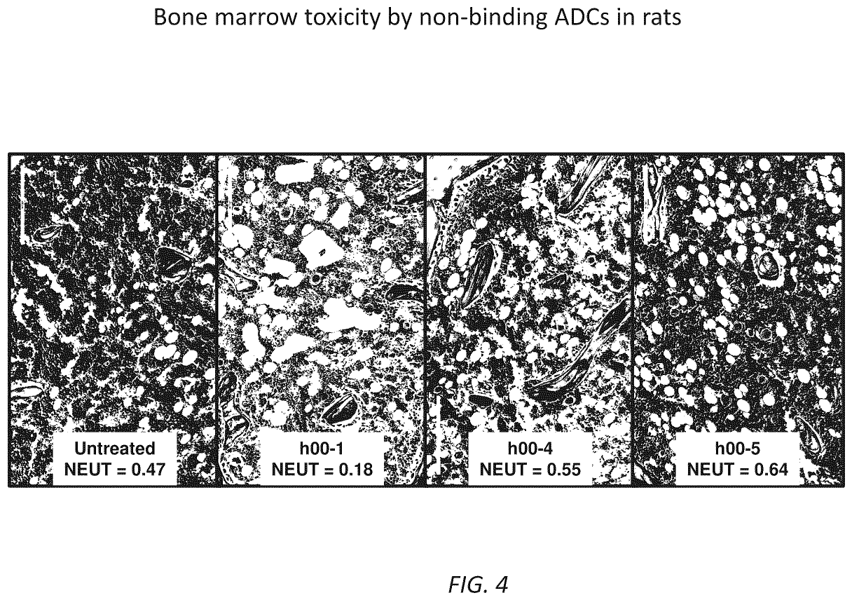

Histopathology of the bone marrow with IHC for mononuclear cells, which is shown in FIG. 4, confirms the preservation of mononuclear bone marrow cells by the tripeptide-based h00-4 and h00-5 conjugate compared with administration of the dipeptide-based comparator h00-1, with the result from administration of h00-5 conjugate being almost indistinguishable from sham treatment.

Included in FIGS. 2 and 3 is data for h00-7 in which the tripeptide sequence is Leu-Ala-Glu. That tripeptide is identical to that of h00-5 except that the stereochemical configuration of the P3 amino acid has been inverted. Both h00-5 and h00-7 appear to be less toxic to bone marrow than the other non-binding control ADCs with h00-5 being superior with respect to preserving the more sensitive reticulocytes.

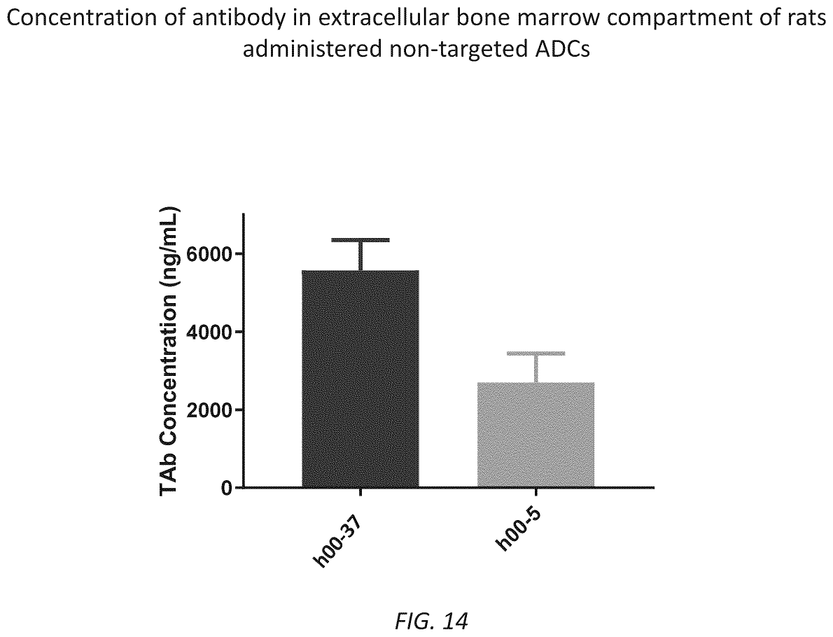

FIG. 14 shows the concentration of antibody in extracellular bone marrow compartment of rats administered non-targeted ADCs (h00-37 and h00-5).

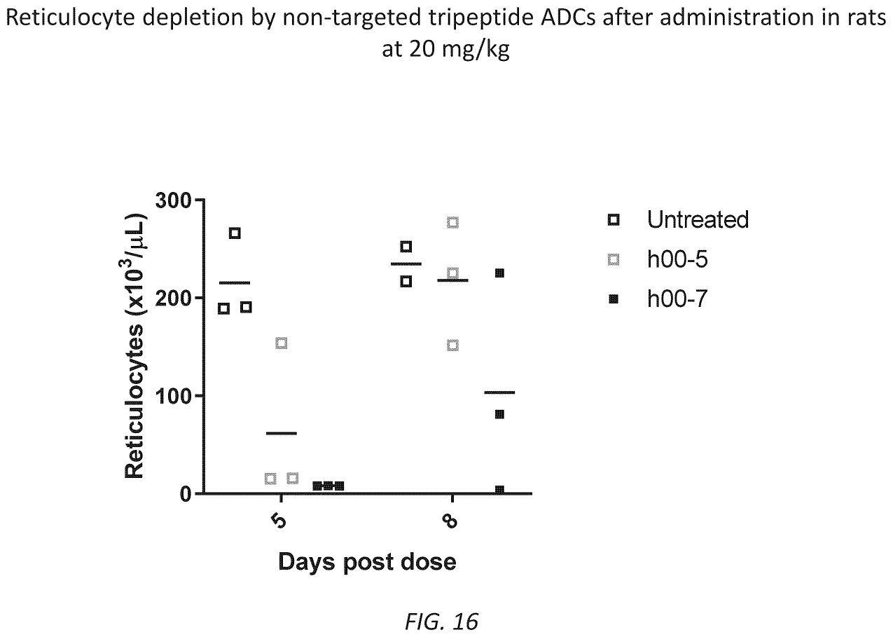

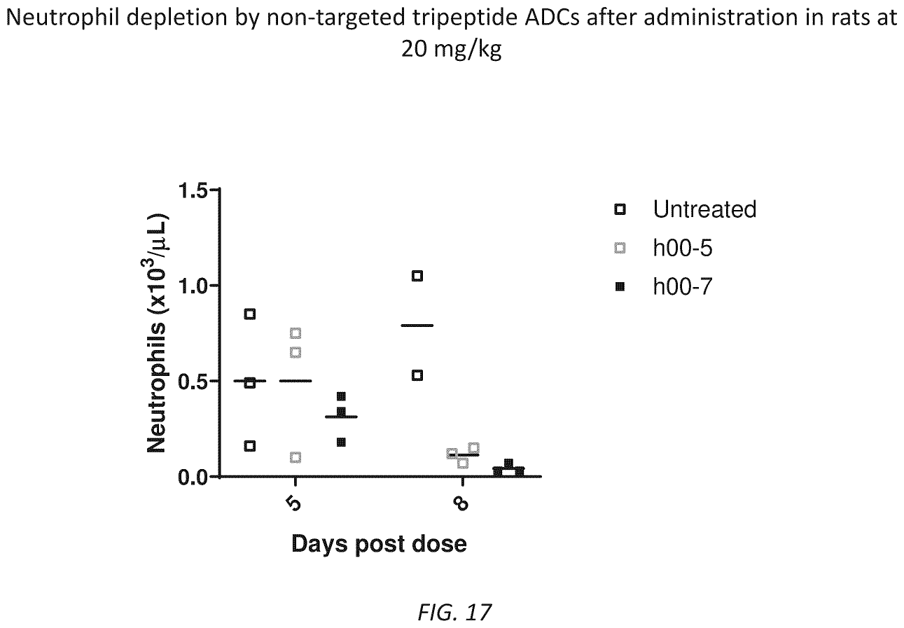

FIG. 16 shows the reticulocyte depletion on days 5 and 8 post dose by h00-5 and h00-7 after administration in rats at 20 mg/kg. FIG. 17 shows the neutrophil depletion on days 5 and 8 post dose by h00-5 and h00-7 after administration in rats at 20 mg/kg.

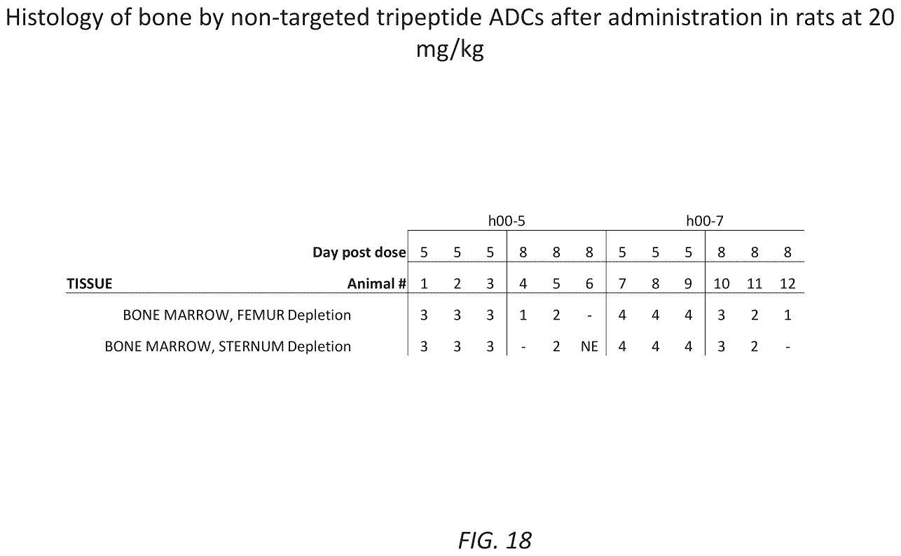

FIG. 18 shows the histology of bone on days 5 and 8 post dose by h00-5 and h00-7 after administration in rats at 20 mg/kg.

### Example 15. In Vivo Metabolism of Tripeptide-Based ADCs

Non-specific release of free drug from an ADC is one mechanism that contributes to off-target toxicity to normal cells. To determine if the preservation of bone marrow observed for the h00-4 and h00-5 ADCs as compared to the h00-1 ADC is due to reduction in release of free MMAE from the tripeptide-based ADCs, plasma from the toxicity study of Example 14 was analyzed for that metabolite by HPLC-MS.

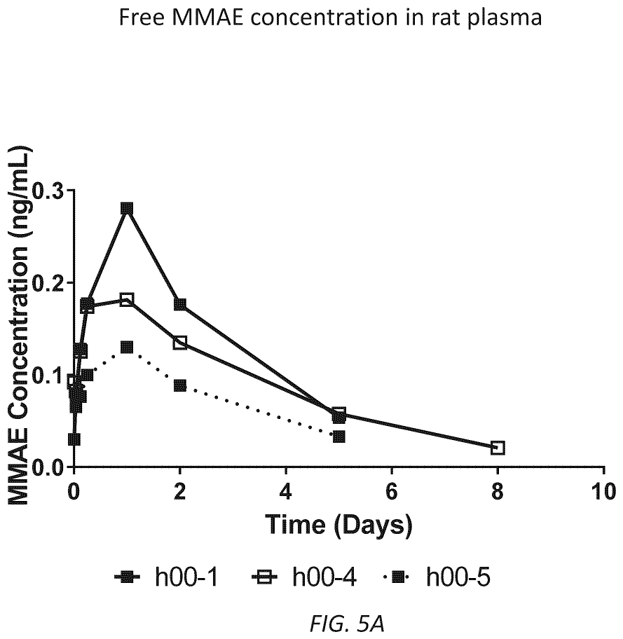

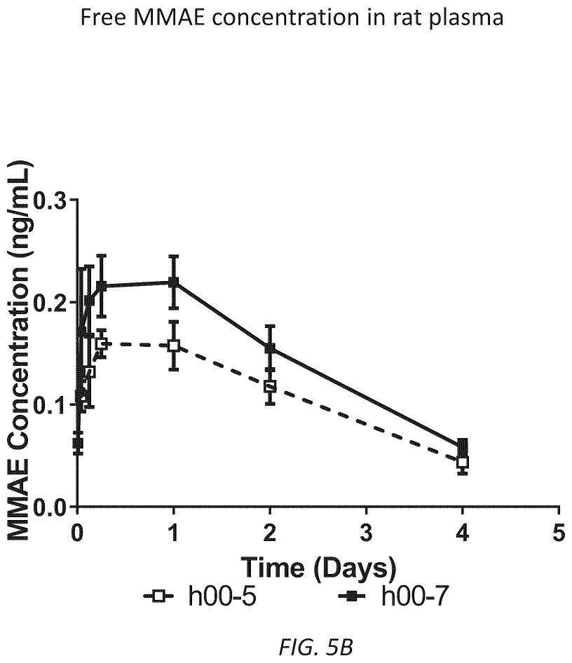

As shown in FIG. 5A, free MMAE concentration after administration of h00-4 or h00-5, remained below that found after administration of h00-1 throughout the course of the toxicity study, with the h00-5 conjugate being superior in that regard. Furthermore, FIG. 5B shows that the h00-5 conjugates, which has the P3 amino acid in the D stereochemical configuration, non-specifically releases less MMAE than h00-7, which is identical to h00-5 except the P3 amino acid is in the opposite stereochemical configuration. It thus appears that having an amino acid with the un-natural configuration at P3 confers improved stability to a tripeptide-based ADC.

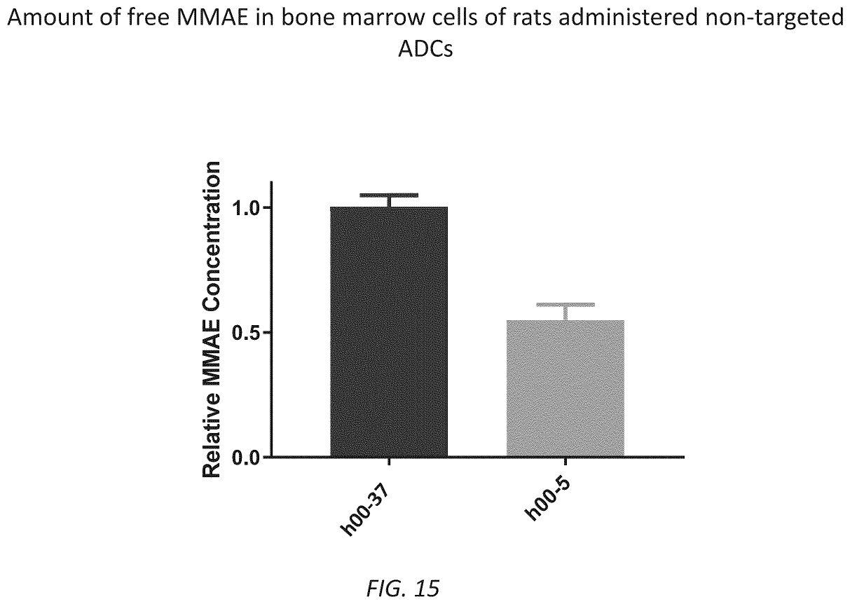

FIG. 15 shows the amount of free MMAE in bone marrow cells of rats administered non-targeted ADCs (h00-37 and h00-5).

### Example 16. Neutrophil Elastase Assay of Tripeptide-Based Antibody Drug Conjugates

To a mixture of 8-load ADC (5 ug), buffer (100 mM tris, 75 mM NaCl, pH 7.5; final concentration), and neutrophil elastase (100 ng) was added water to 20 μL. The reaction was incubated at 37 C for 3 h and then immediately analyzed by a QToF mass spectrometer.

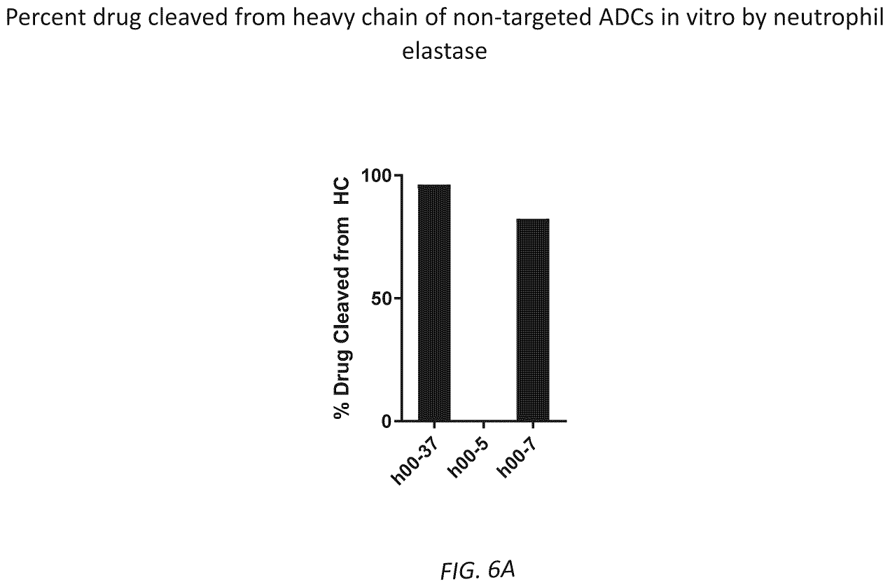

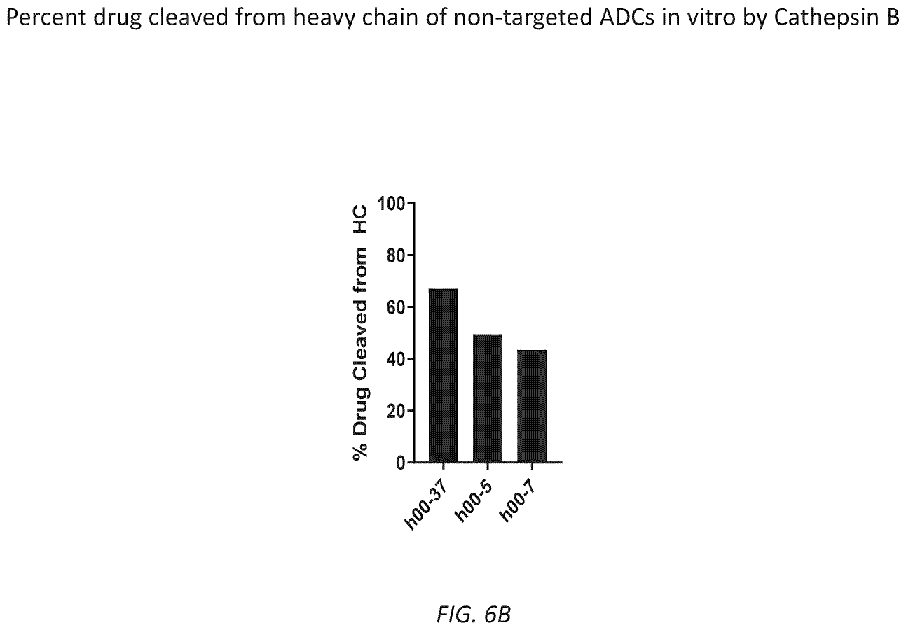

As shown in FIG. 6A, percentage of drug cleaved from the heavy chain of non-targeted ADC 5 in vitro by neutrophil elastase is lower than that found for non-targeted ADC 37. Furthermore, FIG. 6A shows that the h00-5 conjugate, which has the P3 amino acid in the D stereochemical configuration, has its heavy chain cleaved by neutrophil elastase to a significantly lower extent than h00-7, which is identical to h00-5 except the P3 amino acid is in the opposite stereochemical configuration. In fact, no proteolysis of h00-5 by neutrophil elastase was observed. It thus appears that having an amino acid with the un-natural configuration at P3 confers improved stability to a tripeptide-based ADC.

### Example 17. Cathepsin B Assay of Tripeptide-Based Antibody Drug Conjugates

To a mixture of 8-load ADC (5 ug), buffer (50 mM citrate, 75 mM NaCL, pH 4.5; final concentration), cathepsin B (100 ng) and activating buffer (2 mM DTT/1.33 mM EDTA final concentration) was added water to 20 uL. The reaction was incubated at 37 C for 3 h and then immediately analyzed by a QToF mass spectrometer

As shown in FIG. 6B, percentage of drug cleaved from the heavy chain of non-targeted ADCs 5 and 7 in vitro by Cathepsin B is similar to that found for non-targeted ADC 37 suggesting that the D-Leu-Ala-Glu non-binding control conjugate (h00-5) is cleaved similarly to the Val-Cit non-binding control conjugate (h00-37) by a lysosomal protease.

### Example 18. In Vitro Plasma Aggregation Assay of Tripeptide-Based Antibody Drug Conjugates

ADCs were labeled with Alexa Fluor 488 TFP ester (Molecular Probes), desalted, buffer exchanged into PBS, pH 7.4 (Gibco), and sterile filtered. The concentration and degree of labeling of the resulting ADC-AF488 conjugate was determined by UV absorbance prior to freezing at −80° C. On the day of experiment, AF488-ADC was diluted in plasma and incubated at 37° C. At the indicated time points, aliquots were analyzed by SEC-UPLC with fluorescence detection. The resulting chromatograms were analyzed to determine % of high molecular weight species.

The aggregation appears to be lower for tripeptide MMAF than Val-Cit-MMAF. Based on the correlation observed with MMAE, the tripeptide MMAF would be less toxic.

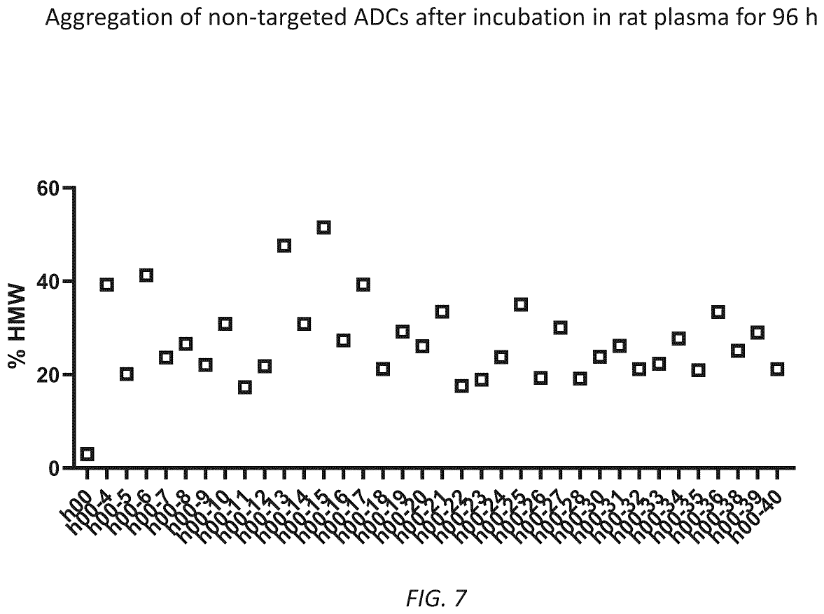

FIG. 7 shows the aggregation of non-targeted ADCs after incubation in rat plasma for 96.

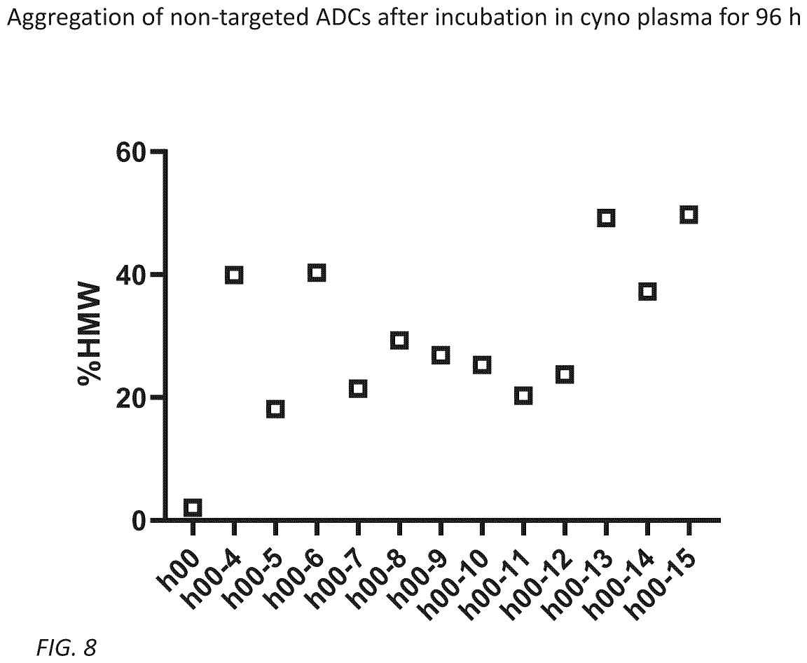

FIG. 8 shows the aggregation of non-targeted ADCs after incubation in cyno plasma for 96.

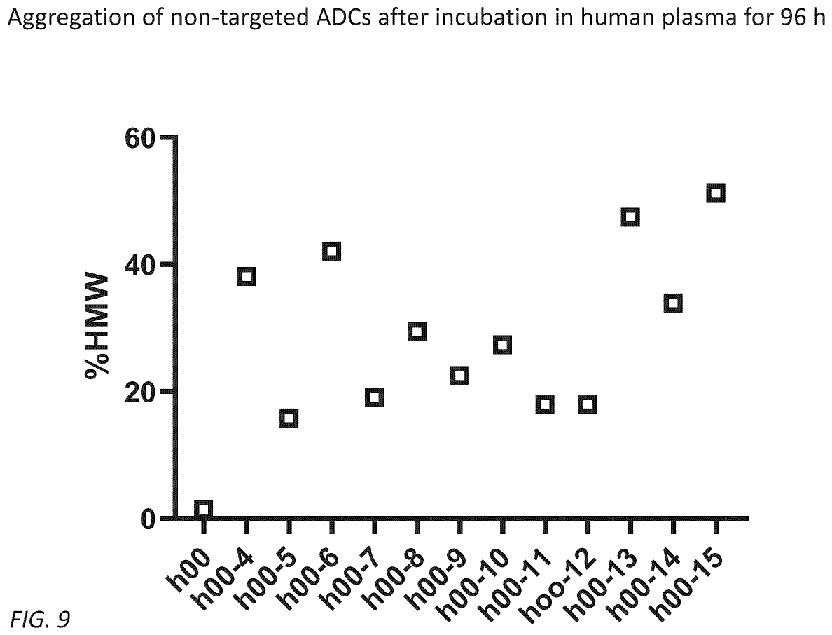

FIG. 9 shows the aggregation of non-targeted ADCs after incubation in human plasma for 96.

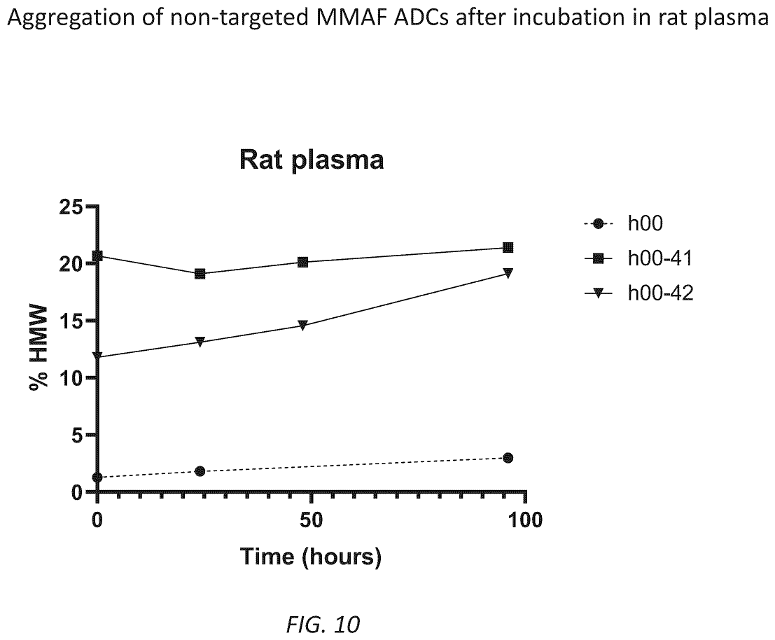

FIG. 10 shows the aggregation of non-targeted MMAF ADCs (h00-41 and h00-42) after incubation in rat plasma.

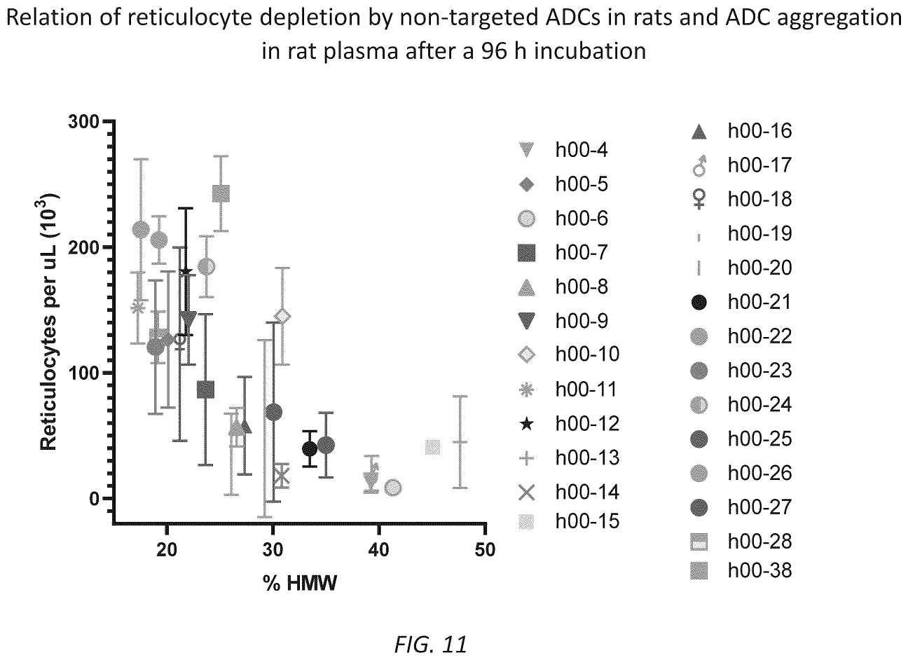

FIG. 11 shows the correlation of reticulocyte depletion by non-targeted ADCs in rats and ADC aggregation in rat plasma after a 96 h incubation.

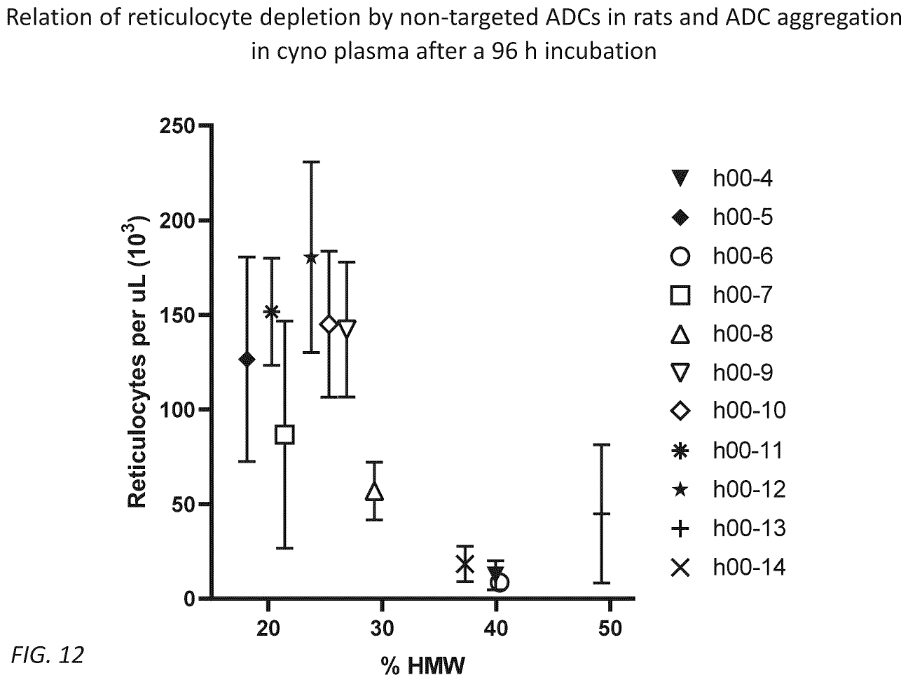

FIG. 12 shows the correlation of reticulocyte depletion by non-targeted ADCs in rats and ADC aggregation in cyno plasma after a 96 h incubation.

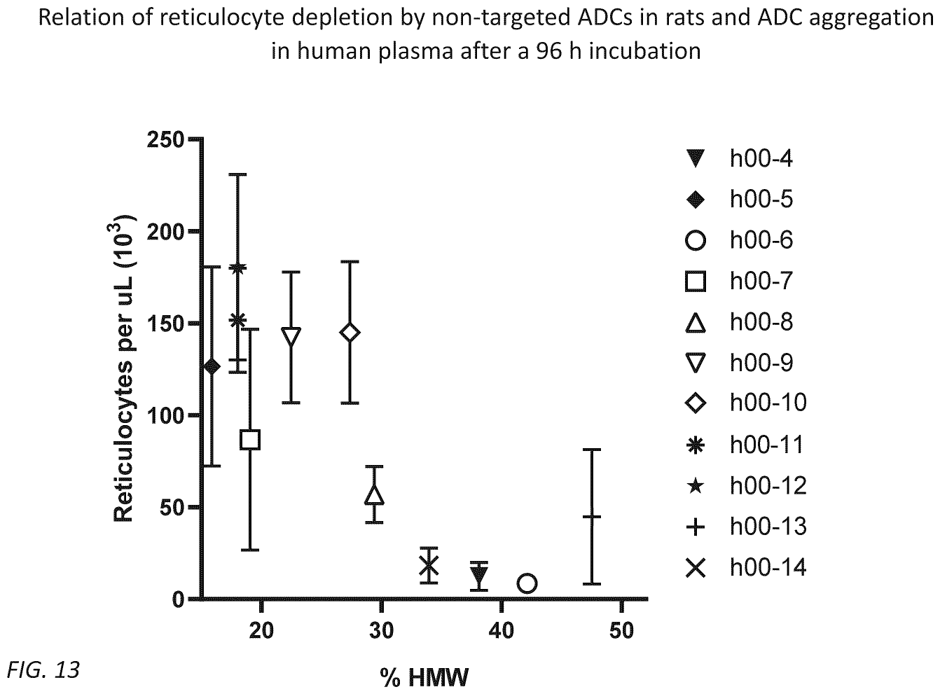

FIG. 13 shows the correlation of reticulocyte depletion by non-targeted ADCs in rats and ADC aggregation in human plasma after a 96 h incubation.

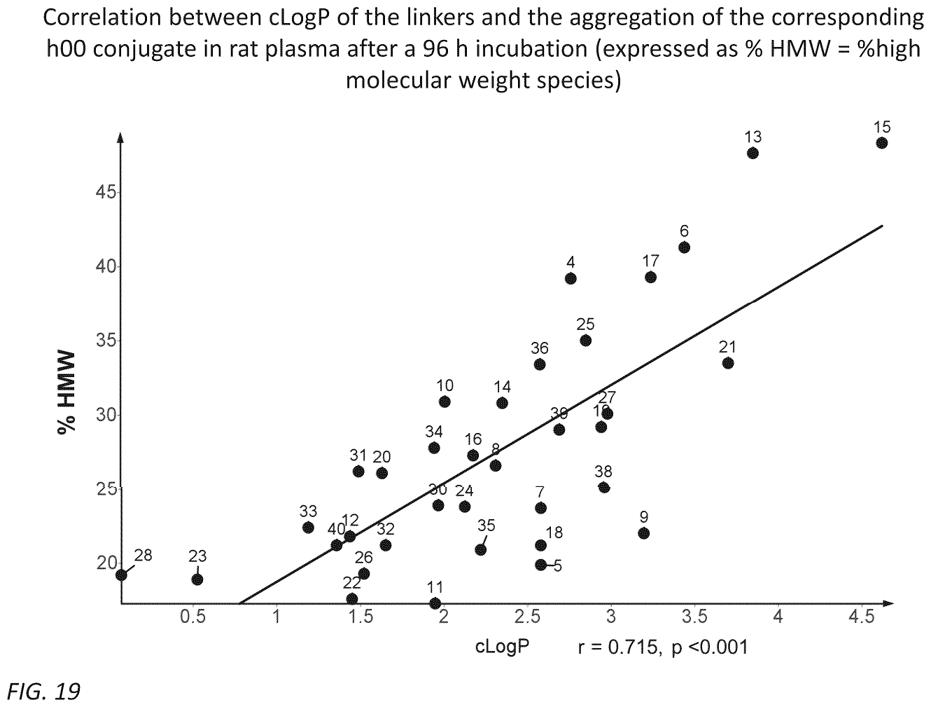

In FIG. 19, wherein the correlation between cLogP of the linkers and the aggregation of the corresponding h00 conjugate in rat plasma is shown, correlation of r=0.715 indicates that presence of HMW positively correlates with the clogP (i.e., the linkers with lower cLogP values show less aggregation than those with higher clogP). Linkers with low cLogP values have low hydrophobicity, which includes linkers with polar amino acids.

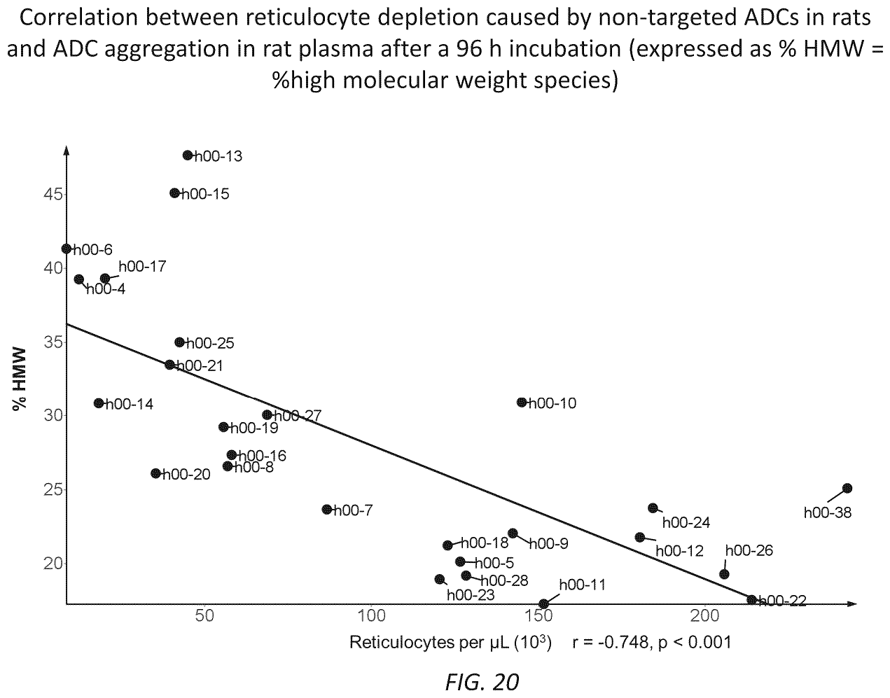

In FIG. 20, wherein the correlation between reticulocyte depletion caused by non-targeted ADCs in rats and ADC aggregation in rat plasma is shown, correlation of r=−0.748 indicates that presence of HMW negatively correlates with reticulocytes (i.e. higher the % HMW, higher is the depletion of reticulocytes).

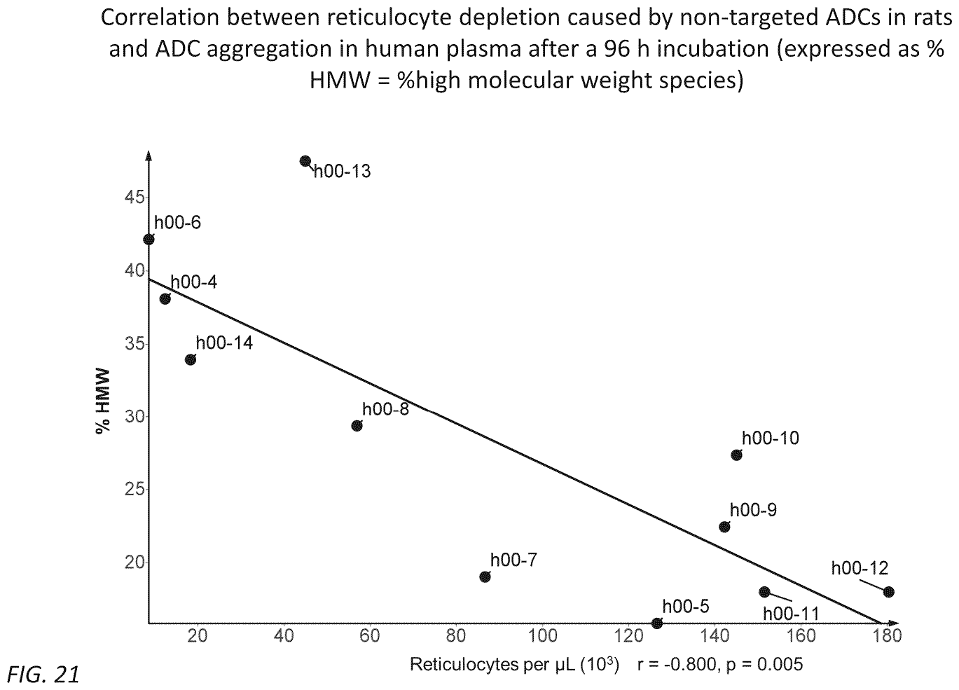

In FIG. 21, wherein the correlation between reticulocyte depletion caused by non-targeted ADCs in rats and ADC aggregation in human plasma is shown, correlation of r=−0.800 indicates that presence of HMW negatively correlates with reticulocytes (i.e. higher the % HMW, higher is the depletion of reticulocytes).

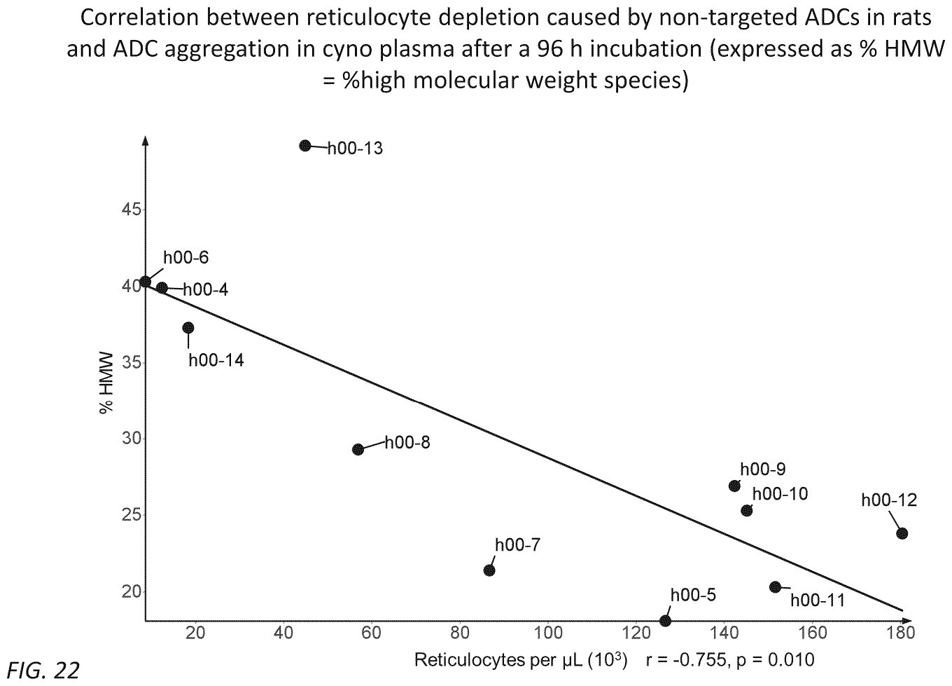

In FIG. 22, wherein the correlation between reticulocyte depletion caused by non-targeted ADCs in rats and ADC aggregation in cyno plasma is shown, correlation of r=−0.755 indicates that presence of HMW negatively correlates with reticulocytes (i.e., higher the % HMW, higher is the depletion of reticulocytes).

1. A Ligand Drug Conjugate composition represented by Formula 1:
L-[LU-D′]ₚ (1)
or a pharmaceutically acceptable salt thereof, wherein
L is a Ligand Unit;
LU is a Linker Unit;
D′ represents from 1 to 4 Drug Units (D) in each drug linker moiety of formula -LU-D′; and
subscript p is a number from 1 to 12, from 1 to 10 or from 1 to 8 or is about 4 or about 8,
wherein the Ligand Unit is from an antibody or an antigen-binding fragment of an antibody that is capable of selective binding to an antigen of tumor tissue for subsequent release of the Drug Unit(s) as free drug,
wherein the drug linker moiety of formula -LU-D′ in each of the Ligand Drug Conjugate compounds of the composition has the structure of Formula 1A: 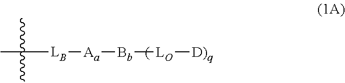
or a salt thereof,
wherein the wavy line indicates covalent attachment to L;
D is the Drug Unit;
L_{B} is a ligand covalent binding moiety;
A is a first optional Stretcher Unit;
subscript a is 0 or 1, indicating the absence or presence of A, respectively;
B is an optional Branching Unit;
subscript b is 0 or 1, indicating the absence or presence of B, respectively;
L_{O} is a secondary linker moiety, wherein the secondary linker has the formula of; 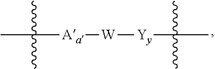
wherein the wavy line adjacent to Y indicates the site of covalent attachment of L_{O} to the Drug Unit and the wavy line adjacent to A′ indicates the site of covalent attachment to the remainder of the drug linker moiety;
A′ is a second optional Stretcher Unit, which in the absence of B becomes a subunit of A,
subscript a′ is 0 or 1, indicating the absence or presence of A′, respectively,
W is a Peptide Cleavable Unit, wherein the Peptide Cleavable Unit comprises a tripeptide having the sequence —P3-P2-P1-, wherein P1, P2, and P3 are each an amino acid, wherein:
a first one of the amino acids P1, P2, or P3 is negatively charged;
a second one of the amino acids P1, P2, or P3 has an aliphatic side chain with hydrophobicity no greater than that of leucine; and
a third one of the amino acids P1, P2, or P3 has hydrophobicity lower than that of leucine,
wherein the first one of the amino acids P1, P2, or P3 corresponds to any one of P1, P2, or P3, the second one of the amino acids P1, P2, or P3 corresponds to one of the two remaining amino acids P1, P2, or P3, and the third one of the amino acids P1, P2, or P3 corresponds to the last remaining amino acids P1, P2, or P3,
provided that —P3-P2-P1- is not -Glu-Val-Cit- or -Asp-Val-Cit-;
Y is a self-immolative Spacer Unit;
subscript y is 0, 1 or 2 indicating the absence or presence of 1 or 2 of Y, respectively; and
subscript q is an integer ranging from 1 to 4,
provided that subscript q is 1 when subscript b is 0 and subscript q is 2, 3 or 4 when subscript b is 1; and
wherein the Ligand Drug Conjugate compounds of the composition have the structure of Formula 1 in which subscript p is replaced by subscript p′, wherein subscript p′ is an integer from 1 to 12, 1 to 10 or 1 to 8 or is 4 or 8.

2. The Ligand Drug Conjugate composition of claim 1, wherein the Ligand Drug Conjugate compounds in the Ligand Drug Conjugate composition predominately have drug linker moieties of Formula 1H: 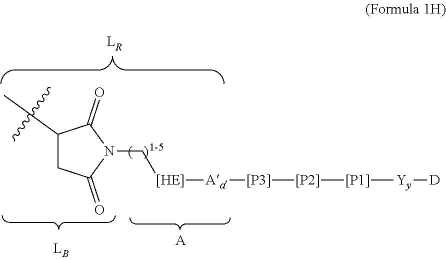
or a pharmaceutically acceptable salt thereof, and optionally having a minority of Ligand Drug Conjugate compounds in which one or more of the drug linker moieties in each of such compounds has its succinimide ring in hydrolyzed form and wherein
HE is a Hydrolysis Enhancing Unit;
A′ is a subunit, when present, of the indicated first Stretcher Unit (A); subscript a′ is 0 or 1, indicating the absence or presence of A′; and
the wavy line indicates the site of covalent binding to a sulfur atom of the Ligand Unit.

3. (canceled)

4. The Ligand Drug Conjugate composition of claim 1 or a pharmaceutically acceptable salt thereof, wherein —Y_{y}-D has the structure of: 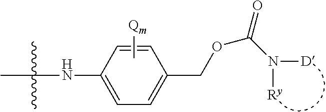
wherein —N(R^{y})D′ represents D, wherein D′ is the remainder of D;
the wavy line indicates the site of covalent attachment to P1;
the dotted line indicates optional cyclization of R^{y} to D′;
R^{y} is optionally substituted C₁-C₆ alkyl in absence of cyclization to D′ or optionally substituted C₁-C₆ alkylene when cyclized to D′;
each Q is independently selected from the group consisting of —C₁-C₈ alkyl, —O—(C₁-C₈ alkyl), halogen, nitro and cyano; and
subscript m is 0, 1, or 2.

5. The Ligand Drug Conjugate composition of claim 1 or a pharmaceutically acceptable salt thereof, wherein D is a cytotoxic drug wherein the cytotoxic drug is a secondary amine-containing auristatin compound wherein the nitrogen atom of the secondary amine is the site of covalent attachment to the drug linker moiety and the secondary amine-containing auristatin compound has the structure of Formula D_{F/E-3}: 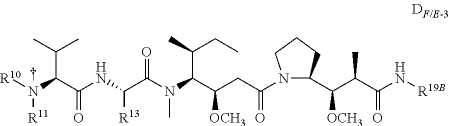
wherein the dagger indicates the site of covalent attachment of the nitrogen atom that provides the carbamate functional group;
one of R¹⁰ and R¹¹ is hydrogen and the other is methyl;
R¹³ is isopropyl or —CH₂—CH(CH₃)₂; and
R^{19B} is —CH(CH₃)—CH(OH)-Ph, —CH(CO₂H)—CH(OH)—CH₃, —CH(CO₂H)—CH₂Ph, —CH(CH₂Ph)-2-thiazolyl, —CH(CH₂Ph)-2-pyridyl, —CH(CH₂-p-Cl-Ph), —CH(CO₂Me)-CH₂Ph, —CH(CO₂Me)-CH₂CH₂SCH₃, —CH(CH₂CH₂SCH₃)C(═O)NH-quinol-3-yl, —CH(CH₂Ph)C(═O)NH-p-Cl-Ph, or
R^{19B} has the structure of 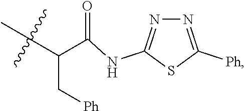 [SMILES: *C(Cc1ccccc1)C(=O)Nc1nnc(-c2ccccc2)s1]
wherein the wavy line indicates covalent attachment to the remainder of the auristatin compound.

6. The Ligand Drug Conjugate composition of claim 5, or a pharmaceutically acceptable salt thereof, wherein the secondary amine-containing auristatin compound is monomethylauristatin E (MMAE) or monomethylauristatin F (MMAF).

7. The Ligand Drug Conjugate composition of claim 1, wherein subscript q is 1 and the Ligand Drug Conjugate compounds in the Ligand Drug Conjugate composition predominately have drug linker moieties of Formula 1H-MMAE: 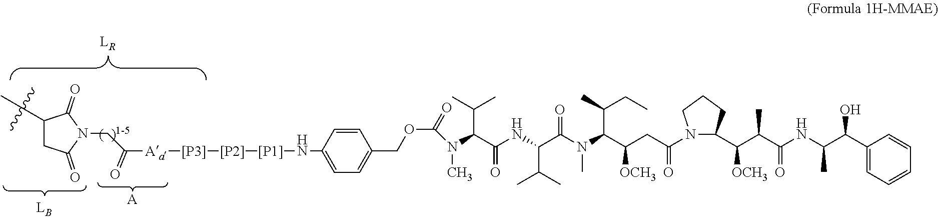
or a pharmaceutical acceptable salt thereof, and optionally having a minority of Ligand Drug Conjugate compounds in which one or more of the drug linker moieties in each of such compounds has its the succinimide ring in hydrolyzed form and wherein:
subscript a′ is 0, and A′ is absent; and
the wavy line indicates the site of covalent binding to a sulfur atom of the Ligand Unit.

8-12. (canceled)

13. The Ligand Drug Conjugate composition of claim 1, or a pharmaceutically acceptable salt thereof, wherein —P3-P2-P1- is -D-Leu-Ala-Asp-, -D-Leu-Ala-Glu-, -D-Ala-Ala-Asp-, or -D-Ala-Ala-Glu-.

14. The Ligand Drug Conjugate composition of claim 1, or a pharmaceutically acceptable salt thereof, wherein the P3 amino acid is D-Leu or D-Ala, the P2 amino acid is Ala, Glu, or Asp, and the P1 amino acid is Ala, Glu, or Asp.

15. The Ligand Drug Conjugate compound of claim 1, wherein the compound has the structure of: 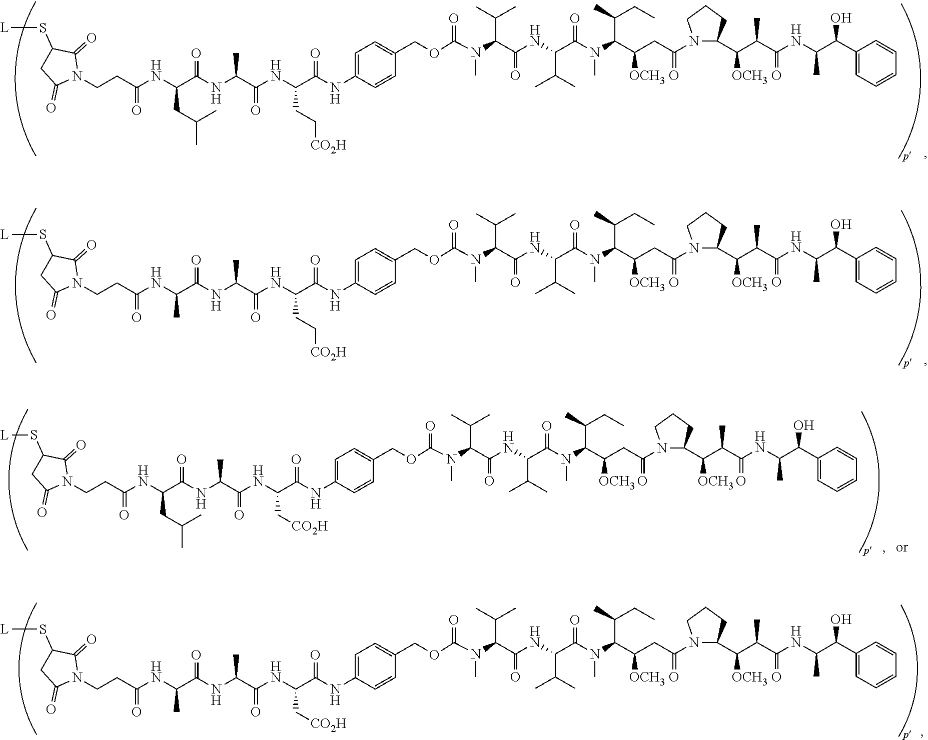
or a pharmaceutically acceptable salt thereof,
wherein L is a Ligand Unit, and subscript p′ is an integer from 1 to 12.

16-19. (canceled)

20. A pharmaceutically acceptable formulation, wherein the formulation comprises an effective amount of the Ligand Drug Conjugate composition of claim 1, or a pharmaceutically acceptable salt thereof and at least one pharmaceutically acceptable excipient.

21-22. (canceled)

23. A Drug Linker compound of Formula IA: 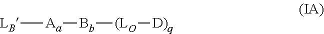
or a salt thereof, wherein
D is a Drug Unit;
L_{B}′ is a ligand covalent binding precursor moiety;
A is a first optional Stretcher Unit;
subscript a is 0 or 1, indicating the absence or presence of A, respectively;
B is an optional Branching Unit;
subscript b is 0 or 1, indicating the absence or presence of B, respectively;
L_{O} is a secondary linker moiety, wherein the secondary linker has the formula of; 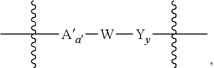
wherein the wavy line adjacent to Y indicates the site of covalent attachment of L_{O} to the Drug Unit and the wavy line adjacent to A′ indicates the site of covalent attachment to the remainder of the Drug Linker compound;
A′ is a second optional Stretcher Unit, which in the absence of B becomes a subunit of A;
subscript a′ is 0 or 1, indicating the absence or presence of A′, respectively,
W is a Peptide Cleavable Unit, wherein the Peptide Cleavable Unit comprises a tripeptide having the sequence —P3-P2-P1-, wherein P1, P2, and P3 are each an amino acid, wherein:
a first one of the amino acids P1, P2, or P3 is negatively charged;
a second one of the amino acids P1, P2, or P3 has an aliphatic side chain with hydrophobicity no greater than that of leucine; and
a third one of the amino acids P1, P2, or P3 has hydrophobicity lower than that of leucine,
wherein the first one of the amino acids P1, P2, or P3 corresponds to any one of P1, P2, or P3, the second one of the amino acids P1, P2, or P3 corresponds to one of the two remaining amino acids P1, P2, or P3, and the third one of the amino acids P1, P2, or P3 corresponds to the last remaining amino acids P1, P2, or P3,
provided that —P3-P2-P1- is not -Glu-Val-Cit- or -Asp-Val-Cit-;
Y is a self-immolative Spacer Unit;
subscript y is 0, 1 or 2 indicating the absence or presence of 1 or 2 of Y, respectively; and
subscript q is an integer ranging from 1 to 4,
provided that subscript q is 1 when subscript b is 0 and subscript q is 2, 3 or 4 when subscript b is 1.

24. The Drug Linker compound of claim 23, wherein the Drug Linker compound has the structure of Formula IH: 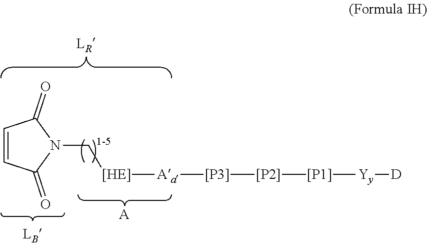
or salt thereof, wherein:
HE is a Hydrolysis Enhancing Unit; and
A′ is a subunit, when present, of the indicated first Stretcher Unit (A); subscript a′ is 0 or 1, indicating the absence or presence of A′.

25. (canceled)

26. The Drug Linker compound of claim 23 or a salt thereof, wherein —Y_{y}-D has the structure of: 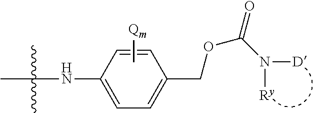
wherein —N(R^{y})D′ represents D, wherein D′ is the remainder of D;
the wavy line indicates the site of covalent attachment to P1;
the dotted line indicates optional cyclization of R^{y} to D′;
R^{y} is optionally substituted C₁-C₆ alkyl in absence of cyclization to D′ or optionally substituted C₁-C₆ alkylene when cyclized to D′;
each Q is independently selected from the group consisting of —C₁-C₈alkyl, —O—(C₁-C₈ alkyl), halogen, nitro and cyano; and
subscript m is 0, 1, or 2.

27. The Drug Linker compound of claim 23 or a salt thereof, wherein D is a cytotoxic drug wherein the cytotoxic drug is a secondary amine-containing auristatin compound wherein the nitrogen atom of the secondary amine is the site of covalent attachment to the drug linker moiety and the secondary amine-containing auristatin compound has the structure of Formula D_{F/E-3}: 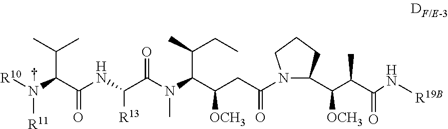
wherein the dagger indicates the site of covalent attachment of the nitrogen atom that provides the carbamate functional group;
one of R¹⁰ and R¹¹ is hydrogen and the other is methyl;
R¹³ is isopropyl or —CH₂—CH(CH₃)₂; and
R^{19B} is —CH(CH₃)—CH(OH)-Ph, —CH(CO₂H)—CH(OH)—CH₃, —CH(CO₂H)—CH₂Ph, —CH(CH₂Ph)-2-thiazolyl, —CH(CH₂Ph)-2-pyridyl, —CH(CH₂-p-Cl-Ph), —CH(CO₂Me)-CH₂Ph, —CH(CO₂Me)-CH₂CH₂SCH₃, —CH(CH₂CH₂SCH₃)C(═O)NH-quinol-3-yl, —CH(CH₂Ph)C(═O)NH-p-Cl-Ph, or
R^{19B} has the structure of 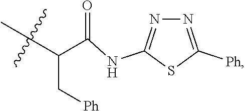 [SMILES: *C(Cc1ccccc1)C(=O)Nc1nnc(-c2ccccc2)s1]
wherein the wavy line indicates covalent attachment to the remainder of the auristatin compound.

28. (canceled)

29. The Drug Linker compound of claim 23, wherein the Drug Linker compound has the structure of Formula IH-MMAE: 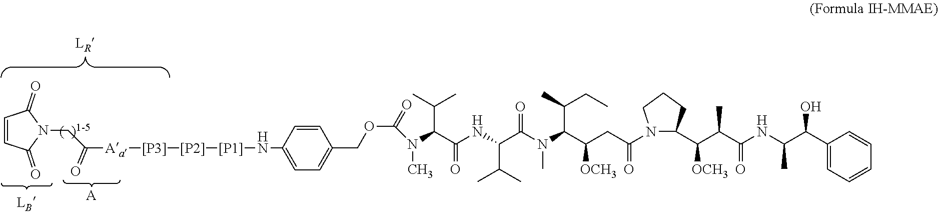
or a salt thereof, wherein
subscript a′ is 0, and A′ is absent.

30-34. (canceled)

35. The Drug Linker compound of claim 23 or a salt thereof, wherein —P3-P2-P1- is -D-Leu-Ala-Asp-, -D-Leu-Ala-Glu-, -D-Ala-Ala-Asp-, or -D-Ala-Ala-Glu-.

36. (canceled)

37. The Drug Linker compound of claim 23, wherein the Drug Linker compound has the structure of: 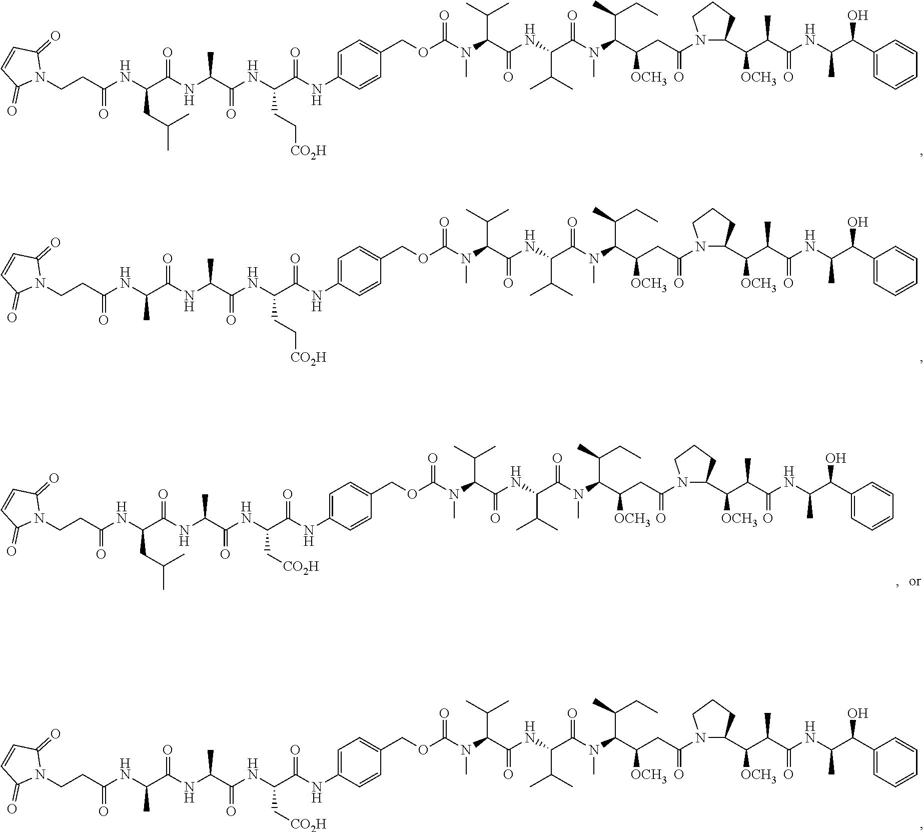
or a salt thereof.

38-39. (canceled)

40. A Linker compound of Formula IA-L-3: 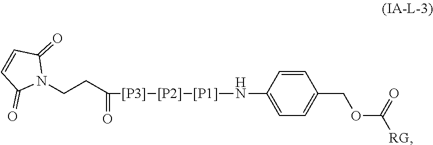
or a salt thereof, wherein:
RG is a reactive group;
P1, P2, and P3 are each an amino acid, wherein:
the P3 amino acid is in the D-amino acid configuration;
one of the P2 and P1 amino acids has an aliphatic side chain with hydrophobicity lower than that of leucine; and
the other of the P2 and P1 amino acids is negatively charged.

41. The Linker compound of claim 38, wherein the Linker compound has the structure of 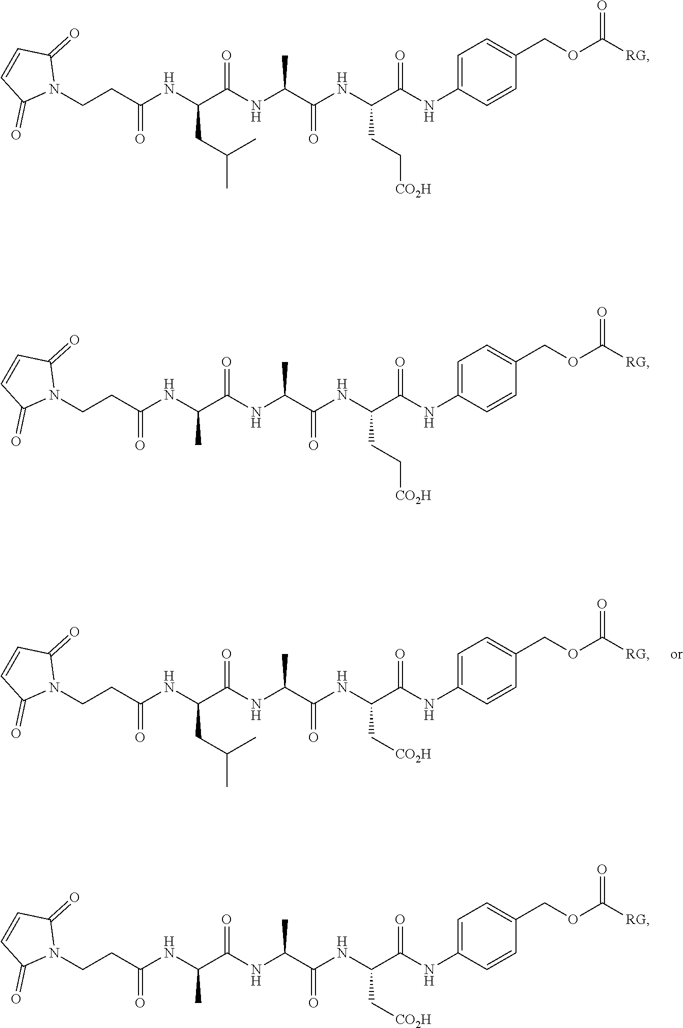
or a salt thereof.
